SEQUENCE LISTING (3 sequences)

SEQ ID NO: 1 - protein, 580 aa, Homo sapiens
MAVRQALGRGLQLGRALLLRFAPKPGPVSGWGKPGPGAAWGRGERPGRVSSPGAQPRPLGLPLPDRYRFFRQSVAGLAARIQRQFVVRARGGAGPCGRAVFLAFGLGLGLIEEKQAESRRAASACQEIQAIFTQKNKQVSDPLDTRRWQGFRLEDYLIGQAIGKGCNAAVYEATMPTLPQHLEKAKHLGLLGKGPDVVSKGADGEQAPGAPAFPFAIKMMWNISAGSSSEAILSKMSQELVPASRMALDGEYGAVTYRRSRDGPKQLAPHPNIIRVFRAFTSSVPLLPGALADYPDMLPPHYYPEGLGHGRTLFLVMKNYPCTLRQYLEEQTPSSRLATMMTLQLLEGVDHLVQQGIAHRDLKSDNILVEWDSDGCPWLVISDFGCCLADERVGLQLPFNSSSVERGGNGSLMAPEVSTAHSGPHAVIDYSKADTWAVGAIAYEIFGLANPFYGQGSAHLESRSYQEAQLPEMPKSVPPETRQLVRSLLQREANKRPSARIAANVLHLSLWGEHLLALKNLKLDKMIAWLLQQSAATLLADRLREKSCVETKLQMLFLANLECEALCQAALLLSSWRAAP

SEQ ID NO: 2 - protein, 580 aa, Mus musculus
MAVRQALGRGLQLGRALLLRFAPKPGPLFGWGKPGPAAAWGRGERPGQVVSPGAQPRPVGLPLPDRYRFFRQSVAGLAARIQRQFMVRARGGAGPCGRAVFLAFGLGLGLIEEKQAEGRRAASACQEIQAIFTQKTKRVSDPLDTRCWQGFRLEDYLIGQAIGKGCNAAVYEATMPTLPQHLEKAKHLGLIGKGPDVVLKGADGEQAPGTPTFPFAIKMMWNISAGSSSEAILSKMSQELVPASRVALAGEYGAVTYRRSRDGPKQLAPHPNIIRVFRAFTSSVPLLPGALADYPDMLPPHYYPEGLGHGRTLFLVMKNYPCTLRQYLEEQTPSSRLATMMTLQLLEGVDHLVQQGIAHRDLKSDNILVEWDSDGCPWLVISDFGCCLADQHVGLRLPFNSSSVERGGNGSLMAPEVSTAHSGPSAVIDYSKADTWAVGAIAYEIFGLANPFYGQGSAHLESRSYQEAQLPEMPESVPPEARRLVRSLLQREASKRPSARLAANVLHLSLWGEHLLALKNLKLDKMIAWLLQQSAATLLADRLREKSCVETKLQMLFLANLECEALCQAALLLSSWRAAP

SEQ ID NO: 3 - protein, 580 aa, Rattus norvegicus; identical to SEQ ID NO: 1

(19) United States
(12) Patent Application Publication
(10) Pub. No.: US 2022/0162215 A1
(43) Pub. Date: May 26, 2022

(54) COMPOSITIONS AND METHODS OF USING THE SAME FOR TREATMENT OF NEURODEGENERATIVE AND MITOCHONDRIAL DISEASE

(71) Applicant: MITOKININ, INC., San Francisco, CA (US)

(72) Inventors: Daniel de Roulet, San Francisco, CA (US); Johan Bartholomeus, Montreal (CA); Shawn Johnstone, Saint Laurent (CA); Randall Marcelo Chin, Union City, CA (US); Nicholas Thomas Hertz, San Francisco, CA (US); Robert DeVita, New York, NY (US)

(21) Appl. No.: 17/601,372

(22) PCT Filed: Apr. 3, 2020
(86) PCT No.: PCT/US20/26732

§ 371 Date: Oct. 4, 2021

Related U.S. Application Data
(60) Provisional application No. 62/933,632, filed on Nov. 11, 2019.
(60) Provisional application No. 62/879,794, filed on Jul. 29, 2019.
(60) Provisional application No. 62/828,995, filed on Apr. 3, 2019.

(51) Int. Cl. C07D 487/04; C07D 473/34; A61P 25/28

(52) U.S. Cl. CPC C07D 487/04; A61P 25/28; C07D 473/34

(57) ABSTRACT

The present disclosure is directed to nitrogen-containing heteroaryl analogs, methods of making nitrogen-containing analogs, and methods of treating disorders associated with PINK1 kinase activity including, but not limited to, neurodegenerative diseases, mitochondrial diseases, fibrosis, and/or cardiomyopathy using these analogs. This abstract is intended as a scanning tool for purposes of searching in the particular art and is not intended to be limiting of the present invention.

### CROSS-REFERENCE TO RELATED APPLICATIONS

This application claims the benefit of U.S. Application No. 62/828,995, filed on Apr. 3, 2019, U.S. Application No. 62/879,794, filed on Jul. 29, 2019, and U.S. Application No. 62/933,632, filed on Nov. 11, 2019, the contents of which are hereby incorporated by reference in their entireties.

### REFERENCE TO SEQUENCE LISTING

The Sequence Listing submitted Apr. 3, 2020 as a text file named “37930_0004P1_ST25.txt,” created on Mar. 31, 2020, and having a size of 15,539 bytes is hereby incorporated by reference pursuant to 37 C.F.R. § 1.52(e)(5).

### BACKGROUND

Maintenance of mitochondrial function is essential for the health and survival of numerous cell types, including cardiomyoctes, hepatocytes, renal cells and neurons. Aberrant mitochondrial quality control has been demonstrated to be an important factor in the development of neurodegenerative diseases, kidney disease, and cardiomyopathy (Schapira, A. H. Mitochondrial disease. Lancet 379, 1825-1834, (2012) and Chen, Y. and Dom, G. PINK1-Phosphorylated Mitofusin-2 Is a Parkin Receptor for Culling Damaged Mitochondria. Science 340, 471-475, (2013)). The mitochondrial kinase PTEN Induced Kinase 1 (PINK1) plays an important role in the mitochondrial quality control processes by responding to damage at the level of individual mitochondria. The PINK1 pathway has also been linked to the induction of mitochondrial biogenesis and, critically, to the reduction of mitochondrially-induced apoptosis. See e.g., Narendra, D. P. et al. PINK1 is selectively stabilized on impaired mitochondria to activate Parkin. PLoS Biol 8, e1000298 (2010), Wang, X., (2011). et al. PINK1 and Parkin target Miro for phosphorylation and degradation to arrest mitochondrial motility. Cell 147, 893-906, (2011), and Shin, J. H. et al. PARIS (ZNF746) repression of PGC-1alpha contributes to neurodegeneration in Parkinson's disease. Cell 144, 689-702, (2011).

Parkinson's Disease (PD) is one of the most common neurodegenerative disorders; however, no disease modifying therapies are currently approved to treat PD. Both environmental and genetic factors lead to progressive apoptosis of dopaminergic neurons, lowered dopamine levels, and, ultimately, PD. PINK1 kinase activity appears to mediate its neuroprotective activity. The regulation of mitochondrial movement, distribution, and clearance is a key part of neuronal oxidative stress response. Disruptions to these regulatory pathways have been shown to contribute to chronic neurodegenerative disease. See Schapira and Chen cited above.

Cardiomyopathy refers to a disease of cardiac muscle tissue, and it is estimated that cardiomyopathy accounts for 5-10% of the 5-6 million patients already diagnosed with heart failure in the United States. Based on etiology and pathophysiology, the World Health Organization created a classification of cardiomyopathy types which includes dilated cardiomyopathy, hypertrophic cardiomyopathy, restrictive cardiomyopathy, arrhythmogenic right ventricular cardiomyopathy, and unclassified cardiomyopathy. See e.g., Richardson P, et al. Report of the 1995 World Health Organization/International Society and Federation of Cardiology Task Force on the Definition and Classification of cardiomyopathies. Circulation 1996; 93:841. PINK1 kinase activity appears to mediate its' cardio-protective activity. The regulation of mitochondrial movement, distribution, and clearance is a part of cardiac cell oxidative stress response. Disruptions to these regulatory pathways have been shown to contribute to cardiomyopathy. See Schapira and Chen cited above.

Neural pathologies frequently result from dysfunctional mitochondria, and Leigh syndrome (LS) is a common clinical phenotype. LS, or subacute necrotizing encephalopathy, is a progressive neurodegenerative disorder affecting 1 in 40,000 live births. LS is regarded as the most common infantile mitochondrial disorder, and most patients exhibit symptoms before 1 month of age. See e.g., Wang, X., (2011) et al. PINK1 and Parkin target Miro for phosphorylation and degradation to arrest mitochondrial motility Cell 147, 893-906, (2011) and Richardson P, et al. Report of the 1995 World Health Organization/International Society and Federation of Cardiology Task Force on the Definition and Classification of cardiomyopathies. Circulation 1996; 93:841. Several cases of adult-onset LS have also been reported recently. See e.g., Longo, D, et al. Harrison's Internal Medicine. 18^{th} ed. (online), Ch. 238 (2011), Petit, A. et al. Wild-type PINK1 prevents basal and induced neuronal apoptosis, a protective effect abrogated by Parkinson disease-related mutations. J Biol Chem 280, 34025-34032 (2005), Koh, H. & Chung, J. PINK1 as a molecular checkpoint in the maintenance of mitochondrial function and integrity, Mol Cells 34, 7-13, (2012), Martins-Branco, D. et al. Ubiquitin proteasome system in Parkinson's disease: a keeper or a witness?Exp Neurol 238, 89-99, (2012), and Geisler, S. et al. The PINK1/Parkin-mediated mitophagy is compromised by PD-associated mutations. Autophagy 6, 871-878, (2010). In vivo imaging techniques such as MRI reveal bilateral hyperintense lesions in the basal ganglia, thalamus, substantia nigra, brainstem, cerebellar white matter and cortex, cerebral white matter, or spinal cord of LS patients. See e.g., Longo cited above and Shin, J. H. et al. PARIS (ZNF746) repression of PGC-1alpha contributes to neurodegeneration in Parkinson's disease. Cell 144, 689-702, (2011), Henchcliffe, C. & Beal, M. F. Mitochondrial biology and oxidative stress in Parkinson disease pathogenesis. Nat Clin Pract Neurol 4, 600-609 (2008), Pridgeon, J. W., Olzmann, J. A., Chin, L. S. & Li, L. PINK1 Protects against Oxidative Stress by Phosphorylating Mitochondrial Chaperone TRAP1. PLoS Biol 5, e172 (2007), and Haque, M. E. et al. Cytoplasmic Pink1 activity protects neurons from dopaminergic neurotoxin MPTP. Proc Natl Acad Sci USA 105, 1716-1721 (2008). The lesions usually correlate with gliosis, demyelination, capillary proliferation, and/or necrosis See Geisler, S. et al. The PINK1/Parkin-mediated mitophagy is compromised by PD-associated mutations. Autophagy 6, 871-878, (2010) and Gautier, C. A., Kitada, T. & Shen, J. Loss of PINK1 causes mitochondrial functional defects and increased sensitivity to oxidative stress. Proc Natl Acad Sci USA 105, 11364-11369 (2008). Behavioral symptoms of LS patients can include (with a wide variety of clinical presentation) developmental retardation, hypotonia, ataxia, spasticity, dystonia, weakness, optic atrophy, defects in eye or eyelid movement, hearing impairment, breathing abnormalities, dysarthria, swallowing difficulties, failure to thrive, and gastrointestinal problems. See e.g., Wang and Richardson cited above, and Samaranch, L. et al. PINK1-linked Parkinsonism is associated with Lewy body pathology. Brain 133, 1128-1142, (2010) and Merrick, K. A. et al. Switching Cdk2 on or off with small molecules to reveal requirements in human cell proliferation. Mol Cell 42, 624-636, (2011). The cause of death in most LS cases is unclear, and the lack of a genetic model to study the disease progression and cause of death has impeded the development of adequate treatment. Prognosis for LS (and most diseases resulting from mitochondrial dysfunction) is very poor; there is no cure and treatment is often ineffective.

Despite the widespread prevalence of disorders associated with PINK1 pathway, compounds capable of selectively targeting this pathway and, thus, treating disorders associated with this pathway have remained elusive.

### SUMMARY

In accordance with the purpose(s) of the invention, as embodied and broadly described herein, the invention, in some embodiments, relates to substituted N-containing heteroaryl compounds useful in the treatment of disorders associated with PINK1 kinase activity such as, for example, a neurodegenerative disease, a mitochondrial disease, fibrosis, and/or cardiomyopathy.

Thus, provided herein are compounds having a structure represented by a formula:

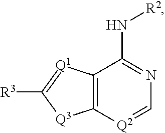

wherein Q¹ is N or CH and R³ is a 3- to 6-membered cycloalkyl, C1-C6 haloalkyl, C1-C6 haloalkoxy, or C1-C6 halohydroxyalkyl; or wherein Q¹ is CR¹ and R³ is hydrogen; R¹ is C1-C6 haloalkyl, C1-C6 haloalkoxy, C1-C6 halohydroxyalkyl, or a structure represented by a formula:

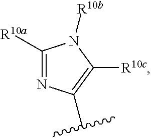

wherein each of R^{10a}, R^{10b}, and R^{10c}, when present, is independently selected from hydrogen and C1-C4 alkyl; wherein Q² is CH or N; wherein Q³ is CH₂ or NH; wherein R² is C1-C6 alkyl, —CR^{11a}R^{11b}Cy¹, or Cy¹; wherein each of R^{11a} and R^{11b}, when present, is independently selected from hydrogen, C1-C5 alkyl, and C1-C4 hydroxyalkyl; or wherein each of R^{11a} and R^{11b}, when present, together comprise a 3-membered cycloalkyl; wherein Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, provided that when R¹ is C1-C6 haloalkyl and R² is Cy¹, then Cy¹ is not a 6-membered carbocycle or a 9-membered heteroaryl, and provided that when R² is —CR^{11a}R^{11b}Cy¹ or Cy¹, one or both of R^{11a} and R^{11b}, when present, is hydrogen, and Cy¹ is a 6-membered aryl or furanyl, then Q¹ is CH and R³ is not a C1-C6 haloalkyl, or a pharmaceutically acceptable salt thereof.

Also provided is a compound having a structure:

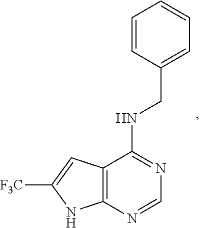 [SMILES: FC(F)(F)c1cc2c(NCc3ccccc3)ncnc2[nH]1]

or a pharmaceutically acceptable salt thereof.

Also provided are compounds having a structure represented by a formula:

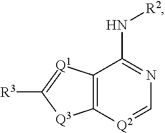

wherein Q¹ is N or CH and R³ is a 3- to 6-membered cycloalkyl or a C1-C6 haloalkyl, C1-C6 haloalkoxy, C1-C6 halohydroxyl, CF₃, CCl₃, CBr₃; or wherein Q¹ is CR¹ and R³ is hydrogen; R¹ is C1-C6 haloalkyl, C1-C6 haloalkoxy, C1-C6 halohydroxy, or a structure represented by a formula:

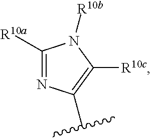

wherein each of R^{10a}, R^{10b}, and R^{10c}, when present, is independently selected from hydrogen and C1-C4 alkyl; wherein Q² is CH or N; wherein Q³ is CH₂ or NH; wherein R² is C1-C6 alkyl, —CR^{11a}R^{11b}Cy¹, or Cy¹; wherein each of R^{11a} and R^{11b}, when present, is independently selected from hydrogen, C1-C5 alkyl, and C1-C5 hydroxyalkyl; or wherein each of R^{11a} and R^{11b} together comprise a 3-membered cycloalkyl; wherein Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, provided that when R¹ is C1-C6 haloalkyl and R² is Cy¹, then Cy¹ is not a 6-membered carbocycle or a 9-membered heteroaryl, or a pharmaceutically acceptable salt thereof.

Also provided are compounds having a structure represented by Formula I:

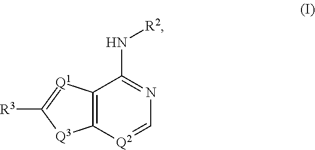

wherein Q¹ is N or CH and R³ is a 3- to 6-membered cycloalkyl or a C1-C6 haloalkyl, C1-C6 haloalkoxy, C1-C6 halohydroxyl, CF₃, CCl₃, CBr₃; or wherein Q¹ is CR¹ and R³ is hydrogen; wherein Q² is CH or N; wherein Q³ is CH₂ or NH; R¹ is (C₁-C₆)alkyl, halo(C₁-C₄)alkyl, (C₁-C₄)alkoxy, halo(C₁-C₄)alkoxy, 5- or 6-membered heteroaryl, or phenyl, wherein said C₁-C₆alkyl and halo(C₁-C₄)alkyl are each optionally and independently substituted with a OR^{a} group, and wherein said phenyl and 5- or 6-membered heteroaryl are each optionally and independently substituted with 1 to 3 groups independently selected from R^{b}; R^{a}, when present, is H, (C₁-C₄)alkyl, or (C₁-C₄)alkoxy; each occurrence of R^{b}, when present, is independently halo, halo(C₁-C₄)alkyl, (C₁-C₄)alkoxy, or halo(C₁-C₄)alkoxy; R² is (C₁-C₆)alkyl, a 9-membered oxygen-containing fused heterocycle, or a 9- to 10-membered carbocycle, wherein said (C₁-C₆)alkyl is optionally substituted with 1 or 2 groups independently selected from R^{c}, and wherein said 9-membered oxygen-containing fused heterocycle and 9- to 10-membered carbocycle are each optionally and independently substituted with 1 to 3 groups independently selected from R^{d}; each occurrence of R^{c}, when present, is phenyl, 3- or 4-membered cycloalkyl, or 5- or 6-membered heteroaryl, wherein said phenyl and 5- or 6-membered heteroaryl are each optionally and independently substituted with 1 to 3 groups independently selected from R^{e}; each occurrence of R^{d} and R^{e}, when present, is independently halo, halo(C₁-C₄)alkyl, (C₁-C₄)alkoxy, or halo(C₁-C₄)alkoxy; and R³ is hydrogen, halogen, (C₁-C₄)alkyl, or 3- to 6-membered cycloalkyl, or pharmaceutically acceptable salts thereof. These compounds are useful in the treatment of conditions associated with PINK1 kinase activity. Such conditions include e.g., neurodegenerative disease, mitochondrial disease, fibrosis, and cardiomyopathy.

Also provided are compounds having a structure represented by a formula:

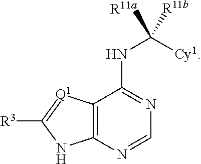

Also provided are compounds having a structure represented by a formula selected from:

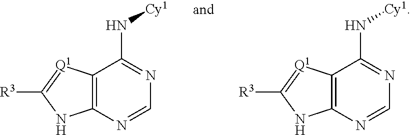

Also provided are compounds having a structure represented by a formula:

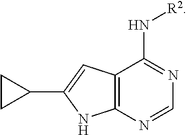

Also provided are compounds having a structure represented by a formula:

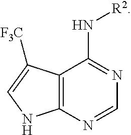

Also provided are compounds having a structure represented by a formula:

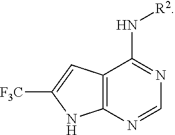

Also provided are compounds selected from:

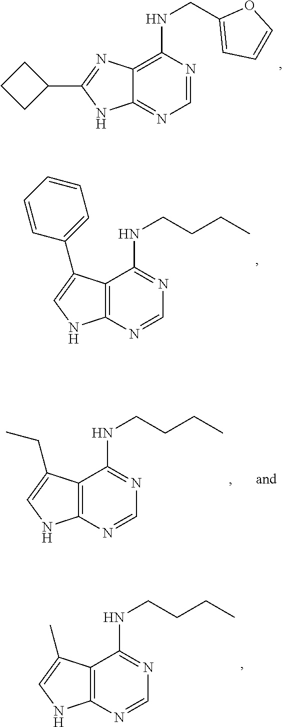

or a pharmaceutically acceptable salt thereof.

Also provided are compounds selected from:

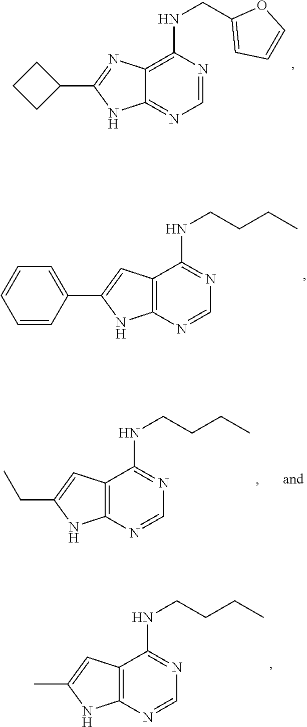

or a pharmaceutically acceptable salt thereof.

Also provided are compounds selected from:

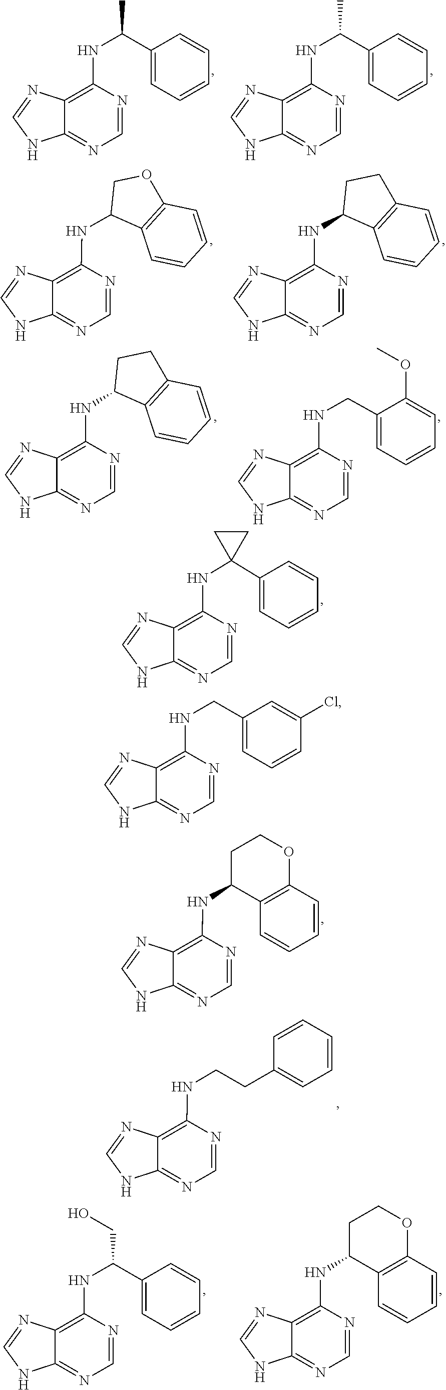

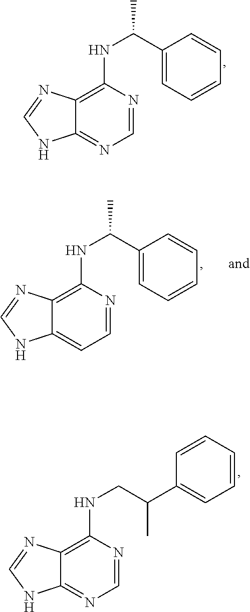 [SMILES: C[C@@H](Nc1ncnc2[nH]cnc12)c1ccccc1 ; C[C@@H](Nc1nccc2[nH]cnc12)c1ccccc1 ; CC(CNc1ncnc2[nH]cnc12)c1ccccc1]

or a pharmaceutically acceptable salt thereof.

Without wishing to be bound by theory, an advantage of the presently described compounds is that they possess improved potency and reduced toxicity. For example, the disclosed compounds can exhibit greater than 80% mitophagy with a toxicity of less than 5%. See, e.g., Table 2, compound no. 12 and Table 3, compound no. 23.

Also provided are methods for making a disclosed compound.

Also provided are pharmaceutical compositions comprising a therapeutically effective amount of a disclosed compound and a pharmaceutically acceptable carrier.

Also provided are methods of modulating PINK1 kinase activity in a subject in need thereof, the method comprising administering to the subject in need thereof an effective amount of at least one disclosed compound.

Also provided are methods of modulating PINK1 kinase activity in a subject in need thereof, the method comprising administering to the subject in need thereof an effective amount of a compound having a structure represented by a formula:

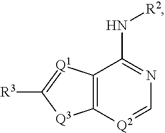

wherein Q¹ is N or CH and R³ is a 3- to 6-membered cycloalkyl, C1-C6 haloalkyl, C1-C6 haloalkoxy, or C1-C6 halohydroxyalkyl; or wherein Q¹ is CR¹ and R³ is hydrogen; R¹ is C1-C6 haloalkyl, C1-C6 haloalkoxy, C1-C6 halohydroxyalkyl, or a structure represented by a formula:

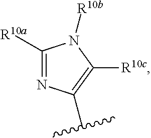

wherein each of R^{10a}, R^{10b}, and R^{10c}, when present, is independently selected from hydrogen and C1-C4 alkyl; wherein Q² is CH or N; wherein Q³ is CH₂ or NH; wherein R² is C1-C6 alkyl, —CR^{11a}R^{11b}Cy¹, or Cy¹; wherein each of R^{11a} and R^{11b}, when present, is independently selected from hydrogen, C1-C5 alkyl, and C1-C4 hydroxyalkyl; or wherein each of R^{11a} and R^{11b}, when present, together comprise a 3-membered cycloalkyl; wherein Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, or a pharmaceutically acceptable salt thereof.

Also disclosed are methods of modulating PINK1 kinase activity in at least one cell, the method comprising contacting the cell with an effective amount of at least one disclosed compound.

Also disclosed are methods of modulating PINK1 kinase activity in at least one cell, the method comprising contacting the cell with an effective amount of a compound having a structure represented by a formula:

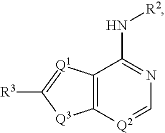

wherein Q¹ is N or CH and R³ is a 3- to 6-membered cycloalkyl, C1-C6 haloalkyl, C1-C6 haloalkoxy, or C1-C6 halohydroxyalkyl; or wherein Q¹ is CR¹ and R³ is hydrogen; R¹ is C1-C6 haloalkyl, C1-C6 haloalkoxy, C1-C6 halohydroxyalkyl, or a structure represented by a formula:

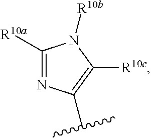

wherein each of R^{10a}, R^{10b}, and R^{10c}, when present, is independently selected from hydrogen and C1-C4 alkyl; wherein Q² is CH or N; wherein Q³ is CH₂ or NH; wherein R² is C1-C6 alkyl, —CR^{11a}R^{11b}Cy¹, or Cy¹; wherein each of R^{11a} and R^{11b}, when present, is independently selected from hydrogen, C1-C5 alkyl, and C1-C4 hydroxyalkyl; or wherein each of R^{11a} and R^{11b}, when present, together comprise a 3-membered cycloalkyl; wherein Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, or a pharmaceutically acceptable salt thereof.

Also provided are methods for treating a disorder in a subject in need thereof, the method comprising administering to the subject in need thereof an effective amount of at least one disclosed compound, wherein the disorder is a neurodegenerative disorder, a mitochondrial disorder, a fibrosis, or cardiomyopathy.

Also provided are methods for treating a disorder in a subject in need thereof, the method comprising administering to the subject in need thereof an effective amount of at least one compound having a structure represented by a formula:

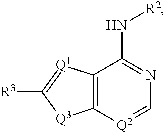

wherein Q¹ is N or CH and R³ is a 3- to 6-membered cycloalkyl, C1-C6 haloalkyl, C1-C6 haloalkoxy, or C1-C6 halohydroxyalkyl; or wherein Q¹ is CR¹ and R³ is hydrogen; R¹ is C1-C6 haloalkyl, C1-C6 haloalkoxy, C1-C6 halohydroxyalkyl, or a structure represented by a formula:

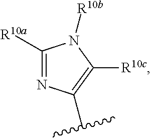

wherein each of R^{10a}, R^{10b}, and R^{10c}, when present, is independently selected from hydrogen and C1-C4 alkyl; wherein Q² is CH or N; wherein Q³ is CH₂ or NH; wherein R² is C1-C6 alkyl, —CR^{11a}R^{11b}Cy¹, or Cy¹; wherein each of R^{11a} and R^{11b}, when present, is independently selected from hydrogen, C1-C5 alkyl, and C1-C4 hydroxyalkyl; or wherein each of R^{11a} and R^{11b}, when present, together comprise a 3-membered cycloalkyl; wherein Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, or a pharmaceutically acceptable salt thereof, wherein the disorder is a neurodegenerative disorder, a mitochondrial disorder, a fibrosis, or cardiomyopathy.

Also provided are kits comprising a disclosed compound and one or more of: (a) at least one agent known for the treatment of a neurodegenerative disorder, a mitochondrial disorder, a fibrosis, and cardiomyopathy; (b) instructions for administering the compound in connection with the neurodegenerative disorder, a mitochondrial disorder, a fibrosis, or cardiomyopathy; and/or (c) instructions for treating the disorder.

Also provided are kits comprising a compound having a structure represented by a formula:

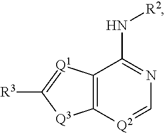

wherein Q¹ is N or CH and R³ is a 3- to 6-membered cycloalkyl, C1-C6 haloalkyl, C1-C6 haloalkoxy, or C1-C6 halohydroxyalkyl; or wherein Q¹ is CR¹ and R³ is hydrogen; R¹ is C1-C6 haloalkyl, C1-C6 haloalkoxy, C1-C6 halohydroxyalkyl, or a structure represented by a formula:

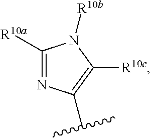

wherein each of R^{10a}, R^{10b}, and R^{10c}, when present, is independently selected from hydrogen and C1-C4 alkyl; wherein Q² is CH or N; wherein Q³ is CH₂ or NH; wherein R² is C1-C6 alkyl, —CR^{11a}R^{11b}Cy¹, or Cy¹; wherein each of R^{11a} and R^{11b}, when present, is independently selected from hydrogen, C1-C5 alkyl, and C1-C4 hydroxyalkyl; or wherein each of R^{11a} and R^{11b}, when present, together comprise a 3-membered cycloalkyl; wherein Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, or a pharmaceutically acceptable salt thereof, and one or more of: (a) at least one agent known for the treatment of a neurodegenerative disorder, a mitochondrial disorder, a fibrosis, and cardiomyopathy; (b) instructions for administering the compound in connection with the neurodegenerative disorder, a mitochondrial disorder, a fibrosis, or cardiomyopathy; and/or (c) instructions for treating the disorder.

Still other objects and advantages of the present disclosure will become readily apparent by those skilled in the art from the following detailed description, wherein it is shown and described only the preferred embodiments, simply by way of illustration of the best mode. As will be realized, the disclosure is capable of other and different embodiments, and its several details are capable of modifications in various obvious respects, without departing from the disclosure. Accordingly, the description is to be regarded as illustrative in nature and not as restrictive.

### BRIEF DESCRIPTION OF THE DRAWINGS

The accompanying figures, which are incorporated in and constitute a part of this specification, illustrate several embodiments and together with the description serve to explain the principles of the invention.

FIG. 1A-E show representative data demonstrating the potency and toxicity of the compound nos. EP-0035910, EP-0036296, EP-0036329, and EP-0036336 in the absence of toxin (no FO) or after treatment with 1 μM FCCP/oligomycin for 6-7 hours. H₂O₂ treatment was performed as a control for cell death as measured by DAPI staining.

FIG. 2A-E show representative data demonstrating the potency and toxicity of the compounds nos. EP-0035910, EP-0036002, EP-0036004, and EP-0036022 in the presence of 1 μM FCCP/oligomycin or with no toxin (no FO) after treatment with H₂O₂ for 1 hr. H₂O₂ treatment was performed as a control for cell death as measured by DAPI staining. EP-0035006 from batch 3 and EP-0035910 from batch 2.

FIG. 3A-F show representative data demonstrating the potency and toxicity of the compounds nos. EP-0035910, EP-0036032, EP-0036050, and EP-0036061 in the absence of toxin (no FO) or after treatment with 1 μM FCCP/oligomycin for 6-7 hours. H₂O₂ treatment was performed as a control for cell death as measured by DAPI staining. EP-0035910 from batch 2.

FIG. 4A-D show representative data demonstrating the potency and toxicity of the compounds nos. EP-0035910, EP-0036032, EP-0036050, EP-0036061, EP-0036078, EP-0036079, and EP-0036080 in the absence of toxin (no FO) or after treatment with 1 μM FCCP/oligomycin for 6-7 hours. H₂O₂ treatment was performed as a control for cell death as measured by DAPI staining EP-0035910 from batch 2.

FIG. 5A-G show representative data demonstrating the potency and toxicity of the compounds nos. EP-0036195, EP-0036194, EP-0036193, and EP-0035910 in the absence of toxin (no FO) or after treatment with 1 μM FCCP/oligomycin for 5.5-6 hours. H₂O₂ treatment was performed as a control for cell death as measured by DAPI staining. EP-0035910 from batch 2.

FIG. 6A-E show representative data demonstrating the potency and toxicity of the compounds nos. EP-0035910, EP-0036202, EP-0036296, and EP-0036297 in the absence of toxin (no FO) or after treatment with 1 μM FCCP/oligomycin for 6 hours. H₂O₂ treatment was performed as a control for cell death as measured by DAPI staining. EP-0035910 from batch 2.

FIG. 7A-G show representative data demonstrating the potency and toxicity of the compounds nos. EP-0035910, EP-0036404, EP-0036405, and EP-0036406 in the absence of toxin (no FO) or after treatment with 1 μM FCCP/oligomycin for 6 hours. H₂O₂ treatment was performed as a control for cell death as measured by DAPI staining. No compounds showed crystallization at 50 μM or caused abnormal round cells.

FIG. 8A-D show representative data demonstrating the potency and toxicity of the compounds nos. EP-0035910, EP-0036411, EP-0036413, and EP-0036414 in the absence of toxin (no FO) or after treatment with 1 μM FCCP/oligomycin for 6 hours. H₂O₂ treatment was performed as a control for cell death as measured by DAPI staining. No compounds showed crystallization at 50 μM or caused abnormal round cells.

FIG. 9A-F show representative data demonstrating the potency and toxicity of the compounds nos. EP-0035910, EP-0036422, EP-0036425, EP-0036426, EP-0036428, and EP-0036437 in the absence of toxin (no FO) or after treatment with 1 μM FCCP/oligomycin for 6.5-7 hours. H₂O₂ treatment was performed as a control for cell death as measured by DAPI staining. No compounds showed crystallization at 50 μM.

FIG. 10A-F shows representative data demonstrating the potency and toxicity of the compounds nos. EP-0035910, EP-0036438, EP-0036439, EP-0036451, and EP-0036453 in the absence of toxin (no FO) or after treatment with 1 μM FCCP/oligomycin for 6.5-7 hours. H₂O₂ treatment was performed as a control for cell death as measured by DAPI staining. No compounds showed crystallization at 50 μM.

FIG. 11A and FIG. 11B show representative data demonstrating the potency and toxicity of the compounds nos. EP-0035910, EP-0036422, EP-0036425, EP-0036426, EP-0036428, EP-0036437, EP-0036438, EP-0036439, EP-0036451, and EP-0036453 in the absence of toxin (no FO) or after treatment with 1 μM FCCP/oligomycin for 6.5-7 hours. H₂O₂ treatment was performed as a control for cell death as measured by DAPI staining. No compounds showed crystallization at 50 μM.

FIG. 12A-H shows representative data demonstrating the potency and toxicity of the compounds nos. EP-0035910, EP-0036463, EP-0036468, EP-0036477, and EP-0035764 in the absence of toxin (no FO) or after treatment with 1 μM FCCP/oligomycin for 6.5-7 hours. H₂O₂ treatment was performed as a control for cell death as measured by DAPI staining. No compounds showed crystallization at 50 μM or caused abnormal round cells.

FIG. 13A-H shows representative data demonstrating the potency and toxicity of the compounds nos. EP-0035985, EP-0036837, EP-0036847, and EP-0036848 in the absence of toxin (no FO) or after treatment with 1 μM FCCP/oligomycin for 6 hours. H₂O₂ treatment was performed as a control for cell death as measured by DAPI staining.

FIG. 14A and FIG. 14B show representative data illustrating the results of in vitro PINK1 kinase assays. Treatment of cells with EP-0035985 (along with other exemplary compounds) in the presence of 0.5 μM FO increases the pS65 Ub signal.

FIG. 15 shows representative data illustrating the activity of exemplary compounds in a LPS assay.

FIG. 16 shows representative data illustrating the activity of exemplary compounds in a dOTC assay. In this cell line, doxycycline (DOX) treatment induces the expression of dOTC, a protein that forms insoluble protein aggregates in the mitochondrial matrix and activates the PINK1/parkin pathway without strong depolarizing agents like CCCP/FCCP. Exemplary compounds like EP-0035985 are able to reduce the accumulated dOTC proteins. As would be understood by one of ordinary skill in the art, a dOTC assay is a type of mitochondrial aggregate assay, and, as such, this assay has implications for methods of inducing mitochondrial clearance and for treatment of disorders associated with mitochondrial protein aggregation (e.g., Alzheimer's disease, Parkinson's dsease, dementia with Lewy bodies, Amytotrphic lateral sclerosis, etc.).

FIG. 17A and FIG. 17 show representative data illustrating the in vitro increase in PINK1 substrate phosphorylation observed upon addition of EP-0035985. Specifically, FIG. 17A shows that compound addition drives a significant increase in pS65 Ub in PINK1^{wt} cells but not in PINK1^{ko} cell lines. FIG. 17B shows that an immunoblotting analysis of pS65 Ub confirms the ELISA results.

FIG. 18 shows representative data illustrating that addition of EP-0035985 increases the rate of Parkin recruitment in PINK1^{wt} but not PINK1^{ko} cells, as measured by live cell imaging.

FIG. 19 shows representative data illustrating that addition of EP-0035985 increases mitophagy as measured by FACS mKeima in PINK1^{wt} but not PINK1^{ko} cell lines.

FIG. 20A and FIG. 20B show representative data illustrating that addition of EP-0035985 reduces delta OTC aggregates from mitochondria that are induced by doxycycline addition.

FIG. 21 shows representative data illustrating that addition of EP-0035985 significantly reduces pS129 α-synuclein from human human iPSC derived neurons.

FIG. 22A-C show representative data illustrating addition of EP-0035985 in vitro decreases pathological synuclein. Specifically, FIG. 22A and FIG. 22B show that compound addition decreases pathological phospho-serine 129 synuclein (pS129) increase driven by PFF addition with an EC₅₀ of 981 nM. FIG. 22C shows that EP-0035985 does not decrease pS129 synuclein in PINK1^{ko} cell lines.

FIG. 23 shows representative data illustrating the in vivo pharmacokinetic properties of EP-0035985.

FIG. 24 shows representative data illustrating that EP-0035985 demonstrates good free fraction in the brain as measured by microdialysis.

FIG. 25 shows representative images depicting the site of injection (ipsilateral striatum, contralateral striatum, and ventral midbrain sections) from a side view (left images) and a cross-sectional view (right images).

FIG. 26A and FIG. 26B show representative biochemical analysis of the ipsilateral striatum illustrating that oral dosing of EP-0035985 drives a decrease in c-terminal truncation of α-synuclein (14 kDA) using a mouse PFF model. Referring to FIG. 26B, the bar graph columns (left to right) represent PBS Vehicle Ipsilateral Striatum, PFF Veh Ipsilateral Striatum 5 μg (2.5 μg/μl), PFF 50 mg/kg Ipsilateral Striatum, PFF 20 mg/kg Ipsilateral Striatum, PFF 10 mg/kg Ipsilateral Striatum, and PFF 5 mg/kg Ipsilateral Striatum.

FIG. 27A and FIG. 27B show representative biochemical analysis of the ipsilateral striatum illustrating that oral dosing of compound EP-0035985 drives a decrease in pS129 monomer of α-synuclein using a mouse pS129 α-synuclein PFF model. Referring to FIG. 27B, the bar graph columns (left to right) represent PBS Vehicle Ipsilateral Striatum, PFF Veh Ipsilateral Striatum 5 μg (2.5 μg/μl), PFF 50 mg/kg Ipsilateral Striatum, PFF 20 mg/kg Ipsilateral Striatum, PFF 10 mg/kg Ipsilateral Striatum, and PFF 5 mg/kg Ipsilateral Striatum.

FIG. 28A and FIG. 28B show representative biochemical analysis of the ipsilateral striatum illustrating that oral dosing of EP-0035985 drives a decrease in total monomer of α-synuclein using a mouse PFF model. Referring to FIG. 28B, the bar graph columns (left to right) represent PBS Vehicle Ipsilateral Striatum, PFF Veh Ipsilateral Striatum 5 μg (2.5 μg/μl), PFF 50 mg/kg Ipsilateral Striatum, PFF 20 mg/kg Ipsilateral Striatum, PFF 10 mg/kg Ipsilateral Striatum, and PFF 5 mg/kg Ipsilateral Striatum.

FIG. 29A-F show representative biochemical analysis of the ipsilateral striatum illustrating that oral dosing of EP-0035985 drives a decrease in all analyzed species of α-synuclein at 50 mg/kg max. Referring to FIG. 29A (top to bottom) and FIG. 29B-F (left to right), the bar graph columns represent PBS Vehicle Ipsilateral Striatum, PFF Veh Ipsilateral Striatum 5 μg (2.5 μg/μl), PFF 50 mg/kg Ipsilateral Striatum, PFF 20 mg/kg Ipsilateral Striatum, PFF 10 mg/kg Ipsilateral Striatum, and PFF 5 mg/kg Ipsilateral Striatum.

FIG. 30A-F show representative biochemical analysis of the contralateral striatum illustrating that oral dosing of EP-0035985 drives a decrease in all analyzed species of α-synuclein at 50 mg/kg max. Referring to FIG. 30A (top to bottom) and FIG. 30B-F (left to right), the bar graph columns represent PBS Vehicle Contralateral Striatum, PFF Veh Contralateral Striatum 5 μg (2.5 μg/μl), PFF 50 mg/kg Contralateral Striatum, PFF 20 mg/kg Contralateral Striatum, PFF 10 mg/kg Contralateral Striatum, and PFF 5 mg/kg Contralateral Striatum.

FIG. 31A-F show representative biochemical analysis of the ventral midbrain illustrating that oral dosing of EP-0035985 drives a decrease in all analyzed species of α-synuclein at 50 mg/kg max. Referring to FIG. 31A (top to bottom) and FIG. 31B-F (left to right), the bar graph columns represent PBS Vehicle Ventral Midbrain, PFF Veh Ventral Midbrain 5 μg (2.5 μg/μl), PFF 50 mg/kg Ventral Midbrain, PFF 20 mg/kg Ventral Midbrain, PFF 10 mg/kg Ventral Midbrain, and PFF 5 mg/kg Ventral Midbrain.

FIG. 32A-C show representative images illustrating a comparison of EP-0035985 to other treatment paradigms. Referring to FIG. 32A (left to right), the bar graph columns represent PBS Vehicle Ventral Midbrain, PFF Veh Ventral Midbrain 5 μg (2.5 μg/μl), PFF 50 mg/kg Ventral Midbrain, PFF 20 mg/kg Ventral Midbrain, PFF 10 mg/kg Ventral Midbrain, and PFF 5 mg/kg Ventral Midbrain.

FIG. 33A-C show representative data illustrating that EP-0035985 increases levels of PINK1. Referring to FIG. 33A, treatment of HeLa cells with 2.8 μM EP-0035985 and 0.5, 1.0, or 2.0 μM FCCP significantly increases the levels of PINK1ₚₕₒₛₚₕₒ as quantified by polyacrylamide gel electrophoresis with the addition of 7 μM PhosTag reagent. Referring to FIG. 33B, quantification of the percentage (%) of PINK1ₚₕₒₛₚₕₒ is shown. Without wishing to be bound by theory, there is a significant increase at 0.5, 1, or 2 μM FCCP. Referring to FIG. 33C, there is a significant increase in pS65 Ubiquitin at 0.5, 1 μM FCCP. *** p<0.0001, * p<0.05.

FIG. 34 shows representative data illustrating that oral dosing of EP-0035985 reduces expression of mitochondrial disease marker GDF15. Specifically, i.p. injection of cisplatin induces mitochondrial damage that drives an increase in mitochondrial disease marker GDF15. Oral dosing of EP-0035984 at 20 to 50 mg/kg significantly reduces the expression of GDF15 as quantified by qPCR. *** p<0.0001, ** p<0.01.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular embodiments only and is not intended to be limiting. Although any methods and materials similar or equivalent to those described herein can be used in the practice or testing of the present invention, example methods and materials are now described.

While embodiments of the present invention can be described and claimed in a particular statutory class, such as the system statutory class, this is for convenience only and one of skill in the art will understand that each embodiment of the present invention can be described and claimed in any statutory class. Unless otherwise expressly stated, it is in no way intended that any method or embodiment set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of embodiments described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. Definitions

Listed below are definitions of various terms used to describe this invention. These definitions apply to the terms as they are used throughout this specification, unless otherwise limited in specific instances, either individually or as part of a larger group.

As used herein, the terms “a” or “an” means that “at least one” or “one or more” unless the context clearly indicates otherwise. The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in various embodiments, to A without B (optionally including elements other than B); in another embodiment, to B without A (optionally including elements other than A); in yet another embodiment, to both A and B (optionally including other elements); etc.

The term “or” as used herein shall only be interpreted as indicating exclusive alternatives (i.e. “one or the other but not both”) when preceded by terms of exclusivity, “either,” “one of,” “only one of,” or “exactly one of.”

As used herein, the terms “comprising” (and any form of comprising, such as “comprise,” “comprises,” and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the term “about” means that the numerical value is approximate and small variations would not significantly affect the practice of the disclosed embodiments. Where a numerical limitation is used, unless indicated otherwise by the context, “about” means the numerical value can vary by ±10%, ±5%, ±2% or ±1% and remain within the scope of the disclosed embodiments.

The abbreviations used herein have their conventional meaning within the chemical and biological arts. The chemical structures and formulae set forth herein are constructed according to the standard rules of chemical valency known in the chemical arts.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” mean that the subsequently described event or circumstance can or cannot occur, and that the description includes instances where said event or circumstance occurs and instances where it does not.

As used herein, the term “diagnosed” means having been subjected to a physical examination by a person of skill, for example, a physician, and found to have a condition that can be diagnosed or treated by the compounds, compositions, or methods disclosed herein. In some embodiments of the disclosed methods, the subject has been diagnosed with a need for treatment of a disorder associated with PINK1 kinase activity such as, for example, a neurodegenerative disease, a mitochondrial disease, fibrosis, and/or cardiomyopathy, prior to the administering step. As used herein, the phrase “identified to be in need of treatment for a disorder,” or the like, refers to selection of a subject based upon need for treatment of the disorder. It is contemplated that the identification can, in some embodiments, be performed by a person different from the person making the diagnosis. It is also contemplated, in further embodiments, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various embodiments, a preparation can be administered therapeutically; that is, administered to treat an existing disease or condition. In further various embodiments, a preparation can be administered prophylactically; that is, administered for prevention of a disease or condition.

The term “contacting” as used herein refers to bringing a disclosed compound and a cell, target receptor, or other biological entity together in such a manner that the compound can affect the activity of the target (e.g., receptor, cell, etc.), either directly; i.e., by interacting with the target itself, or indirectly; i.e., by interacting with another molecule, co-factor, factor, or protein upon which the activity of the target is dependent.

As used herein, “IC₅₀,” is intended to refer to the concentration of a substance (e.g., a compound or a drug) that is required for 50% inhibition of a biological process, or component of a process, including a protein, subunit, organelle, ribonucleoprotein, etc. In some embodiments, an IC₅₀ can refer to the concentration of a substance that is required for 50% inhibition in vivo, as further defined elsewhere herein.

As used herein, “EC₅₀,” is intended to refer to the concentration of a substance (e.g., a compound or a drug) that is results in a half-maximal response (i.e., 50% of the maximum response) of a biological process, or component of a process, including a protein, subunit, organelle, ribonucleoprotein, etc. In some embodiments, an EC₅₀ can refer to the concentration of a substance that is required to achieve 50% of the maximum response in vivo, as further defined elsewhere herein.

The compounds according to this disclosure may form prodrugs at hydroxyl or amino functionalities using alkoxy, amino acids, etc., groups as the prodrug forming moieties. For instance, the hydroxymethyl position may form mono-, di- or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

In some embodiment, the disclosed compositions and pharmaceutical compositions comprise one or a plurality of derivatives of the compounds disclosed herein. “Derivatives” of the compounds disclosed herein are pharmaceutically acceptable salts, prodrugs, deuterated forms, radio-actively labeled forms, isomers, solvates and combinations thereof. The “combinations” mentioned in this context are refer to derivatives falling within at least two of the groups: pharmaceutically acceptable salts, prodrugs, deuterated forms, radio-actively labeled forms, isomers, and solvates. Examples of radio-actively labeled forms include compounds labeled with tritium, phosphorous-32, iodine-129, carbon-11, fluorine-18, and the like.

The term “leaving group” refers to an atom (or a group of atoms) with electron withdrawing ability that can be displaced as a stable species, taking with it the bonding electrons. Examples of suitable leaving groups include sulfonate esters, including triflate, mesylate, tosylate, brosylate, and halides.

As used herein, the term “substituted” is contemplated to include all permissible substituents of organic compounds. In a broad embodiment, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in some embodiments, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The terms “halo” and “halogen” as used herein refer to an atom selected from fluorine (fluoro, —F), chlorine (chloro, —Cl), bromine (bromo, —Br), and iodine (iodo, —I).

The term “alkyl,” as used herein, refers to a monovalent saturated, straight- or branched-chain hydrocarbon radical, having unless otherwise specified, 1-6 carbon atoms. Examples of alkyl radicals include, but are not limited to, methyl, ethyl, n-propyl, isopropyl, n-butyl, iso-butyl, sec-butyl, n-pentyl, tert-pentyl, neopentyl, sec-pentyl, 3-pentyl, sec-isopentyl, hexyl, 2-methylpentane, 3-methylpentane, 2,2-dimethylbutane, 2,3-dimentybutane and the like.

The term “haloalkyl” includes mono, poly, and perhaloalkyl groups where the halogens are independently selected from fluorine, chlorine, bromine, and iodine.

“Alkoxy” is an alkyl group which is attached to another moiety via an oxygen linker (—O(alkyl)). Non-limiting examples include methoxy, ethoxy, propoxy, and butoxy.

“Haloalkoxy” is a haloalkyl group which is attached to another moiety via an oxygen atom such as, e.g., but are not limited to —OCHCF₂ or —OCF₃.

The term “9- to 10-membered carbocyclyl” means a 9- or 10-membered monocyclic, bicyclic (e.g., a bridged or spiro bicyclic ring), polycyclic (e.g., tricyclic), or fused hydrocarbon ring system that is saturated or partially unsaturated. The term “9- to 10-membered carbocyclyl” also includes saturated or partially unsaturated hydrocarbon rings that are fused to one or more aromatic or partically saturated hydrocarbon rings (e.g., dihydroindenyl and tetrahydronaphthalenyl). Bridged bicyclic cycloalkyl groups include, without limitation, bicyclo[4.3.1]decanyl and the like. Spiro bicyclic cycloalkyl groups include, e.g., spiro[3.6]decanyl, spiro[4.5]decanyl, spiro [4.4]nonyl and the like. Fused cycloalkyl rings include, e.g., decahydronaphthalenyl, dihydroindenyl, decahydroazulenyl, octahydroazulenyl, tetrahydronaphthalenyl, and the like. It will be understood that when specified, optional substituents on a carbocyclyl (e.g., in the case of an optionally substituted cycloalkyl) may be present on any substitutable position and, include, e.g., the position at which the carbocyclyl group is attached.

A cycloalkyl is a completely saturated carbocycle and includes e.g., cyclopropyl, cyclobutyl, cyclopentyl, cyclohexyl, cycloheptyl, and cyclooctyl.

The term “9-membered fused heterocyclyl” means a 9-membered saturated or partially unsaturated fused monocyclic heterocyclic ring comprising at least one oxygen heteroatom and optionally two to four additional heteroatoms independently selected from N, O, and S. The terms “heterocycle,” “heterocyclyl,” “heterocyclyl ring,” “heterocyclic group,” “heterocyclic moiety,” and “heterocyclic radical,” are used interchangeably herein. A heterocyclyl ring can be attached to its pendant group at any heteroatom or carbon atom that results in a stable structure. Examples of fused saturated or partially unsaturated heterocyclic radicals compristing at least one oxygen atom include, without limitation, dihydrobenzofuranyl, dihydrofuropyridinyl, octahydrobenzofuranyl, and the like. Where specified as being optionally substituted, substituents on a heterocyclyl (e.g., in the case of an optionally substituted heterocyclyl) may be present on any substitutable position and include, e.g., the position at which the heterocyclyl group is attached.

The term “5- or 6-membered heteroaryl” refers to a 5- or 6-membered aromatic radical containing 1-4 heteroatoms selected from N, O, and S. Nonlimiting examples include thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, etc. When specified, optional substituents on a heteroaryl group may be present on any substitutable position and, include, e.g., the position at which the heteroaryl is attached.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in some embodiments, unless expressly indicated to the contrary, individual substituents can be further optionally substituted (i.e., further substituted or unsubstituted).

In some embodiments, a structure of a compound can be represented by a formula:

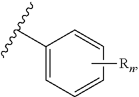

which is understood to be equivalent to a formula:

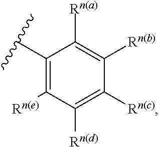

wherein n is typically an integer. That is, Rⁿ is understood to represent five independent substituents, R^{n(a)}, R^{n(b)}, R^{n(c)}, R^{n(d)}, R^{n(e)}. In each such case, each of the five Rⁿ can be hydrogen or a recited substituent. By “independent substituents,” it is meant that each R substituent can be independently defined. For example, if in one instance R^{n(a)} is halogen, then R^{n(b)} is not necessarily halogen in that instance.

In some yet further embodiments, a structure of a compound can be represented by a formula:

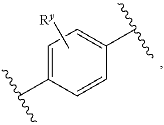

wherein R^{y} represents, for example, 0-2 independent substituents selected from A¹, A², and A³, which is understood to be equivalent to the groups of formulae:
wherein R^{y} represents 0 independent substituents

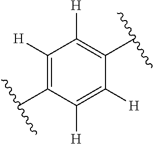

wherein R^{y} represents 1 independent substituent

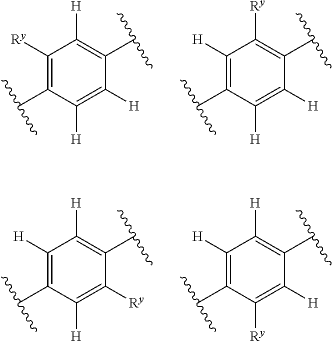

wherein R^{y} represents 2 independent substituents

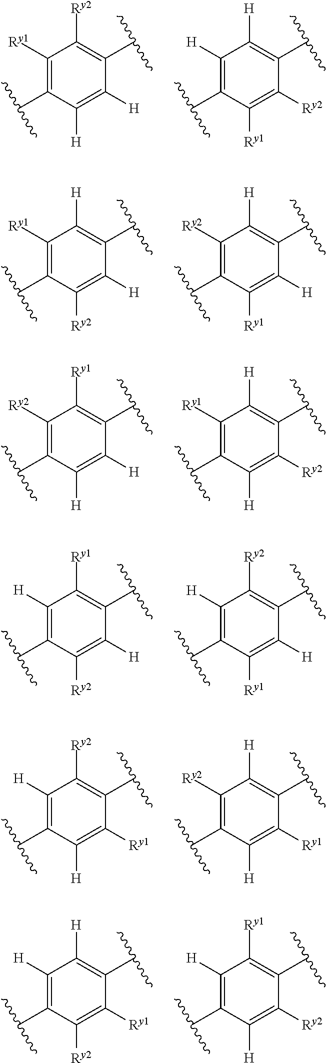

Again, by “independent substituents,” it is meant that each R substituent can be independently defined. For example, if in one instance R^{y1} is A¹, then R^{y2} is not necessarily A¹ in that instance.

In some further embodiments, a structure of a compound can be represented by a formula,

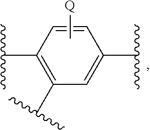

wherein, for example, Q comprises three substituents independently selected from hydrogen and A, which is understood to be equivalent to a formula:

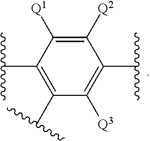

Again, by “independent substituents,” it is meant that each Q substituent is independently defined as hydrogen or A, which is understood to be equivalent to the groups of formulae:
wherein Q comprises three substituents independently selected from H and A

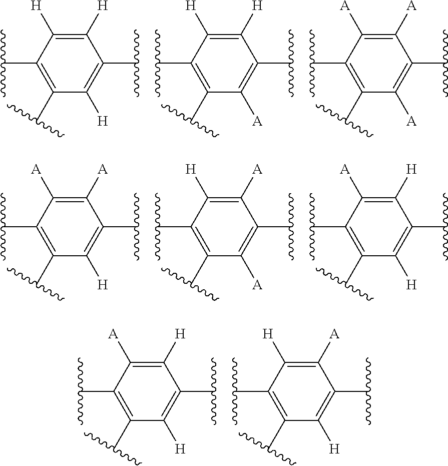

In some embodiment, the disclosed compounds exists as geometric isomers. “Geometric isomer” refers to isomers that differ in the orientation of substituent atoms in relationship to a cycloalkyl ring, i.e., cis or trans isomers. When a disclosed compound is named or depicted by structure without indicating a particular cis or trans geometric isomer form, it is to be understood that the name or structure encompasses one geometric isomer free of other geometric isomers, mixtures of geometric isomers, or mixtures enriched in one geometric isomer relative to its corresponding geometric isomer. When a particular geometric isomer is depicted, i.e., cis or trans, the depicted isomer is at least 60%, 70%, 80%, 90%, 99% or 99.9% by weight pure relative to the other geometric isomer.

The compounds described herein may be present in the form of pharmaceutically acceptable salts. For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Examples of pharmaceutically acceptable base addition salts include e.g., sodium, potassium, calcium, ammonium, organic amino, or magnesium salt.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

As used herein, the phrase “pharmaceutically acceptable” means those compounds, materials, compositions, and/or dosage forms which are, within the scope of sound medical judgment, suitable for use in contact with tissues of humans and animals. In some embodiments, “pharmaceutically acceptable” means approved by a regulatory agency of the Federal or a state government or listed in the U.S. Pharmacopeia or other generally recognized pharmacopeia for use in animals, and more particularly in humans.

Disease, disorder, and condition are used interchangeably herein.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed, i.e., therapeutic treatment. In other embodiments, treatment may be administered in the absence of symptoms. For example, treatment may be administered to a susceptible individual prior to the onset of symptoms (e.g., in light of a history of symptoms and/or in light of exposure to a particular organism, or other susceptibility factors), i.e., prophylactic treatment. Treatment may also be continued after symptoms have resolved, for example to delay their recurrence.

As used herein, the term “prevent” or “preventing” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. The term “preventing” refers to preventing a disease, disorder, or condition from occurring in a human or an animal that may be predisposed to the disease, disorder and/or condition, but has not yet been diagnosed as having it; and/or inhibiting the disease, disorder, or condition, i.e., arresting its development.

The term “effective amount” or “therapeutically effective amount” refers to an amount that is sufficient to achieve the desired result (e.g., that will elicit a biological or medical response of a subject; e.g., a dosage of between 0.01-100 mg/kg body weight/day) or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various embodiments, a preparation can be administered in a “prophylactically effective amount”; that is, an amount effective for prevention of a disease or condition.

As used herein, the term “salt” refers to acid or base salts of the compounds used in the methods of the present disclosure. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). In some embodiments, the subject is a human in need of treatment. In some embodiments, the subject has been diagnosed with a mitchondiral disease. In some embodiments, the subject has not been diagnosed with a mitochondrial disease or is free of a symptom of mitochondrial disease.

The term “associated” or “associated with” in the context of a substance or substance activity or function associated with a disease (e.g., a protein associated disease, a symptom associated with a cardiomyopathy, neurodegenerative disease, or symptom associated with Parkinson's disease) means that the disease (e.g., cardiomyopathy, neurodegenerative disease or Parkinson's disease) is caused by (in whole or in part), or a symptom of the disease is caused by (in whole or in part) the substance or substance activity or function. For example, a symptom of a disease or condition associated with a reduction in the level of PINK1 activity may be a symptom that results (entirely or partially) from a reduction in the level of PINK1 activity (e.g., loss of function mutation or gene deletion or modulation of PINK1 signal transduction pathway). As used herein, what is described as being associated with a disease, if a causative agent, could be a target for treatment of the disease. For example, a disease associated with PINK1, may be treated with an agent (e.g., compound as described herein) effective for increasing the level of activity of PINK1.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

“Contacting” is used in accordance with its plain ordinary meaning and refers to the process of allowing at least two distinct species (e.g., chemical compounds including biomolecules, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme (e.g., PINK1). In some embodiments contacting includes allowing a compound described herein to interact with a protein or enzyme that is involved in a signaling pathway.

As defined herein, the term “inhibition,” “inhibit,” “inhibiting,” and the like in reference to a protein-inhibitor (e.g., antagonist) interaction means negatively affecting (e.g., decreasing or eliminating) the activity or function of the protein relative to the activity or function of the protein in the absence of the inhibitor. In some embodiments inhibition refers to reduction of a disease or symptoms of disease. In some embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein.

The symbol “ [symbol] ” denotes the point of attachment of a chemical moiety to the [symbol] remainder of a molecule or chemical formula.

As defined herein, the term “activation,” “activate,” “activating” and the like in reference to a protein-activator (e.g., agonist) interaction means positively affecting (e.g., increasing) the activity or function of the protein (e.g., PINK1) relative to the activity or function of the protein in the absence of the activator (e.g., compound described herein). In some embodiments, activation refers to an increase in the activity of a signal transduction pathway or signaling pathway (e.g., PINK1 pathway). Thus, activation may include, at least in part, partially or totally increasing stimulation, increasing or enabling activation, or activating, sensitizing, or up-regulating signal transduction or enzymatic activity or the amount of a protein decreased in a disease (e.g., reduction of the level of PINK1 activity or protein associated with a cardiomyopathy or a neurodegenerative disease such as Parkinson's disease). Activation may include, at least in part, partially or totally increasing stimulation, increasing or enabling activation, or activating, sensitizing, or up-regulating signal transduction or enzymatic activity or the amount of a protein (e.g., PINK1) that may modulate the level of another protein or increase cell survival (e.g., increase in PINK1 activity may increase cell survival in cells that may or may not have a reduction in PINK1 activity relative to a non-disease control).

The term “modulator” refers to a composition that increases or decreases the level of a target molecule or the function of a target molecule. In some embodiments, the modulator is a modulator of PINK1. In some embodiments, the modulator is a modulator of PINK1 and is a compound that reduces the severity of one or more symptoms of a disease associated with PINK1 (e.g., reduction of the level of PINK1 activity or protein associated with a cardiomyopathy, neurodegenerative disease such as Parkinson's disease). In some embodiments, a modulator is a compound that reduces the severity of one or more symptoms of a cardiomyopathy or neurodegenerative disease that is not caused or characterized by PINK1 (e.g., loss of PINK1 function) but may benefit from modulation of PINK1 activity (e.g., increase in level of PINK1 or PINK1 activity).

“Patient” or “subject in need thereof” refers to a living organism suffering from or prone to a disease or condition that can be treated by administration of a compound or pharmaceutical composition, as provided herein. Non-limiting examples include humans, other mammals, non-human primates, bovines, rats, mice, dogs, monkeys, goat, sheep, cows, deer, and other non-mammalian animals. In some embodiments, a patient is human.

“Disease” or “condition” refer to a state of being or health status of a patient or subject capable of being treated with a compound, pharmaceutical composition, or method provided herein. In some embodiments, the disease is a disease related to (e.g., characterized by) a reduction in the level of PINK1. In some embodiments, the disease is a disease characterized by loss of dopamine-producing cells (e.g., Parkinson's disease). In some embodiments, the disease is a disease characterized by neurodegeneration. In some embodiments, the disease is a disease characterized by neural cell death. In some embodiments, the disease is a disease characterized by a reduction in the level of PINK1 activity. In some embodiments, the disease is Parkinson's disease. In some embodiments, the disease is a neurodegenerative disease. In some embodiments, the disease is a cardiomyopathy.

As used herein, the term “cardiomyopathy” refers to a disease condition that adversely affects cardiac cell tissue leading to a measurable deterioration in myocardial function (e.g., systolic function, diastolic function). Dilated cardiomyopathy is characterized by ventricular chamber enlargement with systolic dysfunction and no hypertrophy. Hypertrophic cardiomyopathy, is a genetic disease transmitted as an autosomal dominant trait. Hypertrophic cardiomyopathy is morphologically characterized by a hypertrophied and non-dialated left ventricle. Restrictive cardiomyopathy is characterized by nondialated nonhypertrophied morphology with diminished ventricular volume leading to poor ventricular filling. Arrhythmogenic right ventricular cardiomyopathy is an inheritable heart disease characterized by myocardial electric instability. Unclassified cardiomyopathy is a category for cardiomyopathies that do not match the features of any one of the other types. Unclassified cardiomyopathies may have features of multiple types or, for example, have the features of fibroelastosis, noncompacted myocardium, or systolic dysfunction with minimal dilatation.

As used herein, the term “neurodegenerative disease” refers to a disease or condition in which the function of a subject's nervous system becomes impaired. Examples of neurodegenerative diseases that may be treated with a compound or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, epilepsy, Friedreich ataxia, frontotemporal dementia, Gerstmann-Sträussler-Scheinker syndrome, Huntington's disease, HIV-associated dementia, Kennedy's disease, Krabbe's disease, kuru, Leigh's disease (Leigh syndrome), Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoff's disease, Schilder's disease, Shy-Drager syndrome, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, Tabes dorsalis, drug-induced Parkinsonism, progressive supranuclear palsy, corticobasal degeneration, multiple system atrophy, Idiopathic Parkinson's disease, Autosomal dominant Parkinson disease, Parkinson disease, familial, type 1 (PARK1), Parkinson disease 3, autosomal dominant Lewy body (PARK3), Parkinson disease 4, autosomal dominant Lewy body (PARK4), Parkinson disease 5 (PARK5), Parkinson disease 6, autosomal recessive early-onset (PARK6), Parkinson disease 2, autosomal recessive juvenile (PARK2), Parkinson disease 7, autosomal recessive early-onset (PARK7), Parkinson disease 8 (PARK8), Parkinson disease 9 (PARK9), Parkinson disease 10 (PARK10), Parkinson disease 11 (PARK11), Parkinson disease 12 (PARK12), Parkinson disease 13 (PARK13), or Mitochondrial Parkinson's disease. In some embodiments, dysautonomia is not a neurodegenerative disease.

The term “signaling pathway” as used herein refers to a series of interactions between cellular and optionally extra-cellular components (e.g., proteins, nucleic acids, small molecules, ions, lipids) that conveys a change in one component to one or more other components, which in turn may convey a change to additional components, which is optionally propagated to other signaling pathway components.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., cardiomyopathy therapies including, for example, Angiotensin Converting Enzyme Inhibitors (e.g., Enalipril, Lisinopril), Angiotensin Receptor Blockers (e.g., Losartan, Valsartan), Beta Blockers (e.g., Lopressor, Toprol-XL), Digoxin, or Diuretics (e.g., Lasix; or Parkinson's disease therapies including, for example, levodopa, dopamine agonists (e.g., bromocriptine, pergolide, pramipexole, ropinirole, piribedil, cabergoline, apomorphine, lisuride), MAO-B inhibitors (e.g., selegiline or rasagiline), amantadine, anticholinergics, antipsychotics (e.g., clozapine), cholinesterase inhibitors, modafinil, or non-steroidal anti-inflammatory drugs.

The compound of the disclosure can be administered alone or can be coadministered to the patient. Coadministration is meant to include simultaneous or sequential administration of the compound individually or in combination (more than one compound or agent). Thus, the preparations can also be combined, when desired, with other active substances (e.g., to reduce metabolic degradation). The compositions of the present disclosure can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present disclosure may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present disclosure can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In some embodiments, the formulations of the compositions of the present disclosure can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present disclosure into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present disclosure can also be delivered as nanoparticles.

Pharmaceutical compositions provided by the present disclosure include compositions wherein the active ingredient (e.g., compounds described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule (e.g., PINK1), and/or reducing, eliminating, or slowing the progression of disease symptoms (e.g., symptoms of cardiomyopathy or a neurodegeneration such as symptoms of Parkinson's disease). Determination of a therapeutically effective amount of a compound of the disclosure is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g., symptoms of cardiomyopathy or neurodegeneration such as Parkinson's disease and severity of such symptoms), kind of concurrent treatment, complications from the disease being treated or other health-related problems. Other therapeutic regimens or agents can be used in conjunction with the methods and compounds of Applicants' disclosure. Adjustment and manipulation of established dosages (e.g., frequency and duration) are well within the ability of those skilled in the art.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present disclosure should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The compounds described herein can be used in combination with one another, with other active agents known to be useful in treating a disease associated neurodegeneration (e.g., Parkinson's disease such as levodopa, dopamine agonists (e.g., bromocriptine, pergolide, pramipexole, ropinirole, piribedil, cabergoline, apomorphine, lisuride), MAO-B inhibitors (e.g., selegiline or rasagiline), amantadine, anticholinergics, antipsychotics (e.g., clozapine), cholinesterase inhibitors, modafinil, or non-steroidal anti-inflammatory drugs), or with adjunctive agents that may not be effective alone, but may contribute to the efficacy of the active agent.

The compounds described herein can be used in combination with one another, with other active agents known to be useful in treating a cardiomyopathy such as Angiotensin Converting Enzyme Inhibitors (e.g., Enalipril, Lisinopril), Angiotensin Receptor Blockers (e.g., Losartan, Valsartan), Beta Blockers (e.g., Lopressor, Toprol-XL), Digoxin, or Diuretics (e.g., Lasixdisease associated neurodegeneration (e.g., Parkinson's disease such as levodopa, dopamine agonists (e.g., bromocriptine, pergolide, pramipexole, ropinirole, piribedil, cabergoline, apomorphine, lisuride), MAO-B inhibitors (e.g., selegiline or rasagiline), amantadine, anticholinergics, antipsychotics (e.g., clozapine), cholinesterase inhibitors, modafinil, or non-steroidal anti-inflammatory drugs), or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In some embodiments, the active and/or adjunctive agents may be linked or conjugated to one another. In some embodiments, the compounds described herein may be combined with treatments for neurodegeneration such as surgery. In some embodiments, the compounds described herein may be combined with treatments for cardiomyopathy such as surgery.

“PINK1” is used according to its common, ordinary meaning and refers to proteins of the same or similar names and functional fragments and homologs thereof. The term includes and recombinant or naturally occurring form of PINK1 (e.g., “PTEN induced putative kinase 1”; Entrez Gene 65018, OMIM 608309, UniProtKB Q9BXM7, and/or RefSeq (protein) NP_115785.1). The term includes PINK1 and variants thereof that maintain PINK1 activity (e.g., within at least 30%, 40%, 50%, 60%, 70%, 80%, 90%, 95%, or 100% activity as compared to PINK1).

The term “neo-substrate” refers to a composition that is structurally similar to a composition that is a substrate for a protein or enzyme during the normal functioning of the protein or enzyme, but that is structurally distinct from the normal substrate of the protein or enzyme. In some embodiments, the neo-substrate is a better substrate for the protein or enzyme than the normal substrate (e.g., the reaction kinetics are better (e.g., faster), binding is stronger, turnover rate is higher, reaction is more productive, equilibrium favors product formation, etc.). In some embodiments, the neo-substrate is a derivative of adenine, adenosine, AMP, ADP, or ATP. In some embodiments, the neo-substrate is a substrate for PINK1. In some embodiments, the neo-substrate is an N6 substituted adenine, adenosine, AMP, ADP, or ATP.

The term “derivative” as applied to a phosphate containing, monophosphate, diphosphate, or triphosphate group or moiety refers to a chemical modification of such group wherein the modification may include the addition, removal, or substitution of one or more atoms of the phosphate containing, monophosphate, diphosphate, or triphosphate group or moiety. In some embodiments, such a derivative is a prodrug of the phosphate containing, monophosphate, diphosphate, or triphosphate group or moiety, which is converted to the phosphate containing, monophosphate, diphosphate, or triphosphate group or moiety from the derivative following administration to a subject, patient, cell, biological sample, or following contact with a subject, patient, cell, biological sample, or protein (e.g., enzyme). In an embodiment, a triphosphate derivative is a gamma-thio triphosphate. In an embodiment, a derivative is a phosphoramidate. In some embodiments, the derivative of a phosphate containing, monophosphate, diphosphate, or triphosphate group or moiety is as described in Murakami et al. J. Med Chem., 2011, 54, 5902; Sofia et al., J. Med Chem. 2010, 53, 7202; Lam et al. ACC, 2010, 54, 3187; Chang et al., ACS Med Chem Lett., 2011, 2, 130; Furman et al., Antiviral Res., 2011, 91, 120; Vemachio et al., ACC, 2011, 55, 1843; Zhou et al, AAC, 2011, 44, 76; Reddy et al., BMCL, 2010, 20, 7376; Lam et al., J. Virol., 2011, 85, 12334; Sofia et al., J. Med. Chem., 2012, 55, 2481, Hecker et al., J. Med. Chem., 2008, 51, 2328; or Rautio et al., Nature Rev. Drug. Discov. 2008, 7, 255, all of which are incorporated herein by reference in their entirety for all purposes.

The term “mitochondrial dysfunction” is used in accordance with its ordinary meaning and refers to aberrant activity of function of the mitochondria, including for example aberrant respiratory chain activity, reactive oxygen species levels, calcium homeostasis, programmed cell death mediated by the mitochondria, mitochondrial fusion, mitochondrial fission, mitophagy, lipid concentrations in the mitochondrial membrane, mitochondrial protein import, mitochondrial replication, transcription, translation, and/or mitochondrial permeability transition.

As used herein, the term “mitochondrial disease” refers to a disease, disorder, or condition in which the function of a subject's mitochondria becomes impaired or dysfunctional. Examples of mitochondrial diseases that may be treated with a compound or method described herein include Alzheimer's disease, amyotrophic lateral sclerosis, Asperger's Disorder, Autistic Disorder, bipolar disorder, cancer, cardiomyopathy, Charcot Marie Tooth disease (CMT, including various subtypes such as CMT type 2b and 2b), Childhood Disintegrative Disorder (CDD), diabetes, diabetic nephropathy, epilepsy, Friedreich's Ataxia (FA), Hereditary motor and sensory neuropathy (HMSN), Huntington's Disease, Keams-Sayre Syndrome (KSS), Leber's Hereditary Optic Neuropathy (LHON, also referred to as Leber's Disease, Leber's Optic Atrophy (LOA), or Leber's Optic Neuropathy (LON)), Leigh Disease or Leigh Syndrome, macular degeneration, Mitochondrial Myopathy, Lactacidosis, and Stroke (MELAS), mitochondrial neurogastrointestinal encephalomyophathy (MNGIE), motor neuron diseases, Myoclonic Epilepsy With Ragged Red Fibers (MERRF), Neuropathy, ataxia, retinitis pigmentosa, and ptosis (NARP), Parkinson's disease, Peroneal muscular atrophy (PMA), Pervasive Developmental Disorder Not Otherwise Specified (PDD-NOS), renal tubular acidosis, Rett's Disorder, Schizophrenia, and types of stroke.

The term “oxidative stress” is used in accordance with its ordinary meaning and refers to aberrant levels of reactive oxygen species.

As used herein, the term “animal” includes, but is not limited to, humans and non-human vertebrates such as wild, domestic, and farm animals.

As used herein, the term “carrier” means a diluent, adjuvant, or excipient with which a compound is administered. Pharmaceutical carriers can be liquids, such as water and oils, including those of petroleum, animal, vegetable or synthetic origin, such as peanut oil, soybean oil, mineral oil, sesame oil and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used.

As used herein, the terms “comprising” (and any form of comprising, such as “comprise,” “comprises,” and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the term “contacting” means bringing together of two elements in an in vitro system or an in vivo system. For example, “contacting” a compound disclosed herein with an individual or patient or cell includes the administration of the compound to an individual or patient, such as a human, as well as, for example, introducing a compound into a sample containing a cellular or purified preparation containing the compounds or pharmaceutical compositions disclosed herein.

As used herein, the terms “individual,” “subject” or “patient,” used interchangeably, means any animal, including mammals, such as mice, rats, other rodents, rabbits, dogs, cats, swine, cattle, sheep, horses, or primates, such as humans.

As used herein, the phrase “inhibiting activity,” such as enzymatic or receptor activity means reducing by any measurable amount the activity of PINK1.

As used herein, the phrase “in need thereof” means that the animal or mammal has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent.

As used herein, the phrase “integer from X to Y” means any integer that includes the endpoints. For example, the phrase “integer from 1 to 5” means 1, 2, 3, 4, or 5.

As used herein, the term “isolated” means that the compounds described herein are separated from other components of either (a) a natural source, such as a plant or cell, or (b) a synthetic organic chemical reaction mixture, such as by conventional techniques.

As used herein, the term “mammal” means a rodent (i.e., a mouse, a rat, or a guinea pig), a monkey, a cat, a dog, a cow, a horse, a pig, or a human. In some embodiments, the mammal is a human.

As used herein, the term “prodrug” means a derivative of a known direct acting drug, which derivative has enhanced delivery characteristics and therapeutic value as compared to the drug, and is transformed into the active drug by an enzymatic or chemical process. The compounds described herein also include derivatives referred to as prodrugs, which can be prepared by modifying functional groups present in the compounds in such a way that the modifications are cleaved, either in routine manipulation or in vivo, to the parent compounds. Examples of prodrugs include compounds of the disclosure as described herein that contain one or more molecular moieties appended to a hydroxyl, amino, sulfhydryl, or carboxyl group of the compound, and that when administered to a patient, cleaves in vivo to form the free hydroxyl, amino, sulfhydryl, or carboxyl group, respectively. Examples of prodrugs include, but are not limited to, acetate, formate and benzoate derivatives of alcohol and amine functional groups in the compounds of the disclosure. Preparation and use of prodrugs is discussed in T. Higuchi et al., “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are incorporated herein by reference in their entireties.

As used herein, the term “purified” means that when isolated, the isolate contains at least 90%, at least 95%, at least 98%, or at least 99% of a compound described herein by weight of the isolate.

As used herein, the phrase “solubilizing agent” means agents that result in formation of a micellar solution or a true solution of the drug.

As used herein, the term “solution/suspension” means a liquid composition wherein a first portion of the active agent is present in solution and a second portion of the active agent is present in particulate form, in suspension in a liquid matrix.

As used herein, the phrase “substantially isolated” means a compound that is at least partially or substantially separated from the environment in which it is formed or detected.

As used herein, the phrase “therapeutically effective amount” means the amount of active compound or pharmaceutical agent that elicits the biological or medicinal response that is being sought in a tissue, system, animal, individual or human by a researcher, veterinarian, medical doctor or other clinician. The therapeutic effect is dependent upon the disorder being treated or the biological effect desired. As such, the therapeutic effect can be a decrease in the severity of symptoms associated with the disorder and/or inhibition (partial or complete) of progression of the disorder, or improved treatment, healing, prevention or elimination of a disorder, or side-effects. The amount needed to elicit the therapeutic response can be determined based on the age, health, size and sex of the subject. Optimal amounts can also be determined based on monitoring of the subject's response to treatment.

It is further appreciated that certain features described herein, which are, for clarity, described in the context of separate embodiments, can also be provided in combination in a single embodiment. Conversely, various features which are, for brevity, described in the context of a single embodiment, can also be provided separately or in any suitable subcombination.

It should be noted that any embodiment of the invention can optionally exclude one or more embodiment for purposes of claiming the subject matter.

In some embodiments, the compounds, or salts thereof, are substantially isolated. Partial separation can include, for example, a composition enriched in the compound of the disclosure. Substantial separation can include compositions containing at least about 50%, at least about 60%, at least about 70%, at least about 80%, at least about 90%, at least about 95%, at least about 97%, or at least about 99% by weight of the compound of the disclosure, or salt thereof. Methods for isolating compounds and their salts are routine in the art.

### B. Compounds

In various embodiments, the invention relates to compounds useful in treating disorders associated with PINK1 kinase activity such as, for example, a neurodegenerative disease, a mitochondrial disease, fibrosis, and/or cardiomyopathy.

In various embodiments, the compounds are useful in treating a disorder associated with PINK1 kinase activity in a mammal. In a further embodiment, the compounds are useful in treating a disorder associated with PINK1 kinase activity in a human.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

### 1. Structure

In some embodiments, provided are compounds having a structure represented by a formula:

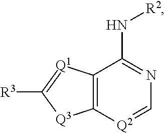

wherein Q¹ is N or CH and R³ is a 3- to 6-membered cycloalkyl, C1-C6 haloalkyl, C1-C6 haloalkoxy, or C1-C6 halohydroxyalkyl; or wherein Q¹ is CR¹ and R³ is hydrogen; R¹ is C1-C6 haloalkyl, C1-C6 haloalkoxy, C1-C6 halohydroxyalkyl, or a structure represented by a formula:

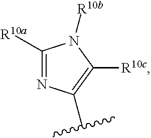

wherein each of R^{10a}, R^{10b}, and R^{10c}, when present, is independently selected from hydrogen and C1-C4 alkyl; wherein Q² is CH or N; wherein Q³ is CH₂ or NH; wherein R² is C1-C6 alkyl, —CR^{11a}R^{11b}Cy¹, or Cy¹; wherein each of R^{11a} and R^{11b}, when present, is independently selected from hydrogen, C1-C5 alkyl, and C1-C4 hydroxyalkyl; or wherein each of R^{11a} and R^{11b}, when present, together comprise a 3-membered cycloalkyl; wherein Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, provided that when R¹ is C1-C6 haloalkyl and R² is Cy¹, then Cy¹ is not a 6-membered carbocycle or a 9-membered heteroaryl, and provided that when R² is —CR^{11a}R^{11b}Cy¹ or Cy¹, one or both of R^{11a} and R^{11b}, when present, is hydrogen, and Cy¹ is a 6-membered aryl or furanyl, then Q¹ is CH and R³ is not a C1-C6 haloalkyl, or a pharmaceutically acceptable salt thereof.

In some embodiments, provided is a compound having a structure:

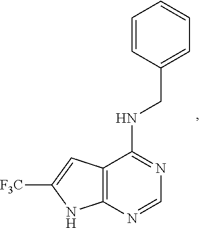 [SMILES: FC(F)(F)c1cc2c(NCc3ccccc3)ncnc2[nH]1]

or a pharmaceutically acceptable salt thereof.

In some embodiments, provided are compounds having a structure represented by a formula:

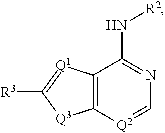

wherein Q¹ is N or CH and R³ is a 3- to 6-membered cycloalkyl or a C1-C6 haloalkyl, C1-C6 haloalkoxy, C1-C6 halohydroxyl, CF₃, CCl₃, CBr₃; or wherein Q¹ is CR¹ and R³ is hydrogen; R¹ is C1-C6 haloalkyl, C1-C6 haloalkoxy, C1-C6 halohydroxy, or a structure represented by a formula:

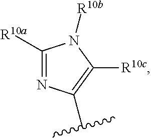

wherein each of R^{10a}, R^{10b}, and R^{10c}, when present, is independently selected from hydrogen and C1-C4 alkyl; wherein Q² is CH or N; wherein Q³ is CH₂ or NH; wherein R² is C1-C6 alkyl, —CR^{11a}R^{11b}Cy¹, or Cy¹; wherein each of R^{11a} and R^{11b}, when present, is independently selected from hydrogen, C1-C5 alkyl, and C1-C5 hydroxyalkyl; or wherein each of R^{11a} and R^{11b} together comprise a 3-membered cycloalkyl; wherein Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, provided that when R¹ is C1-C6 haloalkyl and R² is Cy¹, then Cy¹ is not a 6-membered carbocycle or a 9-membered heteroaryl, or a pharmaceutically acceptable salt thereof.

In some embodiments, provided are compounds selected from:

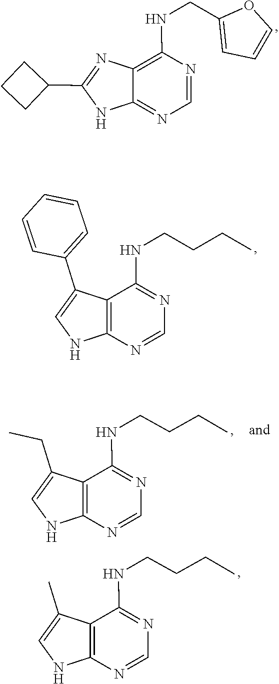

or a pharmaceutically acceptable salt thereof.

In some embodiments, provided are compounds selected from:

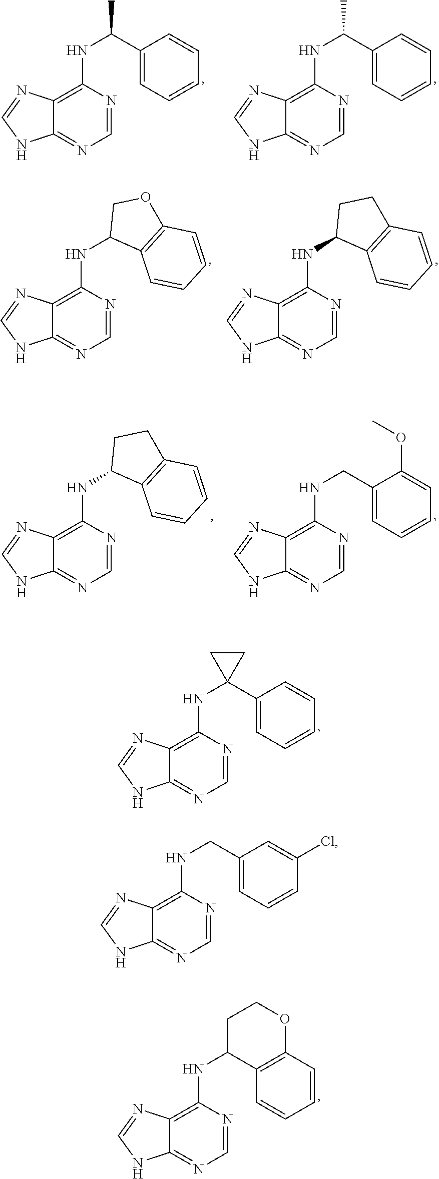

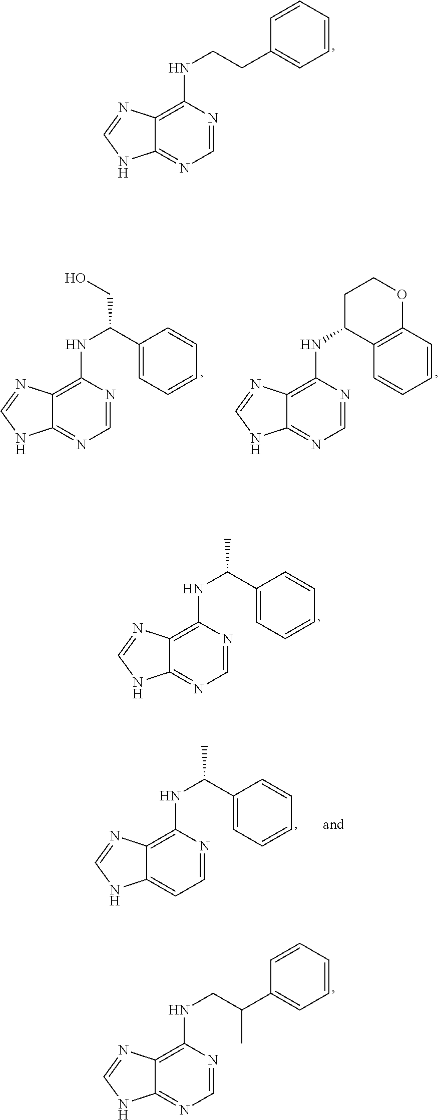 [SMILES: c1ccc(CCNc2ncnc3[nH]cnc23)cc1 ; OC[C@@H](Nc1ncnc2[nH]cnc12)c1ccccc1 ; c1ccc2c(c1)OCC[C@H]2Nc1ncnc2[nH]cnc12 ; C[C@@H](Nc1ncnc2[nH]cnc12)c1ccccc1 ; C[C@@H](Nc1nccc2[nH]cnc12)c1ccccc1 ; CC(CNc1ncnc2[nH]cnc12)c1ccccc1]

or a pharmaceutically acceptable salt thereof.

In some embodiments, provided are compounds having a structure represented by Formula I:

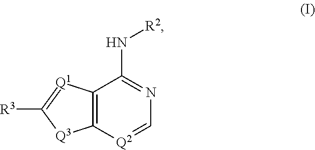

wherein Q¹ is N or CH and R³ is a 3- to 6-membered cycloalkyl or a C1-C6 haloalkyl, C1-C6 haloalkoxy, C1-C6 halohydroxyl, CF₃, CCl₃, CBr₃; or wherein Q¹ is CR¹ and R³ is hydrogen; wherein Q² is CH or N; wherein Q³ is CH₂ or NH; R¹ is (C₁-C₆)alkyl, halo(C₁-C₄)alkyl, (C₁-C₄)alkoxy, halo(C₁-C₄)alkoxy, 5- or 6-membered heteroaryl, or phenyl, wherein said C₁-C₆alkyl and halo(C₁-C₄)alkyl are each optionally and independently substituted with a OR^{a} group, and wherein said phenyl and 5- or 6-membered heteroaryl are each optionally and independently substituted with 1 to 3 groups independently selected from R^{b}; R^{a}, when present, is H, (C₁-C₄)alkyl, or (C₁-C₄)alkoxy; each occurrence of R^{b}, when present, is independently halo, halo(C₁-C₄)alkyl, (C₁-C₄)alkoxy, or halo(C₁-C₄)alkoxy; R² is (C₁-C₆)alkyl, a 9-membered oxygen-containing fused heterocycle, or a 9- to 10-membered carbocycle, wherein said (C₁-C₆)alkyl is optionally substituted with 1 or 2 groups independently selected from R^{c}, and wherein said 9-membered oxygen-containing fused heterocycle and 9- to 10-membered carbocycle are each optionally and independently substituted with 1 to 3 groups independently selected from R^{d}; each occurrence of R^{c}, when present, is phenyl, 3- or 4-membered cycloalkyl, or 5- or 6-membered heteroaryl, wherein said phenyl and 5- or 6-membered heteroaryl are each optionally and independently substituted with 1 to 3 groups independently selected from R^{e}; each occurrence of R^{d} and R^{e}, when present, is independently halo, halo(C₁-C₄)alkyl, (C₁-C₄)alkoxy, or halo(C₁-C₄)alkoxy; and R³ is hydrogen, halogen, (C₁-C₄)alkyl, 3- to 6-membered cycloalkyl, halo, halo(C1-C4)alkyl, halo (C1-C4)alkoxy or pharmaceutically acceptable salts thereof.

Thus, in various embodiments, the present disclosure provides a compound of Formula I:

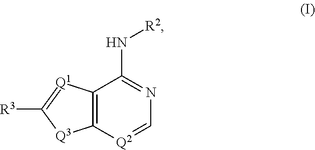

or a pharmaceutically acceptable salt thereof, wherein the variables are as defined above.

In further embodiments, R¹ in the compound of Formula I is (C₁-C₄)alkyl, halo(C₁-C₄)alkyl, 5- or 6-membered heteroaryl, or phenyl, wherein said halo(C₁-C₄)alkyl is optionally substituted with a OR^{a} group, and wherein said 5- or 6-membered heteroaryl is optionally substituted with a R^{b} group; R^{a}, when present, is H or (C₁-C₄)alkoxy; R^{b}, when present, is (C₁-C₄)alkyl; each occurrence of R^{d} and R^{e}, when present, is independently selected from halo and (C₁-C₄)alkoxy, and wherein the remaining variables are as described above for Formula I.

In further embodiments, R¹ in the compound of Formula I is (C₁-C₄)alkyl, halo(C₁-C₃)alkyl, 5-membered nitrogen containing heteroaryl, or phenyl, wherein said halo(C₁-C₃)alkyl is optionally substituted with a OR^{a} group, wherein said 5-membered nitrogen containing heteroaryl is optionally substituted with a (C₁-C₄)alkyl group, and wherein the remaining variables are as described above for Formula I or the second embodiment.

In further embodiments, R¹ is methyl, ethyl, —CF₃, —CH₂CF₃, 1,1,1-trifluoropropanol-3-yl, 2-ethoxy-1,1,1-trifluoropropane-3-yl, phenyl, or pyrazolyl, wherein said pyrazolyl is optionally substituted with a methyl group, and wherein the remaining variables are as described above for Formula I or the second or third embodiment.

In further embodiments, the compound of Formula I is of the Formula II:

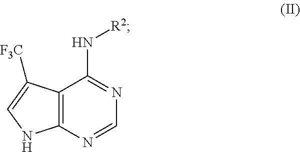

or a pharmaceutically acceptable salt thereof, wherein the variables are as described above for Formula I or the second embodiment.

In further embodiments, the compound of Formula I is of the Formula III:

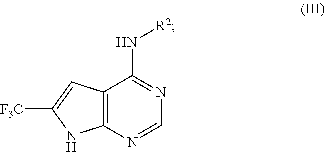

or a pharmaceutically acceptable salt thereof, wherein the variables are as described above for Formula I or the second embodiment.

In further embodiments, R² in the compound of Formula I, II, or III is (C₁-C₄)alkyl, benzofuranyl, dihydro-TH-indenyl, or tetrahydronaphthalenyl, wherein said (C₁-C₄)alkyl is optionally substituted with a R^{c} group, wherein said benzofuranyl, dihydro-TH-indenyl, and tetrahydronaphthalenyl are each optionally and independently substituted with 1 to 3 groups independently selected from R^{d}, and wherein the remaining variables are as described above for Formula I or the second, third, or fourth embodiment.

In further embodiments, each occurrence of R^{c}, when present, in the compound of Formula I, II, III is phenyl, cyclopropyl, pyridinyl, pyrazinyl, or pyrimidinyl, each of which are optionally and independently substituted with 1 to 2 groups independently selected from R^{e}, and wherein the remaining variables are as described above for Formula I or the second, third, fourth, or sixth embodiment.

In further embodiments, each occurrence of R^{e}, when present, in the compound of Formula I, II, or III is chloro, fluoro, or methoxy, and wherein the remaining variables are as described above for Formula I or the second, third, fourth, sixth, or seventh embodiment.

In further embodiments, each occurrence of R^{d}, when present, in the compound of Formula I, II, or III is (C₁-C₄)alkoxy, and wherein the remaining variables are as described above for Formula I or the second, third, fourth, sixth, seventh, or eighth embodiment. Alternatively, each occurrence of R^{d}, when present, in the compound of Formula I, II, or III is methoxy, and wherein the remaining variables are as described above for Formula I or the second, third, fourth, sixth, seventh, or eighth embodiment.

In further embodiments, R² in the compound of Formula I, II, or III is (C₁-C₄)alkyl optionally substituted with phenyl or pyrimidine-5-yl, wherein said phenyl is optionally substituted with 1 to 2 independently selected halo groups, and wherein the remaining variables are as described above for Formula I or the second, third, fourth, sixth, or seventh embodiment.

In further embodiments, provided are compounds having a structure represented by a formula:

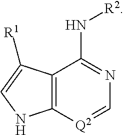

In further embodiments, provided are compounds having a structure represented by a formula:

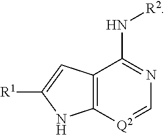

wherein R¹ is a 3- to 6-membered cycloalkyl or a C1-C6 haloalkyl, C1-C6 haloalkoxy, C1-C6 halohydroxyl. In some embodiments, Riis independently selected from: CCl₃, CF₃, or CBr₃.

In further embodiments, provided are compounds having a structure represented by a formula:

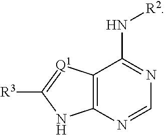

In further embodiments, provided are compounds having a structure represented by a formula:

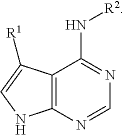

In further embodiments, provided are compounds having a structure represented by a formula:

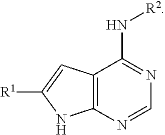

wherein R¹ is a 3- to 6-membered cycloalkyl or a C1-C6 haloalkyl, C1-C6 haloalkoxy, C1-C6 halohydroxyl. In some embodiments, Riis independently selected from: CCl₃, CF₃, or CBr₃.

In further embodiments, provided are compounds having a structure represented by a formula:

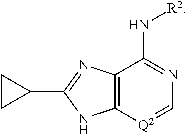

In further embodiments, provided are compounds having a structure represented by a formula:

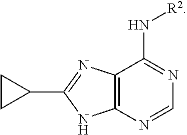

In further embodiments, provided are compounds having a structure represented by a formula:

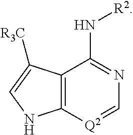

In further embodiments, provided are compounds having a structure represented by a formula:

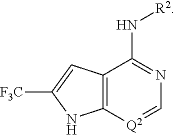

In further embodiments, provided are compounds having a structure represented by a formula:

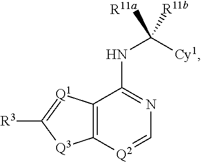

wherein each of R^{11a} and R^{11b} is independently selected from hydrogen, C1-C5 alkyl, and C1-C5 hydroxyalkyl; or wherein each of R^{11a} and R^{11b} together comprise a 3-membered cycloalkyl; and wherein Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino.

In further embodiments, provided are compounds having a structure represented by a formula:

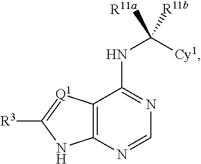

In further embodiments, provided are compounds having a structure represented by a formula selected from:

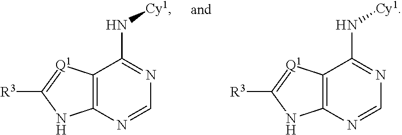

In further embodiments, provided are compounds having a structure represented by a formula:

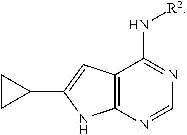

In further embodiments, provided are compounds having a structure represented by a formula:

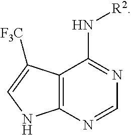

In further embodiments, provided are compounds having a structure represented by a formula:

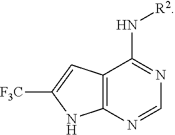

In further embodiments, Q¹ is N and R³ is a 3- to 6-membered cycloalkyl. In still further embodiments, Q¹ is N and R³ is a 3- to 4-membered cycloalkyl.

In further embodiments, Q¹ is CR¹ and R³ is hydrogen.

In further embodiments, Cy¹, when present, is a structure represented by a formula selected from:

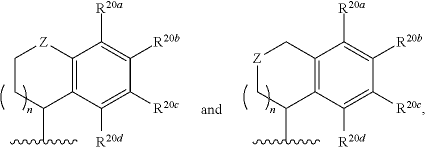

wherein Z is O or CH₂; wherein n is 0 or 1; and wherein each of R^{20a}, R^{20b}, R^{20c}, and R^{20d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino.

In further embodiments, Cy¹, when present, is a structure represented by a formula selected from:

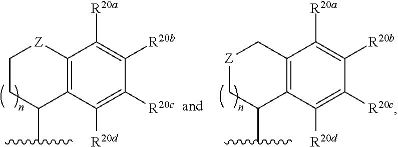

wherein Z is O, CH₂, or NR³⁰; wherein R³⁰, when present, is selected from —C(O)(C1-C4 alkyl), C1-C4 alkyl, and C2-C4 alkenyl; wherein n is 0 or 1; and wherein each of R^{20a}, R^{20b}, R^{20c}, and R^{20d} is independently selected from hydrogen, halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino.

In further embodiments, the compound has a structure represented by a formula:

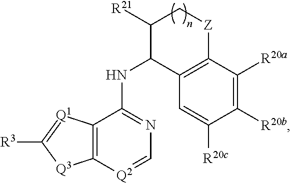

wherein Z is O, CH₂, or NR³⁰; wherein R³⁰, when present, is selected from —C(O)(C1-C4 alkyl), C1-C4 alkyl, and C2-C4 alkenyl; wherein n is 0 or 1; wherein each of R^{20a}, R^{20b}, R^{20c}, and R^{20d} is independently selected from hydrogen, halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and wherein R²¹ is selected from hydrogen, halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino.

In further embodiments, the compound has a structure represented by a formula selected from:

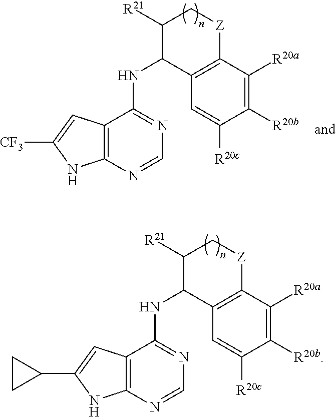

In further embodiments, the compound has a structure represented by a formula:

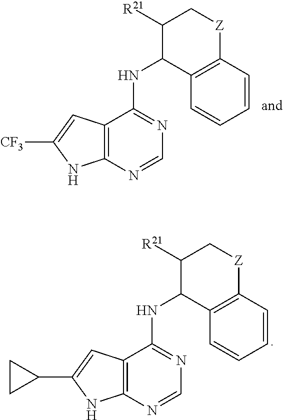

In further embodiments, the compound has a structure selected from:

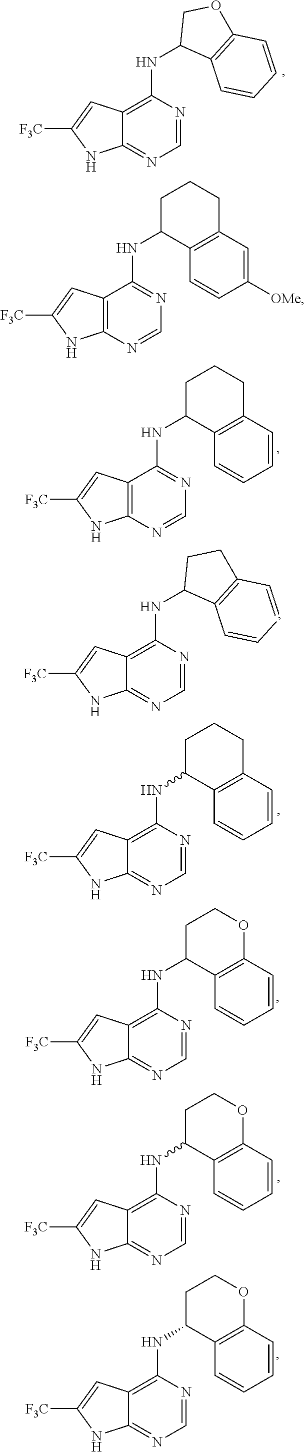

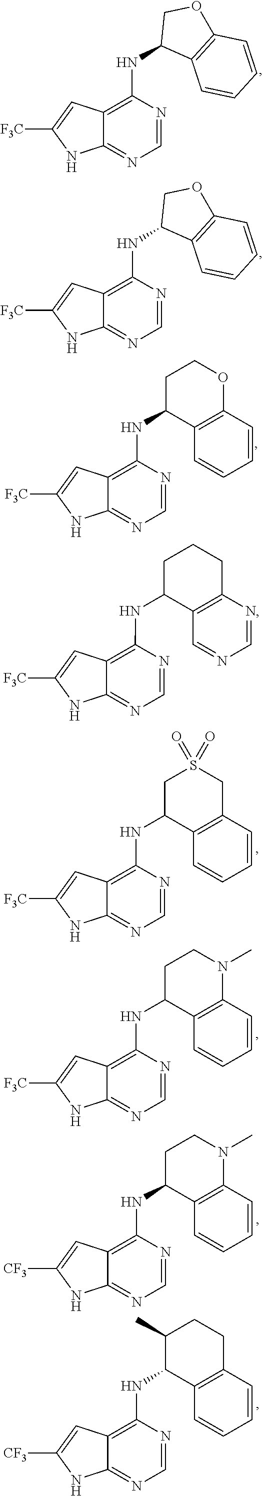

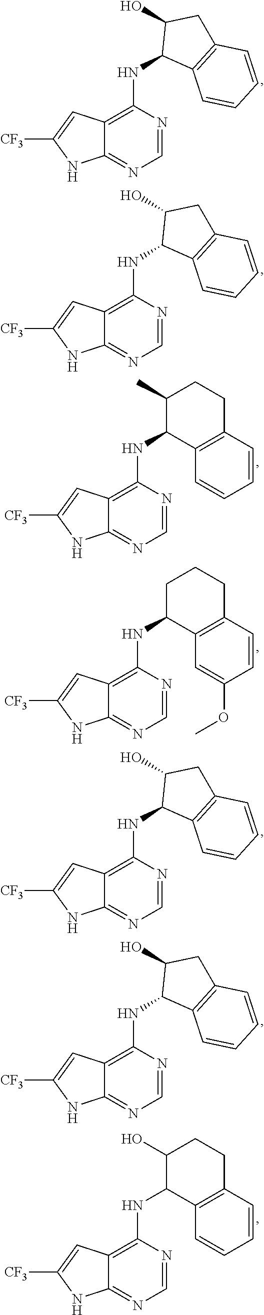

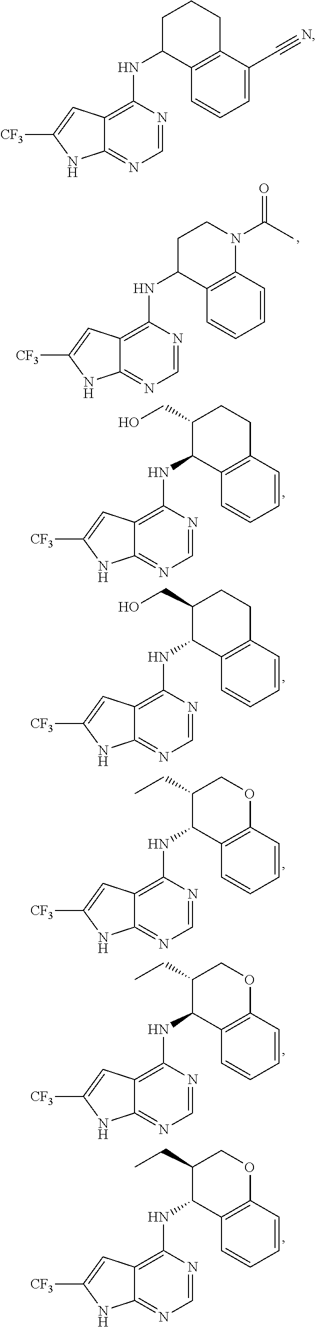

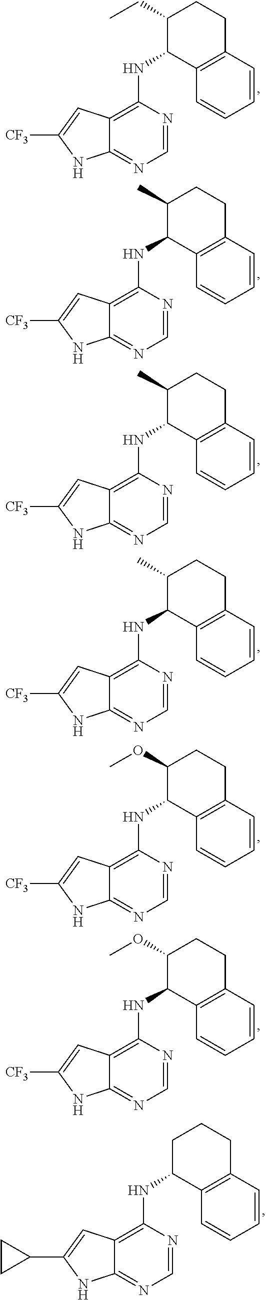

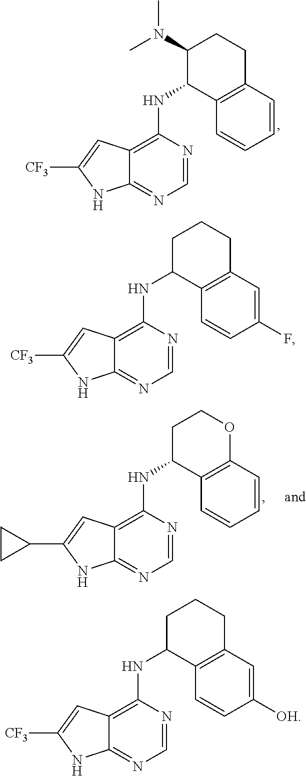

In further embodiments, the compound has a structure:

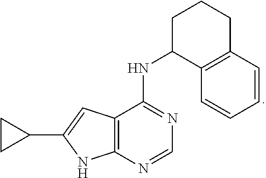 [SMILES: c1ccc2c(c1)CCCC2Nc1ncnc2[nH]c(C3CC3)cc12]

In further embodiments, the compound has a structure selected from:

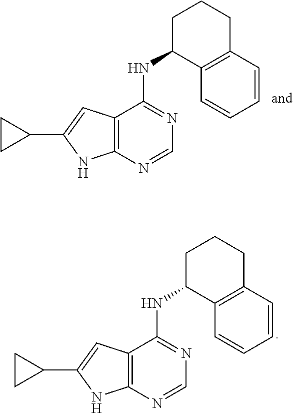 [SMILES: c1ccc2c(c1)CCC[C@@H]2Nc1ncnc2[nH]c(C3CC3)cc12 ; c1ccc2c(c1)CCC[C@H]2Nc1ncnc2[nH]c(C3CC3)cc12]

In further embodiments, the compound has a structure:

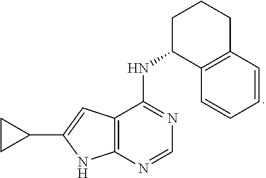 [SMILES: c1ccc2c(c1)CCC[C@H]2Nc1ncnc2[nH]c(C3CC3)cc12]

Thus, in some embodiments, n is 0 or 1. In further embodiments, n is 0. In still further embodiments, n is 1.

Specific examples of compounds are provided in the EXEMPLIFICATION section and are included herein. Pharmaceutically acceptable salts as well as the neutral forms of these compounds are also included.

a. Q¹, Q², and Q³ Groups

In some embodiments, Q¹ is N. In some embodiments, Q¹ is CR¹.

In some embodiments, Q² is CH or N. In further embodiments, Q² is CH. In still further embodiments, Q² is NH.

In some embodiments, Q³ is CH₂ or NH. In further embodiments, Q³ is CH₂. In further embodiments, Q³ is NH.

b. Z Groups

In some embodiments, Z is O, CH₂, or NR³⁰. In further embodiments, Z is O or CH₂. In still further embodiments, Z is O or NR³⁰. In yet further embodiments, Z is CH₂ or NR³⁰. In even further embodiments, Z is O. In still further embodiments, Z is CH₂. In yet further embodiments, Z is NR³⁰.

c. R^{a} Groups

In some embodiments, R^{a}, when present, is H, (C₁-C₄)alkyl, or (C₁-C₄)alkoxy. In further embodiments, R^{a}, when present, is H, methyl, ethyl, n-propyl, isopropyl, methoxy, ethoxy, n-propoxy, or isopropoxy. In still further embodiments, R^{a}, when present, is H, methyl, ethyl, methoxy, or ethoxy. In yet further embodiments, R^{a}, when present, is H, methyl, or methoxy.

In further embodiments, R^{a}, when present, is H.

In various embodiments, R^{a}, when present, is H or (C₁-C₄)alkyl. In further embodiments, R^{a}, when present, is H, methyl, ethyl, n-propyl, or isopropyl. In still further embodiments, R^{a}, when present, is H, methyl, or ethyl. In yet further embodiments, R^{a}, when present, is H or ethyl. In still further embodiments, R^{a}, when present, is H or methyl.

In various embodiments, R^{a}, when present, is (C₁-C₄)alkyl. In further embodiments, R^{a}, when present, is methyl, ethyl, n-propyl, or isopropyl. In still further embodiments, R^{a}, when present, is methyl or ethyl. In yet further embodiments, R^{a}, when present, is ethyl. In still further embodiments, R^{a}, when present, is methyl.

d. R^{b} Groups

In some embodiments, each occurrence of R^{b}, when present, is halo, halo(C₁-C₄)alkyl, (C₁-C₄)alkoxy, or halo(C₁-C₄)alkoxy. In further embodiments, each occurrence of R^{b}, when present, is —F, —Cl, —CF₃, —CHF₂, —CH₂F, —CH₂CF₃, —CH₂CHF₂, —CH₂CH₂F, —CH(CH₃)CH₂F, —CH₂CH₂CH₂F, —CCl₃, —CHCl₂, —CH₂Cl, —CH₂CCl₃, —CH₂CHCl₂, —CH₂CH₂Cl, —CH(CH₃)CH₂Cl, —CH₂CH₂CH₂Cl, methoxy, ethoxy, n-propoxy, isopropoxy, —OCF₃, —OCHF₂, —OCH₂F, —OCH₂CH₂F, —OCH(CH₃)CH₂F, —OCH₂CH₂CH₂F, —OCCl₃, —OCHCl₂, —OCH₂Cl, —OCH₂CH₂Cl, —OCH(CH₃)CH₂Cl, or —OCH₂CH₂CH₂Cl. In still further embodiments, each occurrence of R^{b}, when present, is —F, —Cl, —CH₂F, —CH₂CH₂F, —CH₂Cl, —CH₂CH₂Cl, methoxy, ethoxy, —OCF₃, —OCHF₂, —OCH₂F, —OCH₂CH₂F, —OCCl₃, —OCHCl₂, —OCH₂Cl, or —OCH₂CH₂Cl. In yet further embodiments, each occurrence of R^{b}, when present, is —F, —Cl, —CH₂F, —CH₂Cl, methoxy, —OCF₃, —OCHF₂, —OCH₂F, —OCCl₃, —OCHCl₂, or —OCH₂Cl.

In various embodiments, each occurrence of R^{b}, when present, is halo or halo(C₁-C₄)alkyl. In further embodiments, R^{b} is —F, —Cl, —CH₂F, —CH₂CH₂F, —CH(CH₃)CH₂F, —CH₂CH₂CH₂F, —CH₂Cl, —CH₂CH₂Cl, —CH(CH₃)CH₂Cl, or —CH₂CH₂CH₂Cl. In still further embodiments, each occurrence of R^{b}, when present, is —F, —Cl, —CH₂F, —CH₂CH₂F, —CH₂Cl, or —CH₂CH₂Cl. In yet further embodiments, each occurrence of R^{b}, when present, is —F, —Cl, —CH₂F, or —CH₂Cl.

In various embodiments, each occurrence of R^{b}, when present, is (C₁-C₄)alkoxy or halo(C₁-C₄)alkoxy. In further embodiments, each occurrence of R^{b}, when present, is methoxy, ethoxy, n-propoxy, isopropoxy, —OCF₃, —OCHF₂, —OCH₂F, —OCH₂CH₂F, —OCH(CH₃)CH₂F, —OCH₂CH₂CH₂F, —OCCl₃, —OCHCl₂, —OCH₂Cl, —OCH₂CH₂Cl, —OCH(CH₃)CH₂Cl, or —OCH₂CH₂CH₂Cl. In still further embodiments, each occurrence of R^{b}, when present, is methoxy, ethoxy, —OCF₃, —OCHF₂, —OCH₂F, —OCH₂CH₂F, —OCCl₃, —OCHCl₂, —OCH₂Cl, or —OCH₂CH₂Cl. In yet further embodiments, each occurrence of R^{b}, when present, is methoxy, —OCF₃, —OCHF₂, —OCH₂F, —OCCl₃, —OCHCl₂, or —OCH₂Cl.

In various embodiments, each occurrence of R^{b}, when present, is halo. In further embodiments, each occurrence of R^{b}, when present, is —F, —Cl, or —Br. In still further embodiments, each occurrence of R^{b}, when present, is —F or —Cl. In yet further embodiments, each occurrence of R^{b}, when present, is —F. In an even further embodiment, each occurrence of R^{b}, when present, is —Cl.

e. R^{c} Groups

In some embodiments, each occurrence of R^{c}, when present, is phenyl, 3- or 4-membered cycloalkyl, or 5- or 6-membered heteroaryl, wherein said phenyl and 5- or 6-membered heteroaryl are each optionally and independently substituted with 1 to 3 groups independently selected from R^{e}. In further embodiments, each occurrence of R^{c}, when present, is phenyl, 3- or 4-membered cycloalkyl, or 5- or 6-membered heteroaryl, wherein said phenyl and 5- or 6-membered heteroaryl are each optionally and independently substituted with 1 to 2 groups independently selected from R^{e}. In still further embodiments, each occurrence of R^{c}, when present, is phenyl, 3- or 4-membered cycloalkyl, or 5- or 6-membered heteroaryl, wherein said phenyl and 5- or 6-membered heteroaryl are each optionally and monosubstituted with a group selected from R^{e}. In yet further embodiments, each occurrence of R^{c}, when present, is phenyl, 3- or 4-membered cycloalkyl, or 5- or 6-membered heteroaryl, wherein said phenyl and 5- or 6-membered heteroaryl are each unsubstituted.

In various embodiments, each occurrence of R^{c}, when present, is phenyl optionally substituted with 1 to 3 groups independently selected from R^{e}. In further embodiments, each occurrence of R^{c}, when present, is phenyl optionally substituted with 1 to 2 groups independently selected from R^{e}. In still further embodiments, each occurrence of R^{c}, when present, is phenyl optionally monosubstituted with a group selected from R^{e}. In yet further embodiments, each occurrence of R^{c}, when present, is unsubstituted phenyl.

In various embodiments, each occurrence of R^{c}, when present, is 3- or 4-membered cycloalkyl. In further embodiments, each occurrence of R^{c}, when present, is 3-membered cycloalkyl. In still further embodiments, each occurrence of R^{c}, when present, is 4-membered cycloalkyl. In yet further embodiments, each occurrence of R^{c}, when present, is - or 4-membered cycloalkyl, and is unsubstituted.

In various embodiments, each occurrence of R^{c}, when present, is 5- or 6-membered heteroaryl optionally substituted with 1 to 3 groups independently selected from R^{e}. Examples of 5- or 6-membered heteroaryls include, but are not limited to, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, and pyrazinyl. In further embodiments, each occurrence of R^{c}, when present, is 5- or 6-membered heteroaryl optionally substituted with 1 to 2 groups independently selected from R^{e}. In still further embodiments, each occurrence of R^{c}, when present, is 5- or 6-membered heteroaryl optionally monosubstituted with a group selected from R^{e}. In yet further embodiments, each occurrence of R^{c}, when present, is unsubstituted 5- or 6-membered heteroaryl.

In various embodiments, each occurrence of R^{c}, when present, is 5-membered heteroaryl optionally substituted with 1 to 3 groups independently selected from R^{e}. In further embodiments, each occurrence of R^{c}, when present, is 5-membered heteroaryl optionally substituted with 1 to 2 groups independently selected from R^{e}. In still further embodiments, each occurrence of R^{e}, when present, is 5-membered heteroaryl optionally monosubstituted with a group selected from R^{e}. In yet further embodiments, each occurrence of R^{e}, when present, is unsubstituted 5-membered heteroaryl.

In various embodiments, each occurrence of R^{c}, when present, is 6-membered heteroaryl optionally substituted with 1 to 3 groups independently selected from R^{e}. In further embodiments, each occurrence of R^{c}, when present, is 6-membered heteroaryl optionally substituted with 1 to 2 groups independently selected from R^{e}. In still further embodiments, each occurrence of R^{c}, when present, is 6-membered heteroaryl optionally monosubstituted with a group selected from R^{e}. In yet further embodiments, each occurrence of R^{c}, when present, is unsubstituted 6-membered heteroaryl.

In various embodiments, each occurrence of R^{c}, when present, is pyridinyl, pyrimidinyl, or pyrazinyl, and is optionally substituted with 1 to 3 groups independently selected from R^{e}. In further embodiments, each occurrence of R^{c}, when present, is pyridinyl, pyrimidinyl, or pyrazinyl, and is optionally substituted with 1 to 2 groups independently selected from R^{e}. In still further embodiments, each occurrence of R^{e}, when present, is pyridinyl, pyrimidinyl, or pyrazinyl, and is optionally monosubstituted with a group selected from R^{e}. In yet further embodiments, each occurrence of R^{e}, when present, is pyridinyl, pyrimidinyl, or pyrazinyl, and is unsubstituted.

f. R^{d} and R^{e} Groups

In some embodiments, each occurrence of R^{d} and R^{e}, when present, is independently halo, halo(C₁-C₄)alkyl, (C₁-C₄)alkoxy, or halo(C₁-C₄)alkoxy. In further embodiments, each occurrence of R^{d} and R^{e}, when present, is independently —F, —Cl, —CH₂F, —CH₂CH₂F, —CH(CH₃)CH₂F, —CH₂CH₂CH₂F, —CH₂Cl, —CH₂CH₂Cl, —CH(CH₃)CH₂Cl, —CH₂CH₂CH₂Cl, methoxy, ethoxy, n-propoxy, isopropoxy, —OCF₃, —OCHF₂, —OCH₂F, —OCH₂CH₂F, —OCH(CH₃)CH₂F, —OCH₂CH₂CH₂F, —OCCl₃, —OCHCl₂, —OCH₂Cl, —OCH₂CH₂Cl, —OCH(CH₃)CH₂Cl, or —OCH₂CH₂CH₂Cl. In still further embodiments, each occurrence of R^{d} and R^{e}, when present, is independently —F, —Cl, —CH₂F, —CH₂CH₂F, —CH₂Cl, —CH₂CH₂Cl, methoxy, ethoxy, —OCF₃, —OCHF₂, —OCH₂F, —OCH₂CH₂F, —OCCl₃, —OCHCl₂, —OCH₂Cl, or —OCH₂CH₂Cl. In yet further embodiments, each occurrence of R^{d} and R^{e}, when present, is independently —F, —Cl, —CH₂F, —CH₂Cl, methoxy, —OCF₃, —OCHF₂, —OCH₂F, —OCCl₃, —OCHCl₂, or —OCH₂Cl.

In various embodiments, each occurrence of R^{d} and R^{e}, when present, is independently halo or halo(C₁-C₄)alkyl. In further embodiments, each occurrence of R^{d} and R^{e}, when present, is independently —F, —Cl, —CH₂F, —CH₂CH₂F, —CH(CH₃)CH₂F, —CH₂CH₂CH₂F, —CH₂Cl, —CH₂CH₂Cl, —CH(CH₃)CH₂Cl, or —CH₂CH₂CH₂Cl. In still further embodiments, each occurrence of R^{d} and R^{e}, when present, is independently —F, —Cl, —CH₂F, —CH₂CH₂F, —CH₂Cl, or —CH₂CH₂Cl. In yet further embodiments, each occurrence of R^{d} and R^{e}, when present, is independently —F, —Cl, —CH₂F, or —CH₂Cl.

In various embodiments, each occurrence of R^{d} and R^{e}, when present, is independently (C₁-C₄)alkoxy or halo(C₁-C₄)alkoxy. In further embodiments each occurrence of R^{d} and R^{e}, when present, is independently methoxy, ethoxy, n-propoxy, isopropoxy, —OCF₃, —OCHF₂, —OCH₂F, —OCH₂CH₂F, —OCH(CH₃)CH₂F, —OCH₂CH₂CH₂F, —OCCl₃, —OCHCl₂, —OCH₂Cl, —OCH₂CH₂Cl, —OCH(CH₃)CH₂Cl, or —OCH₂CH₂CH₂Cl. In still further embodiments, each occurrence of R^{d} and R^{e}, when present, is independently methoxy, ethoxy, —OCF₃, —OCHF₂, —OCH₂F, —OCH₂CH₂F, —OCCl₃, —OCHCl₂, —OCH₂Cl, or —OCH₂CH₂Cl. In yet further embodiments, each occurrence of R^{d} and R^{e}, when present, is independently methoxy, —OCF₃, —OCHF₂, —OCH₂F, —OCCl₃, —OCHCl₂, or —OCH₂Cl.

In various embodiments, each occurrence of R^{d} and R^{e}, when present, is independently halo. In further embodiments, each occurrence of R^{d} and R^{e}, when present, is independently —F, —Cl, or —Br. In still further embodiments, each occurrence of R^{d} and R^{e}, when present, is independently —F or —Cl. In yet further embodiments, each occurrence of R^{d} and R^{e}, when present, is —F. In an even further embodiment, each occurrence of R^{d} and R^{e}, when present, is —Cl.

g. R¹ Groups

In some embodiments, R¹ is C1-C6 haloalkyl, C1-C6 haloalkoxy, C1-C6 halohydroxy, or a structure represented by a formula:

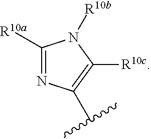

In further embodiments, R¹ is C1-C3 haloalkyl, C1-C3 haloalkoxy, C1-C3 halohydroxy, or a structure represented by a formula:

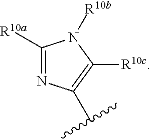

In still further embodiments, R¹ is —CF₃, —CHF₂, —CH₂F, —CH₂CF₃, —CH₂CHF₂, —CH₂CH₂F, —CCl₃, —CHCl₂, —CH₂Cl, —CH₂CCl₃, —CH₂CHCl₂, —CH₂CH₂Cl, —CH(OCH₃)CF₃, —CH(OCH₃)CHF₂, —CH(OCH₃)CH₂F, —CH(OCH₃)CCl₃, —CH(OCH₃)CHCl₂, —CH(OCH₃)CH₂Cl, —CH(OH)CF₃, —CH(OH)CHF₂, —CH(OH)CH₂F, —CH(OH)CCl₃, —CH(OH)CHCl₂, —CH(OH)CH₂Cl, or a structure represented by a formula:

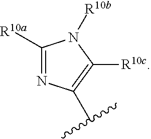

In yet further embodiments, R¹ is —CF₃, —CHF₂, —CH₂F, —CCl₃, —CHCl₂, —CH₂Cl, or a structure represented by a formula:

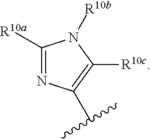

In various embodiments, R¹ is a structure represented by a formula:

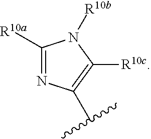

In various embodiments, R¹ is C1-C6 haloalkyl, C1-C6 haloalkoxy, or C1-C6 halohydroxy. In further embodiments, R¹ is C1-C3 haloalkyl, C1-C3 haloalkoxy, or C1-C3 halohydroxy. In still further embodiments, R¹ is —CF₃, —CHF₂, —CH₂F, —CH₂CF₃, —CH₂CHF₂, —CH₂CH₂F, —CCl₃, —CHCl₂, —CH₂Cl, —CH₂CCl₃, —CH₂CHCl₂, —CH₂CH₂Cl, —CH(OCH₃)CF₃, —CH(OCH₃)CHF₂, —CH(OCH₃)CH₂F, —CH(OCH₃)CCl₃, —CH(OCH₃)CHCl₂, —CH(OCH₃)CH₂Cl, —CH(OH)CF₃, —CH(OH)CHF₂, —CH(OH)CH₂F, —CH(OH)CCl₃, —CH(OH)CHCl₂, or —CH(OH)CH₂Cl. In yet further embodiments, R¹ is —CF₃, —CHF₂, —CH₂F, —CCl₃, —CHCl₂, or —CH₂Cl.

In various embodiments, R¹ is C1-C6 haloalkoxy or C1-C6 halohydroxy. In further embodiments, R¹ is —CH(OCH₂CH₃)CF₃, —CH(OCH₂CH₃)CHF₂, —CH(OCH₂CH₃)CH₂F, —CH(OCH₂CH₃)CCl₃, —CH(OCH₂CH₃)CHCl₂, —CH(OCH₂CH₃)CH₂Cl, —CH₂CH(OH)CF₃, —CH₂CH(OH)CHF₂, —CH₂CH(OH)CH₂F, —CH₂CH(OH)CCl₃, —CH₂CH(OH)CHCl₂, or —CH₂CH(OH)CH₂Cl. In still further embodiments, R¹ is —CH(OCH₃)CF₃, —CH(OCH₃)CHF₂, —CH(OCH₃)CH₂F, —CH(OCH₃)CCl₃, —CH(OCH₃)CHCl₂, —CH(OCH₃)CH₂Cl, —CH(OH)CF₃, —CH(OH)CHF₂, —CH(OH)CH₂F, —CH(OH)CCl₃, —CH(OH)CHCl₂, or —CH(OH)CH₂Cl. In yet further embodiments, —CH(OCH₂CH₃)CF₃ or —CH(OH)CF₃.

In various embodiments, R¹ is C1-C6 haloalkyl. In further embodiments, R¹ is C1-C3 haloalkyl. In still further embodiments, R¹ is —CF₃, —CHF₂, —CH₂F, —CH₂CF₃, —CH₂CHF₂, —CH₂CH₂F, —CCl₃, —CHCl₂, —CH₂Cl, —CH₂CCl₃, —CH₂CHCl₂, or —CH₂CH₂Cl. In yet further embodiments, R¹ is —CF₃, —CHF₂, —CH₂F, —CCl₃, —CHCl₂, or —CH₂Cl. In an even further embodiment, R¹ is —CF₃ or —CCl₃. In still further embodiments, R¹ is —CF₃.

In some embodiments, R¹ is (C₁-C₆)alkyl, halo(C₁-C₄)alkyl, (C₁-C₄)alkoxy, halo(C₁-C₄)alkoxy, 5- or 6-membered heteroaryl, or phenyl, wherein said (C₁-C₆)alkyl and halo(C₁-C₄)alkyl are each optionally and independently substituted with a OR^{a} group, and wherein said phenyl and 5- or 6-membered heteroaryl are each optionally and independently substituted with 1 to 3 groups independently selected from R^{b}.

In various embodiments, R¹ is (C₁-C₆)alkyl, halo(C₁-C₄)alkyl, (C₁-C₄)alkoxy, halo(C₁-C₄)alkoxy, 5- or 6-membered heteroaryl, or phenyl. In further embodiments, R¹ is methyl, ethyl, n-propyl, isopropyl, —CF₃, —CHF₂, —CH₂F, —CH₂CF₃, —CH₂CHF₂, —CH₂CH₂F, —CH(CH₃)CH₂F, —CH₂CH₂CH₂F, —CCl₃, —CHCl₂, —CH₂Cl, —CH₂CCl₃, —CH₂CHCl₂, —CH₂CH₂Cl, —CH(CH₃)CH₂Cl, —CH₂CH₂CH₂Cl, methoxy, ethoxy, n-propoxy, isopropoxy, —OCF₃, —OCHF₂, —OCH₂F, —OCH₂CH₂F, —OCH(CH₃)CH₂F, —OCH₂CH₂CH₂F, —OCCl₃, —OCHCl₂, —OCH₂Cl, —OCH₂CH₂Cl, —OCH(CH₃)CH₂Cl, —OCH₂CH₂CH₂Cl, 5- or 6-membered heteroaryl, or phenyl. In still further embodiments, R¹ is methyl, ethyl, —CF₃, —CHF₂, —CH₂F, —CH₂CF₃, —CH₂CHF₂, —CH₂CH₂F, —CCl₃, —CHCl₂, —CH₂Cl, —CH₂CCl₃, —CH₂CHCl₂, —CH₂CH₂Cl, methoxy, ethoxy, —OCF₃, —OCHF₂, —OCH₂F, —OCH₂CH₂F, —OCCl₃, —OCHCl₂, —OCH₂Cl, —OCH₂CH₂Cl, 5- or 6-membered heteroaryl, or phenyl. In yet further embodiments, R¹ is methyl, —CF₃, —CHF₂, —CH₂F, —CCl₃, —CHCl₂, —CH₂Cl, methoxy, —OCF₃, —OCHF₂, —OCH₂F, —OCCl₃, —OCHCl₂, —OCH₂Cl, 5- or 6-membered heteroaryl, or phenyl.

In various embodiments, R¹ is (C₁-C₆)alkyl, halo(C₁-C₄)alkyl, (C₁-C₄)alkoxy, or halo(C₁-C₄)alkoxy. In further embodiments, R¹ is methyl, ethyl, n-propyl, isopropyl, —CF₃, —CHF₂, —CH₂F, —CH₂CF₃, —CH₂CHF₂, —CH₂CH₂F, —CH(CH₃)CH₂F, —CH₂CH₂CH₂F, —CCl₃, —CHCl₂, —CH₂Cl, —CH₂CCl₃, —CH₂CHCl₂, —CH₂CH₂Cl, —CH(CH₃)CH₂Cl, —CH₂CH₂CH₂Cl, methoxy, ethoxy, n-propoxy, isopropoxy, —OCF₃, —OCHF₂, —OCH₂F, —OCH₂CH₂F, —OCH(CH₃)CH₂F, —OCH₂CH₂CH₂F, —OCCl₃, —OCHCl₂, —OCH₂Cl, —OCH₂CH₂Cl, —OCH(CH₃)CH₂Cl, or —OCH₂CH₂CH₂Cl. In still further embodiments, R¹ is methyl, ethyl, —CF₃, —CHF₂, —CH₂F, —CH₂CF₃, —CH₂CHF₂, —CH₂CH₂F, —CCl₃, —CHCl₂, —CH₂Cl, —CH₂CCl₃, —CH₂CHCl₂, —CH₂CH₂Cl, methoxy, ethoxy, —OCF₃, —OCHF₂, —OCH₂F, —OCH₂CH₂F, —OCCl₃, —OCHCl₂, —OCH₂Cl, or —OCH₂CH₂Cl. In yet further embodiments, RI is methyl, —CF₃, —CHF₂, —CH₂F, —CCl₃, —CHCl₂, —CH₂Cl, methoxy, —OCF₃, —OCHF₂, —OCH₂F, —OCCl₃, —OCHCl₂, or —OCH₂Cl.

In various embodiments, R¹ is (C₁-C₆)alkyl or halo(C₁-C₄)alkyl and is optionally and independently substituted with a OR^{a} group. In further embodiments, R¹ is (C₁-C₆)alkyl or halo(C₁-C₄)alkyl and is unsubstituted.

In various embodiments, R¹ is 5- or 6-membered heteroaryl, or phenyl. Examples of 5- or 6-membered heteroaryls include, but are not limited to, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, and pyrazinyl. Thus, in various embodiments, R¹ is 5-membered heteroaryl or phenyl. In further embodiments, R¹ is 6-membered heteroaryl or phenyl. In still further embodiments, R¹ is 5-membered heteroaryl. In yet further embodiments, R¹ is 6-membered heteroaryl. In an even further embodiment, R¹ is phenyl.

In various embodiments, R¹ is 5- or 6-membered heteroaryl, or phenyl, and is optionally and independently substituted with 1 to 3 groups independently selected from R^{b}. In further embodiments, R¹ is 5- or 6-membered heteroaryl, or phenyl, and is optionally and independently substituted with 1 to 2 groups independently selected from R^{b}. In still further embodiments, R¹ is 5- or 6-membered heteroaryl, or phenyl, and is optionally monosubstituted with a R^{b} group. In yet further embodiments, R¹ is 5- or 6-membered heteroaryl, or phenyl, and is unsubstituted.

In various embodiments, R¹ is (C₁-C₆)alkyl or (C₁-C₄)alkoxy. In further embodiments, R¹ is methyl, ethyl, n-propyl, isopropyl, methoxy, ethoxy, n-propoxy, or isopropoxy. In still further embodiments, R¹ is methyl, ethyl, methoxy, or ethoxy. In yet further embodiments, R¹ is methyl or methoxy.

In various embodiments, R¹ is halo(C₁-C₄)alkyl or halo(C₁-C₄)alkoxy. In further embodiments, R¹ is —CF₃, —CHF₂, —CH₂F, —CH₂CF₃, —CH₂CHF₂, —CH₂CH₂F, —CH(CH₃)CH₂F, —CH₂CH₂CH₂F, —CCl₃, —CHCl₂, —CH₂Cl, —CH₂CCl₃, —CH₂CHCl₂, —CH₂CH₂Cl, —CH(CH₃)CH₂Cl, —CH₂CH₂CH₂Cl, —OCF₃, —OCHF₂, —OCH₂F, —OCH₂CH₂F, —OCH(CH₃)CH₂F, —OCH₂CH₂CH₂F, —OCCl₃, —OCHCl₂, —OCH₂Cl, —OCH₂CH₂Cl, —OCH(CH₃)CH₂Cl, or —OCH₂CH₂CH₂Cl. In still further embodiments, R¹ is —CF₃, —CHF₂, —CH₂F, —CH₂CF₃, —CH₂CHF₂, —CH₂CH₂F, —CCl₃, —CHCl₂, —CH₂Cl, —CH₂CCl₃, —CH₂CHCl₂, —CH₂CH₂Cl, —OCF₃, —OCHF₂, —OCH₂F, —OCH₂CH₂F, —OCCl₃, —OCHCl₂, —OCH₂Cl, or —OCH₂CH₂Cl. In yet further embodiments, R¹ is —CF₃, —CHF₂, —CH₂F, —CCl₃, —CHCl₂, —CH₂Cl, —OCF₃, —OCHF₂, —OCH₂F, —OCCl₃, —OCHCl₂, or —OCH₂Cl.

In various embodiments, R¹ is halo(C₁-C₄)alkyl. In further embodiments, R¹ is —CF₃, —CHF₂, —CH₂F, —CH₂CF₃, —CH₂CHF₂, —CH₂CH₂F, —CH(CH₃)CH₂F, —CH₂CH₂CH₂F, —CCl₃, —CHCl₂, —CH₂Cl, —CH₂CCl₃, —CH₂CHCl₂, —CH₂CH₂Cl, —CH(CH₃)CH₂Cl, or —CH₂CH₂CH₂Cl. In still further embodiments, R¹ is —CF₃, —CHF₂, —CH₂F, —CH₂CF₃, —CH₂CHF₂, —CH₂CH₂F, —CCl₃, —CHCl₂, —CH₂Cl, —CH₂CCl₃, —CH₂CHCl₂, or —CH₂CH₂Cl. In yet further embodiments, R¹ is —CF₃, —CHF₂, —CH₂F, —CCl₃, —CHCl₂, or —CH₂Cl.

In various embodiments, R¹ is —CF₃ or —CH₂CF₃. In further embodiments, R¹ is —CH₂CF₃. In still further embodiments, R¹ is —CF₃.

h. R² Groups

In some embodiments, R² is C₁-C₆ alkyl, —CR^{11a}R^{11b}Cy¹, or Cy¹. In further embodiments, R² is C₁-C₃ alkyl, —CR^{11a}R^{11b}Cy¹, or Cy¹. In still further embodiments, R² is methyl, ethyl, —CR^{11a}R^{11b}Cy¹, or Cy¹. In yet further embodiments, R² is methyl, —CR^{11a}R^{11b}Cy¹, or Cy¹.

In further embodiments, R² is C₁-C₆ alkyl. In still further embodiments, R² is methyl, ethyl, n-propyl, isopropyl, n-butyl, isobutyl, sec-butyl, or tert-butyl. In yet further embodiments, R² is methyl, ethyl, n-propyl, or isopropyl. In an even further embodiment, R² is methyl or ethyl. In still further embodiments, R² is n-butyl.

In further embodiments, R² is —CR^{11a}R^{11b}Cy¹ or Cy¹. In still further embodiments, R² is —CR^{11a}R^{11b}Cy¹. In yet further embodiments, R² is —CH₂Cy¹, —CH(CH₃)Cy¹, or —C(CH₃)₂Cy¹. In an even further embodiment, R² is Cy¹.

In some embodiments, R² is (C₁-C₆)alkyl, 9-membered oxygen-containing fused heterocycle, or 9- to 10-membered carbocycle, wherein said (C₁-C₆)alkyl is optionally substituted with 1 or 2 groups independently selected from R^{c}, and wherein said 9-membered oxygen-containing fused heterocycle and 9- to 10-membered carbocycle are each optionally and independently substituted with 1 to 3 groups independently selected from R^{d}.

In various embodiments, R² is (C₁-C₄)alkyl, 9-membered oxygen-containing fused heterocycle, or 9- to 10-membered carbocycle. In further embodiments, R² is methyl, ethyl, n-propyl, isopropyl, 9-membered oxygen-containing fused heterocycle, or 9- to 10-membered carbocycle. In still further embodiments, R² is methyl, ethyl, 9-membered oxygen-containing fused heterocycle, or 9- to 10-membered carbocycle. In yet further embodiments, R² is methyl, 9-membered oxygen-containing fused heterocycle, or 9- to 10-membered carbocycle.

In various embodiments, R² is (C₁-C₆)alkyl optionally substituted with 1 or 2 groups independently selected from R^{c}. In further embodiments, R² is (C₁-C₆)alkyl optionally monosubstituted with a R^{c} group. In still further embodiments, R² is unsubstituted (C₁-C₆)alkyl.

In various embodiments, R² is (C₁-C₆)alkyl. In further embodiments, R² is (C₁-C₄)alkyl. In still further embodiments, R² is methyl, ethyl, n-propyl, or isopropyl. In yet further embodiments, R² is methyl or ethyl. In an even further embodiment, R² is ethyl. In still further embodiments, R² is methyl.

In various embodiments, R² is 9-membered oxygen-containing fused heterocycle or 9- to 10-membered carbocycle, and is optionally and independently substituted with 1 to 3 groups independently selected from R^{d}. In further embodiments, R² is 9-membered oxygen-containing fused heterocycle or 9- to 10-membered carbocycle, and is optionally and independently substituted with 1 to 2 groups independently selected from R^{d}. In still further embodiments, R² is 9-membered oxygen-containing fused heterocycle or 9- to 10-membered carbocycle, and is optionally monosubstituted with a R^{d} group. In yet further embodiments, R² is 9-membered oxygen-containing fused heterocycle or 9- to 10-membered carbocycle, and is unsubstituted.

In various embodiments, R² is 9-membered oxygen-containing fused heterocycle or 9- to 10-membered carbocycle. In further embodiments, R² is 9-membered oxygen-containing fused heterocycle. In still further embodiments, R² is 9- to 10-membered carbocycle. In yet further embodiments, R² is 9-membered carbocycle. In an even further embodiment, R² is 10-membered carbocycle.

i. R³ Groups

In some embodiments, R³ is a 3- to 6-membered cycloalkyl, C1-C6 haloalkyl, C1-C6 haloalkoxy, or C1-C6 halohydroxyalkyl. In further embodiments, R³ is a 3- to 6-membered cycloalkyl, C1-C4 haloalkyl, C1-C4 haloalkoxy, or C1-C4 halohydroxyalkyl. In still further embodiments, R³ is a 3- to 6-membered cycloalkyl, —CF₃, —CHF₂, —CH₂F, —CH₂CF₃, —CH₂CHF₂, —CH₂CH₂F, —CCl₃, —CHCl₂, —CH₂Cl, —CH₂CCl₃, —CH₂CHCl₂, —CH₂CH₂Cl, —OCF₃, —OCHF₂, —OCH₂F, —OCH₂CF₃, —OCH₂CHF₂, —OCH₂CH₂F, —OCCl₃, —OCHCl₂, —OCH₂Cl, —OCH₂CCl₃, —OCH₂CHCl₂, —OCH₂CH₂Cl, —CH(OH)CF₃, —CH(OH)CHF₂, —CH(OH)CH₂F, —CH(OH)CCl₃, —CH(OH)CHCl₂, or —CH(OH)CH₂Cl. In yet further embodiments, R³ is a 3- to 6-membered cycloalkyl,_-CF₃, —CHF₂, —CH₂F, —CCl₃, —CHCl₂, —CH₂Cl, —OCF₃, —OCHF₂, —OCH₂F, —OCCl₃, —OCHCl₂, or —OCH₂Cl.

In some embodiments, R³ is C1-C6 haloalkyl, C1-C6 haloalkoxy, or C1-C6 halohydroxyalkyl. In further embodiments, R³ is C1-C4 haloalkyl, C1-C4 haloalkoxy, or C1-C4 halohydroxyalkyl. In still further embodiments, R³ is —CF₃, —CHF₂, —CH₂F, —CH₂CF₃, —CH₂CHF₂, —CH₂CH₂F, —CCl₃, —CHCl₂, —CH₂Cl, —CH₂CCl₃, —CH₂CHCl₂, —CH₂CH₂Cl, —OCF₃, —OCHF₂, —OCH₂F, —OCH₂CF₃, —OCH₂CHF₂, —OCH₂CH₂F, —OCCl₃, —OCHCl₂, —OCH₂Cl, —OCH₂CCl₃, —OCH₂CHCl₂, —OCH₂CH₂Cl, —CH(OH)CF₃, —CH(OH)CHF₂, —CH(OH)CH₂F, —CH(OH)CCl₃, —CH(OH)CHCl₂, or —CH(OH)CH₂Cl. In yet further embodiments, R³ is —CF₃, —CHF₂, —CH₂F, —CCl₃, —CHCl₂, —CH₂Cl, —OCF₃, —OCHF₂, —OCH₂F, —OCCl₃, —OCHCl₂, or —OCH₂Cl.

In some embodiments, R³ is C1-C6 haloalkyl. In further embodiments, R³ is C1-C4 haloalkyl. In still further embodiments, R³ is —CF₃, —CHF₂, —CH₂F, —CH₂CF₃, —CH₂CHF₂, —CH₂CH₂F, —CCl₃, —CHCl₂, —CH₂Cl, —CH₂CCl₃, —CH₂CHCl₂, or —CH₂CH₂Cl. In yet further embodiments, R³ is —CF₃, —CHF₂, —CH₂F, —CCl₃, —CHCl₂, or —CH₂Cl.

In some embodiments, R³ is a 3- to 6-membered cycloalkyl. In further embodiments, R³ is a 3- to 5-membered cycloalkyl. In still further embodiments, R³ is a 3- to 4-membered cycloalkyl. In yet further embodiments, R³ is a 3-membered cycloalkyl. In an even further embodiment, R³ is a 4-membered cycloalkyl.

In some embodiments, R³ is hydrogen.

In some embodiments, R³ is hydrogen, halogen, (C₁-C₄)alkyl, or 3- to 6-membered cycloalkyl. In further embodiments, R³ is hydrogen.

In further embodiments, R³ is hydrogen, —F, —Cl, methyl, ethyl, n-propyl, isopropyl, or 3- to 6-membered cycloalkyl. In still further embodiments, R³ is hydrogen, —F, —Cl, methyl, ethyl, or 3- to 6-membered cycloalkyl. In yet further embodiments, R³ is hydrogen, —F, —Cl, methyl, or 3- to 6-membered cycloalkyl.

In further embodiments, R³ is hydrogen or (C₁-C₄)alkyl. In still further embodiments, R³ is hydrogen, methyl, ethyl, n-propyl, or isopropyl. In yet further embodiments, R³ is hydrogen, methyl, or ethyl. In an even further embodiment, R³ is hydrogen or ethyl. In still further embodiments, R³ is hydrogen or methyl.

In further embodiments, R³ is (C₁-C₄)alkyl. In still further embodiments, R³ is methyl, ethyl, n-propyl, or isopropyl. In yet further embodiments, R³ is methyl or ethyl. In an even further embodiment, R³ is ethyl. In still further embodiments, R³ is methyl.

In further embodiments, R³ is (C₁-C₄)alkyl. In still further embodiments, R³ is methyl, ethyl, n-propyl, isopropyl, halogenated methyl, halogenated ethyl, halogenated propyl, CF₃, CCl₃, or CBr₃. In yet further embodiments, R³ is methyl or ethyl. In an even further embodiment, R³ is ethyl. In still further embodiments, R³ is methyl. In still further embodiments, R³ is CF₃, CCl₃, or CBr₃.

In further embodiments, R³ is hydrogen or halogen. In still further embodiments, R³ is hydrogen, —F, —Cl, or —Br. In yet further embodiments, R³ is hydrogen, —F, or —Cl. In an even further embodiment, R³ is hydrogen or —F. In still further embodiments, R³ is hydrogen or —Cl.

In further embodiments, R³ is halogen. In still further embodiments, R³ is —F, —Cl, or —Br. In yet further embodiments, R³ is —F or —Cl. In an even further embodiment, R³ is —F. In still further embodiments, R³ is —Cl.

In further embodiments, R³ is hydrogen or 3- to 6-membered cycloalkyl. In still further embodiments, R³ is hydrogen, cyclopropyl, cyclobutyl, or cyclopentyl. In yet further embodiments, R³ is hydrogen, cyclopropyl, or cyclobutyl. In an even further embodiment, R³ is hydrogen or cyclopropyl. In some embodiments, R³ is not a methyl, ethyl or butyl. In some embodiments, R³ is not an acyclic alkyl chain comprising from about 1 to about 5 substituted or unsubstituted carbons.

In further embodiments, R³ is 3- to 6-membered cycloalkyl. In still further embodiments, R³ is 3- to 5-membered cycloalkyl. In yet further embodiments, R³ is 3- to 4-membered cycloalkyl. In an even further embodiment, R³ is cyclohexyl. In still further embodiments, R³ is cyclopentyl. In yet further embodiments, R³ is cyclobutyl. In an even further embodiment, R³ is cyclopropyl.

j. R^{10a}, R^{10b}, and R^{10c} Groups (R¹⁰ Groups)

In some embodiments, each of R^{10a}, R^{10b}, and R^{10c}, when present, is independently selected from hydrogen and C1-C4 alkyl. In further embodiments, each of R^{10a}, R^{10b}, and R^{10c}, when present, is independently selected from hydrogen, methyl, ethyl, n-propyl, and isopropyl. In still further embodiments, each of R^{10a}, R^{10b}, and R^{10c}, when present, is independently selected from hydrogen, methyl, and ethyl. In yet further embodiments, each of R^{10a}, R^{10b}, and R^{10c}, when present, is independently selected from hydrogen and methyl.

In some embodiments, each occurrence of R¹⁰, when present, is independently hydrogen or (C₁-C₄)alkyl. In further embodiments, each occurrence of R¹⁰, when present, is hydrogen.

In further embodiments, each occurrence of R¹⁰, when present, is independently hydrogen, methyl, ethyl, n-propyl, or isopropyl. In still further embodiments, each occurrence of R¹⁰, when present, is independently hydrogen, methyl, or ethyl. In yet further embodiments, each occurrence of R¹⁰, when present, is independently hydrogen or ethyl. In an even further embodiment, each occurrence of R¹⁰, when present, is independently hydrogen or methyl.

In further embodiments, each occurrence of R¹⁰, when present, is (C₁-C₄)alkyl. In an even further embodiment, each occurrence of R¹⁰, when present, is independently methyl, ethyl, n-propyl, or isopropyl. In still further embodiments, each occurrence of R¹⁰, when present, is independently methyl or ethyl. In yet further embodiments, each occurrence of R¹⁰, when present, is ethyl. In an even further embodiment, each occurrence of R¹⁰, when present, is methyl.

k. R^{11a} and R^{11b} Groups (R¹¹ Groups)

In some embodiments, each of R^{11a} and R^{11b}, when present, is independently selected from hydrogen, C1-C5 alkyl, and C1-C5 hydroxyalkyl. In further embodiments, each of R^{11a} and R^{11b}, when present, is independently selected from hydrogen, methyl, ethyl, n-propyl, isopropyl, n-butyl, isobutyl, sec-butyl, tert-butyl, —CH₂OH, —CH₂CH₂OH, —CH(CH₃)CH₂OH, —CH₂CH₂CH₂OH, —CH(CH₃)CH₂CH₂OH, —CH₂CH(CH₃)CH₂OH, —CH₂CH₂CH₂CH₂OH, and —C(CH₃)₂CH₂OH. In still further embodiments, each of R^{11a} and R^{11b}, when present, is independently selected from hydrogen, methyl, ethyl, n-propyl, isopropyl, —CH₂OH, —CH₂CH₂OH, —CH(CH₃)CH₂OH, and —CH₂CH₂CH₂OH. In yet further embodiments, each of R^{11a} and R^{11b}, when present, is independently selected from hydrogen, methyl, ethyl, —CH₂OH, and —CH₂CH₂OH. In an even further embodiment, each of R^{11a} and R^{11b}, when present, is independently selected from hydrogen, methyl, and —CH₂OH. In still further embodiments, each of R^{11a} and R^{11b}, when present, is hydrogen.

In some embodiments, each of R^{11a} and R^{11b}, when present, is independently selected from hydrogen and C1-C5 alkyl. In further embodiments, each of R^{11a} and R^{11b}, when present, is independently selected from hydrogen, methyl, ethyl, n-propyl, isopropyl, n-butyl, isobutyl, sec-butyl, and tert-butyl. In still further embodiments, each of R^{11a} and R^{11b}, when present, is independently selected from hydrogen, methyl, ethyl, n-propyl, and isopropyl. In yet further embodiments, each of R^{11a} and R^{11b}, when present, is independently selected from hydrogen, methyl, and ethyl. In an even further embodiment, each of R^{11a} and R^{11b}, when present, is independently selected from hydrogen and methyl.

In some embodiments, each of R^{11a} and R^{11b}, when present, is independently selected from hydrogen and C1-C5 hydroxyalkyl. In further embodiments, each of R^{11a} and R^{11b}, when present, is independently selected from hydrogen, —CH₂OH, —CH₂CH₂OH, —CH(CH₃)CH₂OH, —CH₂CH₂CH₂OH, —CH(CH₃)CH₂CH₂OH, —CH₂CH(CH₃)CH₂OH, —CH₂CH₂CH₂CH₂OH, and —C(CH₃)₂CH₂OH. In still further embodiments, each of R^{11a} and R^{11b}, when present, is independently selected from hydrogen, —CH₂OH, —CH₂CH₂OH, —CH(CH₃)CH₂OH, and —CH₂CH₂CH₂OH. In yet further embodiments, each of R^{11a} and R^{11b}, when present, is independently selected from hydrogen, —CH₂OH, and —CH₂CH₂OH. In an even further embodiment, each of R^{11a} and R^{11b}, when present, is independently selected from hydrogen and —CH₂OH.

In some embodiments, each of R^{11a} and R^{11b} together comprise a 3-membered cycloalkyl.

In some embodiments, R¹¹ is hydrogen or (C₁-C₅)alkyl. In further embodiments, R¹¹ is hydrogen.

In further embodiments, R¹¹ is hydrogen, methyl, ethyl, n-propyl, isopropyl, n-butyl, isobutyl, sec-butyl, or tert-butyl. In still further embodiments, R¹¹ is hydrogen, methyl, ethyl, n-propyl, or isopropyl. In yet further embodiments, R¹¹ is hydrogen, methyl, or ethyl. In an even further embodiment, R¹¹ is hydrogen or ethyl. In still further embodiments, R¹¹ is hydrogen or methyl.

l. R^{20a}, R^{20b}, R^{20c}, and R^{20d} Groups

In some embodiments, each of R^{20a}, R^{20b}, R^{20c}, and R^{20d} is independently selected from hydrogen, halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In further embodiments, each of R^{20a}, R^{20b}, R^{20c}, and R^{20d} is independently selected from hydrogen, F, —Cl, —CN, —NH₂, —OH, —NO₂, —C(O)CH₃, —C(O)CH₂CH₃, —C(O)CH(CH₃)CH₃, —C(O)CH₂CH₂CH₃, methyl, ethyl, n-propyl, isopropyl, ethenyl, propenyl, —CH₂F, —CH₂CH₂F, —CH(CH₃)CH₂F, —CH₂CH₂CH₂F, —CH₂Cl, —CH₂CH₂Cl, —CH(CH₃)CH₂Cl, —CH₂CH₂CH₂Cl, —CH₂CN, —CH₂CH₂CN, —CH(CH₃)CH₂CN, —CH₂CH₂CH₂CN, —CH₂OH, —CH₂CH₂OH, —CH(CH₃)CH₂OH, —CH₂CH₂CH₂OH, methoxy, ethoxy, n-propoxy, isopropoxy, —OCF₃, —OCHF₂, —OCH₂F, —OCH₂CH₂F, —OCH(CH₃)CH₂F, —OCH₂CH₂CH₂F, —OCCl₃, —OCHCl₂, —OCH₂Cl, —OCH₂CH₂Cl, —OCH(CH₃)CH₂Cl, —OCH₂CH₂CH₂Cl, —NHCH₃, —NHCH₂CH₃, —NHCH(CH₃)CH₃, —NHCH₂CH₂CH₃, —N(CH₃)₂, —N(CH₃)CH₂CH₃, —N(CH₃)CH(CH₃)CH₃, and —N(CH₃)CH₂CH₂CH₃. In still further embodiments, each of R^{20a}, R^{20b}, R^{20c}, and R^{20d} is independently selected from hydrogen, F, —Cl, —CN, —NH₂, —OH, —NO₂, —C(O)CH₃, —C(O)CH₂CH₃, methyl, ethyl, ethenyl, —CH₂F, —CH₂CH₂F, —CH₂Cl, —CH₂CH₂Cl, —CH₂CN, —CH₂CH₂CN, —CH₂OH, —CH₂CH₂OH, methoxy, ethoxy, —OCF₃, —OCHF₂, —OCH₂F, —OCH₂CH₂F, —OCCl₃, —OCHCl₂, —OCH₂Cl, —OCH₂CH₂Cl, —NHCH₃, —NHCH₂CH₃, —N(CH₃)₂, and —N(CH₃)CH₂CH₃. In yet further embodiments, each of R^{20a}, R^{20b}, R^{20c}, and R^{20d} is independently selected from hydrogen, F, —Cl, —CN, —NH₂, —OH, —NO₂, —C(O)CH₃, methyl, —CH₂F, —CH₂Cl, —CH₂CN, —CH₂OH, methoxy, —OCF₃, —OCHF₂, —OCH₂F, —OCCl₃, —OCHCl₂, —OCH₂Cl, —NHCH₃, and —N(CH₃)₂.

In some embodiments, each of R^{20a}, R^{20b}, R^{20c}, and R^{20d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In further embodiments, each of R^{20a}, R^{20b}, R^{20c}, and R^{20d} is independently selected from hydrogen, F, —Cl, —CN, —NH₂, —OH, —NO₂, methyl, ethyl, n-propyl, isopropyl, ethenyl, propenyl, —CH₂F, —CH₂CH₂F, —CH(CH₃)CH₂F, —CH₂CH₂CH₂F, —CH₂Cl, —CH₂CH₂Cl, —CH(CH₃)CH₂Cl, —CH₂CH₂CH₂Cl, —CH₂CN, —CH₂CH₂CN, —CH(CH₃)CH₂CN, —CH₂CH₂CH₂CN, —CH₂OH, —CH₂CH₂OH, —CH(CH₃)CH₂OH, —CH₂CH₂CH₂OH, methoxy, ethoxy, n-propoxy, isopropoxy, —OCF₃, —OCHF₂, —OCH₂F, —OCH₂CH₂F, —OCH(CH₃)CH₂F, —OCH₂CH₂CH₂F, —OCCl₃, —OCHCl₂, —OCH₂Cl, —OCH₂CH₂Cl, —OCH(CH₃)CH₂Cl, —OCH₂CH₂CH₂Cl, —NHCH₃, —NHCH₂CH₃, —NHCH(CH₃)CH₃, —NHCH₂CH₂CH₃, —N(CH₃)₂, —N(CH₃)CH₂CH₃, —N(CH₃)CH(CH₃)CH₃, and —N(CH₃)CH₂CH₂CH₃. In still further embodiments, each of R^{20a}, R^{20b}, R^{20c}, and R^{20d} is independently selected from hydrogen, F, —Cl, —CN, —NH₂, —OH, —NO₂, methyl, ethyl, ethenyl, —CH₂F, —CH₂CH₂F, —CH₂Cl, —CH₂CH₂Cl, —CH₂CN, —CH₂CH₂CN, —CH₂OH, —CH₂CH₂OH, methoxy, ethoxy, —OCF₃, —OCHF₂, —OCH₂F, —OCH₂CH₂F, —OCCl₃, —OCHCl₂, —OCH₂Cl, —OCH₂CH₂Cl, —NHCH₃, —NHCH₂CH₃, —N(CH₃)₂, and —N(CH₃)CH₂CH₃. In yet further embodiments, each of R^{20a}, R^{20b}, R^{20c}, and R^{20d} is independently selected from hydrogen, F, —Cl, —CN, —NH₂, —OH, —NO₂, methyl, —CH₂F, —CH₂Cl, —CH₂CN, —CH₂OH, methoxy, —OCF₃, —OCHF₂, —OCH₂F, —OCCl₃, —OCHCl₂, —OCH₂Cl, —NHCH₃, and —N(CH₃)₂.

In further embodiments, each of R^{20a}, R^{20b}, R^{20c}, and R^{20d} is hydrogen.

In various embodiments, each of R^{20a}, R^{20b}, R^{20c}, and R^{20d} is independently selected from hydrogen, halogen, —CN, —NH₂, —OH, —NO₂, C1-C4 hydroxyalkyl, C1-C4 haloalkoxy, C1-C4 alkoxy. In further embodiments, each of R^{20a}, R^{20b}, R^{20c}, and R^{20d} is independently selected from hydrogen, F, —Cl, —CN, —NH₂, —OH, —NO₂, —CH₂OH, —CH₂CH₂OH, —CH(CH₃)CH₂OH, —CH₂CH₂CH₂OH, methoxy, ethoxy, n-propoxy, isopropoxy, —OCF₃, —OCHF₂, —OCH₂F, —OCH₂CH₂F, —OCH(CH₃)CH₂F, —OCH₂CH₂CH₂F, —OCCl₃, —OCHCl₂, —OCH₂Cl, —OCH₂CH₂Cl, —OCH(CH₃)CH₂Cl, and —OCH₂CH₂CH₂Cl. In still further embodiments, each of R^{20a}, R^{20b}, R^{20c}, and R^{20d} is independently selected from hydrogen, F, —Cl, —CN, —NH₂, —OH, —NO₂, —CH₂OH, —CH₂CH₂OH, methoxy, ethoxy, —OCF₃, —OCHF₂, —OCH₂F, —OCH₂CH₂F, —OCCl₃, —OCHCl₂, —OCH₂Cl, and —OCH₂CH₂Cl. In yet further embodiments, each of R^{20a}, R^{20b}, R^{20c}, and R^{20d} is independently selected from hydrogen, F, —Cl, —CN, —NH₂, —OH, —NO₂, —CH₂OH, methoxy, —OCF₃, —OCHF₂, —OCH₂F, —OCCl₃, —OCHCl₂, and —OCH₂Cl.

In various embodiments, each of R^{20a}, R^{20b}, R^{20c}, and R^{20d} is independently selected from hydrogen, halogen, —CN, —NH₂, —OH, —NO₂, C1-C4 alkylamino, and (C1-C4)(C1-C4) dialkylamino. In further embodiments, each of R^{20a}, R^{20b}, R^{20c}, and R^{20d} is independently selected from hydrogen, F, —Cl, —CN, —NH₂, —OH, —NO₂, —NHCH₃, —NHCH₂CH₃, —NHCH(CH₃)CH₃, —NHCH₂CH₂CH₃, —N(CH₃)₂, —N(CH₃)CH₂CH₃, —N(CH₃)CH(CH₃)CH₃, and —N(CH₃)CH₂CH₂CH₃. In still further embodiments, each of R^{20a}, R^{20b}, R^{20c}, and R^{20d} is independently selected from hydrogen, F, —Cl, —CN, —NH₂, —OH, —NO₂, —NHCH₃, —NHCH₂CH₃, —N(CH₃)₂, and —N(CH₃)CH₂CH₃. In yet further embodiments, each of R^{20a}, R^{20b}, R^{20c}, and R^{20d} is independently selected from hydrogen, F, —Cl, —CN, —NH₂, —OH, —NO₂, —NHCH₃, and —N(CH₃)₂.

In various embodiments, each of R^{20a}, R^{20b}, R^{20c}, and R^{20d} is independently selected from hydrogen, halogen, —CN, —NH₂, —OH, —NO₂, C1-C4 haloalkyl, and C1-C4 cyanoalkyl.

In further embodiments, each of R^{20a}, R^{20b}, R^{20c}, and R^{20d} is independently selected from hydrogen, F, —Cl, —CN, —NH₂, —OH, —NO₂, —CH₂F, —CH₂CH₂F, —CH(CH₃)CH₂F, —CH₂CH₂CH₂F, —CH₂Cl, —CH₂CH₂Cl, —CH(CH₃)CH₂Cl, —CH₂CH₂CH₂Cl, —CH₂CN, —CH₂CH₂CN, —CH(CH₃)CH₂CN, and —CH₂CH₂CH₂CN. In still further embodiments, each of R^{20a}, R^{20b}, R^{20c}, and R^{20d} is independently selected from hydrogen, F, —Cl, —CN, —NH₂, —OH, —NO₂, —CH₂F, —CH₂CH₂F, —CH₂Cl, —CH₂CH₂Cl, —CH₂CN, and —CH₂CH₂CN. In yet further embodiments, each of R^{20a}, R^{20b}, R^{20c}, and R^{20d} is independently selected from hydrogen, F, —Cl, —CN, —NH₂, —OH, —NO₂, —CH₂F, —CH₂Cl, and —CH₂CN.

In various embodiments, each of R^{20a}, R^{20b}, R^{20c}, and R^{20d} is independently selected from hydrogen, halogen, —CN, —NH₂, —OH, —NO₂, C1-C4 alkyl, and C2-C4 alkenyl. In further embodiments, each of R^{20a}, R^{20b}, R^{20c}, and R^{20d} is independently selected from hydrogen, F, —Cl, —CN, —NH₂, —OH, —NO₂, methyl, ethyl, n-propyl, isopropyl, ethenyl, and propenyl. In still further embodiments, each of R^{20a}, R^{20b}, R^{20c}, and R^{20d} is independently selected from hydrogen, F, —Cl, —CN, —NH₂, —OH, —NO₂, methyl, ethyl, and ethenyl. In yet further embodiments, each of R^{20a}, R^{20b}, R^{20c}, and R^{20d} is independently selected from hydrogen, F, —Cl, —CN, —NH₂, —OH, —NO₂, and methyl.

In various embodiments, each of R^{20a}, R^{20b}, R^{20c}, and R^{20d} is independently selected from hydrogen and C1-C4 alkyl. In further embodiments, each of R^{20a}, R^{20b}, R^{20c}, and R^{20d} is independently selected from hydrogen, methyl, ethyl, n-propyl, and isopropyl. In still further embodiments, each of R^{20a}, R^{20b}, R^{20c}, and R^{20d} is independently selected from hydrogen, methyl, and ethyl. In yet further embodiments, each of R^{20a}, R^{20b}, R^{20c}, and R^{20d} is independently selected from hydrogen and methyl.

In various embodiments, each of R^{20a}, R^{20b}, R^{20c}, and R^{20d} is independently selected from hydrogen and halogen. In further embodiments, each of R^{20a}, R^{20b}, R^{20c}, and R^{20d} is independently selected from hydrogen, F, —Cl, and —Br. In still further embodiments, each of R^{20a}, R^{20b}, R^{20c}, and R^{20d} is independently selected from hydrogen, F, and —Cl. In yet further embodiments, each of R^{20a}, R^{20b}, R^{20c}, and R^{20d} is independently selected from hydrogen and —Cl. In still further embodiments, each of R^{20a}, R^{20b}, R^{20c}, and R^{20d} is independently selected from hydrogen and —F.

m. R²¹ Groups

In some embodiments, R²¹ is selected from hydrogen, halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In further embodiments, R²¹ is selected from hydrogen, F, —Cl, —CN, —NH₂, —OH, —NO₂, —C(O)CH₃, —C(O)CH₂CH₃, —C(O)CH(CH₃)CH₃, —C(O)CH₂CH₂CH₃, methyl, ethyl, n-propyl, isopropyl, ethenyl, propenyl, —CH₂F, —CH₂CH₂F, —CH(CH₃)CH₂F, —CH₂CH₂CH₂F, —CH₂Cl, —CH₂CH₂Cl, —CH(CH₃)CH₂Cl, —CH₂CH₂CH₂Cl, —CH₂CN, —CH₂CH₂CN, —CH(CH₃)CH₂CN, —CH₂CH₂CH₂CN, —CH₂OH, —CH₂CH₂OH, —CH(CH₃)CH₂OH, —CH₂CH₂CH₂OH, methoxy, ethoxy, n-propoxy, isopropoxy, —OCF₃, —OCHF₂, —OCH₂F, —OCH₂CH₂F, —OCH(CH₃)CH₂F, —OCH₂CH₂CH₂F, —OCCl₃, —OCHCl₂, —OCH₂Cl, —OCH₂CH₂Cl, —OCH(CH₃)CH₂Cl, —OCH₂CH₂CH₂Cl, —NHCH₃, —NHCH₂CH₃, —NHCH(CH₃)CH₃, —NHCH₂CH₂CH₃, —N(CH₃)₂, —N(CH₃)CH₂CH₃, —N(CH₃)CH(CH₃)CH₃, and —N(CH₃)CH₂CH₂CH₃. In still further embodiments, e R²¹ is selected from hydrogen, F, —Cl, —CN, —NH₂, —OH, —NO₂, —C(O)CH₃, —C(O)CH₂CH₃, methyl, ethyl, ethenyl, —CH₂F, —CH₂CH₂F, —CH₂Cl, —CH₂CH₂Cl, —CH₂CN, —CH₂CH₂CN, —CH₂OH, —CH₂CH₂OH, methoxy, ethoxy, —OCF₃, —OCHF₂, —OCH₂F, —OCH₂CH₂F, —OCCl₃, —OCHCl₂, —OCH₂Cl, —OCH₂CH₂Cl, —NHCH₃, —NHCH₂CH₃, —N(CH₃)₂, and —N(CH₃)CH₂CH₃. In yet further embodiments, R²¹ is selected from hydrogen, F, —Cl, —CN, —NH₂, —OH, —NO₂, —C(O)CH₃, methyl, —CH₂F, —CH₂Cl, —CH₂CN, —CH₂OH, methoxy, —OCF₃, —OCHF₂, —OCH₂F, —OCCl₃, —OCHCl₂, —OCH₂Cl, —NHCH₃, and —N(CH₃)₂.

In some embodiments, R²¹ is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In further embodiments, R²¹ is independently selected from hydrogen, F, —Cl, —CN, —NH₂, —OH, —NO₂, methyl, ethyl, n-propyl, isopropyl, ethenyl, propenyl, —CH₂F, —CH₂CH₂F, —CH(CH₃)CH₂F, —CH₂CH₂CH₂F, —CH₂Cl, —CH₂CH₂Cl, —CH(CH₃)CH₂Cl, —CH₂CH₂CH₂Cl, —CH₂CN, —CH₂CH₂CN, —CH(CH₃)CH₂CN, —CH₂CH₂CH₂CN, —CH₂OH, —CH₂CH₂OH, —CH(CH₃)CH₂OH, —CH₂CH₂CH₂OH, methoxy, ethoxy, n-propoxy, isopropoxy, —OCF₃, —OCHF₂, —OCH₂F, —OCH₂CH₂F, —OCH(CH₃)CH₂F, —OCH₂CH₂CH₂F, —OCCl₃, —OCHCl₂, —OCH₂Cl, —OCH₂CH₂Cl, —OCH(CH₃)CH₂Cl, —OCH₂CH₂CH₂Cl, —NHCH₃, —NHCH₂CH₃, —NHCH(CH₃)CH₃, —NHCH₂CH₂CH₃, —N(CH₃)₂, —N(CH₃)CH₂CH₃, —N(CH₃)CH(CH₃)CH₃, and —N(CH₃)CH₂CH₂CH₃. In still further embodiments, R²¹ is independently selected from hydrogen, F, —Cl, —CN, —NH₂, —OH, —NO₂, methyl, ethyl, ethenyl, —CH₂F, —CH₂CH₂F, —CH₂Cl, —CH₂CH₂Cl, —CH₂CN, —CH₂CH₂CN, —CH₂OH, —CH₂CH₂OH, methoxy, ethoxy, —OCF₃, —OCHF₂, —OCH₂F, —OCH₂CH₂F, —OCCl₃, —OCHCl₂, —OCH₂Cl, —OCH₂CH₂Cl, —NHCH₃, —NHCH₂CH₃, —N(CH₃)₂, and —N(CH₃)CH₂CH₃. In yet further embodiments, R²¹ is selected from hydrogen, F, —Cl, —CN, —NH₂, —OH, —NO₂, methyl, —CH₂F, —CH₂Cl, —CH₂CN, —CH₂OH, methoxy, —OCF₃, —OCHF₂, —OCH₂F, —OCCl₃, —OCHCl₂, —OCH₂Cl, —NHCH₃, and —N(CH₃)₂.

In further embodiments, R²¹ is hydrogen.

In various embodiments, R²¹ is selected from hydrogen, halogen, —CN, —NH₂, —OH, —NO₂, C1-C4 hydroxyalkyl, C1-C4 haloalkoxy, C1-C4 alkoxy. In further embodiments, R²¹ is selected from hydrogen, F, —Cl, —CN, —NH₂, —OH, —NO₂, —CH₂OH, —CH₂CH₂OH, —CH(CH₃)CH₂OH, —CH₂CH₂CH₂OH, methoxy, ethoxy, n-propoxy, isopropoxy, —OCF₃, —OCHF₂, —OCH₂F, —OCH₂CH₂F, —OCH(CH₃)CH₂F, —OCH₂CH₂CH₂F, —OCCl₃, —OCHCl₂, —OCH₂Cl, —OCH₂CH₂Cl, —OCH(CH₃)CH₂Cl, and —OCH₂CH₂CH₂Cl. In still further embodiments, R²¹ is selected from hydrogen, F, —Cl, —CN, —NH₂, —OH, —NO₂, —CH₂OH, —CH₂CH₂OH, methoxy, ethoxy, —OCF₃, —OCHF₂, —OCH₂F, —OCH₂CH₂F, —OCCl₃, —OCHCl₂, —OCH₂Cl, and —OCH₂CH₂Cl. In yet further embodiments, R²¹ is selected from hydrogen, F, —Cl, —CN, —NH₂, —OH, —NO₂, —CH₂OH, methoxy, —OCF₃, —OCHF₂, —OCH₂F, —OCCl₃, —OCHCl₂, and —OCH₂Cl.

In various embodiments, R²¹ is selected from hydrogen, halogen, —CN, —NH₂, —OH, —NO₂, C1-C4 alkylamino, and (C1-C4)(C1-C4) dialkylamino. In further embodiments, R²¹ is selected from hydrogen, F, —Cl, —CN, —NH₂, —OH, —NO₂, —NHCH₃, —NHCH₂CH₃, —NHCH(CH₃)CH₃, —NHCH₂CH₂CH₃, —N(CH₃)₂, —N(CH₃)CH₂CH₃, —N(CH₃)CH(CH₃)CH₃, and —N(CH₃)CH₂CH₂CH₃. In still further embodiments, R²¹ is selected from hydrogen, F, —Cl, —CN, —NH₂, —OH, —NO₂, —NHCH₃, —NHCH₂CH₃, —N(CH₃)₂, and —N(CH₃)CH₂CH₃. In yet further embodiments, R²¹ is selected from hydrogen, F, —Cl, —CN, —NH₂, —OH, —NO₂, —NHCH₃, and —N(CH₃)₂.

In various embodiments, R²¹ is selected from hydrogen, halogen, —CN, —NH₂, —OH, —NO₂, C1-C4 haloalkyl, and C1-C4 cyanoalkyl. In further embodiments, R²¹ is selected from hydrogen, F, —Cl, —CN, —NH₂, —OH, —NO₂, —CH₂F, —CH₂CH₂F, —CH(CH₃)CH₂F, —CH₂CH₂CH₂F, —CH₂Cl, —CH₂CH₂Cl, —CH(CH₃)CH₂Cl, —CH₂CH₂CH₂Cl, —CH₂CN, —CH₂CH₂CN, —CH(CH₃)CH₂CN, and —CH₂CH₂CH₂CN. In still further embodiments, R²¹ is selected from hydrogen, F, —Cl, —CN, —NH₂, —OH, —NO₂, —CH₂F, —CH₂CH₂F, —CH₂Cl, —CH₂CH₂Cl, —CH₂CN, and —CH₂CH₂CN. In yet further embodiments, R²¹ is selected from hydrogen, F, —Cl, —CN, —NH₂, —OH, —NO₂, —CH₂F, —CH₂Cl, and —CH₂CN.

In various embodiments, R²¹ is selected from hydrogen, halogen, —CN, —NH₂, —OH, —NO₂, C1-C4 alkyl, and C2-C4 alkenyl. In further embodiments, R²¹ is selected from hydrogen, F, —Cl, —CN, —NH₂, —OH, —NO₂, methyl, ethyl, n-propyl, isopropyl, ethenyl, and propenyl. In still further embodiments, R²¹ is selected from hydrogen, F, —Cl, —CN, —NH₂, —OH, —NO₂, methyl, ethyl, and ethenyl. In yet further embodiments, R²¹ is selected from hydrogen, F, —Cl, —CN, —NH₂, —OH, —NO₂, and methyl.

In various embodiments, R²¹ is selected from hydrogen and C1-C4 alkyl. In further embodiments, R²¹ is selected from hydrogen, methyl, ethyl, n-propyl, and isopropyl. In still further embodiments, R²¹ is selected from hydrogen, methyl, and ethyl. In yet further embodiments, R²¹ is selected from hydrogen and methyl.

In various embodiments, R²¹ is selected from hydrogen and halogen. In further embodiments, R²¹ is selected from hydrogen, F, —Cl, and —Br. In still further embodiments, R²¹ is selected from hydrogen, F, and —Cl. In yet further embodiments, R²¹ is selected from hydrogen and —Cl. In still further embodiments, R²¹ is selected from hydrogen and —F.

n. R³⁰ Groups

In some embodiments, R³⁰, when present, is selected from —C(O)(C1-C4 alkyl), C1-C4 alkyl, and C2-C4 alkenyl. In further embodiments, R³⁰, when present, is selected from —C(O)CH₃, —C(O)CH₂CH₃, —C(O)CH(CH₃)CH₃, —C(O)CH₂CH₂CH₃, methyl, ethyl, n-propyl, isopropyl, ethenyl, and propenyl. In still further embodiments, R³⁰, when present, is selected from —C(O)CH₃, —C(O)CH₂CH₃, methyl, ethyl, and ethenyl. In yet further embodiments, R³⁰, when present, is selected from —C(O)CH₃ and methyl.

In some embodiments, R³⁰, when present, is selected from C1-C4 alkyl and C2-C4 alkenyl. In further embodiments, R³⁰, when present, is selected from methyl, ethyl, n-propyl, isopropyl, ethenyl, and propenyl. In still further embodiments, R³⁰, when present, is selected from methyl, ethyl, and ethenyl. In yet further embodiments, R³⁰, when present, is methyl.

In some embodiments, R³⁰, when present, is —C(O)(C1-C4 alkyl). In further embodiments, R³⁰, when present, is selected from —C(O)CH₃, —C(O)CH₂CH₃, —C(O)CH(CH₃)CH₃, and —C(O)CH₂CH₂CH₃. In still further embodiments, R³⁰, when present, is selected from —C(O)CH₃ and —C(O)CH₂CH₃. In yet further embodiments, R³⁰, when present, —C(O)CH₃.

o. Cy¹ Groups

In some embodiments, Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, In further embodiments, Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, or 3 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is selected from a 3- to 10-membered carbocycle, a 3- to 10-membered heterocycle, a 6- to 10-membered aryl, and a 6- to 10-membered heteroaryl, and is substituted with 0, 1, or 2 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In yet further embodiments, Cy¹, when present, is selected from a 3- to 10-membered carbocycle, a 3- to 10-membered heterocycle, a 6- to 10-membered aryl, and a 6- to 10-membered heteroaryl, and is substituted with 0 or 1 group selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In even further embodiments, Cy¹, when present, is selected from a 3- to 10-membered carbocycle, a 3- to 10-membered heterocycle, a 6- to 10-membered aryl, and a 6- to 10-membered heteroaryl, and is monosubstituted with a group selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino,

In some embodiments, Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In further embodiments, Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is selected from a 3- to 10-membered carbocycle, a 3- to 10-membered heterocycle, a 6- to 10-membered aryl, and a 6- to 10-membered heteroaryl, and is substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In yet further embodiments, Cy¹, when present, is selected from a 3- to 10-membered carbocycle, a 3- to 10-membered heterocycle, a 6- to 10-membered aryl, and a 6- to 10-membered heteroaryl, and is substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In an even further embodiment, Cy¹, when present, is selected from a 3- to 10-membered carbocycle, a 3- to 10-membered heterocycle, a 6- to 10-membered aryl, and a 6- to 10-membered heteroaryl, and is monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is selected from a 3- to 10-membered carbocycle, a 3- to 10-membered heterocycle, a 6- to 10-membered aryl, and a 6- to 10-membered heteroaryl, and is unsubstituted.

In various embodiments, Cy¹, when present, is selected from a 3- to 10-membered carbocycle and a 3- to 10-membered heterocycle, and is substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In further embodiments, Cy¹, when present, is selected from a 3- to 10-membered carbocycle and a 3- to 10-membered heterocycle, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is selected from a 3- to 10-membered carbocycle and a 3- to 10-membered heterocycle, and is substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In yet further embodiments, Cy¹, when present, is selected from a 3- to 10-membered carbocycle and a 3- to 10-membered heterocycle, and is substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In an even further embodiment, Cy¹, when present, is selected from a 3- to 10-membered carbocycle and a 3- to 10-membered heterocycle, and is monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is selected from a 3- to 10-membered carbocycle and a 3- to 10-membered heterocycle, and is unsubstituted.

In various embodiments, Cy¹, when present, is a 3- to 10-membered carbocycle substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In further embodiments, Cy¹, when present, is a 3- to 10-membered carbocycle substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is a 3- to 10-membered carbocycle substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In yet further embodiments, Cy¹, when present, is a 3- to 10-membered carbocycle substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In an even further embodiment, Cy¹, when present, is a 3- to 10-membered carbocycle monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is an unsubstituted 3- to 10-membered carbocycle.

In various embodiments, Cy¹, when present, is a 9- to 10-membered carbocycle substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In further embodiments, Cy¹, when present, is a 9- to 10-membered carbocycle substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is a 9- to 10-membered carbocycle substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In yet further embodiments, Cy¹, when present, is a 9- to 10-membered carbocycle substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In an even further embodiment, Cy¹, when present, is a 9- to 10-membered carbocycle monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is an unsubstituted 9- to 10-membered carbocycle.

In various embodiments, Cy¹, when present, is a 3- to 10-membered heterocycle substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In further embodiments, Cy¹, when present, is a 3- to 10-membered heterocycle substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is a 3- to 10-membered heterocycle substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In yet further embodiments, Cy¹, when present, is a 3- to 10-membered heterocycle substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In an even further embodiment, Cy¹, when present, is a 3- to 10-membered heterocycle monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is an unsubstituted 3- to 10-membered heterocycle.

In various embodiments, Cy¹, when present, is a 9- to 10-membered heterocycle substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In further embodiments, Cy¹, when present, is a 9- to 10-membered heterocycle substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is a 9- to 10-membered heterocycle substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In yet further embodiments, Cy¹, when present, is a 9- to 10-membered heterocycle substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In an even further embodiment, Cy¹, when present, is a 9- to 10-membered heterocycle monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is an unsubstituted 9- to 10-membered heterocycle.

In various embodiments, Cy¹, when present, is selected from a 6- to 10-membered aryl and a 6- to 10-membered heteroaryl, and is substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In further embodiments, Cy¹, when present, is selected from a 6- to 10-membered aryl and a 6- to 10-membered heteroaryl, and is substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is selected from a 6- to 10-membered aryl and a 6- to 10-membered heteroaryl, and is substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In yet further embodiments, Cy¹, when present, is selected from a 6- to 10-membered aryl and a 6- to 10-membered heteroaryl, and is substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In an even further embodiment, Cy¹, when present, is selected from a 6- to 10-membered aryl and a 6- to 10-membered heteroaryl, and is monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is selected from a 6- to 10-membered aryl and a 6- to 10-membered heteroaryl, and is unsubstituted.

In various embodiments, Cy¹, when present, is a 6- to 10-membered aryl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. Examples of 6- to 10-membered aryls include, but are not limited to, phenyl and naphthyl. In further embodiments, Cy¹, when present, is a 6- to 10-membered aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is a 6- to 10-membered aryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In yet further embodiments, Cy¹, when present, is a 6- to 10-membered aryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In an even further embodiment, Cy¹, when present, is a 6- to 10-membered aryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is an unsubstituted 6- to 10-membered aryl.

In various embodiments, Cy¹, when present, is a 6-membered aryl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In further embodiments, Cy¹, when present, is a 6-membered aryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is a 6-membered aryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In yet further embodiments, Cy¹, when present, is a 6-membered aryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In an even further embodiment, Cy¹, when present, is a 6-membered aryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is an unsubstituted 6-membered aryl.

In various embodiments, Cy¹, when present, is a 6- to 10-membered heteroaryl substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. Examples of 6- to 10-membered heteroaryls include, but are not limited to, indolyl, benzofuranyl, benzothiophenyl, triazolyl, imidazolyl, oxazolyl, thiazolyl, pyrazolyl, isoxazolyl, isothiazolyl, pyridinyl, quinolinyl, and isoquinolinyl. In further embodiments, Cy¹, when present, is a 6- to 10-membered heteroaryl substituted with 0, 1, 2, or 3 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is a 6- to 10-membered heteroaryl substituted with 0, 1, or 2 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In yet further embodiments, Cy¹, when present, is a 6- to 10-membered heteroaryl substituted with 0 or 1 group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In an even further embodiment, Cy¹, when present, is a 6- to 10-membered heteroaryl monosubstituted with a group selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino. In still further embodiments, Cy¹, when present, is an unsubstituted 6- to 10-membered heteroaryl.

In further embodiments, Cy¹, when present, is a structure represented by a formula selected from:

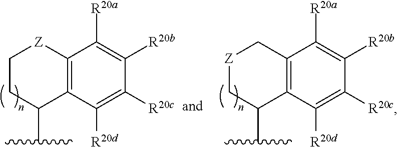

wherein Z is O, CH₂, or NR³⁰; wherein R³⁰, when present, is selected from —C(O)(C1-C4 alkyl), C1-C4 alkyl, and C2-C4 alkenyl; wherein n is 0 or 1; and wherein each of R^{20a}, R^{20b}, R^{20c}, and R^{20d} is independently selected from hydrogen, halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino.

In further embodiments, Cy¹, when present, is a structure represented by a formula selected from:

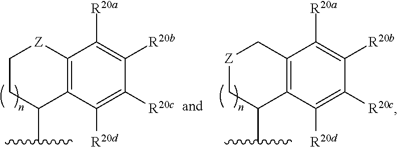

wherein Z is O or CH₂; wherein n is 0 or 1; and wherein each of R^{20a}, R^{20b}, R^{20c}, and R^{20d} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino.

Thus, in some embodiments, n is 0 or 1. In further embodiments, n is 0. In still further embodiments, n is 1.

In further embodiments, Cy¹, when present, is a structure represented by a formula:

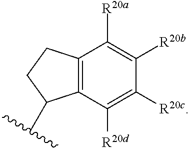

In further embodiments, Cy¹, when present, is a structure represented by a formula selected from:

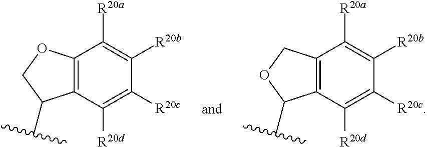

In further embodiments, Cy¹, when present, is a structure represented by a formula:

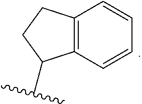 [SMILES: *C1CCc2ccccc21]

In further embodiments, Cy¹, when present, is a structure represented by a formula selected from:

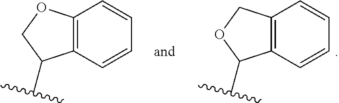 [SMILES: *C1COc2ccccc21 ; *C1OCc2ccccc21]

In further embodiments, Cy¹, when present, is a structure represented by a formula selected from:

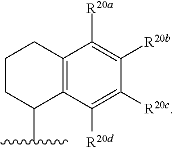

In further embodiments, Cy¹, when present, is a structure represented by a formula selected from:

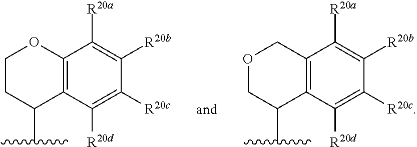

In further embodiments, Cy¹, when present, is a structure represented by a formula selected from:

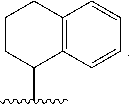 [SMILES: *C1CCCc2ccccc21]

In further embodiments, Cy¹, when present, is a structure represented by a formula selected from:

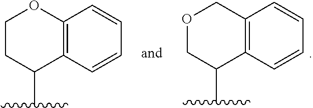 [SMILES: *C1CCOc2ccccc21 ; *C1COCc2ccccc21]

### 2. Example Compounds

In some embodiments, a compound can be present as one or more of the following structures:

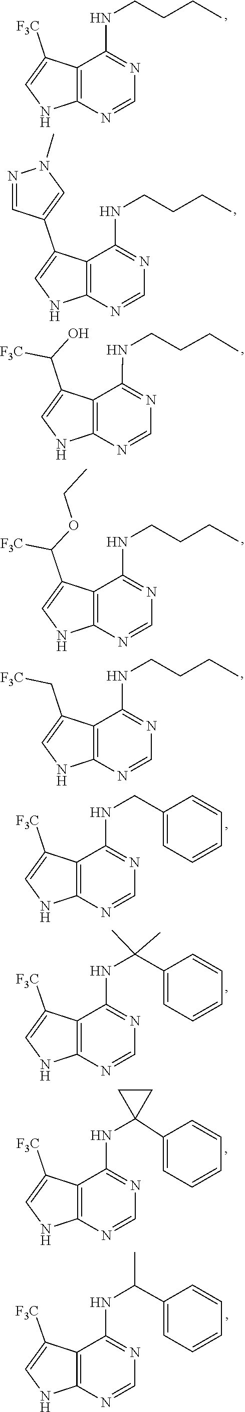

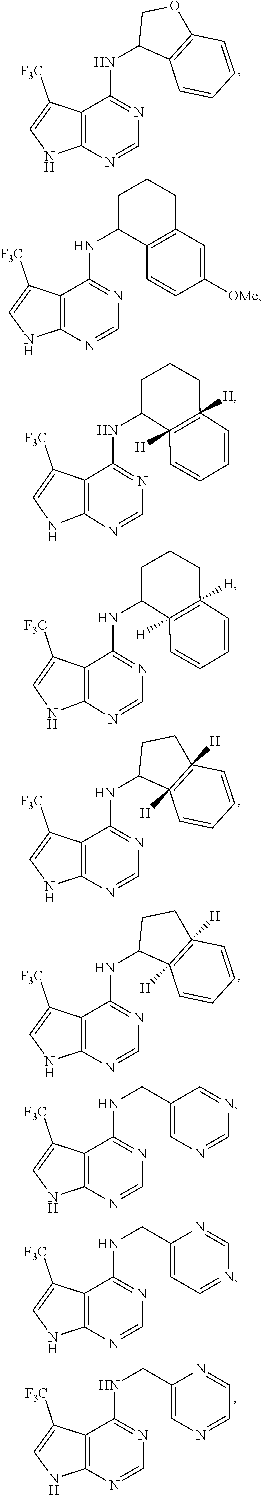

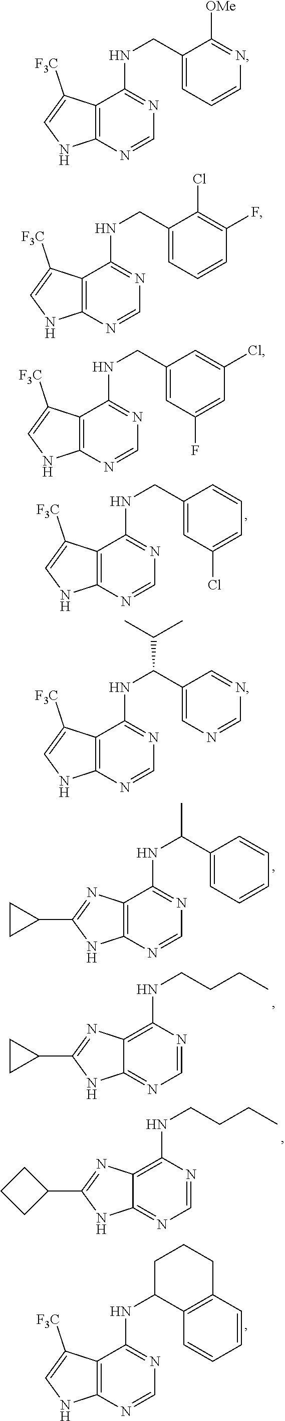

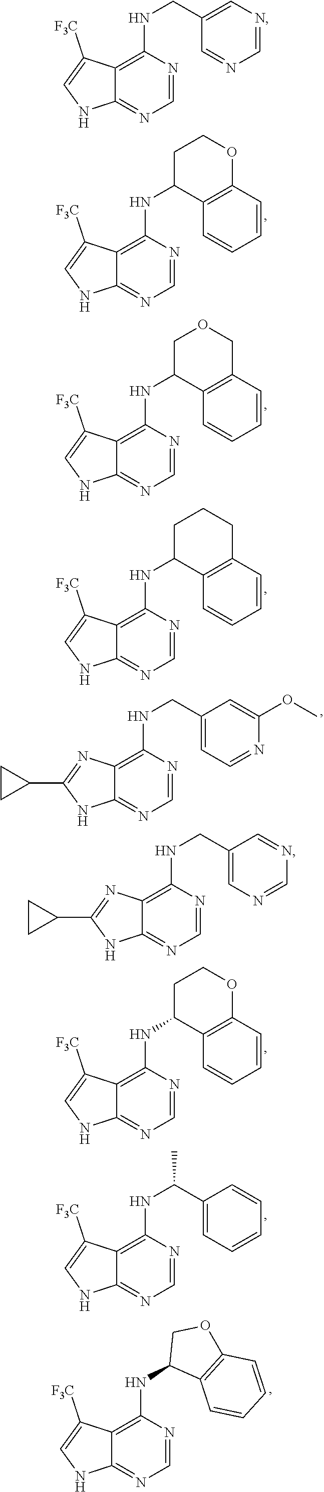

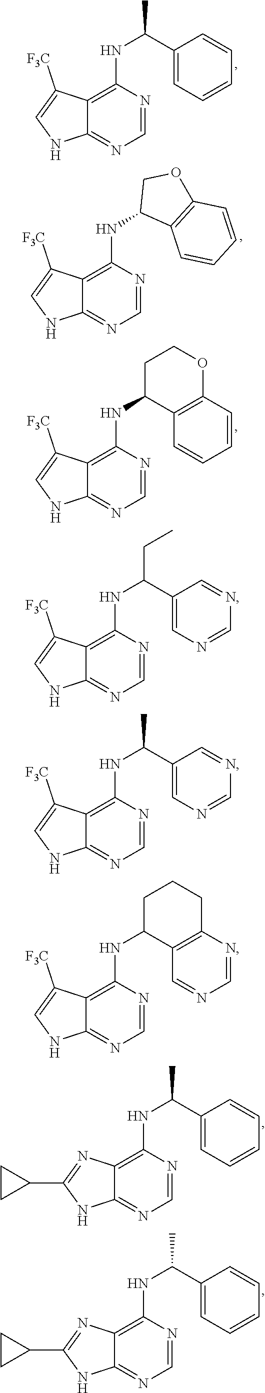

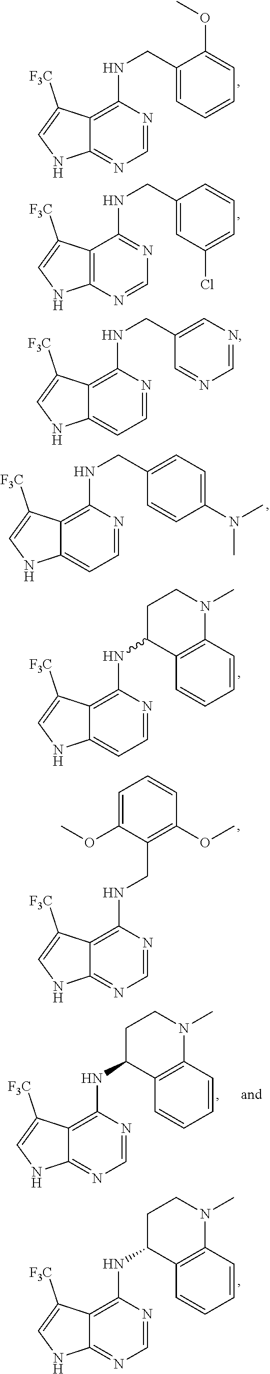

or a pharmaceutically acceptable salt thereof.

In some embodiments, a compound can be present as one or more of the following structures:

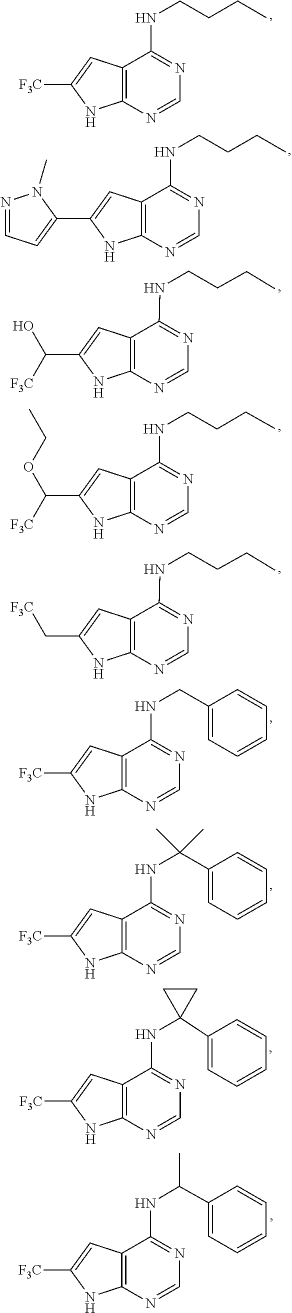

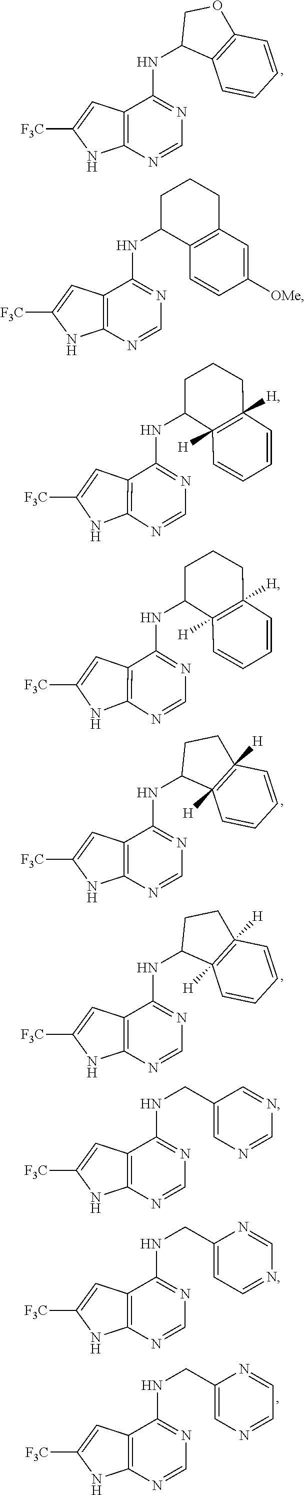

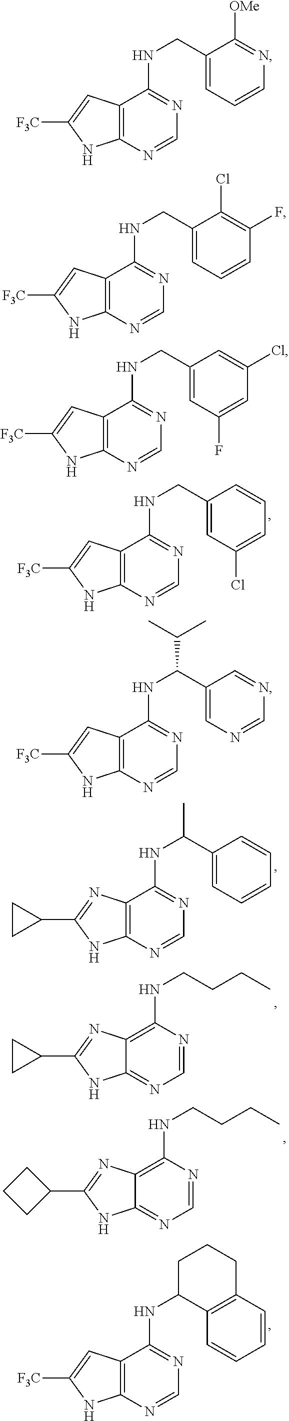

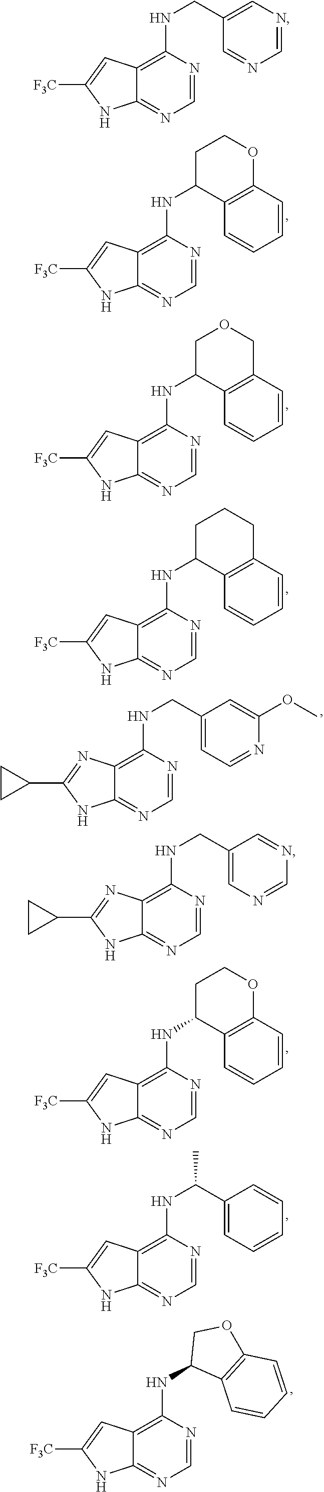

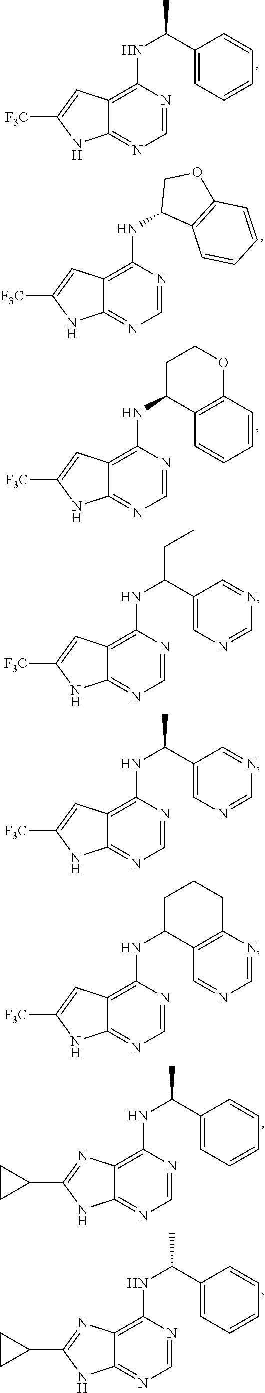

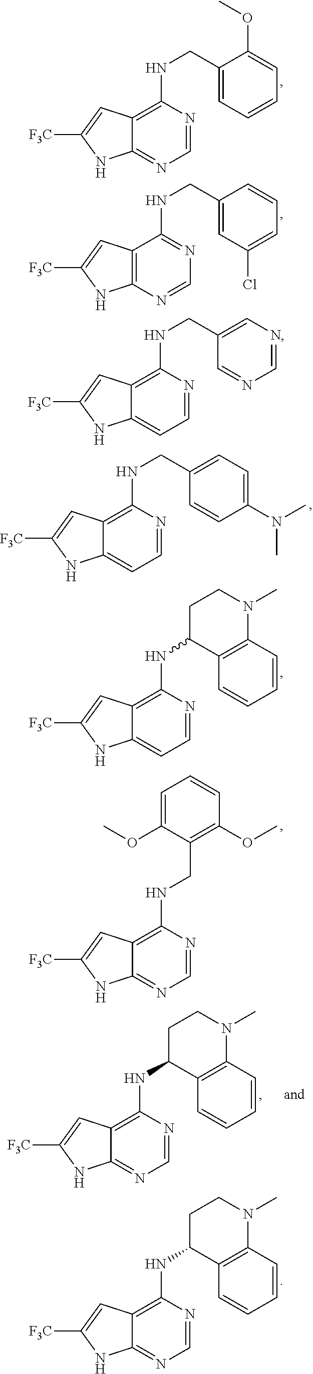

or a pharmaceutically acceptable salt thereof.

In some embodiments, a compound can be present as one or more of the following structures:

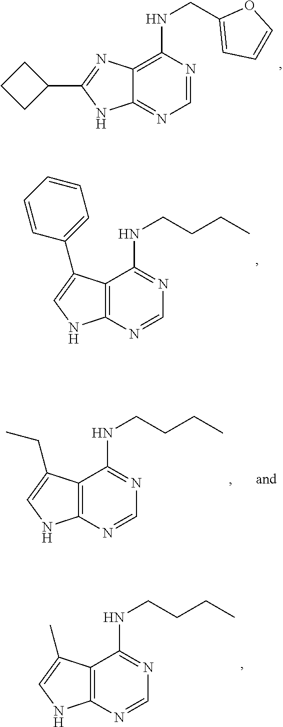

In some embodiments, a compound can be present as one or more of the following structures:

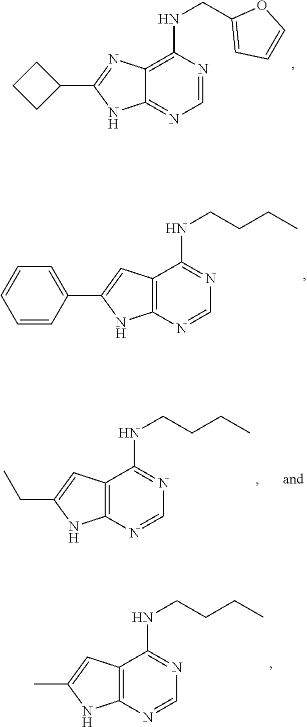

or a pharmaceutically acceptable salt thereof.

In some embodiments, a compound can be present as one or more of the following structures:

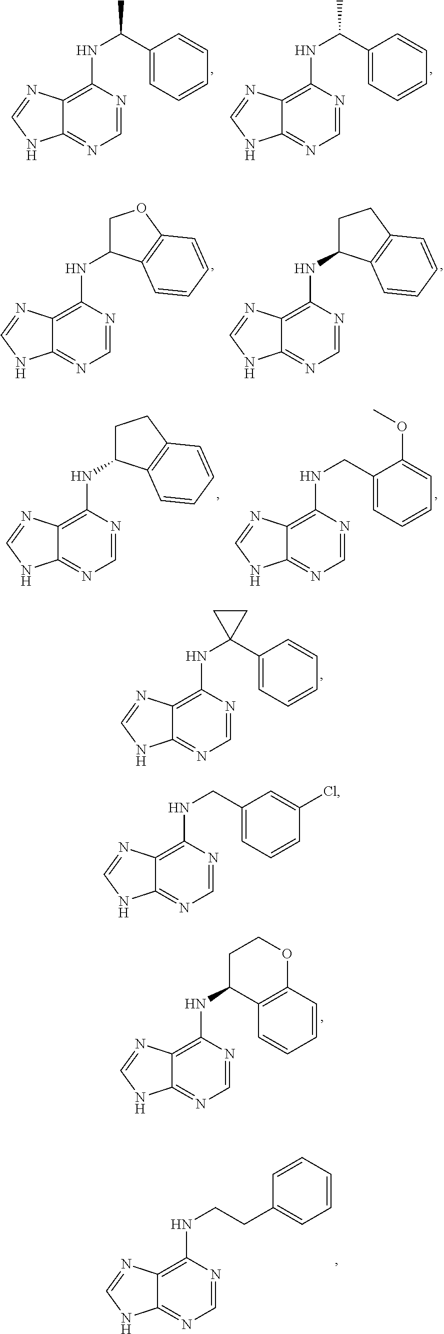

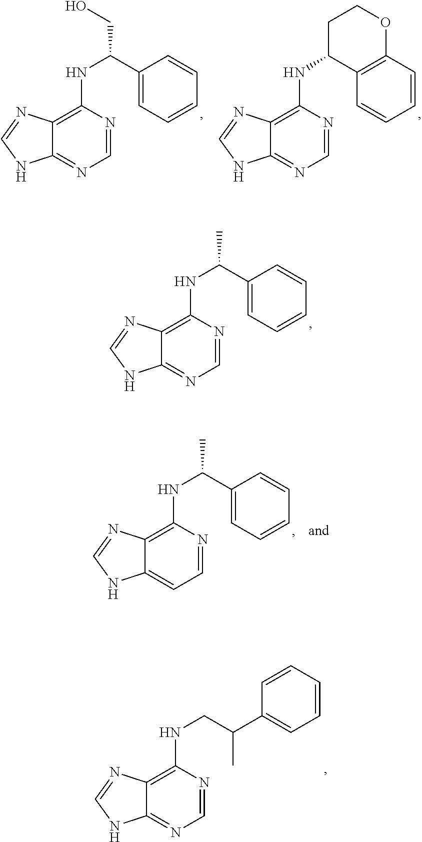

or a pharmaceutically acceptable salt thereof.

In some embodiments, a compound can be present as one or more of the following structures:

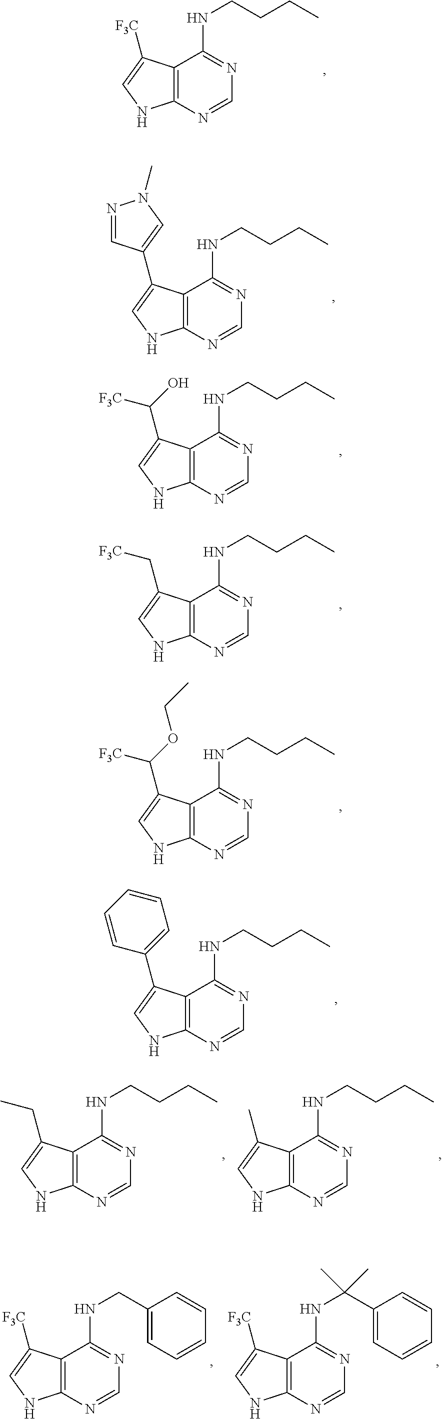

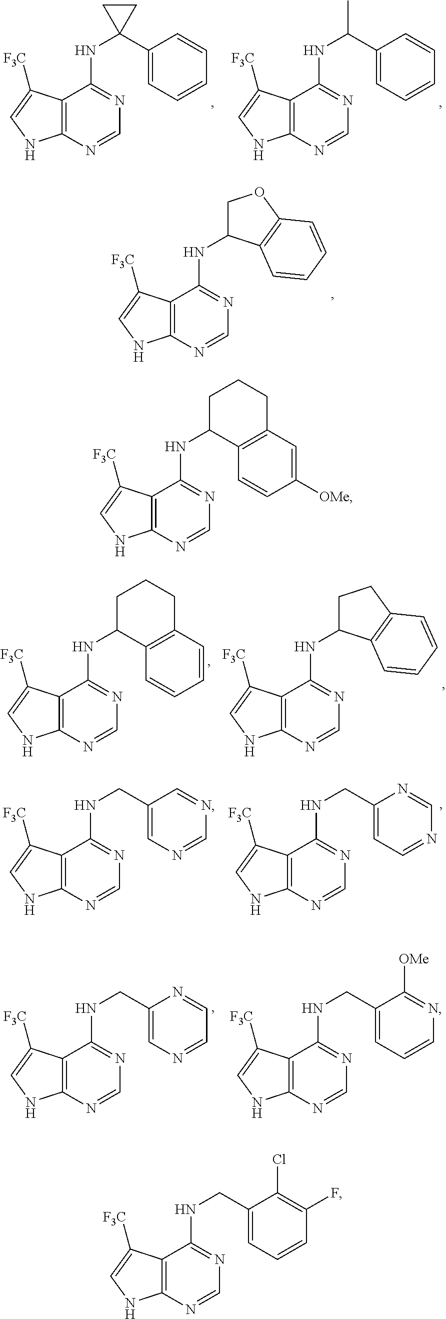

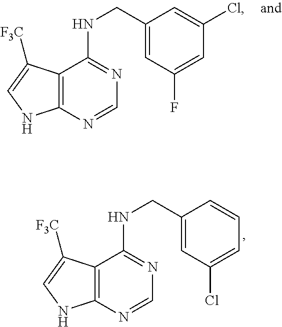 [SMILES: Fc1cc(Cl)cc(CNc2ncnc3[nH]cc(C(F)(F)F)c23)c1 ; FC(F)(F)c1c[nH]c2ncnc(NCc3cccc(Cl)c3)c12]

or a pharmaceutically acceptable salt thereof.

In some embodiments, a compound can be present as one or more of the following structures:

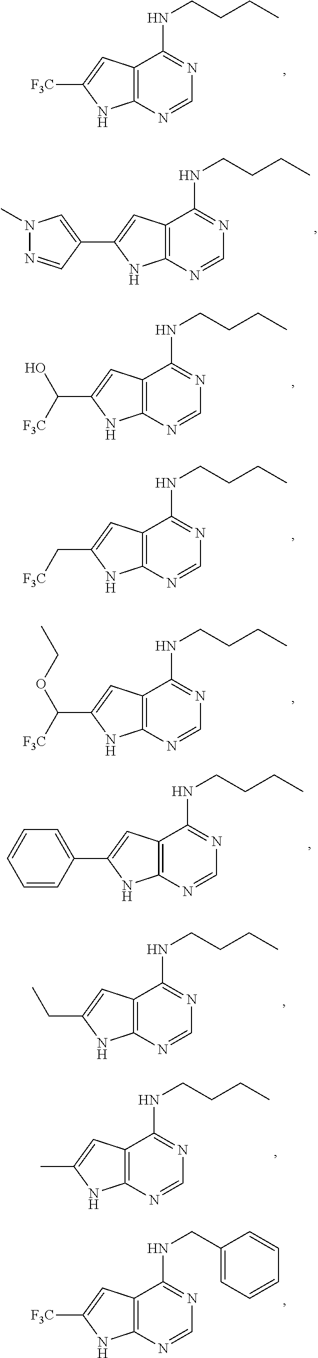

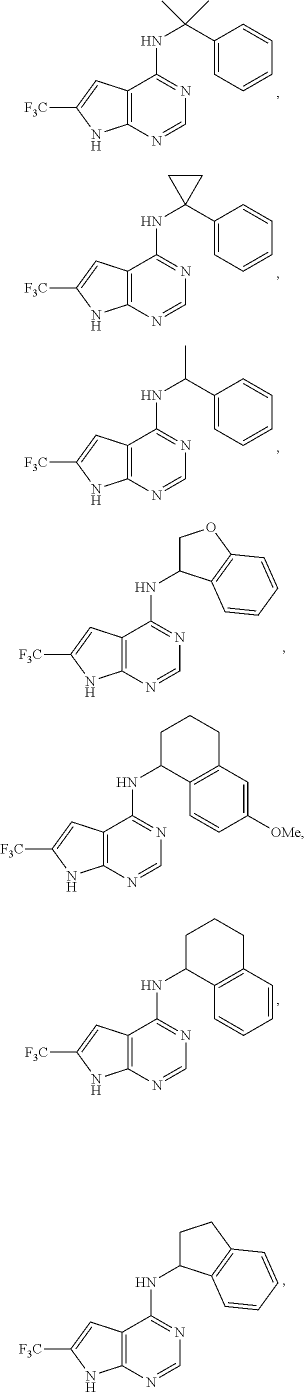

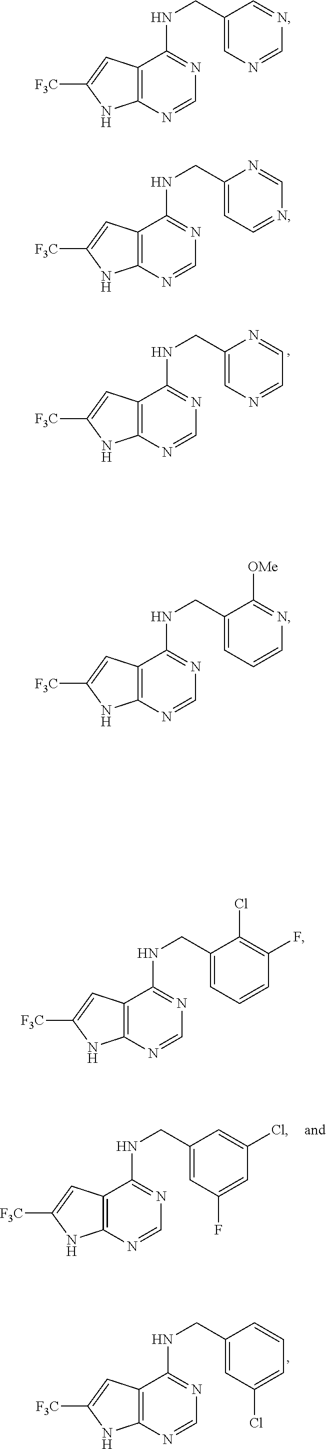

or a pharmaceutically acceptable salt thereof.

In some embodiments, a compound can be present as one or more of the following structures:

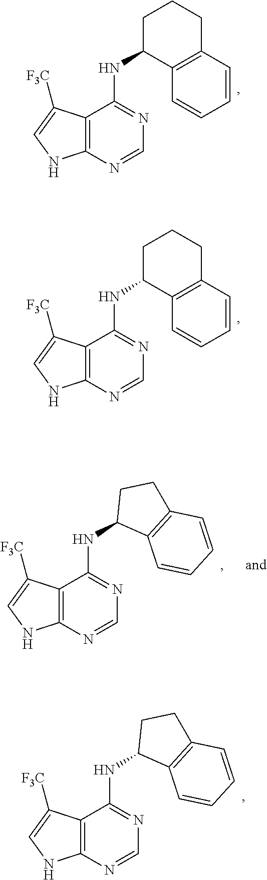 [SMILES: FC(F)(F)c1c[nH]c2ncnc(N[C@H]3CCCc4ccccc43)c12 ; FC(F)(F)c1c[nH]c2ncnc(N[C@@H]3CCCc4ccccc43)c12 ; FC(F)(F)c1c[nH]c2ncnc(N[C@H]3CCc4ccccc43)c12 ; FC(F)(F)c1c[nH]c2ncnc(N[C@@H]3CCc4ccccc43)c12]

or a pharmaceutically acceptable salt thereof.

In some embodiments, a compound can be present as one or more of the following structures:

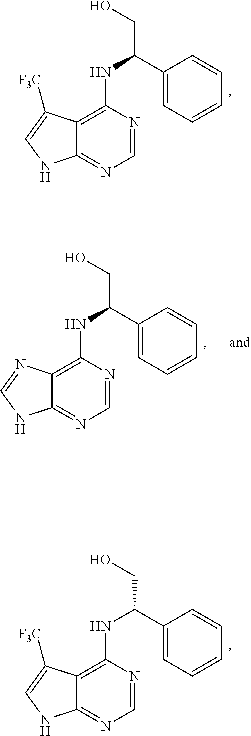 [SMILES: OC[C@H](Nc1ncnc2[nH]cc(C(F)(F)F)c12)c1ccccc1 ; OC[C@H](Nc1ncnc2[nH]cnc12)c1ccccc1 ; OC[C@@H](Nc1ncnc2[nH]cc(C(F)(F)F)c12)c1ccccc1]

or a pharmaceutically acceptable salt thereof.

In some embodiments, a compound can be present as one or more of the following structures:

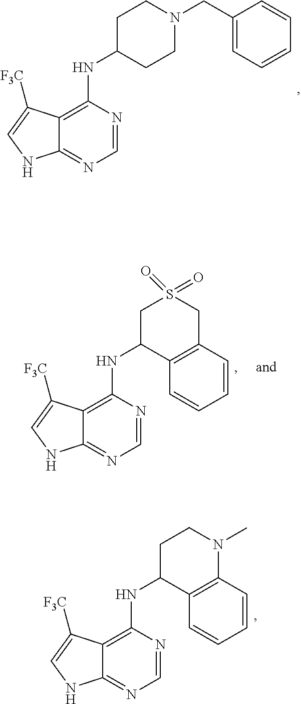 [SMILES: FC(F)(F)c1c[nH]c2ncnc(NC3CCN(Cc4ccccc4)CC3)c12 ; O=S1(=O)Cc2ccccc2C(Nc2ncnc3[nH]cc(C(F)(F)F)c23)C1 ; CN1CCC(Nc2ncnc3[nH]cc(C(F)(F)F)c23)c2ccccc21]

or a pharmaceutically acceptable salt thereof.

In some embodiments, a compound can be present as one or more of the following structures:

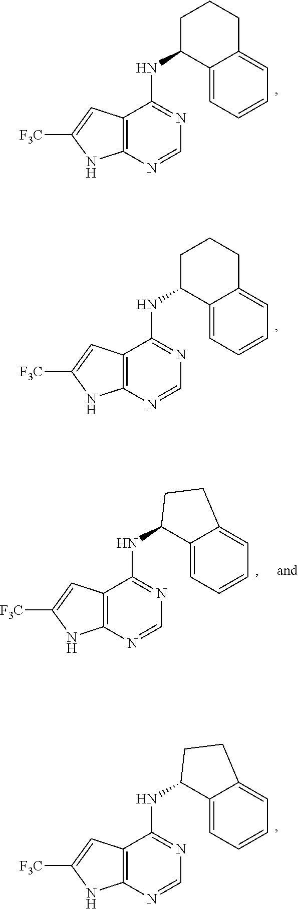 [SMILES: FC(F)(F)c1cc2c(N[C@H]3CCCc4ccccc43)ncnc2[nH]1 ; FC(F)(F)c1cc2c(N[C@@H]3CCCc4ccccc43)ncnc2[nH]1 ; FC(F)(F)c1cc2c(N[C@H]3CCc4ccccc43)ncnc2[nH]1 ; FC(F)(F)c1cc2c(N[C@@H]3CCc4ccccc43)ncnc2[nH]1]

or a pharmaceutically acceptable salt thereof.

In some embodiments, a compound can be present as one or more of the following structures:

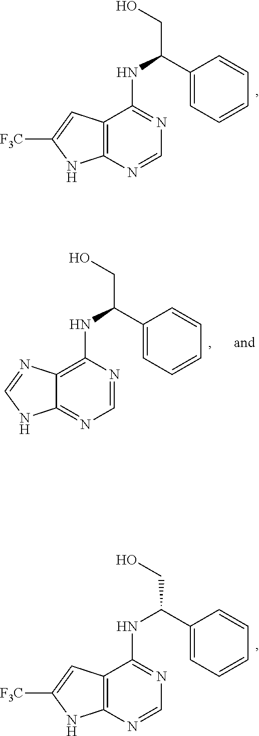 [SMILES: OC[C@H](Nc1ncnc2[nH]c(C(F)(F)F)cc12)c1ccccc1 ; OC[C@H](Nc1ncnc2[nH]cnc12)c1ccccc1 ; OC[C@@H](Nc1ncnc2[nH]c(C(F)(F)F)cc12)c1ccccc1]

or a pharmaceutically acceptable salt thereof.

In some embodiments, a compound can be present as one or more of the following structures:

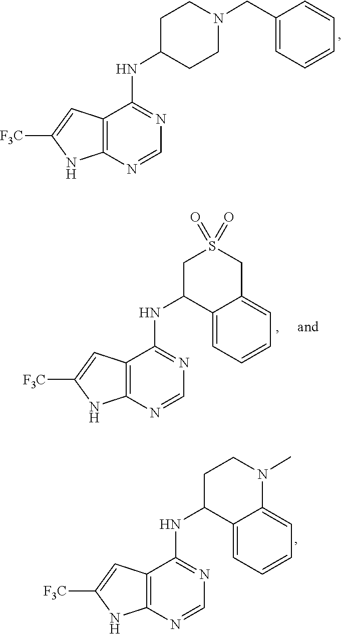 [SMILES: FC(F)(F)c1cc2c(NC3CCN(Cc4ccccc4)CC3)ncnc2[nH]1 ; O=S1(=O)Cc2ccccc2C(Nc2ncnc3[nH]c(C(F)(F)F)cc23)C1 ; CN1CCC(Nc2ncnc3[nH]c(C(F)(F)F)cc23)c2ccccc21]

or a pharmaceutically acceptable salt thereof.

In some embodiments, a compound can be present as one or more of the following structures:

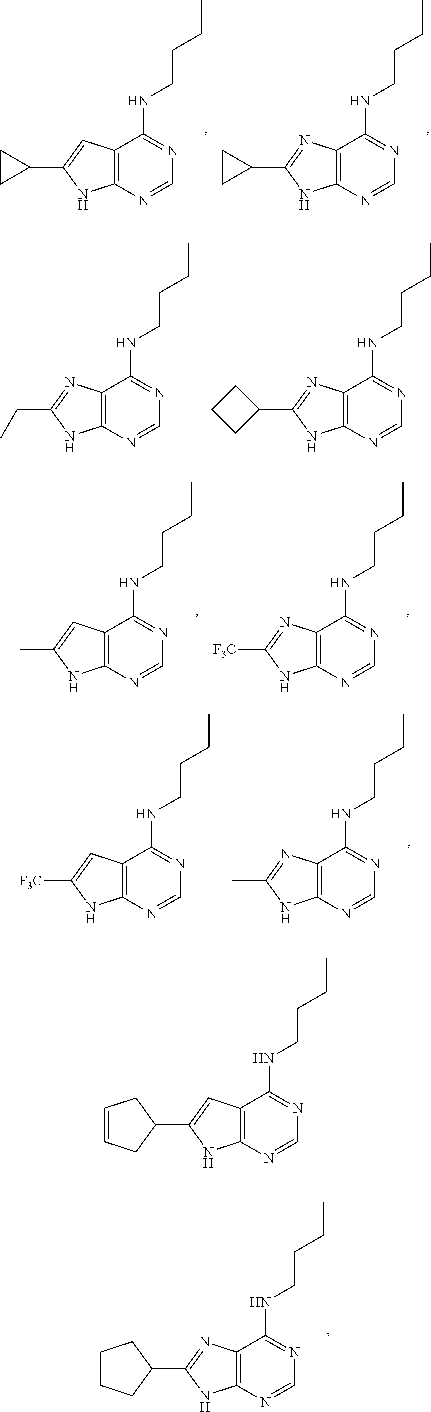

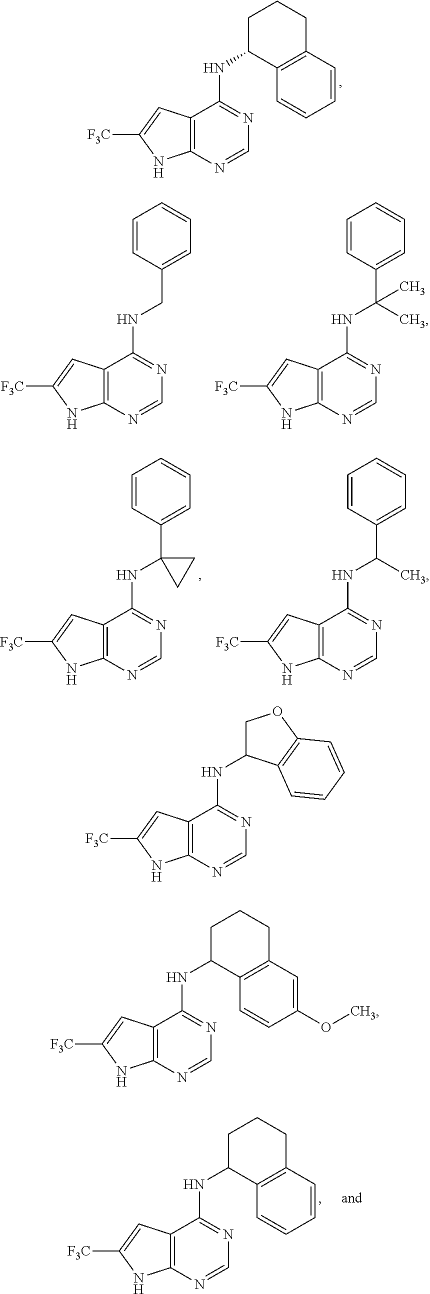

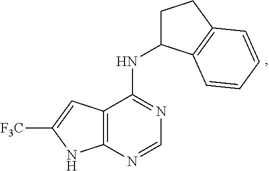 [SMILES: FC(F)(F)c1cc2c(NC3CCc4ccccc43)ncnc2[nH]1]

or a pharmaceutically acceptable salt thereof.

In some embodiments, a compound can be present as:

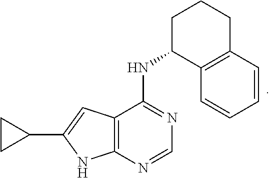 [SMILES: c1ccc2c(c1)CCC[C@H]2Nc1ncnc2[nH]c(C3CC3)cc12]

In some embodiments, a compound can be present as:

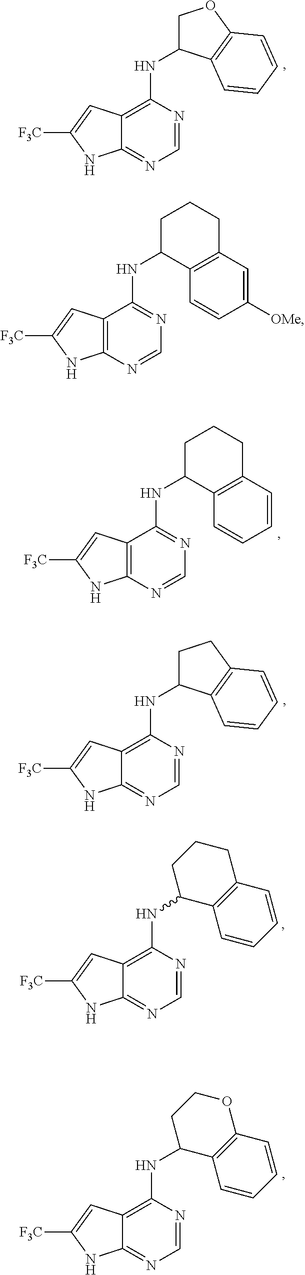 [SMILES: FC(F)(F)c1cc2c(NC3COc4ccccc43)ncnc2[nH]1 ; COc1ccc2c(c1)CCCC2Nc1ncnc2[nH]c(C(F)(F)F)cc12 ; FC(F)(F)c1cc2c(NC3CCCc4ccccc43)ncnc2[nH]1 ; FC(F)(F)c1cc2c(NC3CCc4ccccc43)ncnc2[nH]1 ; FC(F)(F)c1cc2c(NC3CCCc4ccccc43)ncnc2[nH]1 ; FC(F)(F)c1cc2c(NC3CCOc4ccccc43)ncnc2[nH]1]

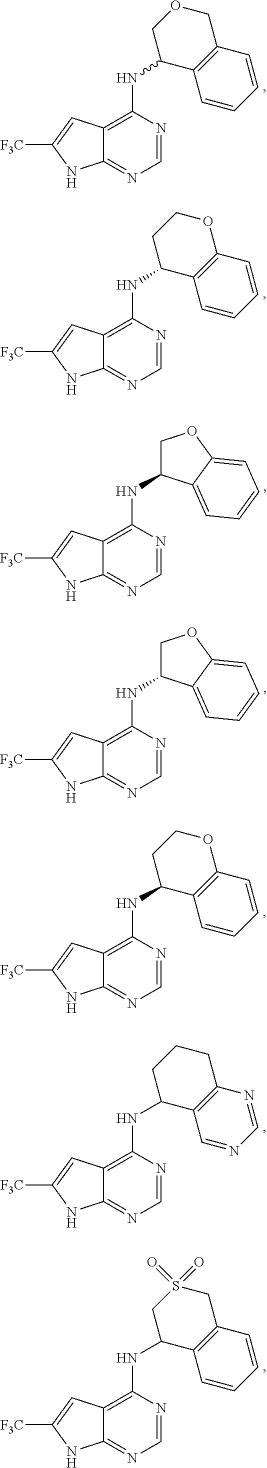

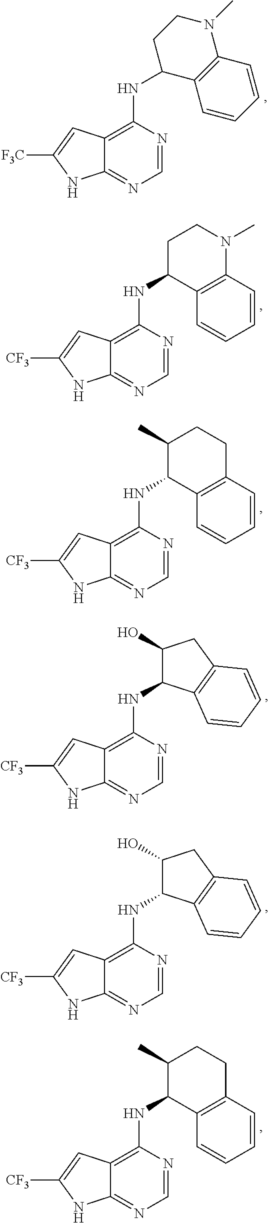

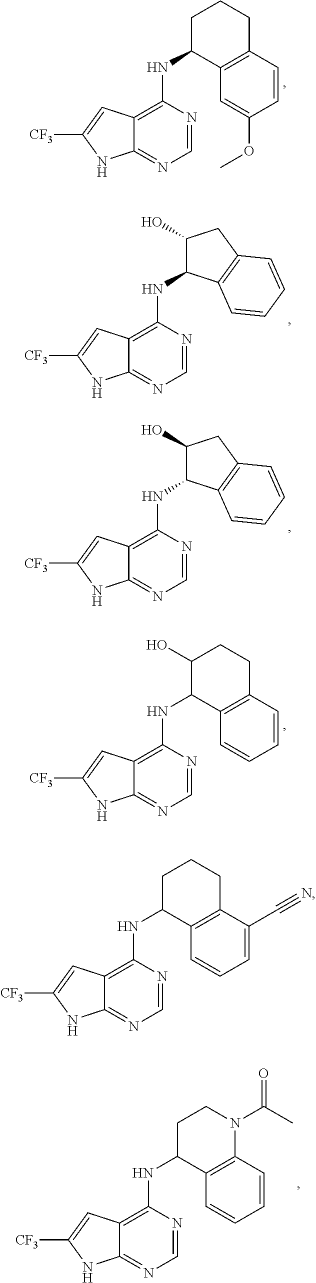

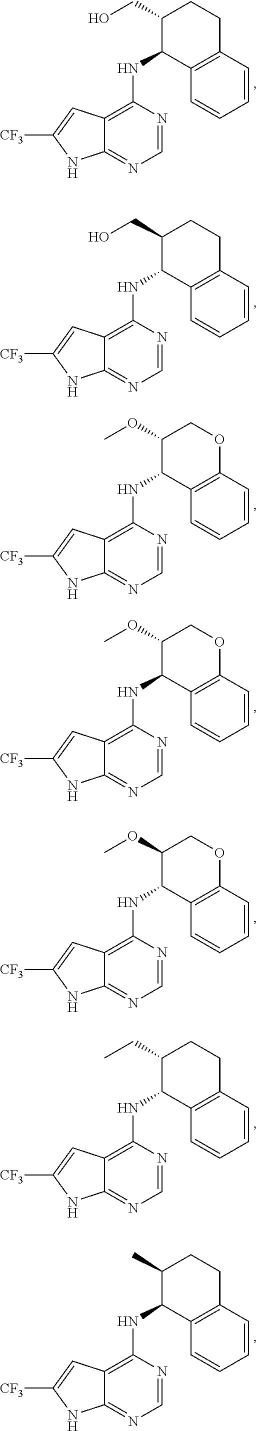

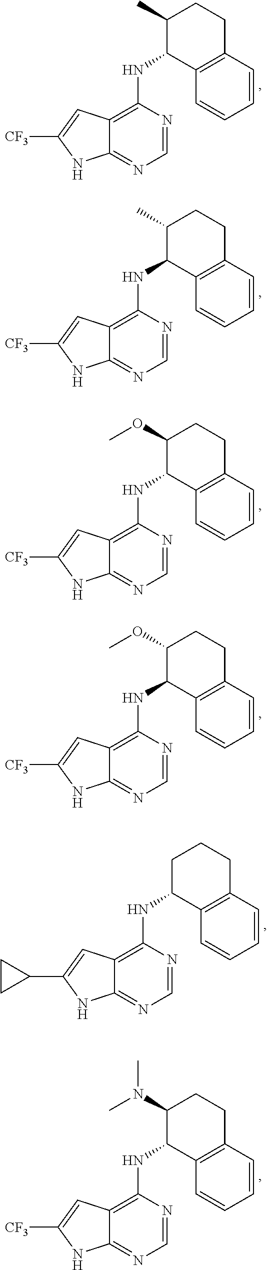

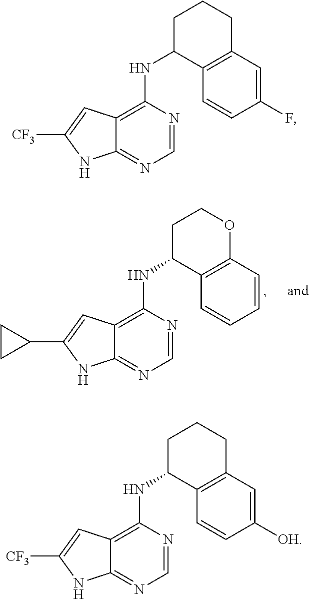 [SMILES: Fc1ccc2c(c1)CCCC2Nc1ncnc2[nH]c(C(F)(F)F)cc12 ; c1ccc2c(c1)OCC[C@H]2Nc1ncnc2[nH]c(C3CC3)cc12 ; Oc1ccc2c(c1)CCC[C@H]2Nc1ncnc2[nH]c(C(F)(F)F)cc12]

### C. Pharmaceutical Compositions

Also provided herein are pharmaceutical compositions comprising a disclosed compound, or pharmaceutically acceptable salt thereof; and a pharmaceutically acceptable carrier. Thus, in various embodiments, disclosed are pharmaceutical compositions comprising a therapeutically effective amount at least one disclosed compound and a pharmaceutically acceptable carrier. In a further embodiment, a pharmaceutical composition can be provided comprising a therapeutically effective amount of at least one disclosed compound. In a still further embodiment, a pharmaceutical composition can be provided comprising a prophylactically effective amount of at least one disclosed compound. In yet a further embodiment, the invention relates to pharmaceutical compositions comprising a pharmaceutically acceptable carrier and a compound, wherein the compound is present in an effective amount.

Thus, in various embodiments, provided herein are pharmaceutical compositions comprising a therapeutically effective amount of a compound having a structure represented by a formula:

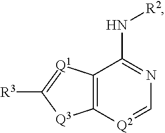

wherein Q¹ is N or CH and R³ is a 3- to 6-membered cycloalkyl, C1-C6 haloalkyl, C1-C6 haloalkoxy, or C1-C6 halohydroxyalkyl; or wherein Q¹ is CR¹ and R³ is hydrogen; R¹ is C1-C6 haloalkyl, C1-C6 haloalkoxy, C1-C6 halohydroxyalkyl, or a structure represented by a formula:

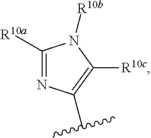

wherein each of R^{10a}, R^{10b}, and R^{10c}, when present, is independently selected from hydrogen and C1-C4 alkyl; wherein Q² is CH or N; wherein Q³ is CH₂ or NH; wherein R² is C1-C6 alkyl, —CR^{11a}R^{11b}Cy¹, or Cy¹; wherein each of R^{11a} and R^{11b}, when present, is independently selected from hydrogen, C1-C5 alkyl, and C1-C4 hydroxyalkyl; or wherein each of R^{11a} and R^{11b}, when present, together comprise a 3-membered cycloalkyl; wherein Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, provided that when R¹ is C1-C6 haloalkyl and R² is Cy¹, then Cy¹ is not a 6-membered carbocycle or a 9-membered heteroaryl, and provided that when R² is —CR^{11a}R^{11b}Cy¹ or Cy¹, one or both of R^{11a} and R^{11b}, when present, is hydrogen, and Cy¹ is a 6-membered aryl or furanyl, then Q¹ is CH and R³ is not a C1-C6 haloalkyl, or a pharmaceutically acceptable salt thereof, and a pharmaceutically acceptable carrier.

Also provided herein are pharmaceutical compositions comprising a therapeutically effective amount of a compound having a structure:

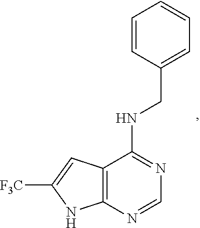 [SMILES: FC(F)(F)c1cc2c(NCc3ccccc3)ncnc2[nH]1]

or a pharmaceutically acceptable salt thereof, and a pharmaceutically acceptable carrier.

Also provided herein are pharmaceutical compositions comprising a therapeutically effective amount of a compound having a structure represented by a formula:

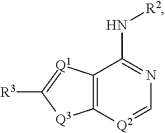

wherein Q¹ is N or CH and R³ is a 3- to 6-membered cycloalkyl, C1-C4 haloalkyl, C1-C4 cyanoalkyl, C1-C4 hydroxyalkyl, C1-C4 haloalkoxy, CF₃, CBr₃ or CCl₃; or wherein Q¹ is CR¹ and R³ is hydrogen; R¹ is C1-C6 haloalkyl, C1-C6 haloalkoxy, C1-C6 halohydroxy, or a structure represented by a formula:

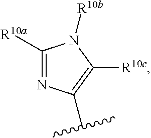

wherein each of R^{10a}, R^{10b}, and R^{10c}, when present, is independently selected from hydrogen and C1-C4 alkyl; wherein Q² is CH or N; wherein Q³ is CH₂ or NH; wherein R² is C1-C6 alkyl, —CR^{11a}R^{11b}Cy¹, or Cy¹; wherein each of R^{11a} and R^{11b}, when present, is independently selected from hydrogen, C1-C5 alkyl, and C1-C5 hydroxyalkyl; or wherein each of R^{11a} and R^{11b} together comprise a 3-membered cycloalkyl; wherein Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, provided that when R¹ is C1-C6 haloalkyl and R² is Cy¹, then Cy¹ is not a 6-membered carbocycle or a 9-membered heteroaryl, or a pharmaceutically acceptable salt thereof, and a pharmaceutically acceptable carrier.

Also provided herein are pharmaceutical compositions comprising a therapeutically effective amount of a compound selected from:

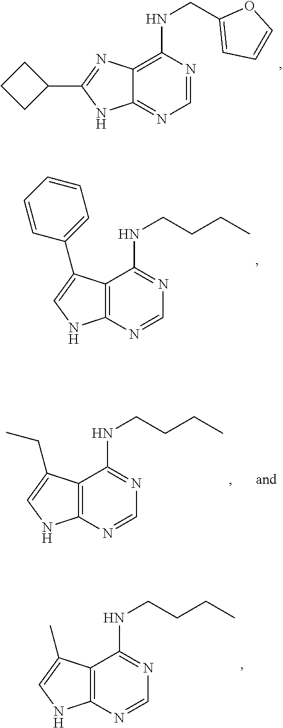

or a pharmaceutically acceptable salt thereof, and a pharmaceutically acceptable carrier.

Also provided herein are pharmaceutical compositions comprising a therapeutically effective amount of a compound selected from:

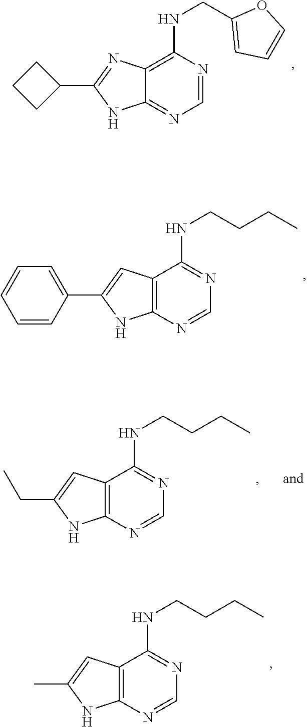

or a pharmaceutically acceptable salt thereof, and a pharmaceutically acceptable carrier.

Also provided herein are pharmaceutical compositions comprising a therapeutically effective amount of a compound selected from:

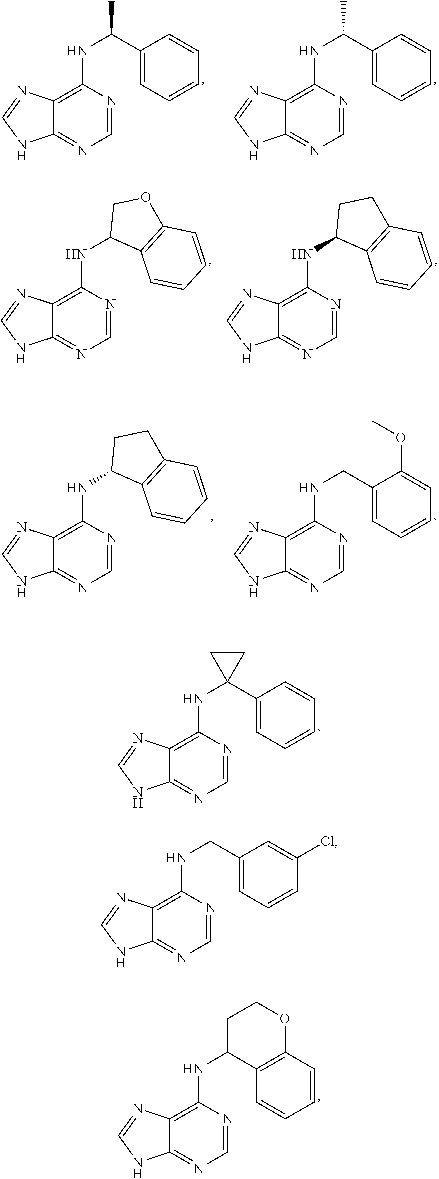

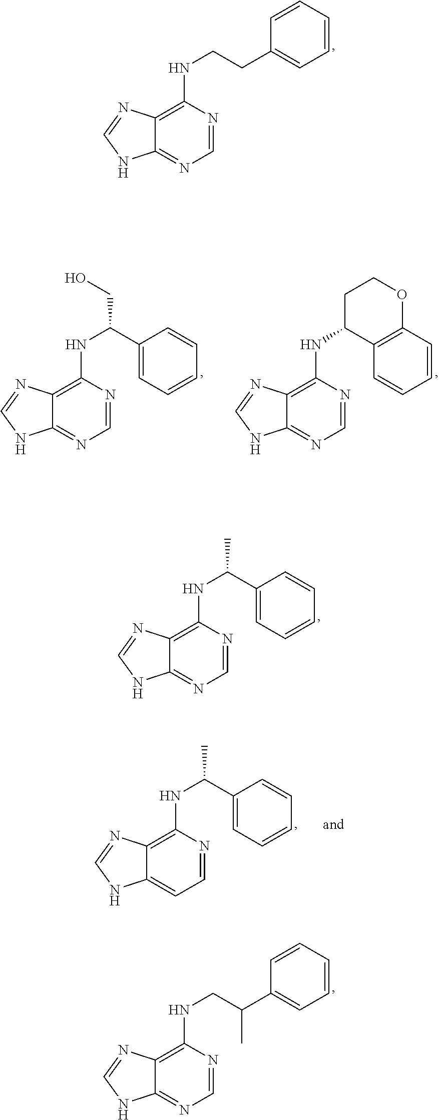 [SMILES: c1ccc(CCNc2ncnc3[nH]cnc23)cc1 ; OC[C@@H](Nc1ncnc2[nH]cnc12)c1ccccc1 ; c1ccc2c(c1)OCC[C@H]2Nc1ncnc2[nH]cnc12 ; C[C@@H](Nc1ncnc2[nH]cnc12)c1ccccc1 ; C[C@@H](Nc1nccc2[nH]cnc12)c1ccccc1 ; CC(CNc1ncnc2[nH]cnc12)c1ccccc1]

or a pharmaceutically acceptable salt thereof, and a pharmaceutically acceptable carrier.

Also provided herein are pharmaceutical compositions comprising a therapeutically effective amount of a compound having a structure represented by Formula I:

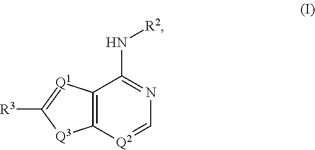

wherein Q¹ is N or CH and R³ is a 3- to 6-membered cycloalkyl or a C1-C6 haloalkyl, C1-C6 haloalkoxy, C1-C6 halohydroxyl, CF₃, CCl₃, CBr₃; or wherein Q¹ is CR¹ and R³ is hydrogen; wherein Q² is CH or N; wherein Q³ is CH₂ or NH; R¹ is (C1-C6)alkyl, halo(C1-C4)alkyl, (C1-C4)alkoxy, halo(C1-C4)alkoxy, 5- or 6-membered heteroaryl, or phenyl, wherein said C1-C6alkyl and halo(C1-C4)alkyl are each optionally and independently substituted with a OR^{a} group, and wherein said phenyl and 5- or 6-membered heteroaryl are each optionally and independently substituted with 1 to 3 groups independently selected from R^{b}; R^{a}, when present, is H, (C1-C4)alkyl, or (C1-C4)alkoxy; each occurrence of R^{b}, when present, is independently halo, halo(C1-C4)alkyl, (C1-C4)alkoxy, or halo(C1-C4)alkoxy; R² is (C1-C6)alkyl, a 9-membered oxygen-containing fused heterocycle, or a 9- to 10-membered carbocycle, wherein said (C1-C6)alkyl is optionally substituted with 1 or 2 groups independently selected from R^{c}, and wherein said 9-membered oxygen-containing fused heterocycle and 9- to 10-membered carbocycle are each optionally and independently substituted with 1 to 3 groups independently selected from R^{d}; each occurrence of R^{c}, when present, is phenyl, 3- or 4-membered cycloalkyl, or 5- or 6-membered heteroaryl, wherein said phenyl and 5- or 6-membered heteroaryl are each optionally and independently substituted with 1 to 3 groups independently selected from R^{e}; each occurrence of R^{d} and R^{e}, when present, is independently halo, halo(C₁-C₄)alkyl, (C₁-C₄)alkoxy, or halo(C₁-C₄)alkoxy; and R³ is hydrogen, halogen, (C₁-C₄)alkyl, or 3- to 6-membered cycloalkyl, or pharmaceutically acceptable salts thereof, and a pharmaceutically acceptable carrier.

In some embodiments, the disclosed pharmaceutical composition can contain a compound having a formula as recited herein, wherein the compound has an EC₅₀ of from about 0.01 μM to about 5.0 μM, about 0.01 μM to about 4.0 μM, about μM to about 3.0 μM, about 0.01 μM to about 2.0 μM, about 0.01 μM to about 1.0 μM, about 0.01 μM to about 0.5 μM, about 0.1 μM to about 5.0 μM, about 0.5 μM to about 5.0 μM, about 1.0 μM to about 5.0 μM, about 2.0 μM to about 5.0 μM, about 3.0 μM to about 5.0 μM, about 4.0 μM to about 5.0 μM, about 0.1 μM to about 4.0 μM, about 0.1 μM to about 3.0 μM, about 0.1 μM to about 2.0 μM, about 0.1 μM to about 1.0 μM, about 0.1 μM to about 0.5 μM, or about 0.2 μM to about 0.5 μM.

In some embodiments, the compounds described herein may be present in the form of pharmaceutically acceptable salts. For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Examples of pharmaceutically acceptable base addition salts include e.g., sodium, potassium, calcium, ammonium, organic amino, or magnesium salt. The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. In some embodiments, the “pharmaceuticallt acceptable carrier” includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro, (Lippincott, Williams & Wilkins, Baltimore, Md., 2006) discloses various excipients used in formulating pharmaceutical compositions and known techniques for the preparation thereof. Except insofar as any conventional excipient is incompatible with a substance or its derivatives, such as by producing any undesirable biological effect or otherwise interacting in a deleterious manner with any other component(s) of the pharmaceutical composition, its use is contemplated to be within the scope of this invention. In some embodiments, the pharmaceutically acceptable excipient or carrier is at least 95%, 96%, 97%, 98%, 99%, or 100% pure. In some embodiments, the excipient is approved for use in humans and for veterinary use. In some embodiments, the excipient is approved by United States Food and Drug Administration. In some embodiments, the excipient is pharmaceutical grade. In some embodiments, the excipient meets the standards of the United States Pharmacopoeia (USP), the European Pharmacopoeia (EP), the British Pharmacopoeia, and/or the International Pharmacopoeia. Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in the inventive formulations. Excipients such as cocoa butter and suppository waxes, coloring agents, coating agents, sweetening, flavoring, and perfuming agents can be present in the composition, according to the judgment of the formulator. Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof. Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone), (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof. Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydro xymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include, but are not limited to, starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan); alginates; polyethylene oxide; polyethylene glycol; inorganic calcium salts; silicic acid; polymethacrylates; waxes; water; alcohol; etc.; and combinations thereof.

Pharmaceutically acceptable salts of the compounds are conventional acid-addition salts or base-addition salts that retain the biological effectiveness and properties of the compounds and are formed from suitable non-toxic organic or inorganic acids or organic or inorganic bases. Exemplary acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Example base-addition salts include those derived from ammonium, potassium, sodium and, quaternary ammonium hydroxides, such as for example, tetramethylammonium hydroxide. Chemical modification of a pharmaceutical compound into a salt is a known technique to obtain improved physical and chemical stability, hygroscopicity, flowability and solubility of compounds. See, e.g., H. Ansel et al., Pharmaceutical Dosage Forms and Drug Delivery Systems (6th Ed. 1995) at pp. 196 and 1456-1457.

The pharmaceutical compositions comprise the compounds in a pharmaceutically acceptable carrier. A pharmaceutically acceptable carrier refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. The compounds can be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995.

In further embodiments, the pharmaceutical composition is administered to a mammal. In still further embodiments, the mammal is a human. In an even further embodiment, the human is a patient.

In further embodiments, the pharmaceutical composition is administered following identification of the mammal in need of treatment of a disorder associated with PINK1 kinase activity. In still further embodiments, the mammal has been diagnosed with a need for treatment of a disorder associated with PINK1 kinase activity prior to the administering step.

In various embodiments, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

The choice of carrier will be determined in part by the particular method used to administer the composition. Accordingly, there is a wide variety of suitable formulations of the pharmaceutical composition of the present invention. The following formulations for oral, aerosol, parenteral, subcutaneous, intravenous, intraarterial, intramuscular, intraperitoneal, intrathecal, rectal, and vaginal administration are merely exemplary and are in no way limiting.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granule; (c) powders; (d) suspensions in an appropriate liquid; and (e) suitable emulsions. Liquid formulations may include diluents, such as water, cyclodextrin, dimethyl sulfoxide and alcohols, for example, ethanol, benzyl alcohol, propylene glycol, glycerin, and the polyethylene alcohols including polyethylene glycol, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, the addition to the active ingredient in an inert base, such as gelatin and glycerin, or sucrose and acadia, emulsions, and gels containing, in addition to the active ingredient, such carriers as are known in the art.

The compounds of the present disclosure alone or in combination with other suitable components, can be made into aerosol formulations to be administered via inhalation. These aerosol formulations can be placed into pressurized acceptable propellants, such as dichlorodifluoromethane, propane, and nitrogen. They also may be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1, 3-dioxolane-4-methanol, ethers, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcelluslose, or emulsifying agents and other pharmaceutical adjuvants.

Oils which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example. dimethyldialkylammonium halides, and alkylpyridinium halides, (b) anionic detergents such as, for example, alkyl, aryl, and olefin sulfonates, alkyl olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkyl β-aminopropionates, and 2-alkylimidazoline quaternary ammonium salts, and (e) mixtures thereof.

The parenteral formulations typically contain from about 0.5% to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

Pharmaceutically acceptable excipients are also well-known to those who are skilled in the art. The choice of excipient will be determined in part by the particular compound, as well as by the particular method used to administer the composition. Accordingly, there is a wide variety of suitable formulations of the pharmaceutical composition of the present disclosure. The following methods and excipients are merely exemplary and are in no way limiting. The pharmaceutically acceptable excipients preferably do not interfere with the action of the active ingredients and do not cause adverse side-effects. Suitable carriers and excipients include solvents such as water, alcohol, and propylene glycol, solid absorbants and diluents, surface active agents, suspending agent, tableting binders, lubricants, flavors, and coloring agents.

The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets. The requirements for effective pharmaceutical carriers for injectable compositions are well known to those of ordinary skill in the art. See Pharmaceutics and Pharmacy Practice, J.B. Lippincott Co., Philadelphia, Pa., Banker and Chalmers, Eds., 238-250 (1982) and ASHP Handbook on Injectable Drugs, Toissel, 4^{th} ed., 622-630 (1986).

Formulations suitable for topical administration include lozenges comprising the active ingredient in a flavor, usually sucrose and acacia or tragacanth; pastilles comprising the active ingredient in an inert base, such as gelatin and glycerin, or sucrose and acacia; and mouthwashes comprising the active ingredient in a suitable liquid carrier; as well as creams, emulsions, and gels containing, in addition to the active ingredient, such carriers as are known in the art.

Additionally, formulations suitable for rectal administration may be presented as suppositories by mixing with a variety of bases such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

One skilled in the art will appreciate that suitable methods of exogenously administering a compound of the present disclosure to an animal are available, and, although more than one route can be used to administer a particular compound, a particular route can provide a more immediate and more effective reaction than another route.

As regards these applications, the present method includes the administration to an animal, particularly a mammal, and more particularly a human, of a therapeutically effective amount of the compound effective in the treatment (e.g., prophylactic or therapeutic) of a disorder associated with PINK1 kinase activity. The method also includes the administration of a therapeutically effect amount of the compound for the treatment of patient having a predisposition for being afflicted with a disorder associated with PINK1 kinase activity. The dose administered to an animal, particularly a human, in the context of the present invention should be sufficient to affect a therapeutic response in the animal over a reasonable timeframe. One skilled in the art will recognize that dosage will depend upon a variety of factors including the condition of the animal, the body weight of the animal, as well as the severity and stage of the disorder.

The total amount of the compound of the present disclosure administered in a typical treatment is preferably from about 5 mg/kg to about 80 mg/kg, 5 mg/kg to about 70 mg/kg, 5 mg/kg to about 60 mg/kg, 5 mg/kg to about 50 mg/kg, 5 mg/kg to about 40 mg/kg, 5 mg/kg to about 30 mg/kg, 5 mg/kg to about 20 mg/kg, 5 mg/kg to about 10 mg/kg, 10 mg/kg to about 80 mg/kg, 20 mg/kg to about 80 mg/kg, 30 mg/kg to about 80 mg/kg, 40 mg/kg to about 80 mg/kg, 50 mg/kg to about 80 mg/kg, 60 mg/kg to about 80 mg/kg, or 70 mg/kg to about 80 mg/kg of body weight for mice, and from about 0.5 mg/kg to about 20 mg/kg, 0.5 mg/kg to about 15 mg/kg, 0.5 mg/kg to about 10 mg/kg, 0.5 mg/kg to about 5 mg/kg, 0.5 mg/kg to about 1 mg/kg, 1 mg/kg to about 20 mg/kg, 5 mg/kg to about 20 mg/kg, 10 mg/kg to about 20 mg/kg, or 15 mg/kg to about 20 mg/kg of body weight for humans per daily dose. This total amount is typically, but not necessarily, administered as a series of doses over a period of about one time per day to about three times per day, continuing for the duration of the disease.

The size of the dose also will be determined by the route, timing and frequency of administration as well as the existence, nature and extent of any adverse side effects that might accompany the administration of the compound and the desired physiological effect. It will be appreciated by one of skill in the art that various conditions or disease states, in particular chronic conditions or disease states, may require prolonged treatment involving multiple administrations.

In some embodiments, a composition described herein is formulated for administration to a patient in need of such composition. Compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the compositions described herein may be aqueous or oleaginous suspension. These suspensions may be formulated according to techniques known in the art using suitable dispersing or wetting agents and suspending agents.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the composition.

A compound described herein can be administered alone or can be coadministered with an additional therapeutic agent. Thus, the preparations can also be combined, when desired, with other active substances (e.g., to reduce metabolic degradation). Additional therapeutic agents include, but are not limited to, other active agents known to be useful in treating a disease associated neurodegeneration (e.g., Parkinson's disease such as levodopa), dopamine agonists (e.g., bromocriptine, pergolide, pramipexole, ropinirole, piribedil, cabergoline, apomorphine, lisuride), MAO-B inhibitors (e.g., selegiline or rasagiline), amantadine, anticholinergics, antipsychotics (e.g., clozapine), cholinesterase inhibitors, modafinil, or non-steroidal anti-inflammatory drugs), Angiotensin Converting Enzyme Inhibitors (e.g., Enalipril, Lisinopril), Angiotensin Receptor Blockers (e.g., Losartan, Valsartan), Beta Blockers (e.g., Lopressor, Toprol-XL), Digoxin, or Diuretics.

In some embodiments, the compounds described herein can be delivered in a vesicle, in particular a liposome (see, Langer, Science, 1990, 249, 1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

Suitable compositions include, but are not limited to, oral non-absorbed compositions. Suitable compositions also include, but are not limited to saline, water, cyclodextrin solutions, and buffered solutions of pH 3-9.

The compounds described herein, or pharmaceutically acceptable salts thereof, can be formulated with numerous excipients including, but not limited to, purified water, propylene glycol, PEG 400, glycerin, DMA, ethanol, benzyl alcohol, citric acid/sodium citrate (pH3), citric acid/sodium citrate (pH5), tris(hydroxymethyl)amino methane HCl (pH7.0), 0.9% saline, and 1.2% saline, and any combination thereof. In some embodiments, excipient is chosen from propylene glycol, purified water, and glycerin.

In some embodiments, the formulation can be lyophilized to a solid and reconstituted with, for example, water prior to use.

When administered to a mammal (e.g., to an animal for veterinary use or to a human for clinical use) the compounds can be administered in isolated form.

When administered to a human, the compounds can be sterile. Water is a suitable carrier when the compound of Formula I is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The compositions described herein can take the form of a solution, suspension, emulsion, tablet, pill, pellet, capsule, capsule containing a liquid, powder, sustained-release formulation, suppository, aerosol, spray, or any other form suitable for use. Examples of suitable pharmaceutical carriers are described in Remington's Pharmaceutical Sciences, A. R. Gennaro (Editor) Mack Publishing Co.

In some embodiments, the compounds are formulated in accordance with routine procedures as a pharmaceutical composition adapted for administration to humans. Typically, compounds are solutions in sterile isotonic aqueous buffer. Where necessary, the compositions can also include a solubilizing agent. Compositions for intravenous administration may optionally include a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound is to be administered by infusion, it can be dispensed, for example, with an infusion bottle containing sterile pharmaceutical grade water or saline. Where the compound is administered by injection, an ampoule of sterile water for injection or saline can be provided so that the ingredients may be mixed prior to administration.

The pharmaceutical compositions can be in unit dosage form. In such form, the composition can be divided into unit doses containing appropriate quantities of the active component. The unit dosage form can be a packaged preparation, the package containing discrete quantities of the preparations, for example, packeted tablets, capsules, and powders in vials or ampules. The unit dosage form can also be a capsule, cachet, or tablet itself, or it can be the appropriate number of any of these packaged forms.

In some embodiments, a composition of the present disclosure is in the form of a liquid wherein the active agent is present in solution, in suspension, as an emulsion, or as a solution/suspension. In some embodiments, the liquid composition is in the form of a gel. In other embodiments, the liquid composition is aqueous. In other embodiments, the composition is in the form of an ointment.

In some embodiments, the composition is in the form of a solid article. For example, in some embodiments, the ophthalmic composition is a solid article that can be inserted in a suitable location in the eye, such as between the eye and eyelid or in the conjunctival sac, where it releases the active agent as described, for example, U.S. Pat. Nos. 3,863,633; 3,867,519; 3,868,445; 3,960,150; 3,963,025; 4,186,184; 4,303,637; 5,443,505; and 5,869,079. Release from such an article is usually to the cornea, either via the lacrimal fluid that bathes the surface of the cornea, or directly to the cornea itself, with which the solid article is generally in intimate contact. Solid articles suitable for implantation in the eye in such fashion are generally composed primarily of polymers and can be bioerodible or non-bioerodible. Bioerodible polymers that can be used in the preparation of ocular implants carrying one or more of the compounds described herein in accordance with the present disclosure include, but are not limited to, aliphatic polyesters such as polymers and copolymers of poly(glycolide), poly(lactide), poly(epsilon-caprolactone), poly-(hydroxybutyrate) and poly(hydroxyvalerate), polyamino acids, polyorthoesters, polyanhydrides, aliphatic polycarbonates and polyether lactones. Suitable non-bioerodible polymers include silicone elastomers.

The compositions described herein can contain preservatives. Suitable preservatives include, but are not limited to, mercury-containing substances such as phenylmercuric salts (e.g., phenylmercuric acetate, borate and nitrate) and thimerosal; stabilized chlorine dioxide; quaternary ammonium compounds such as benzalkonium chloride, cetyltrimethylammonium bromide and cetylpyridinium chloride; imidazolidinyl urea; parabens such as methylparaben, ethylparaben, propylparaben and butylparaben, and salts thereof; phenoxyethanol; chlorophenoxyethanol; phenoxypropanol; chlorobutanol; chlorocresol; phenylethyl alcohol; disodium EDTA; and sorbic acid and salts thereof.

In some embodiments, the compound or pharmaceutical composition comprising the compounds disclosed herein, or the pharmaceutically acceptable salts herein, are neo-substrates of PINK1 such as, for example, the following compounds:

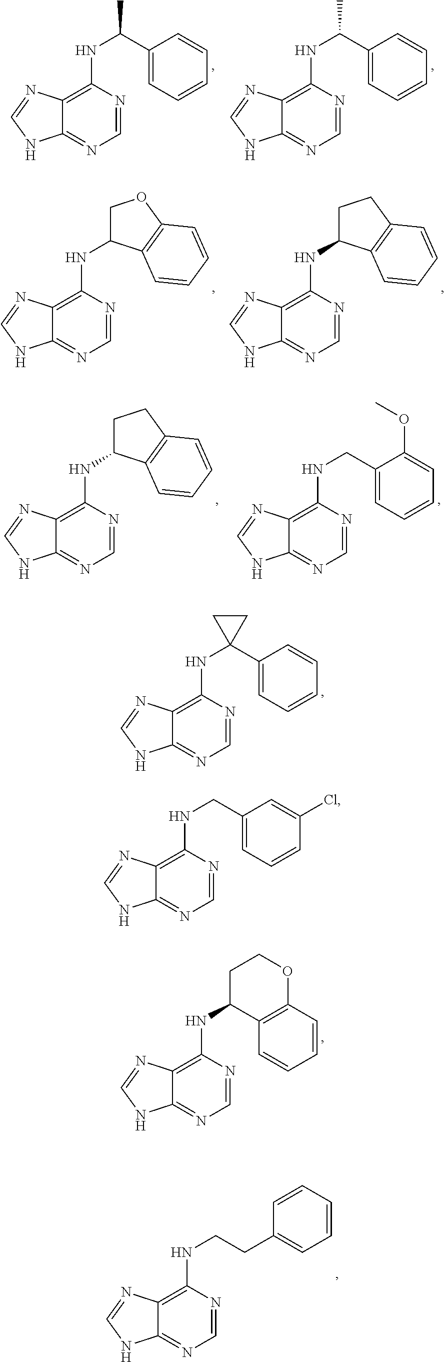

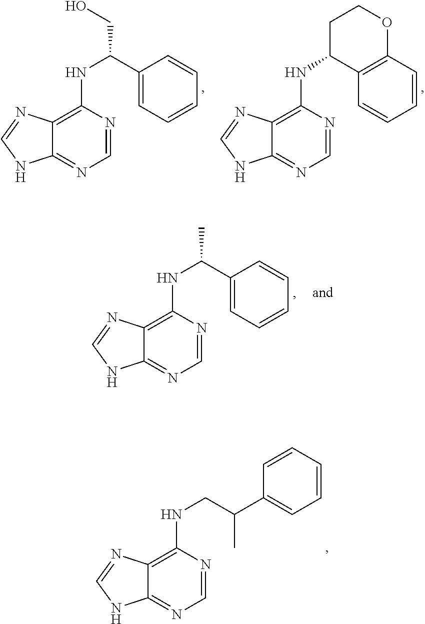

In some embodiments, the neo-substrate is not kinetin. In some embodiments, the neo-substrate is not kinetin riboside. In some embodiments, the neo-substrate is not kinetin riboside 5′ monophosphate. In some embodiments, the neo-substrate is not kinetin riboside 5′ diphosphate. In some embodiments, the neo-substrate is not kinetin riboside 5′ triphosphate. In some embodiments, the neo-substrate is not a derivative (e.g., prodrug) of kinetin, kinetin riboside, kinetin riboside 5′ monophosphate, kinetin riboside 5′ diphosphate, or kinetin riboside 5′ triphosphate. In some embodiments, the neo-substrate is not N6-(delta 2-Isopentenyl)-adenine. In some embodiments, the neo-substrate is not N6-(delta 2-Isopentenyl)-adenosine, N6-(delta 2-Isopentenyl)-adenosine 5′ monophosphate, N6-(delta 2-Isopentenyl)-adenosine 5′ diphosphate, N6-(delta 2-Isopentenyl)-adenosine 5′ triphosphate, or a derivative (e.g., prodrug) thereof. In some embodiments, the neo-substrate is not a cytokinin. In some embodiments, the neo-substrate is not a cytokinin riboside, cytokinin riboside 5′ monophosphate, cytokinin riboside 5′ diphosphate, cytokinin riboside 5′ triphosphate, or a derivative (e.g., prodrug) thereof.

Also provided are methods of treating any cardiomyopathy, fibrosis or mitochondrial disorder by administering one or more of the compositions as described above in combination with other drugs for the treatment of cardiovascular and/or mitochondrial disorders. These other drugs include cholinesterase inhibitors such as donepezil (Aricept), galantamine (Razadyne) and rivastigmine (Exelon). or analogues thereof; Memantine (Namenda); and antidepressants such as citalopram (Celexa), escitalopram (Lexapro); fluoxetine (Prozac, Sarafem, Selfemra, Prozac Weekly); fluvoxamine (Luvox); paroxetine (Paxil, Paxil CR, Pexeva); sertraline (Zoloft); vortioxetine (Trintellix, formerly known as Brintellix) and vilazodone (Viibryd). In the combination therapies, one or more compounds or compositions are coadministered with one or more drugs for the treatment of cardiovascular and/or mitochondrial 1 disorders to increase efficacy of treatment of cardiovascular and/or mitochondrial disorders and to reduce side effects associated with high doses of these therapeutics. The combination therapies described above have synergistic and additive therapeutic effects. Synergy is defined as the interaction of two or more agents so that their combined effect is greater than the sum of their individual effects. For example, if the effect of drug A alone in treating a disease is 25%, and the effect of drug B alone in treating a disease is 25%, but when the two drugs are combined the effect in treating the disease is 75%, the effect of A and B is synergistic. Additivity is defined as the interaction of two or more agents so that their combined effect is the same as the sum of their individual effects. For example, if the effect of drug A alone in treating a disease is 25%, and the effect of drug B alone in treating a disease is 25%, but when the two drugs are combined the effect in treating the disease is 50%, the effect of A and B is additive. An improvement in the drug therapeutic regimen can be described as the interaction of two or more agents so that their combined effect reduces the incidence of adverse event (AE) of either or both agents used in co-therapy. This reduction in the incidence of adverse effects can be a result of, e.g., administration of lower dosages of either or both agent used in the co-therapy. For example, if the effect of Drug A alone is 25% and has an adverse event incidence of 45% at labeled dose; and the effect of Drug B alone is 25% and has an adverse event incidence of 30% at labeled dose, but when the two drugs are combined at lower than labeled doses of each, if the overall effect is 35% (an improvement, but not synergistic or additive) and the adverse incidence rate is 20%, there is an improvement in the drug therapeutic regimen.

According to some embodiments, pharmaceutical compositions are provided comprising effective amounts of one or more compound(s) of the present invention together with, for example, pharmaceutically acceptable diluents, preservatives, solubilizers, emulsifiers, adjuvants and/or other carriers. Such compositions include diluents of various buffer content (e.g., TRIS or other amines, carbonates, phosphates, amino acids, for example, glycinamide hydrochloride (especially in the physiological pH range), N-glycylglycine, sodium or potassium phosphate (dibasic, tribasic), etc. or TRIS-HCl or acetate), pH and ionic strength; additives such as detergents and solubilizing agents (e.g., surfactants such as Pluronics, Tween 20, Tween 80 (Polysorbate 80), Cremophor, polyols such as polyethylene glycol, propylene glycol, etc.), anti-oxidants (e.g., ascorbic acid, sodium metabisulfite), preservatives (e.g., Thimersol, benzyl alcohol, parabens, etc.) and bulking substances (e.g., sugars such as sucrose, lactose, mannitol, polymers such as polyvinylpyrrolidones or dextran, etc.); and/or incorporation of the material into particulate preparations of polymeric compounds such as polylactic acid, polyglycolic acid, etc. or into liposomes. Hyaluronic acid may also be used. Such compositions can be employed to influence the physical state, stability, rate of in vivo release, and rate of in vivo clearance of a compound of the present invention. See, e.g., Remington's Pharmaceutical Sciences, 18th Ed. (1990, Mack Publishing Co., Easton, Pa. 18042) pages 1435-1712 which are herein incorporated by reference. The compositions can, for example, be prepared in liquid form, or can be in dried powder, such as lyophilized form. Particular methods of administering such compositions are described infra. Where a buffer is to be included in the formulations of the invention, the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethane, or mixtures thereof. Each one of these specific buffers constitutes an alternative embodiment of the invention. In a preferred embodiment of the invention the buffer is glycylglycine, sodium dihydrogen phosphate, disodium hydrogen phosphate, sodium phosphate or mixtures thereof. Where a pharmaceutically acceptable preservative is to be included in the formulations of the invention, the preservative is selected from the group consisting of phenol, m-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, or mixtures thereof. Each one of these specific preservatives constitutes an alternative embodiment of the invention. In a preferred embodiment of the invention the preservative is phenol or m-cresol.

In a further embodiment of the invention the preservative is present in a concentration from about 0.1 mg/ml to about 50 mg/ml, more preferably in a concentration from about 0.1 mg/ml to about 25 mg/ml, and most preferably in a concentration from about 0.1 mg/ml to about 10 mg/ml. The use of a preservative in pharmaceutical compositions is well-known to the skilled person. For convenience reference is made to Remington: The Science and Practice of Pharmacy, 19th edition, 1995. In a further embodiment of the invention the formulation may further comprise a chelating agent where the chelating agent may be selected from salts of ethlenediaminetetraacetic acid (EDTA), citric acid, and aspartic acid, and mixtures thereof. Each one of these specific chelating agents constitutes an alternative embodiment of the invention.

In a further embodiment of the invention the chelating agent is present in a concentration from 0.1 mg/ml to 5 mg/ml. In a further embodiment of the invention the chelating agent is present in a concentration from 0.1 mg/ml to 2 mg/ml. In a further embodiment of the invention the chelating agent is present in a concentration from 2 mg/ml to 5 mg/ml.

The use of a chelating agent in pharmaceutical compositions is well-known to the skilled person. For convenience reference is made to Remington: The Science and Practice of Pharmacy, 19th edition, 1995.

In a further embodiment of the invention the formulation may further comprise a stabilizer selected from the group of high molecular weight polymers or low molecular compounds where such stabilizers include, but are not limited to, polyethylene glycol (e.g. PEG 3350), polyvinylalcohol (PVA), polyvinylpyrrolidone, carboxymethylcellulose, different salts (e.g. sodium chloride), L-glycine, L-histidine, imidazole, arginine, lysine, isoleucine, aspartic acid, tryptophan, threonine and mixtures thereof. Each one of these specific stabilizers constitutes an alternative embodiment of the invention. In a preferred embodiment of the invention the stabilizer is selected from the group consisting of L-histidine, imidazole and arginine.

In a further embodiment of the invention the high molecular weight polymer is present in a concentration from 0.1 mg/ml to 50 mg/ml. In a further embodiment of the invention the high molecular weight polymer is present in a concentration from 0.1 mg/ml to 5 mg/ml. In a further embodiment of the invention the high molecular weight polymer is present in a concentration from 5 mg/ml to 10 mg/ml. In a further embodiment of the invention the high molecular weight polymer is present in a concentration from 10 mg/ml to 20 mg/ml. In a further embodiment of the invention the high molecular weight polymer is present in a concentration from 20 mg/ml to 30 mg/ml. In a further embodiment of the invention the high molecular weight polymer is present in a concentration from 30 mg/ml to 50 mg/ml.

In a further embodiment of the invention the low molecular weight compound is present in a concentration from 0.1 mg/ml to 50 mg/ml. In a further embodiment of the invention the low molecular weight compound is present in a concentration from 0.1 mg/ml to 5 mg/ml. In a further embodiment of the invention the low molecular weight compound is present in a concentration from 5 mg/ml to 10 mg/ml. In a further embodiment of the invention the low molecular weight compound is present in a concentration from 10 mg/ml to 20 mg/ml. In a further embodiment of the invention the low molecular weight compound is present in a concentration from 20 mg/ml to 30 mg/ml. In a further embodiment of the invention the low molecular weight compound is present in a concentration from 30 mg/ml to 50 mg/ml.

The use of a stabilizer in pharmaceutical compositions is well-known to the skilled person. For convenience reference is made to Remington: The Science and Practice of Pharmacy, 19th edition, 1995.

In a further embodiment of the invention the formulation of the invention may further comprise a surfactant where a surfactant may be selected from a detergent, ethoxylated castor oil, polyglycolyzed glycerides, acetylated monoglycerides, sorbitan fatty acid esters, poloxamers, such as 188 and 407, polyoxyethylene sorbitan fatty acid esters, polyoxyethylene derivatives such as alkylated and alkoxylated derivatives (tweens, e.g. Tween-20, or Tween-80), monoglycerides or ethoxylated derivatives thereof, diglycerides or polyoxyethylene derivatives thereof, glycerol, cholic acid or derivatives thereof, lecithins, alcohols and phospholipids, glycerophospholipids (lecithins, kephalins, phosphatidyl serine), glyceroglycolipids (galactopyransoide), sphingophospholipids (sphingomyelin), and sphingoglycolipids (ceramides, gangliosides), DSS (docusate sodium, docusate calcium, docusate potassium, SDS (sodium dodecyl sulfate or sodium lauryl sulfate), dipalmitoyl phosphatidic acid, sodium caprylate, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-1-propanesulfonate, anionic (alkyl-aryl-sulphonates) monovalent surfactants, palmitoyl lysophosphatidyl-L-serine, lysophospholipids (e.g. 1-acyl-sn-glycero-3-phosphate esters of ethanolamine, choline, serine or threonine), alkyl, alkoxyl (alkyl ester), alkoxy (alkyl ether)-derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the postively charged DODAC, DOTMA, DCP, BISHOP, lysophosphatidylserine and lysophosphatidylthreonine, zwitterionic surfactants (e.g. N-alkyl-N,N-dimethylammonio-1-propanesulfonates, 3-cholamido-1-propyldimethylammonio-1-propanesulfonate, dodecylphosphocholine, myristoyl lysophosphatidylcholine, hen egg lysolecithin), cationic surfactants (quartemary ammonium bases) (e.g. cetyl-trimethylammonium bromide, cetylpyridinium chloride), non-ionic surfactants, polyethyleneoxide/polypropyleneoxide block copolymers (Pluronics/Tetronics, Triton X-100, Dodecyl β-D-glucopyranoside) or polymeric surfactants (Tween-40, Tween-80, Brij-35), fusidic acid derivatives—(e.g. sodium tauro-dihydrofusidate etc.), long-chain fatty acids and salts thereof C6-C12 (e.g. oleic acid and caprylic acid), acylcamitines and derivatives, N_{α}-acylated derivatives of lysine, arginine or histidine, or side-chain acylated derivatives of lysine or arginine, N_{α}-acylated derivatives of dipeptides comprising any combination of lysine, arginine or histidine and a neutral or acidic amino acid, N_{α}-acylated derivative of a tripeptide comprising any combination of a neutral amino acid and two charged amino acids, or the surfactant may be selected from the group of imidazoline derivatives, or mixtures thereof. Each one of these specific surfactants constitutes an alternative embodiment of the disclosure.

The use of a surfactant in pharmaceutical compositions is well-known to the skilled person. For convenience reference is made to Remington: The Science and Practice of Pharmacy, 19th edition, 1995. Pharmaceutically acceptable sweeteners comprise preferably at least one intense sweetener such as saccharin, sodium or calcium saccharin, aspartame, acesulfame potassium, sodium cyclamate, alitame, a dihydrochalcone sweetener, monellin, stevioside or sucralose (4, 1′,6′-trichloro-4,1′,6′-trideoxygalactosucrose), preferably saccharin, sodium or calcium saccharin, and optionally a bulk sweetener such as sorbitol, mannitol, fructose, sucrose, maltose, isomalt, glucose, hydrogenated glucose syrup, xylitol, caramel or honey.

In some embodiments, the disclosure relates to a pharmaceutical composition comprising: (i) a therapeutically effective amount of one or a plurality of compounds disclosed herein; and (ii) a pharmaceutically acceptable carrier for treatment of a mitochondrial disease.

It is understood that the disclosed compositions can be prepared from the disclosed compounds. It is also understood that the disclosed compositions can be employed in the disclosed methods of using.

### D. Methods of Making the Compounds

In various embodiments, the inventions relates to methods of making compounds useful to treat a disorder associated with PINK1 kinase activity. Thus, in some embodiments, disclosed are methods of making a disclosed compound.

Compounds according to the present disclosure can, for example, be prepared by the several methods outlined below. A practitioner skilled in the art will understand the appropriate use of protecting groups [see: Greene and Wuts, Protective Groups in Organic Synthesis] and the preparation of known compounds found in the literature using the standard methods of organic synthesis. There may come from time to time the need to rearrange the order of the recommended synthetic steps, however this will be apparent to the judgment of a chemist skilled in the art of organic synthesis. The following examples are provided so that the invention might be more fully understood, are illustrative only, and should not be construed as limiting.

In some embodiments, the disclosed compounds comprise the products of the synthetic methods described herein. In further embodiments, the disclosed compounds comprise a compound produced by a synthetic method described herein. In still further embodiments, the invention comprises a pharmaceutical composition comprising a therapeutically effective amount of the product of the disclosed methods and a pharmaceutically acceptable carrier. In still further embodiments, the invention comprises a method for manufacturing a medicament comprising combining at least one compound of any of disclosed compounds or at least one product of the disclosed methods with a pharmaceutically acceptable carrier or diluent.

1. Route I

In some embodiments, N-containing heteroaryl analogs can be prepared as shown below.

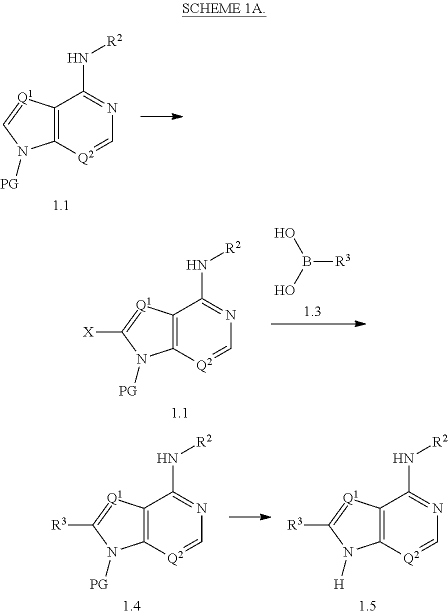

Compounds are represented in generic form, wherein X is a halogen, wherein PG is an amine protecting group, and with substituents as noted in compound descriptions elsewhere herein. A more specific example is set forth below.

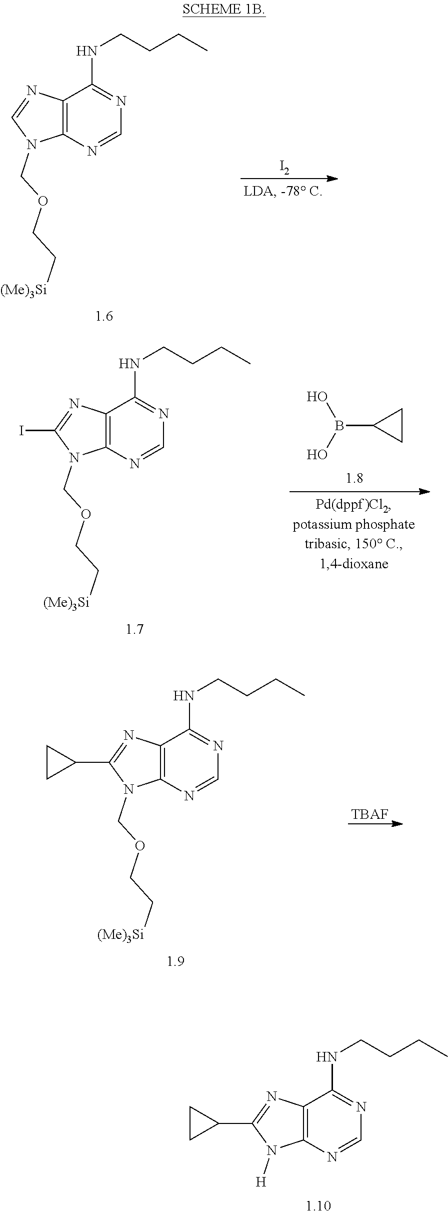

In some embodiments, compounds of type 1.5, and similar compounds, can be prepared according to reaction Scheme 1B above. Thus, compounds of type 1.7 can be prepared by a halogenation reaction of an appropriate adenine analog, e.g., 1.6 as shown above. Appropriate adenine analogs are commercially available or prepared by methods known to one skilled in the art. The halogenation reaction is carried out in the presence of an appropriate halide source, e.g., iodine, and an appropriate base, e.g., lithium diisopropylamide (LDA) at an appropriate temperature, e.g., −78° C. Compounds of type 1.9 can be prepared by a coupling reaction of an appropriate halide, e.g., 1.7 as shown above, and an appropriate boronic acid, e.g., 1.8 as shown above. Appropriate boronic acids are commercially available or prepared by methods known to one skilled in the art. The coupling reaction is carried out in the presence of an appropriate catalyst, e.g., [1,1′-Bis(diphenylphosphino)ferrocene]dichloropalladium(II), and an appropriate ligand, e.g., potassium phosphate tribasic, in an appropriate solvent, e.g., 1,4-dioxane, at an appropriate temperature, e.g., 150° C. Compounds of type 1.10 can be prepared by deprotection of an appropriate protected amine, e.g., 1.9 as shown above. The deprotection is carried out in the presence of an appropriate deprotecting agent, e.g., tetrabutylammonium fluoride (TBAF). As can be appreciated by one skilled in the art, the above reaction provides an example of a generalized approach wherein compounds similar in structure to the specific reactants above (compounds similar to compounds of type 1.1, 1.2, 1.3, and 1.4), can be substituted in the reaction to provide substituted N-containing heteroaryl analogs similar to Formula 1.5.

2. Route II

In some embodiments, N-containing heteroaryl analogs can be prepared as shown below.

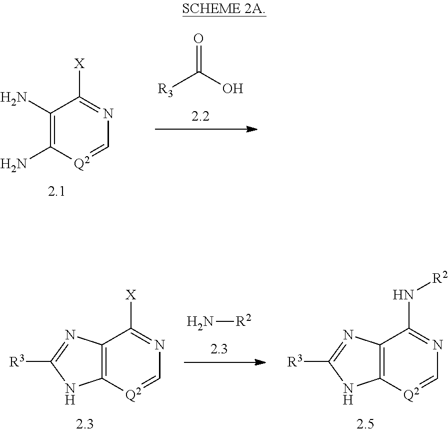

Compounds are represented in generic form, wherein X is a halogen and with substituents as noted in compound descriptions elsewhere herein. A more specific example is set forth below.

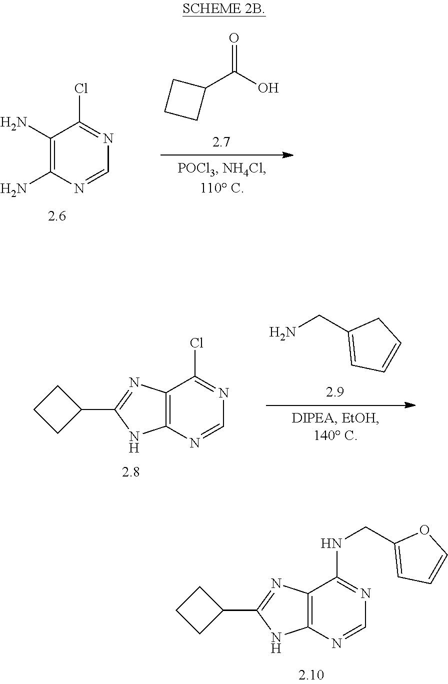 [SMILES: Nc1ncnc(Cl)c1N ; O=C(O)C1CCC1 ; Clc1ncnc2[nH]c(C3CCC3)nc12 ; NCC1=CC=CC1 ; c1coc(CNc2ncnc3[nH]c(C4CCC4)nc23)c1]

In some embodiments, compounds of type 2.5, and similar compounds, can be prepared according to reaction Scheme 2B above. Thus, compounds of type 2.8 can be prepared by a cyclization reaction of an appropriate diamine, e.g., 2.6 as shown above, and an appropriate carboxylic acid, e.g., 2.7 as shown above. Appropriate diamines and appropriate carboxylic acids are commercially available or prepared by methods known to one skilled in the art. The cyclization reaction is carried out in the presence of an appropriate oxidant, e.g., phosphorous oxychloride, and an appropriate base, e.g., ammonium chloride, at an appropriate temperature, e.g., 110° C. Compounds of type 2.10 can be prepared by a coupling reaction of an appropriate halide, e.g., 2.8 as shown above, and an appropriate amine, e.g., 2.9 as shown above. Appropriate amines are commercially available or prepared by methods known to one skilled in the art. The coupling reaction is carried out in the presence of an appropriate base, e.g., diisopropylethylamine (DIPEA), in an appropriate solvent, e.g., ethanol, at an appropriate temperature, e.g., 140° C. As can be appreciated by one skilled in the art, the above reaction provides an example of a generalized approach wherein compounds similar in structure to the specific reactants above (compounds similar to compounds of type 2.1, 2.2, 2.3, and 2.4), can be substituted in the reaction to provide substituted N-containing heteroaryl analogs similar to Formula 2.5.

3. Route III

In some embodiments, N-containing heteroaryl analogs can be prepared as shown below.

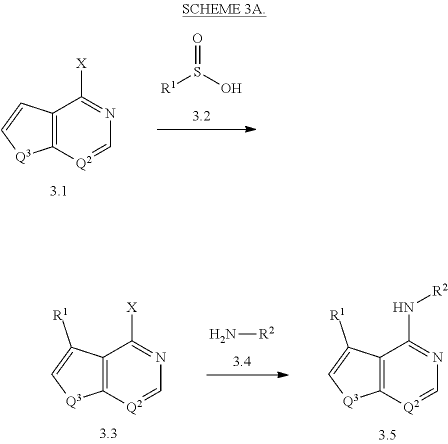

Compounds are represented in generic form, wherein Z is a halogen and with substituents as noted in compound descriptions elsewhere herein. A more specific example is set forth below.

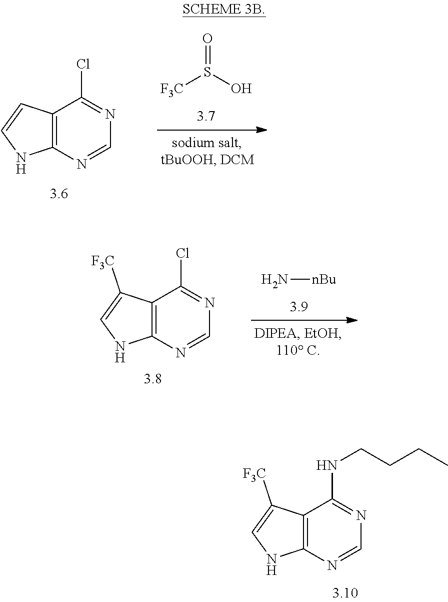

In some embodiments, N-containing heteroaryl analogs can be prepared as shown below.

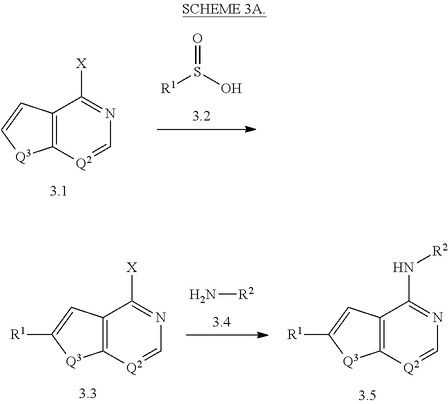

Compounds are represented in generic form, wherein Z is a halogen and with substituents as noted in compound descriptions elsewhere herein. A more specific example is set forth below.

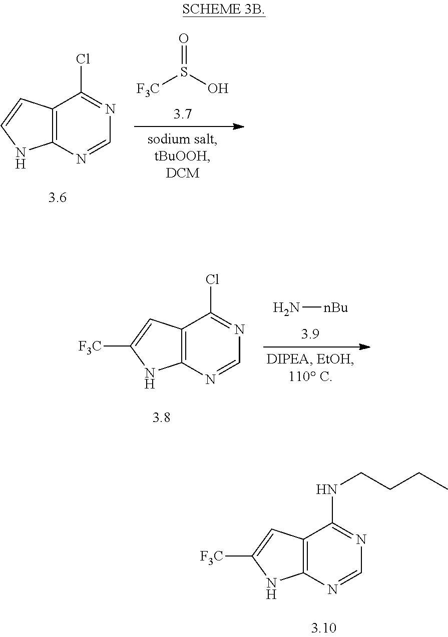

In some embodiments, compounds of type 3.5, and similar compounds, can be prepared according to reaction Scheme 3B above. Thus, compounds of type 3.3 can be prepared by a substitution reaction between an appropriate adenine analog, e.g., 3.6 as shown above, and an appropriate sulfonic acid, e.g., 3.7 as shown above. Appropriate adenine analogs and appropriate sulfonic acids are commercially available or prepared by methods known to one skilled in the art. The substitution reaction is carried out in the presence of an appropriate salt, e.g., a sodium salt, and an appropriate peroxide, e.g., tert-butyl hydrogen peroxide, in an appropriate solve, e.g., dichloromethane (DCM). Compounds of type 3.10 can be prepared by a coupling reaction of an appropriate halide, e.g., 3.8 as shown above, and an appropriate amine, e.g., 3.9 as shown above. Appropriate amines are commercially available or prepared by methods known to one skilled in the art. The coupling reaction is carried out in the presence of an appropriate base, e.g., diisopropylethylamine (DIPEA), in an appropriate solvent, e.g., ethanol, at an appropriate temperature, e.g., 110° C. As can be appreciated by one skilled in the art, the above reaction provides an example of a generalized approach wherein compounds similar in structure to the specific reactants above (compounds similar to compounds of type 3.1, 3.2, 3.3, and 3.4), can be substituted in the reaction to provide substituted N-containing heteroaryl analogs similar to Formula 3.5.

Compounds and compositions described herein are generally useful for modulating the activity of PINK1. In some embodiments, the compounds and compositions described herein inhibit the activity of PINK1.

### E. Methods of Using the Compounds

The compounds and pharmaceutical compositions of the invention are useful in treating or controlling disorders associated with PINK1 kinase activity. To treat or control the disorder, the compounds and pharmaceutical compositions comprising the compounds are administered to a subject in need thereof, such as a vertebrate, e.g., a mammal, a fish, a bird, a reptile, or an amphibian. The subject can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. The subject is preferably a mammal, such as a human. Prior to administering the compounds or compositions, the subject can be diagnosed with a need for treatment of the disorder associated with PINK1 kinase activity.

The compounds or compositions can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically; that is, administered to treat an existing disease or condition. A preparation can also be administered prophylactically; that is, administered for prevention of a disease or condition.

The therapeutically effective amount or dosage of the compound can vary within wide limits. Such a dosage is adjusted to the individual requirements in each particular case including the specific compound(s) being administered, the route of administration, the condition being treated, as well as the patient being treated. In general, in the case of oral or parenteral administration to adult humans weighing approximately 70 Kg or more, a daily dosage of about 10 mg to about 10,000 mg, preferably from about 200 mg to about 1,000 mg, should be appropriate, although the upper limit may be exceeded. The daily dosage can be administered as a single dose or in divided doses, or for parenteral administration, as a continuous infusion. Single dose compositions can contain such amounts or submultiples thereof of the compound or composition to make up the daily dose. The dosage can be adjusted by the individual physician in the event of any contraindications. Dosage can vary, and can be administered in one or more dose administrations daily, for one or several days.

1. Treatment Methods

The compounds disclosed herein are useful for treating or controlling disorders associated with PINK1 kinase activity. Thus, provided is a method comprising administering a therapeutically effective amount of a composition comprising a disclosed compound to a subject.

Accordingly, in some embodiments, the present disclosure provides methods of treating or preventing Parkinson's disease in a subject comprising administering to the subject one or more compounds, or a pharmaceutically acceptable salt thereof, of any one of the compounds described herein or a pharmaceutical composition comprising one or more of the compounds described herein, or pharmaceutically acceptable salt thereof. In some embodiments, the present disclosure provides methods of treating or preventing Leigh's disease in a subject comprising administering to the subject one or more compounds, or a pharmaceutically acceptable salt thereof, of any one of the compounds described herein or a pharmaceutical composition comprising one or more of the compounds described herein, or pharmaceutically acceptable salt thereof. In some embodiments, the treating of Parkinson's or Leigh's disease comprises ameliorating symptoms by stimulating PINK1 or a mutated PINK1.

In some embodiments, a method of treating one or more of the following mitochondrial diseases in a subject is provided. LHON, MELAS, and Charcot Marie Tooth. In some embodiments, the method comprises administering to a subject one or more compounds described herein, or a pharmaceutically acceptable salt thereof, or a pharmaceutical composition comprising one or more compounds described herein, or pharmaceutically acceptable salt thereof. In some embodiments, the method comprises administering to a subject a compound or pharmaceutically acceptable salt thereof that acts as a PINK1 substrate with one or more compounds described herein, or a pharmaceutically acceptable salt thereof, or a pharmaceutical composition comprising one or more compounds described herein, or pharmaceutically acceptable salt thereof. In some embodiments, the cholesterol therapeutic is niacin or acifran. In some embodiments, the subject is a subject in need thereof.

In some embodiments, the disclosure relates to a method of inhibiting mitochondrial aggregation comprising: contacting one or a plurality of: (i) compounds disclosed herein; or (ii) compositions or pharmaceutical compositions comprising compounds disclosed herein to one or a plurality of cells. In some embodiments, the method further comprises allowing the compounds, compositions or pharmaceutical compositions comprising the one or plurality of compounds to interact or to contact with the cell for a time period and under conditions sufficient for inhibiting mitochondrial aggregation in the cell.

The compositions are useful for treating any mitochondrial disorder (such as a neurodegenerative disease, cardiomyopathy or fibrosis that will respond favorably to a PINK1 inhibitor. Intravenous injection is one non-limiting method for treating acute mitochondrial disorders. Such a method would comprise administering a therapeutically effective amount of one or more compounds to a subject or subject in need thereof. Examples of mitochondrial disorders include, but are not limited to, cardiomyopathy, Alzheimer's Disease, Baton's Disease, Leigh's Disease, Acute Lateral Sclerosis and Huntingdon's Disease.

The disclosure also relates to a method of treating and/or preventing mitochondrial disease comprising administering a therapeutically effective amount of one or more compounds to a subject or subject in need thereof. The disclosure relates to a method of manufacturing a medicament comprising any one or plurality of compounds disclosed herein for the treatment of mitochondrial disease.

a. Treating a Disorder Associated with PINK1 Activity

In some embodiments, compounds and compositions described herein are useful in treating a disorder associated with PINK1 function. Thus, provided herein are methods of treating a disorder associated with PINK1 function, comprising administering to a subject in need thereof, a therapeutically effective amount of a compound described herein, or a pharmaceutically acceptable salt thereof, or a composition comprising a disclosed compound or pharmaceutically acceptable salt thereof. Disorders treatable by the present compounds and compositions include e.g., a neurodegenerative disease, a mitochondrial disease, fibrosis, or cardiomyopathy.

Thus, in various embodiments, disclosed are methods of treating a disorder in a subject in need thereof, the method comprising administering to the subject in need thereof an effective amount of a compound having a structure represented by a formula:

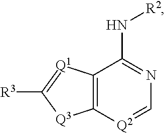

wherein Q¹ is N or CH and R³ is a 3- to 6-membered cycloalkyl, C1-C6 haloalkyl, C1-C6 haloalkoxy, or C1-C6 halohydroxyalkyl; or wherein Q¹ is CR¹ and R³ is hydrogen; R¹ is C1-C6 haloalkyl, C1-C6 haloalkoxy, C1-C6 halohydroxyalkyl, or a structure represented by a formula:

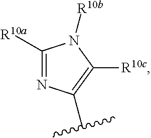

wherein each of R^{10a}, R^{10b}, and R^{10c}, when present, is independently selected from hydrogen and C1-C4 alkyl; wherein Q² is CH or N; wherein Q³ is CH₂ or NH; wherein R² is C1-C6 alkyl, —CR^{11a}R^{11b}Cy¹, or Cy¹; wherein each of R^{11a} and R^{11b}, when present, is independently selected from hydrogen, C1-C5 alkyl, and C1-C4 hydroxyalkyl; or wherein each of R^{11a} and R^{11b}, when present, together comprise a 3-membered cycloalkyl; wherein Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, or a pharmaceutically acceptable salt thereof, wherein the disorder is a neurodegenerative disorder, a mitochondrial disorder, a fibrosis, or cardiomyopathy.

In various embodiments, disclosed are methods of treating a disorder in a subject in need thereof, the method comprising administering to the subject in need thereof an effective amount of a compound having a structure represented by a formula:

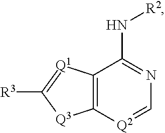

wherein Q¹ is N or CH and R³ is a 3- to 6-membered cycloalkyl, C1-C4 haloalkyl, C1-C4 cyanoalkyl, C1-C4 hydroxyalkyl, C1-C4 haloalkoxy, CF₃, CBr₃ or CCl₃; or wherein Q¹ is CR¹ and R³ is hydrogen; R¹ is C1-C6 haloalkyl, C1-C6 haloalkoxy, C1-C6 halohydroxy, or a structure represented by a formula:

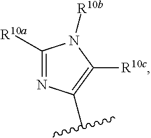

wherein each of R^{10a}, R^{10b}, and R^{10c}, when present, is independently selected from hydrogen and C1-C4 alkyl; wherein Q² is CH or N; wherein Q³ is CH₂ or NH; wherein R² is C1-C6 alkyl, —CR^{11a}R^{11b}Cy¹, or Cy¹; wherein each of R^{11a} and R^{11b}, when present, is independently selected from hydrogen, C1-C5 alkyl, and C1-C5 hydroxyalkyl; or wherein each of R^{11a} and R^{11b} together comprise a 3-membered cycloalkyl; wherein Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, provided that when R¹ is C1-C6 haloalkyl and R² is Cy¹, then Cy¹ is not a 6-membered carbocycle or a 9-membered heteroaryl, or a pharmaceutically acceptable salt thereof, wherein the disorder is a neurodegenerative disorder, a mitochondrial disorder, a fibrosis, or cardiomyopathy.

In various embodiments, disclosed are methods of treating a disorder in a subject in need thereof, the method comprising administering to the subject in need thereof an effective amount of a compound selected from:

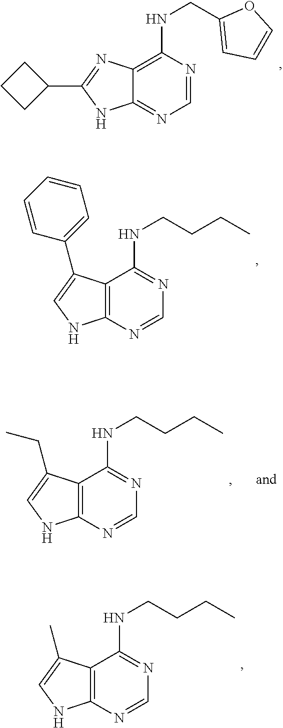

or a pharmaceutically acceptable salt thereof, wherein the disorder is a neurodegenerative disorder, a mitochondrial disorder, a fibrosis, or cardiomyopathy.

In various embodiments, disclosed are methods of treating a disorder in a subject in need thereof, the method comprising administering to the subject in need thereof an effective amount of a compound selected from:

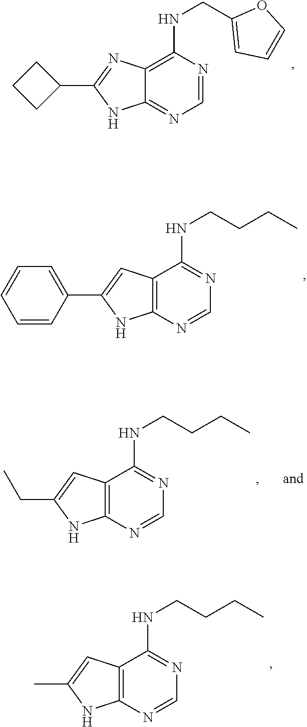

or a pharmaceutically acceptable salt thereof, wherein the disorder is a neurodegenerative disorder, a mitochondrial disorder, a fibrosis, or cardiomyopathy.

In various embodiments, disclosed are methods of treating a disorder in a subject in need thereof, the method comprising administering to the subject in need thereof an effective amount of a compound selected from:

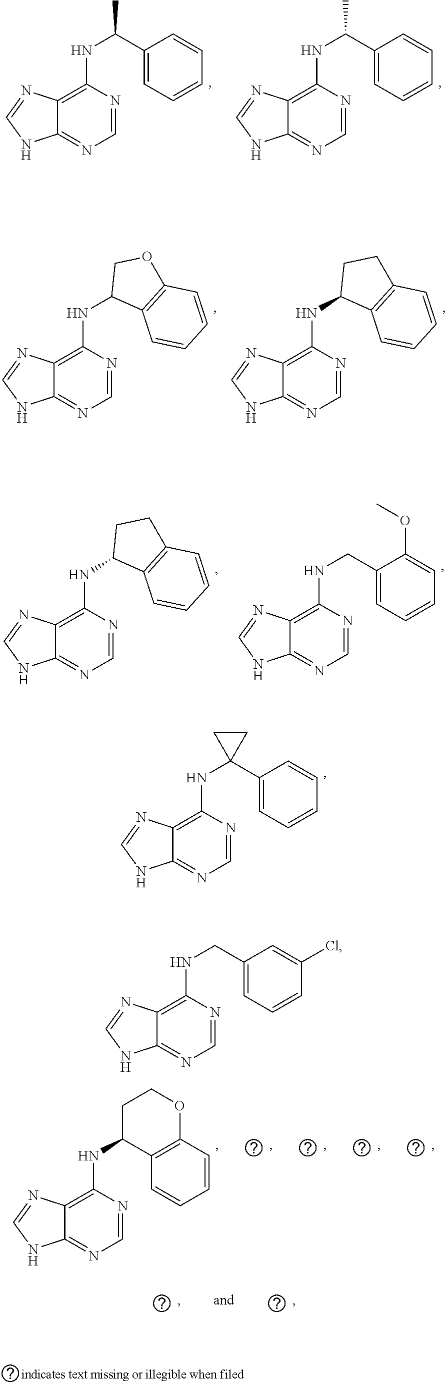

or a pharmaceutically acceptable salt thereof, wherein the disorder is a neurodegenerative disorder, a mitochondrial disorder, a fibrosis, or cardiomyopathy.

In various embodiments, disclosed are methods for treating a disorder associated with PINK1 kinase activity in a subject, the method comprising the step of administering to the subject an effective amount of a compound having a structure represented by Formula I:

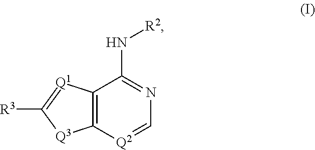

wherein Q¹ is N or CH and R³ is a 3- to 6-membered cycloalkyl, C1-C4 haloalkyl, C1-C4 cyanoalkyl, C1-C4 hydroxyalkyl, C1-C4 haloalkoxy, CF₃, CBr₃ or CCl₃; or wherein Q¹ is CR¹ and R³ is hydrogen; wherein Q² is CH or N; wherein Q³ is CH₂ or NH; R¹ is (C₁-C₆)alkyl, halo(C₁-C₄)alkyl, (C₁-C₄)alkoxy, halo(C₁-C₄)alkoxy, 5- or 6-membered heteroaryl, or phenyl, wherein said C₁-C₆alkyl and halo(C₁-C₄)alkyl are each optionally and independently substituted with a OR^{a} group, and wherein said phenyl and 5- or 6-membered heteroaryl are each optionally and independently substituted with 1 to 3 groups independently selected from R^{b}; R^{a}, when present, is H, (C₁-C₄)alkyl, or (C₁-C₄)alkoxy; each occurrence of R^{b}, when present, is independently halo, halo(C₁-C₄)alkyl, (C₁-C₄)alkoxy, or halo(C₁-C₄)alkoxy; R² is (C₁-C₆)alkyl, a 9-membered oxygen-containing fused heterocycle, or a 9- to 10-membered carbocycle, wherein said (C₁-C₆)alkyl is optionally substituted with 1 or 2 groups independently selected from R^{c}, and wherein said 9-membered oxygen-containing fused heterocycle and 9- to 10-membered carbocycle are each optionally and independently substituted with 1 to 3 groups independently selected from R^{d}; each occurrence of R^{c}, when present, is phenyl, 3- or 4-membered cycloalkyl, or 5- or 6-membered heteroaryl, wherein said phenyl and 5- or 6-membered heteroaryl are each optionally and independently substituted with 1 to 3 groups independently selected from R^{e}; each occurrence of R^{d} and R^{e}, when present, is independently halo, halo(C₁-C₄)alkyl, (C₁-C₄)alkoxy, or halo(C₁-C₄)alkoxy; and R³ is hydrogen, halogen, (C₁-C₄)alkyl, or 3- to 6-membered cycloalkyl, or pharmaceutically acceptable salts thereof.

Examples of neurodegenerative diseases that may be treated with a compound or composition described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, epilepsy, Friedreich ataxia, frontotemporal dementia, Gerstmann-Straussler-Scheinker syndrome, Huntington's disease, HIV-associated dementia, Kennedy's disease, Krabbe's disease, kuru, Leigh's disease (Leigh syndrome), Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoffs disease, Schilder's disease, Shy-Drager syndrome, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, Tabes dorsalis, drug-induced Parkinsonism, progressive supranuclear palsy, corticobasal degeneration, multiple system atrophy, Idiopathic Parkinson's disease, Autosomal dominant Parkinson disease, Parkinson disease, familial, type 1 (PARK1), Parkinson disease 3, autosomal dominant Lewy body (PARK3), Parkinson disease 4, autosomal dominant Lewy body (PARK4), Parkinson disease 5 (PARK5), Parkinson disease 6, autosomal recessive early-onset (PARK6), Parkinson disease 2, autosomal recessive juvenile (PARK2), Parkinson disease 7, autosomal recessive early-onset (PARK7), Parkinson disease 8 (PARK8), Parkinson disease 9 (PARK9), Parkinson disease 10 (PARK10), Parkinson disease 11 (PARK11), Parkinson disease 12 (PARK12), Parkinson disease 13 (PARK13), or Mitochondrial Parkinson's disease. In some embodiments, dysautonomia is not a neurodegenerative disease.

Examples of mitochondrial diseases that may be treated with a compound or composition described herein include Alzheimer's disease, amyotrophic lateral sclerosis, Asperger's Disorder, Autistic Disorder, bipolar disorder, cancer, cardiomyopathy, Charcot Marie Tooth disease (CMT, including various subtypes such as CMT type 2b and 2b), Childhood Disintegrative Disorder (CDD), diabetes, diabetic nephropathy, epilepsy, Friedreich's Ataxia (FA), Hereditary motor and sensory neuropathy (HMSN), Huntington's Disease, Keams-Sayre Syndrome (KSS), Leber's Hereditary Optic Neuropathy (LHON, also referred to as Leber's Disease, Leber's Optic Atrophy (LOA), or Leber's Optic Neuropathy (LON)), Leigh Disease or Leigh Syndrome, macular degeneration, Mitochondrial Myopathy, Lactacidosis, and Stroke (MELAS), mitochondrial neurogastrointestinal encephalomyophathy (MNGIE), motor neuron diseases, Myoclonic Epilepsy With Ragged Red Fibers (MERRF), Neuropathy, ataxia, retinitis pigmentosa, and ptosis (NARP), Parkinson's disease, Peroneal muscular atrophy (PMA), Pervasive Developmental Disorder Not Otherwise Specified (PDD-NOS), renal tubular acidosis, Rett's Disorder, Schizophrenia, and types of stroke.

Cardiomyopathy refers to a disease condition that adversely affects cardiac cell tissue leading to a measurable deterioration in myocardial function (e.g., systolic function, diastolic function). Dilated cardiomyopathy is characterized by ventricular chamber enlargement with systolic dysfunction and no hypertrophy. Hypertrophic cardiomyopathy, is a genetic disease transmitted as an autosomal dominant trait. Hypertrophic cardiomyopathy is morphologically characterized by a hypertrophied and non-dialated left ventricle. Restrictive cardiomyopathy is characterized by nondialated nonhypertrophied morphology with diminished ventricular volume leading to poor ventricular filling. Arrhythmogenic right ventricular cardiomyopathy is an inheritable heart disease characterized by myocardial electric instability. Unclassified cardiomyopathy is a category for cardiomyopathies that do not match the features of any one of the other types. Unclassified cardiomyopathies may have features of multiple types or, for example, have the features of fibroelastosis, noncompacted myocardium, or systolic dysfunction with minimal dilatation.

In some embodiments, the compounds and compositions described herein can be used to treat Parkinson's disease by decreasing the production of Lewy bodies, decreasing the accumulation of alpha-synuclein, decreasing cell death, decreasing loss of dopamine-generating cells, decreasing loss of cells in the substantia nigra, decreasing loss of dopamine production, decreasing a symptom of Parkinson's disease, decreasing loss of motor function, decreasing shaking or slowing an increase in shaking (tremor), decreasing rigidity or an increase in rigidity, decreasing slowness (bradykinesia) of movement or a slowing of movement, decreasing sensory symptoms, decreasing insomnia, decreasing sleepiness, increasing mental wellbeing, increasing mental function, slowing the decrease of mental function, decreasing dementia, delaying the onset of dementia, improving cognitive skills, decreasing the loss of cognitive skills, improving memory, decreasing the degradation of memory, or extending survival. In some embodiments, the compounds and compositions described herein can be used to treat cardiomyopathy by increasing cardiac performance, improving exercise tolerance, preventing heart failure, increasing blood oxygen content, or improving respiratory function.

In some embodiments, the disease treated by a disclosed compound or composition is one that is characterized by a reduction in the level of PINK1. In some embodiments, the disease is one characterized by loss of dopamine-producing cells (e.g., Parkinson's disease). In some embodiments, the disease is one characterized by neurodegeneration. In some embodiments, the disease is one characterized by neural cell death. In some embodiments, the disease is one characterized by a reduction in the level of PINK1 activity. In some embodiments, the disease is Parkinson's disease. In some embodiments, the disease is a neurodegenerative disease. In some embodiments, the disease is a cardiomyopathy.

In further embodiments, the neurodegenerative disorder is Parkinson's disease, Huntington's disease, or amyotrophic lateral sclerosis.

In further embodiments, the subject has been diagnosed with a need for treatment of a disorder associated with PINK1 kinase activity prior to the administering step.

In further embodiments, the subject is a mammal. In still further embodiments, the mammal is a human.

In further embodiments, the method further comprises the step of identifying a subject in need of treatment of a disorder associated with PINK1 kinase activity.

In further embodiments, the administering is accomplished by oral adminstration, parenteral administration, sublingual administration, transdermal administration, rectal administration, transmucosal administration, topical administration, inhalation, buccal administration, intrapleural administration, intravenous administration, intraarterial administration, intraperitoneal administration, subcutaneous administration, intramuscular administration, intranasal administration, intrathecal administration, and intraarticular administration, or combinations thereof.

In further embodiments, the administering comprises administering from about 1 to about 2000 milligrams of compound disclosed herein. In still further embodiments, the administering comprises administering from about 1 to about 1500 milligrams of compound disclosed herein. In yet further embodiments, the administering comprises administering from about 1 to about 1000 milligrams of compound disclosed herein. In an even further embodiment, the administering comprises administering from about 1 to about 500 milligrams of compound disclosed herein. In still further embodiments, the administering comprises administering from about 500 to about 2000 milligrams of compound disclosed herein. In yet further embodiments, the administering comprises administering from about 1000 to about 2000 milligrams of compound disclosed herein. In an even further embodiment, the administering comprises administering from about 1500 to about 2000 milligrams of compound disclosed herein.

2. Methods of Modulating PINK1 Kinase Activity in a Mammal

In some embodiments, disclosed are methods of modulating PINK1 kinase activity in a mammal, the method comprising the step of administering to the mammal a therapeutically effective amount of at least one disclosed compound, or a pharmaceutically acceptable salt thereof.

Thus, in various embodiments, disclosed are methods of modulating PINK1 kinase activity in a subject in need thereof, the method comprising administering to the subject in need thereof an effective amount of compound having a structure represented by a formula:

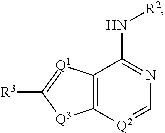

wherein Q¹ is N or CH and R³ is a 3- to 6-membered cycloalkyl, C1-C6 haloalkyl, C1-C6 haloalkoxy, or C1-C6 halohydroxyalkyl; or wherein Q¹ is CR¹ and R³ is hydrogen; R¹ is C1-C6 haloalkyl, C1-C6 haloalkoxy, C1-C6 halohydroxyalkyl, or a structure represented by a formula:

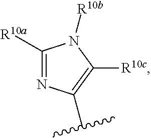

wherein each of R^{10a}, R^{10b}, and R^{10c}, when present, is independently selected from hydrogen and C1-C4 alkyl; wherein Q² is CH or N; wherein Q³ is CH₂ or NH; wherein R² is C1-C6 alkyl, —CR^{11a}R^{11b}Cy¹, or Cy¹; wherein each of R^{11a} and R^{11b}, when present, is independently selected from hydrogen, C1-C5 alkyl, and C1-C4 hydroxyalkyl; or wherein each of R^{11a} and R^{11b}, when present, together comprise a 3-membered cycloalkyl; wherein Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, or a pharmaceutically acceptable salt thereof.

In various embodiments, disclosed are methods of modulating PINK1 kinase activity in a subject in need thereof, the method comprising administering to the subject in need thereof an effective amount of compound having a structure represented by a formula:

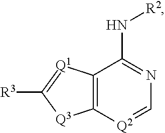

wherein Q¹ is N or CH and R³ is a 3- to 6-membered cycloalkyl, C1-C4 haloalkyl, C1-C4 cyanoalkyl, C1-C4 hydroxyalkyl, C1-C4 haloalkoxy, CF₃, CBr₃ or CCl₃; or wherein Q¹ is CR¹ and R³ is hydrogen; R¹ is C1-C6 haloalkyl, C1-C6 haloalkoxy, C1-C6 halohydroxy, or a structure represented by a formula:

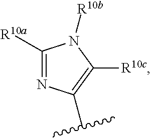

wherein each of R^{10a}, R^{10b}, and R^{10c}, when present, is independently selected from hydrogen and C1-C4 alkyl; wherein Q² is CH or N; wherein Q³ is CH₂ or NH; wherein R² is C1-C6 alkyl, —CR^{11a}R^{11b}Cy¹, or Cy¹; wherein each of R^{11a} and R^{11b}, when present, is independently selected from hydrogen, C1-C5 alkyl, and C1-C5 hydroxyalkyl; or wherein each of R^{11a} and R^{11b} together comprise a 3-membered cycloalkyl; wherein Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, provided that when R¹ is C1-C6 haloalkyl and R² is Cy¹, then Cy¹ is not a 6-membered carbocycle or a 9-membered heteroaryl, or a pharmaceutically acceptable salt thereof.

In various embodiments, disclosed are methods of modulating PINK1 kinase activity in a subject in need thereof, the method comprising administering to the subject in need thereof an effective amount of compound selected from:

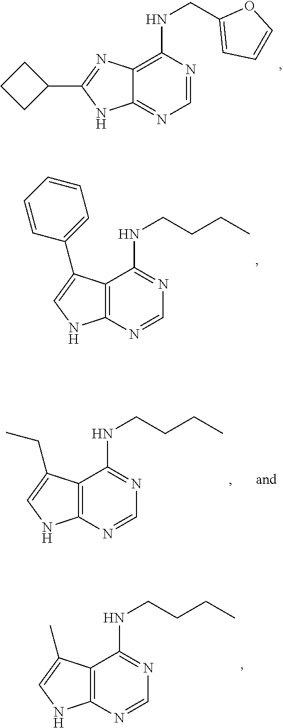

or a pharmaceutically acceptable salt thereof.

In various embodiments, disclosed are methods of modulating PINK1 kinase activity in a subject in need thereof, the method comprising administering to the subject in need thereof an effective amount of compound selected from:

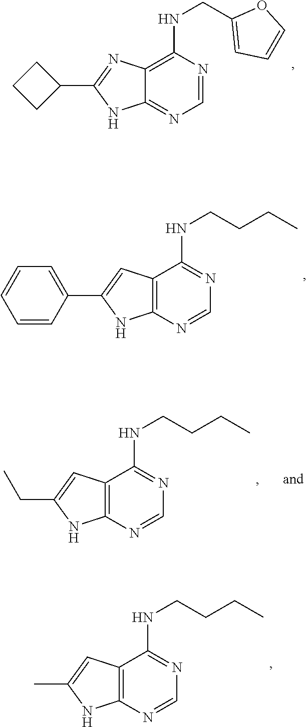

or a pharmaceutically acceptable salt thereof.

In various embodiments, disclosed are methods of modulating PINK1 kinase activity in a subject in need thereof, the method comprising administering to the subject in need thereof an effective amount of compound selected from:

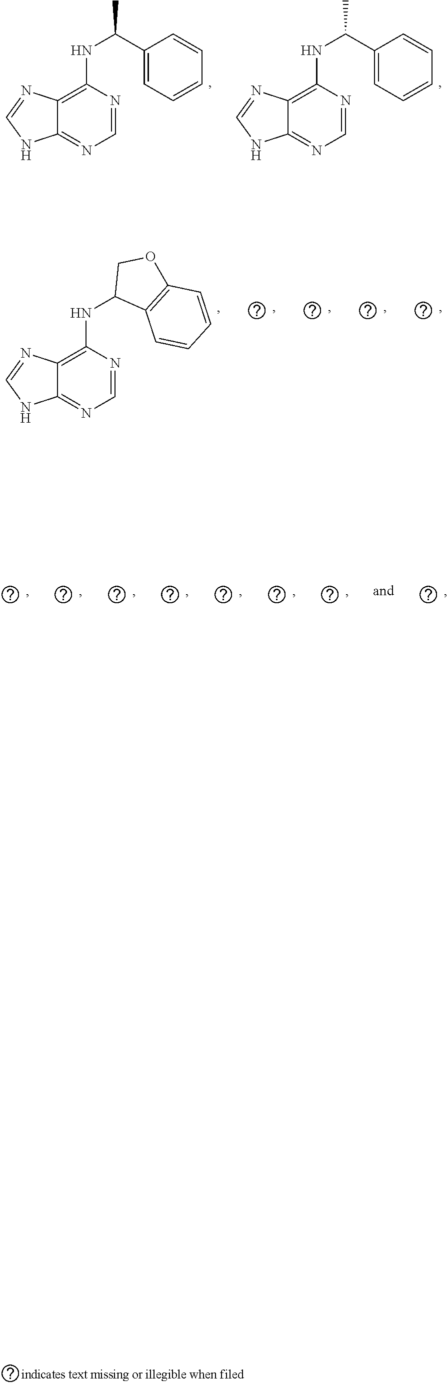 [SMILES: C[C@H](Nc1ncnc2[nH]cnc12)c1ccccc1 ; C[C@@H](Nc1ncnc2[nH]cnc12)c1ccccc1 ; c1ccc2c(c1)OCC2Nc1ncnc2[nH]cnc12]

or a pharmaceutically acceptable salt thereof.

In various embodiments, disclosed are methods for modulating PINK1 kinase activity in a mammal, the method comprising to the mammal an effective amount of a compound having a structure represented by Formula I:

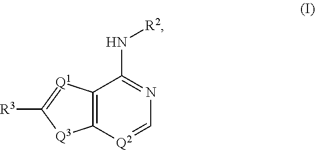

wherein Q¹ is N or CH and R³ is a 3- to 6-membered cycloalkyl, C1-C4 haloalkyl, C1-C4 cyanoalkyl, C1-C4 hydroxyalkyl, C1-C4 haloalkoxy, CF₃, CBr₃ or CCl₃; or wherein Q¹ is CR¹ and R³ is hydrogen; wherein Q² is CH or N; wherein Q³ is CH₂ or NH; R¹ is (C₁-C₆)alkyl, halo(C₁-C₄)alkyl, (C₁-C₄)alkoxy, halo(C₁-C₄)alkoxy, 5- or 6-membered heteroaryl, or phenyl, wherein said C₁-C₆alkyl and halo(C₁-C₄)alkyl are each optionally and independently substituted with a OR^{a} group, and wherein said phenyl and 5- or 6-membered heteroaryl are each optionally and independently substituted with 1 to 3 groups independently selected from R^{b}; R^{a}, when present, is H, (C₁-C₄)alkyl, or (C₁-C₄)alkoxy; each occurrence of R^{b}, when present, is independently halo, halo(C₁-C₄)alkyl, (C₁-C₄)alkoxy, or halo(C₁-C₄)alkoxy; R² is (C₁-C₆)alkyl, a 9-membered oxygen-containing fused heterocycle, or a 9- to 10-membered carbocycle, wherein said (C₁-C₆)alkyl is optionally substituted with 1 or 2 groups independently selected from R^{c}, and wherein said 9-membered oxygen-containing fused heterocycle and 9- to 10-membered carbocycle are each optionally and independently substituted with 1 to 3 groups independently selected from R^{d}; each occurrence of R^{c}, when present, is phenyl, 3- or 4-membered cycloalkyl, or 5- or 6-membered heteroaryl, wherein said phenyl and 5- or 6-membered heteroaryl are each optionally and independently substituted with 1 to 3 groups independently selected from R^{e}; each occurrence of R^{d} and R^{e}, when present, is independently halo, halo(C₁-C₄)alkyl, (C₁-C₄)alkoxy, or halo(C₁-C₄)alkoxy; and R³ is hydrogen, halogen, (C₁-C₄)alkyl, or 3- to 6-membered cycloalkyl, or pharmaceutically acceptable salts thereof.

As used herein, “modulation” can refer to either inhibition or enhancement of a specific activity. For example, the modulation of PINK1 activity can refer to the inhibition and/or activation of PINK1 dependent activities, such as a decrease or increase in Parkin recruitment. In some embodiments, the modulation refers to the inhibition or activation of Parkin recruitment. In some embodiments, the compounds described herein activate PINK1 activity by a factor from about 1% to about 50%. The activity of PINK1 can be measured by any method including but not limited to the methods described herein.

In some embodiments, the compounds described herein may be neo-substrates of PINK1, such as, for example, the following compounds:

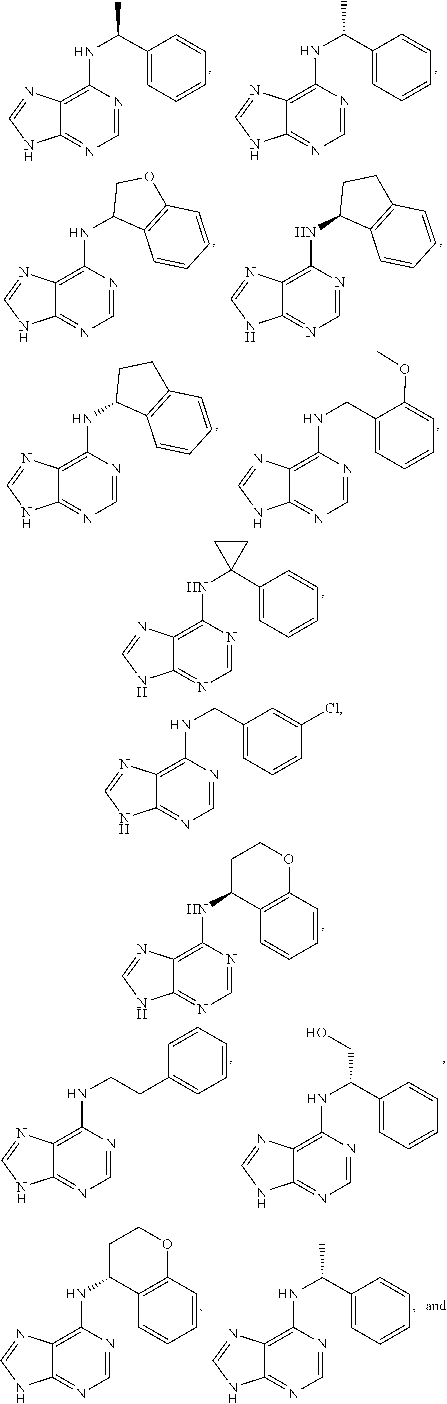

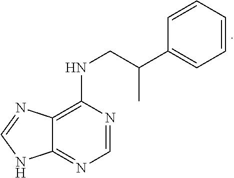 [SMILES: CC(CNc1ncnc2[nH]cnc12)c1ccccc1]

Without wishing to be bound by theory, the ability of the compounds to stimulate or inhibit PINK1 activity may be measured using any assay known in the art used to detect Parkin recruitment or PINK1 phosphorylation, or the absence of such signaling/activity. “PINK1 activity” refers to the ability of PINK1 to phosphorylate any substrate. Such activity can be measured, e.g., in a cell(s), by expressing mutant PINK1, administering the compounds disclosed herein and measuring the degree to which cells expressing the mutant PINK1 were able to phosphorylate an enzymatically active substrate as compared to a cell(s) expressing wild-type PINK1.

PINK1 activity can be measured by changes in the time necessary to recruit 50% of a substrate (“R₅₀”). In some embodiments, the compounds reduce a R50 by a factor of about 2%, 3%, 4%, 5%, 6%, 7%, 8%, 9%, 10, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%. In some embodiments, the compounds reduce a R₅₀ by a factor from about 1% to about 50%. In some embodiments, the compounds reduce a R₅₀ by a factor from about 2% to about 50%. In some embodiments, the compounds reduce a R₅₀ by a factor from about 3% to about 50%. In some embodiments, the compounds reduce a R₅₀ by a factor from about 4% to about 50%. In some embodiments, the compounds reduce a R₅₀ by a factor from about 5% to about 50%. In some embodiments, the compounds reduce a R₅₀ by a factor from about 6% to about 50%. In some embodiments, the compounds reduce a R₅₀ by a factor from about 7% to about 50%. In some embodiments, the compounds reduce a R₅₀ by a factor from about 8% to about 50%. In some embodiments, the compounds reduce a R₅₀ by a factor from about 9% to about 50%. In some embodiments, the compounds reduce a R₅₀ by a factor from about 10% to about 50%. In some embodiments, the compounds reduce a R₅₀ by a factor from about 15% to about 50%. In some embodiments, the compounds reduce a R₅₀ by a factor from about 20% to about 50%. In some embodiments, the compounds reduce a R₅₀ by a factor from about 25% to about 50%. In some embodiments, the compounds reduce a R₅₀ by a factor from about 30% to about 50%. In some embodiments, the compounds reduce a R₅₀ by a factor from about 35% to about 50%. In some embodiments, the compounds reduce a R₅₀ by a factor from about 40% to about 50%. In some embodiments, the compounds reduce a R₅₀ by a factor from about 45% to about 50%. In some embodiments, the compounds reduce a R₅₀ by a factor from about 10% to about 40%. In some embodiments, the compounds reduce a R₅₀ by a factor from about 10% to about 30%. In some embodiments, the compounds reduce a R₅₀ by a factor from about 10% to about 20%.

Plasmids expressing PINK1 can be transfected into an isolated cell and expressed in an isolated cell, expressed in a membrane derived from a cell, expressed in tissue or in an animal. For example, neuronal cells, cells of the immune system, transformed cells, or membranes can be used to test the PINK1 activity described above. Modulation is tested using one of the in vitro or in vivo assays described herein. Other assays generally known can also be used to test the compounds. Signal transduction can also be examined in vitro with soluble or solid state reactions, using a chimeric molecule such as an extracellular domain of a receptor covalently linked to a heterologous signal transduction domain, or a heterologous extracellular domain covalently linked to the transmembrane and or cytoplasmic domain of a receptor. Furthermore, ligand-binding domains of the protein of interest can be used in vitro in soluble or solid state reactions to assay for ligand binding.

Ligand binding can be performed in solution, in a bilayer membrane, attached to a solid phase, in a lipid monolayer, or in vesicles. For example, in an assay, the binding of the natural ligand to its receptor is measured in the presence of a candidate modulator, such as the compound described herein. Alternatively, the binding of the candidate modulator may be measured in the presence of the natural ligand. Often, competitive assays that measure the ability of a compound to compete with binding of the natural ligand to the receptor are used. Binding can be tested by measuring, e.g., changes in spectroscopic characteristics (e.g., fluorescence, absorbance, refractive index), hydrodynamic (e.g., shape) changes, or changes in chromatographic or solubility properties.

In some embodiments, the activity of the compounds to activate PINK1 can also be measured using assays involving Parkin recruitment. Parkin is a mitochondrial quality control regulatory protein that is distributed throughout the cytoplasm in cells with healthy mitochondria and no active PINK1. Upon mitochondrial damage, Parkin is recruited to damaged mitochondria by PINK1 activity. Thus, measuring the effect of PINK1 compound treatment on Parkin recruitment to the mitochondrial surface serves as a measurement of the compound's ability to increase the activity of PINK1. In some embodiments, this is performed by transfecting a labeled Parkin fusion protein (e.g., Parkin-yellow fluorescent protein (YFP)) into cells and monitoring Parkin's distribution using confocal microscopy (see, e.g., Narendra et al., PLOS Biol. 2010 8(1): e1000298. In still other embodiments the cells expressing YFP Parkin can be introduced into the cell by stable transfection and selection with G418 (Geneticin) or Puromycin and the stable expressing cells can be used to quantify the level of Parkin recruitment. After application of the PINK1 activating compound the level of PINK1 is read out by the level of YFP-Parkin on the mitochondria.

Another technology that can be used to evaluate PINK1 activity in cells is phospho-ubiquitin enzyme-linked immunosorbent assay (ELISA). Upon PINK1 activation, the level of phospho-serine 65 (pS65) ubiquitin on mitochondria dramatically increases. In some embodiments, this is done by using traditional Western blotting techniques familiar to those skilled in the art. In some embodiments, a pS65 ubiquitin capture antibody pulls down phospho-ubiquitin from a cellular lysate of cells treated with the compound of interest. Following the wash, a detection antibody is applied to read the signal. The methods, described in Hou et al Autophagy 2018, 14, NO. 8, 1404-1418, may be used to design and make the ELISA to measure the effect of compounds that modulate PINK1 activity. The increase in p65 ubiquitin seen by either Western blot or ELISA upon compound treatment indicates that the compound has increased PINK1 activity.

In another embodiment, transcription levels can be measured to assess the effects of a test compound on PINK1 activation. A host cell containing the protein of interest is treated with a test compound in the presence of the mitochondrial damaging agent, then the level of gene expression is measured. In some embodiments, the test gene could be GDF15, TNFRSF12a, PLK3, PINK1, PARKIN, and/or ATF3. The amount of time to effect such interactions may be empirically determined, such as by running a time course and measuring the level of transcription as a function of time. The amount of transcription may be measured by using any method known to those of skill in the art to be suitable. For example, mRNA expression of the protein of interest may be detected using quantitative PCR assays or their polypeptide products may be identified using immunoassays. Alternatively, transcription-based assays using reporter genes may be used as described in U.S. Pat. No. 5,436,128, herein incorporated by reference. Reporter genes examples include chloramphenicol acetyltransferase, firefly luciferase, bacterial luciferase, β-galactosidase, and alkaline phosphatase. Furthermore, the protein of interest can be used as an indirect reporter via attachment to a second reporter such as green fluorescent protein (see, e.g., Mistili & Spector, Nature Biotechnology 15:961 964 (1997)). The amount of transcription is then compared to the amount of transcription in either the same cell in the absence of the test compound, or it may be compared with the amount of transcription in a substantially identical cell that lacks the protein of interest. A substantially identical cell may be derived from the same cells from which the recombinant cell was prepared but which had not been modified by the introduction of heterologous DNA. Any difference in the amount of transcription indicates that the test compound has in some manner altered the expression level of the protein of interest.

Additional assays can also be used. For example, the activity of the compound can be measured in a cell-based assay that can measure the colocalization of mitochondria with lysosomes, and indicator of mitophagy. For example, a nucleic acid molecule encoding mKeima, such as Accession AB209969, can be incorporated into an expression vector and transfected or transformed into a cell. In some embodiments, the expression vector is a plasmid or virus. In some embodiments, the expression of the nucleic acid molecule is operably linked a mitochondrial localization sequence to ensure mitochondrial localization. The promoter can be constitutive or respond to a drug or other response element so that the expression can be controlled. The type of expression vector is not critical and any expression vector can be used that is suitable for the cell type. In some embodiments, the cell is a mammalian cell-like HeLa cell available from the ATCC CCL-2 or SKOV3 HTB-77. The expression of the reporter protein can be stable so that that stable cell lines can be selected. The selection of stably expressing receptor cell lines can be done to routine methods, such as selecting for expression under G418 (Geneticin) or Puromycin. The expression of the reporter protein can also be transient.

After the lysosome localization reporter “mKeima” is expressed in a cell, the cells can be grown in appropriate media in the appropriate cell plate. The cells can be plated, for example at 5000-10000 cells per well in a 384 well plate. In some embodiments, the cells are plated at about 1000, 2000, 3000, 4000, 5000, 6000, 7000, 8000, 9000, or 10000 cells/per well. The plates can have any number of wells and the number of cells can be modified accordingly. The cells can then be treated with the compound as described in this patent along with a mitochondrial toxin, then analyzed by techniques known to those skilled in the art. In some embodiments, the cells can be trypsinized and analysed by fluorescent activated cell sorting. In other embodiments, the cells can be analyzed in a microscope to visualize the location of the mitochondrial reporter protein and the pH of the subcellular compartment. An increase in mitochondria localization in lysosomes induced by compound treatment would indicate an increase in the level of mitophagy.

Another embodiment is a method for inhibiting (preventing, stopping) aggregation of α-synuclein molecule(s) (e.g. a monomer, small aggregate, oligomer, or fibril of a-synuclein) in primary neurons derived from mice. In this embodiment, aggregated α-synuclein molecules such as pre-formed α-synuclein fibrils are applied to primary hippocampal neurons along with an effective amount of a PINK1 enhancing compound. The α-synuclein molecule can be in solution or in a cell, which is in culture or in a subject. In one embodiment, the contacting of an α-synuclein molecule which is an oligomer or small aggregate creates a severely aggregated form (oligomerization, further oligomerization, and/or fibril formation) of the α-synuclein molecule which can be blocked by the pharmaceutical compositions of the compound described herein. In some embodiments, the cells can then be fixed, harvested and processed to analyze levels of phosphorylated pathogenic α-synuclein. In some embodiments, the level of α-synuclein can be assessed by immunoblotting. In other embodiments, the levels of α-synuclein can be assessed by immunofluorescence. In still other embodiments the levels of α-synuclein can be assessed by ELISA.

Another embodiment is a method for inhibiting (preventing, stopping) aggregation of α-synuclein molecule(s) (e.g. a monomer, small aggregate, oligomer, or fibril of a-synuclein), in the brain of a mouse injected with a pharmaceutical form of the compound invention. In this embodiment, aggregated α-synuclein molecules such as pre-formed α-synuclein fibrils are injected into the striatum of a mouse and the mouse is treated by oral dosing with an effective amount of a pharmaceutical composition of the invention. The a-synuclein molecule can be in solution when injected into a mouse. In one embodiment, the contacting of an α-synuclein molecule which is an oligomer or small aggregate creates a severely aggregated form (oligomerization, further oligomerization, and/or fibril formation) of the α-synuclein molecule which can be blocked by the pharmaceutical composition of the compound described herein. In some embodiments, the brain can then be harvested and processed to analyze levels of pathogenic phosphorylated α-synuclein. In some embodiments, the level of α-synuclein can be assessed by immunoblotting. In other embodiments, the levels of α-synuclein can be assessed by immunofluorescence. In still other embodiments the levels of α-synuclein can be assessed by ELISA.

In some embodiments, a compound's effect on the modulation of PINK1 will be measured using cells expressing mutant and wild-type verisons of PINK1. PINK1 is generally known. In some embodiments, the enzymatic rescue is measured. Enzymatic rescue experiments are experiments in which cells expressing mutated forms of the PINK1 with reduced or deficient enzymatic activity are contacted with compounds of the present invention and are able to re-activate the mutated PINK1 enzymatic activity. PINK1 molecules are known. In some embodiments, the compounds of the present invention are able to enzymatically rescue human PINK1 (accession number NM 032409.3, which is incorporated by reference in its entirety) having the following amino acid sequence:

(SEQ ID NO: 1)
MAVRQALGRGLQLGRALLLRFAPKPGPVSGWGKPG
PGAAWGRGERPGRVSSPGAQPRPLGLPLPDRYRFF
RQSVAGLAARIQRQFVVRARGGAGPCGRAVFLAFG
LGLGLIEEKQAESRRAASACQEIQAIFTQKNKQVS
DPLDTRRWQGFRLEDYLIGQAIGKGCNAAVYEATM
PTLPQHLEKAKHLGLLGKGPDVVSKGADGEQAPGA
PAFPFAIKMMWNISAGSSSEAILSKMSQELVPASR
MALDGEYGAVTYRRSRDGPKQLAPHPNIIRVFRAF
TSSVPLLPGALADYPDMLPPHYYPEGLGHGRTLFL
VMKNYPCTLRQYLEEQTPSSRLATMMTLQLLEGVD
HLVQQGIAHRDLKSDNILVEWDSDGCPWLVISDFG
CCLADERVGLQLPFNSSSVERGGNGSLMAPEVSTA
HSGPHAVIDYSKADTWAVGAIAYEIFGLANPFYGQ
GSAHLESRSYQEAQLPEMPKSVPPETRQLVRSLLQ
REANKRPSARIAANVLHLSLWGEHLLALKNLKLDK
MIAWLLQQSAATLLADRLREKSCVETKLQMLFLAN
LECEALCQAALLLSSWRAAP.

In some embodiment, the compounds of the present invention are able to enzymatically rescue mouse PINK1 (accession number NM_026880.2, which is incorporated by reference in its entirety) having the following amino acid sequence:

(SEQ ID NO: 2)
MAVRQALGRGLQLGRALLLRFAPKPGPLFGWGKPG
PAAAWGRGERPGQVVSPGAQPRPVGLPLPDRYRFF
RQSVAGLAARIQRQFMVRARGGAGPCGRAVFLAFG
LGLGLIEEKQAEGRRAASACQEIQAIFTQKTKRVS
DPLDTRCWQGFRLEDYLIGQAIGKGCNAAVYEATM
PTLPQHLEKAKHLGLIGKGPDVVLKGADGEQAPGT
PTFPFAIKMMWNISAGSSSEAILSKMSQELVPASR
VALAGEYGAVTYRRSRDGPKQLAPHPNIIRVFRAF
TSSVPLLPGALADYPDMLPPHYYPEGLGHGRTLFL
VMKNYPCTLRQYLEEQTPSSRLATMMTLQLLEGVD
HLVQQGIAHRDLKSDNILVEWDSDGCPWLVISDFG
CCLADQHVGLRLPFNSSSVERGGNGSLMAPEVSTA
HSGPSAVIDYSKADTWAVGAIAYEIFGLANPFYGQ
GSAHLESRSYQEAQLPEMPESVPPEARRLVRSLLQ
REASKRPSARLAANVLHLSLWGEHLLALKNLKLDK
MIAWLLQQSAATLLADRLREKSCVETKLQMLFLAN
LECEALCQAALLLSSWRAAP.

In some embodiments, the compounds of the present invention are able to enzymatically rescue rat PINK1 (accession number BC169047.1, which is incorporated by reference in its entirety) having the following amino acid sequence:

(SEQ ID NO: 3)
MAVRQALGRGLQLGRALLLRFAPKPGPVSGWGKPG
PGAAWGRGERPGRVSSPGAQPRPLGLPLPDRYRFF
RQSVAGLAARIQRQFVVRARGGAGPCGRAVFLAFG
LGLGLIEEKQAESRRAASACQEIQAIFTQKNKQVS
DPLDTRRWQGFRLEDYLIGQAIGKGCNAAVYEATM
PTLPQHLEKAKHLGLLGKGPDVVSKGADGEQAPGA
PAFPFAIKMMWNISAGSSSEAILSKMSQELVPASR
MALDGEYGAVTYRRSRDGPKQLAPHPNIIRVFRAF
TSSVPLLPGALADYPDMLPPHYYPEGLGHGRTLFL
VMKNYPCTLRQYLEEQTPSSRLATMMTLQLLEGVD
HLVQQGIAHRDLKSDNILVEWDSDGCPWLVISDFG
CCLADERVGLQLPFNSSSVERGGNGSLMAPEVSTA
HSGPHAVIDYSKADTWAVGAIAYEIFGLANPFYGQ
GSAHLESRSYQEAQLPEMPKSVPPETRQLVRSLLQ
REANKRPSARIAANVLHLSLWGEHLLALKNLKLDK
MIAWLLQQSAATLLADRLREKSCVETKLQMLFLAN
LECEALCQAALLLSSWRAAP.

In further embodiments, modulating is inhibiting. In still further embodiments, modulating is decreasing.

In further embodiments, the compound exhibits inhibition of PINK1 kinase activity with an IC₅₀ of less than about 30 μM. In still further embodiments, the compound exhibits inhibition of PINK1 kinase activity with an IC₅₀ of less than about 25 μM. In yet further embodiments, the compound exhibits inhibition of PINK1 kinase activity with an IC₅₀ of less than about 20 μM. In an even further embodiment, the compound exhibits inhibition of PINK1 kinase activity with an IC₅₀ of less than about 15 μM. In still further embodiments, the compound exhibits inhibition of PINK1 kinase activity with an IC₅₀ of less than about 10 μM. In yet further embodiments, the compound exhibits inhibition of PINK1 kinase activity with an IC₅₀ of less than about 5 μM. In an even further embodiment, the compound exhibits inhibition of PINK1 kinase activity with an IC₅₀ of less than about 1 μM. In still further embodiments, the compound exhibits inhibition of PINK1 kinase activity with an IC₅₀ of less than about 0.5 μM.

In further embodiments, modulating is activating. In still further embodiments, modulating is increasing. In further embodiments, the compound exhibits activation of PINK1 kinase activity with an EC₅₀ of less than about 30 μM. In still further embodiments, the compound exhibits activation of PINK1 kinase activity with an EC₅₀ of less than about 25 μM. In yet further embodiments, the compound exhibits activation of PINK1 kinase activity with an EC₅₀ of less than about 20 μM. In an even further embodiment, the compound exhibits activation of PINK1 kinase activity with an EC₅₀ of less than about 15 μM. In still further embodiments, the compound exhibits activation of PINK1 kinase activity with an EC₅₀ of less than about 10 μM. In yet further embodiments, the compound exhibits activation of PINK1 kinase activity with an EC₅₀ of less than about 5 μM. In an even further embodiment, the compound exhibits activation of PINK1 kinase activity with an EC₅₀ of less than about 1 μM. In still further embodiments, the compound exhibits activation of PINK1 kinase activity with an EC₅₀ of less than about 0.5 μM. In further embodiments, the subject is a mammal. In still further embodiments, the subject is a human.

In further embodiments, the subject has been diagnosed with a need for treatment of a disorder associated with PINK1 kinase dysfunction prior to the administering step. In still further embodiments, the method further comprises the step of identifying a subject at risk of becoming infected with a disorder associated with PINK1 kinase dysfunction prior to treatment of the disorder.

3. Methods of Modulating PINK1 Kinase Activity in at Least One Cell

In some embodiments, disclosed are methods for modulating PINK1 kinase activity in at least one cell, the method comprising the step of contacting the at least one cell with an effective amount of at least one disclosed compound, or a pharmaceutically acceptable salt thereof. Thus, in various embodiments, disclosed are methods for modulating PINK1 kinase activity in at least one cell, the method comprising contacting the cell with an effective amount of a compound having a structure represented by a formula:

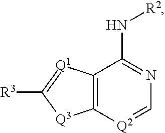

wherein Q¹ is N or CH and R³ is a 3- to 6-membered cycloalkyl, C1-C6 haloalkyl, C1-C6 haloalkoxy, or C1-C6 halohydroxyalkyl; or wherein Q¹ is CR¹ and R³ is hydrogen; R¹ is C1-C6 haloalkyl, C1-C6 haloalkoxy, C1-C6 halohydroxyalkyl, or a structure represented by a formula:

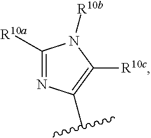

wherein each of R^{10a}, R^{10b}, and R^{10c}, when present, is independently selected from hydrogen and C1-C4 alkyl; wherein Q² is CH or N; wherein Q³ is CH₂ or NH; wherein R² is C1-C6 alkyl, —CR^{11a}R^{11b}Cy¹, or Cy¹; wherein each of R^{11a} and R^{11b}, when present, is independently selected from hydrogen, C1-C5 alkyl, and C1-C4 hydroxyalkyl; or wherein each of R^{11a} and R^{11b}, when present, together comprise a 3-membered cycloalkyl; wherein Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, or a pharmaceutically acceptable salt thereof.

In various embodiments, disclosed are methods for modulating PINK1 kinase activity in at least one cell, the method comprising contacting the cell with an effective amount of a compound having a structure represented by a formula:

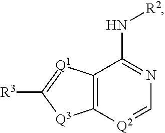

wherein Q¹ is N or CH and R³ is a 3- to 6-membered cycloalkyl, C1-C4 haloalkyl, C1-C4 cyanoalkyl, C1-C4 hydroxyalkyl, C1-C4 haloalkoxy, CF₃, CBr₃ or CCl₃; or wherein Q¹ is CR¹ and R³ is hydrogen; R¹ is C1-C6 haloalkyl, C1-C6 haloalkoxy, C1-C6 halohydroxy, or a structure represented by a formula:

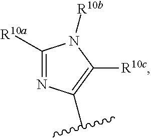

wherein each of R^{10a}, R^{10b}, and R^{10c}, when present, is independently selected from hydrogen and C1-C4 alkyl; wherein Q² is CH or N; wherein Q³ is CH₂ or NH; wherein R² is C1-C6 alkyl, —CR^{11a}R^{11b}Cy¹, or Cy¹; wherein each of R^{11a} and R^{11b}, when present, is independently selected from hydrogen, C1-C5 alkyl, and C1-C5 hydroxyalkyl; or wherein each of R^{11a} and R^{11b} together comprise a 3-membered cycloalkyl; wherein Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, provided that when R¹ is C1-C6 haloalkyl and R² is Cy¹, then Cy¹ is not a 6-membered carbocycle or a 9-membered heteroaryl, or a pharmaceutically acceptable salt thereof.

In some embodiments, Q¹ is N or CH.

In various embodiments, disclosed are methods for modulating PINK1 kinase activity in at least one cell, the method comprising contacting the cell with an effective amount of a compound selected from:

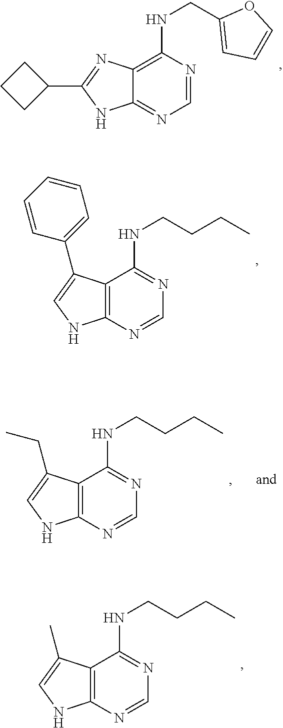

or a pharmaceutically acceptable salt thereof.

In various embodiments, disclosed are methods for modulating PINK1 kinase activity in at least one cell, the method comprising contacting the cell with an effective amount of a compound selected from:

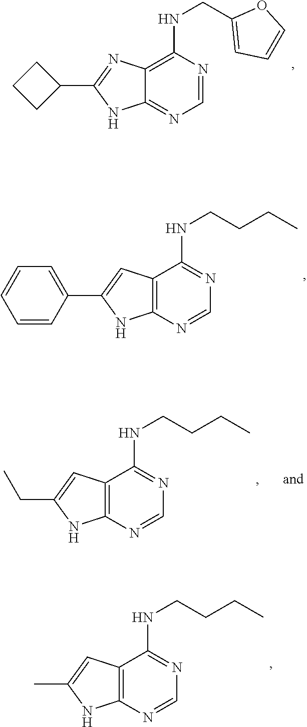

or a pharmaceutically acceptable salt thereof.

In various embodiments, disclosed are methods for modulating PINK1 kinase activity in at least one cell, the method comprising contacting the cell with an effective amount of a compound selected from:

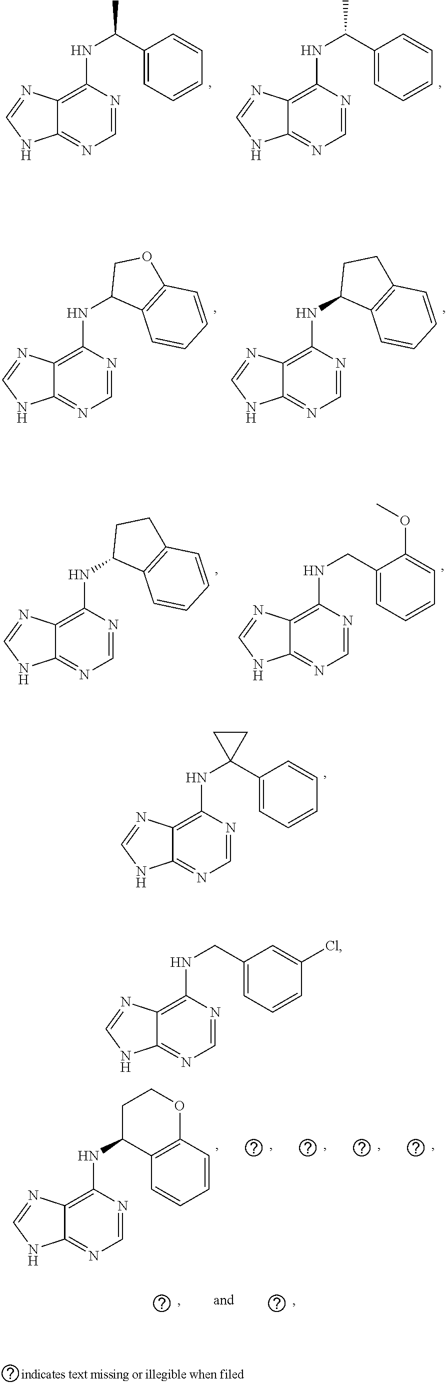

or a pharmaceutically acceptable salt thereof.

In various embodiments, disclosed are methods for treating a disorder associated with PINK1 kinase activity in at least one cell, the method comprising the step of contacting the at least one cell with an effective amount of a compound having a structure represented by Formula I:

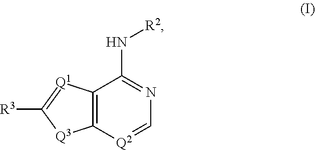

wherein Q¹ is N or CH and R³ is a 3- to 6-membered cycloalkyl, C1-C4 haloalkyl, C1-C4 cyanoalkyl, C1-C4 hydroxyalkyl, C1-C4 haloalkoxy, CF₃, CBr₃ or CCl₃; wherein Q² is CH or N; wherein Q³ is CH₂ or NH; R¹ is (C1-C6)alkyl, halo(C1-C4)alkyl, (C1-C4)alkoxy, halo(C1-C4)alkoxy, 5- or 6-membered heteroaryl, or phenyl, wherein said C1-C6alkyl and halo(C1-C4)alkyl are each optionally and independently substituted with a OR^{a} group, and wherein said phenyl and 5- or 6-membered heteroaryl are each optionally and independently substituted with 1 to 3 groups independently selected from R^{b}; R^{a}, when present, is H, (C₁-C₄)alkyl, or (C₁-C₄)alkoxy; each occurrence of R^{b}, when present, is independently halo, halo(C₁-C₄)alkyl, (C₁-C₄)alkoxy, or halo(C₁-C₄)alkoxy; R² is (C₁-C₆)alkyl, a 9-membered oxygen-containing fused heterocycle, or a 9- to 10-membered carbocycle, wherein said (C₁-C₆)alkyl is optionally substituted with 1 or 2 groups independently selected from R^{c}, and wherein said 9-membered oxygen-containing fused heterocycle and 9- to 10-membered carbocycle are each optionally and independently substituted with 1 to 3 groups independently selected from R^{d}; each occurrence of R^{c}, when present, is phenyl, 3- or 4-membered cycloalkyl, or 5- or 6-membered heteroaryl, wherein said phenyl and 5- or 6-membered heteroaryl are each optionally and independently substituted with 1 to 3 groups independently selected from R^{e}; each occurrence of R^{d} and R^{e}, when present, is independently halo, halo(C₁-C₄)alkyl, (C₁-C₄)alkoxy, or halo(C₁-C₄)alkoxy; and R³ is hydrogen, halogen, (C₁-C₄)alkyl, or 3- to 6-membered cycloalkyl, or pharmaceutically acceptable salts thereof.

In various embodiments, disclosed are methods for treating a disorder associated with PINK1 kinase activity in at least one cell, the method comprising the step of contacting the at least one cell with an effective amount of a compound having a structure represented by Formula I:

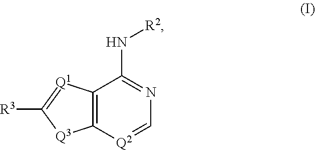

wherein Q¹ is N or CH and R³ is a 3- to 6-membered cycloalkyl, C1-C4 haloalkyl, C1-C4 cyanoalkyl, C1-C4 hydroxyalkyl, C1-C4 haloalkoxy, CF₃, CBr₃ or CCl₃; or wherein Q¹ is CR¹ and R³ is hydrogen; wherein Q² is CH or N; wherein Q³ is CH₂ or NH; R¹ is (C₁-C₆)alkyl, halo(C₁-C₄)alkyl, (C₁-C₄)alkoxy, halo(C₁-C₄)alkoxy, 5- or 6-membered heteroaryl, or phenyl, wherein said C₁-C₆alkyl and halo(C₁-C₄)alkyl are each optionally and independently substituted with a OR^{a} group, and wherein said phenyl and 5- or 6-membered heteroaryl are each optionally and independently substituted with 1 to 3 groups independently selected from R^{b}; R^{a}, when present, is H, (C₁-C₄)alkyl, or (C₁-C₄)alkoxy; each occurrence of R^{b}, when present, is independently halo, halo(C₁-C₄)alkyl, (C₁-C₄)alkoxy, or halo(C₁-C₄)alkoxy; R² is (C₁-C₆)alkyl, a 9-membered oxygen-containing fused heterocycle, or a 9- to 10-membered carbocycle, wherein said (C₁-C₆)alkyl is optionally substituted with 1 or 2 groups independently selected from R^{c}, and wherein said 9-membered oxygen-containing fused heterocycle and 9- to 10-membered carbocycle are each optionally and independently substituted with 1 to 3 groups independently selected from R^{d}; each occurrence of R^{c}, when present, is phenyl, 3- or 4-membered cycloalkyl, or 5- or 6-membered heteroaryl, wherein said phenyl and 5- or 6-membered heteroaryl are each optionally and independently substituted with 1 to 3 groups independently selected from R^{e}; each occurrence of R^{d} and R^{e}, when present, is independently halo, halo(C₁-C₄)alkyl, (C₁-C₄)alkoxy, or halo(C₁-C₄)alkoxy; and R³ is hydrogen, halogen, (C₁-C₄)alkyl, or 3- to 6-membered cycloalkyl, or pharmaceutically acceptable salts thereof.

In further embodiments, the cell is mammalian. In still further embodiments, the cell is human. In yet further embodiments, the cell has been isolated from a mammal prior to the contacting step. In further embodiments, modulating is inhibiting. In still further embodiments, modulating is decreasing. In further embodiments, modulating is activating.

In still further embodiments, modulating is increasing. In further embodiments, contacting is via administration to a mammal. In further embodiments, the step of contacting is performed in vitro.

4. Use of Compounds

Also provided herein is the use of a compound described herein, or a pharmaceutically acceptable salt thereof, or a composition comprising a disclosed compound or pharmaceutically acceptable salt thereof, for the manufacture of a medicament for treating a disorder described herein. Also provided is a compound described herein, or a pharmaceutically acceptable salt thereof, or a composition comprising a disclosed compound or pharmaceutically acceptable salt thereof, for use in treating a disorder described herein.

Thus, in some embodiments, the invention relates to the use of a disclosed compound or a product of a disclosed method. In further embodiments, a use relates to the manufacture of a medicament for the treatment of a disorder associated with PINK1 kinase activity in a mammal.

Also provided are the uses of the disclosed compounds and products. In some embodiments, the invention relates to use of at least one disclosed compound; or a pharmaceutically acceptable salt, hydrate, solvate, or polymorph thereof. In further embodiments, the compound used is a product of a disclosed method of making.

In further embodiments, the use relates to a process for preparing a pharmaceutical composition comprising a therapeutically effective amount of a disclosed compound or a product of a disclosed method of making, or a pharmaceutically acceptable salt, solvate, or polymorph thereof, for use as a medicament. In further embodiments, the use relates to a process for preparing a pharmaceutical composition comprising a therapeutically effective amount of a disclosed compound or a product of a disclosed method of making, or a pharmaceutically acceptable salt, solvate, or polymorph thereof, wherein a pharmaceutically acceptable carrier is intimately mixed with a therapeutically effective amount of the compound or the product of a disclosed method of making.

In various embodiments, the use relates to a treatment of a disorder associated with PINK1 kinase activity in a mammal. In some embodiments, the use is characterized in that the mammal is a human. In some embodiments, the use is characterized in that the disorder associated with PINK1 kinase activity is a neurodegenerative disease, a mitochondrial disease, fibrosis, and/or cardiomyopathy.

In further embodiments, the use relates to the manufacture of a medicament for the treatment of a disorder associated with PINK1 kinase activity in a mammal. It is understood that the disclosed uses can be employed in connection with the disclosed compounds, products of disclosed methods of making, methods, compositions, and kits. In further embodiments, the invention relates to the use of a disclosed compound or a disclosed product in the manufacture of a medicament for the treatment of a disorder associated with PINK1 kinase activity in a mammal.

5. Manufacture of a Medicament

In some embodiments, the invention relates to a method for the manufacture of a medicament for treating a disorder associated with PINK1 kinase activity in a mammal, the method comprising combining a therapeutically effective amount of a disclosed compound or product of a disclosed method with a pharmaceutically acceptable carrier or diluent. In some embodiments, the invention relates to a method for the manufacture of a medicament for treating a mitochondrial disease in a mammal, the method comprising combining a therapeutically effective amount of a disclosed compound or product of a disclosed method with a pharmaceutically acceptable carrier or diluent.

As regards these applications, the present method includes the administration to an animal or subject in need of treatment, particularly a mammal, and more particularly a human, of a therapeutically effective amount of the compound effective in treatment of a disorder associated with PINK1 kinase activity. The dose administered to an animal, particularly a human, in the context of the present invention should be sufficient to affect a therapeutic response in the animal over a reasonable timeframe. One skilled in the art will recognize that dosage will depend upon a variety of factors including the condition of the animal and the body weight of the animal.

The total amount of the compound of the present disclosure administered in a typical treatment is preferably between about 10 mg/kg and about 1000 mg/kg of body weight for mice, and between about 100 mg/kg and about 500 mg/kg of body weight, and more preferably between 200 mg/kg and about 400 mg/kg of body weight for humans per daily dose. This total amount is typically, but not necessarily, administered as a series of smaller doses over a period of about one time per day to about three times per day for about 24 months, and preferably over a period of twice per day for about 12 months.

The size of the dose also will be determined by the route, timing and frequency of administration as well as the existence, nature and extent of any adverse side effects that might accompany the administration of the compound and the desired physiological effect. It will be appreciated by one of skill in the art that various conditions or disease states, in particular chronic conditions or disease states, may require prolonged treatment involving multiple administrations.

Any medicament having utility in an application described herein can be used in co-therapy, co-administration or co-formulation with a composition as described above. Such additional medicaments include, medicines for cholesterol, such as but not limited to niacin, acifran, a statin, such as, but not limited to, lovastatin, atorvastatin, fluvastatin, pitavastatin, rosuvastatin, simvastatin, and the like. Other additional medicaments include, but are not limited to, ezetimibe, Trilipix (fenofibric acid), and the like. Other medicaments and compositions include, but are not limited to, fish oil, red yeast rice, omega fatty acids, and the like.

The additional medicament can be administered in co-therapy (including co-formulation) with the one or more of the compounds described herein.

In some embodiments, the response of the disease or disorder to the treatment is monitored and the treatment regimen is adjusted if necessary in light of such monitoring.

Frequency of administration is typically such that the dosing interval, for example, the period of time between one dose and the next, during waking hours is from about 2 to about 12 hours, from about 3 to about 8 hours, or from about 4 to about 6 hours. It will be understood by those of skill in the art that an appropriate dosing interval is dependent to some degree on the length of time for which the selected composition is capable of maintaining a concentration of the compound(s) in the subject and/or in the target tissue (e.g., above the EC₅₀ (the minimum concentration of the compound which modulates the receptor's activity by 90%). Ideally the concentration remains above the EC₅₀ for at least 100% of the dosing interval. Where this is not achievable it is desired that the concentration should remain above the EC₅₀ for at least about 60% of the dosing interval, or should remain above the EC₅₀ for at least about 40% of the dosing interval.

Thus, in some embodiments, the invention relates to the manufacture of a medicament comprising combining a disclosed compound or a product of a disclosed method of making, or a pharmaceutically acceptable salt, solvate, or polymorph thereof, with a pharmaceutically acceptable carrier or diluent.

6. Kits

In some embodiments, disclosed are kits comprising a compound having a structure represented by a formula:

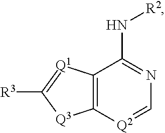

wherein Q¹ is N or CH and R³ is a 3- to 6-membered cycloalkyl, C1-C6 haloalkyl, C1-C6 haloalkoxy, or C1-C6 halohydroxyalkyl; or wherein Q¹ is CR¹ and R³ is hydrogen; R¹ is C1-C6 haloalkyl, C1-C6 haloalkoxy, C1-C6 halohydroxyalkyl, or a structure represented by a formula:

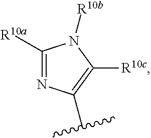

wherein each of R^{10a}, R^{10b}, and R^{10c}, when present, is independently selected from hydrogen and C1-C4 alkyl; wherein Q² is CH or N; wherein Q³ is CH₂ or NH; wherein R² is C1-C6 alkyl, —CR^{11a}R^{11b}Cy¹, or Cy¹; wherein each of R^{11a} and R^{11b}, when present, is independently selected from hydrogen, C1-C5 alkyl, and C1-C4 hydroxyalkyl; or wherein each of R^{11a} and R^{11b}, when present, together comprise a 3-membered cycloalkyl; wherein Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, or a pharmaceutically acceptable salt thereof, and one or more of: (a) at least one agent known for the treatment of a neurodegenerative disorder, a mitochondrial disorder, a fibrosis, and cardiomyopathy; (b) instructions for administering the compound in connection with the neurodegenerative disorder, a mitochondrial disorder, a fibrosis, or cardiomyopathy; and/or (c) instructions for treating the disorder.

In some embodiments, disclosed are kits comprising a compound having a structure represented by a formula:

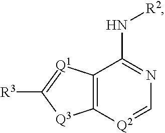

wherein Q¹ is N or CH and R³ is a 3- to 6-membered cycloalkyl, C1-C4 haloalkyl, C1-C4 cyanoalkyl, C1-C4 hydroxyalkyl, C1-C4 haloalkoxy, CF₃, CBr₃ or CCl₃; or wherein Q¹ is CR¹ and R³ is hydrogen; R¹ is C1-C6 haloalkyl, C1-C6 haloalkoxy, C1-C6 halohydroxy, or a structure represented by a formula:

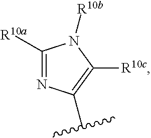

wherein each of R^{10a}, R^{10b}, and R^{10c}, when present, is independently selected from hydrogen and C1-C4 alkyl; wherein Q² is CH or N; wherein Q³ is CH₂ or NH; wherein R² is C1-C6 alkyl, —CR^{11a}R^{11b}Cy¹, or Cy¹; wherein each of R^{11a} and R^{11b}, when present, is independently selected from hydrogen, C1-C5 alkyl, and C1-C5 hydroxyalkyl; or wherein each of R^{11a} and R^{11b} together comprise a 3-membered cycloalkyl; wherein Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino, provided that when R¹ is C1-C6 haloalkyl and R² is Cy¹, then Cy¹ is not a 6-membered carbocycle or a 9-membered heteroaryl, or a pharmaceutically acceptable salt thereof, and one or more of: (a) at least one agent known for the treatment of a neurodegenerative disorder, a mitochondrial disorder, a fibrosis, and cardiomyopathy; (b) instructions for administering the compound in connection with the neurodegenerative disorder, a mitochondrial disorder, a fibrosis, or cardiomyopathy; and/or (c) instructions for treating the disorder.

In some embodiments, disclosed are kits comprising a compound selected from:

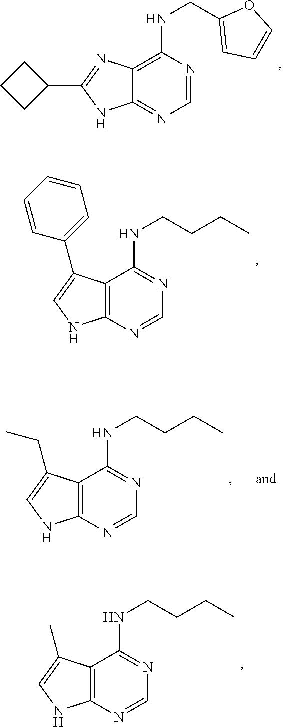

or a pharmaceutically acceptable salt thereof, and one or more of: (a) at least one agent known for the treatment of a neurodegenerative disorder, a mitochondrial disorder, a fibrosis, and cardiomyopathy; (b) instructions for administering the compound in connection with the neurodegenerative disorder, a mitochondrial disorder, a fibrosis, or cardiomyopathy; and/or (c) instructions for treating the disorder.

In some embodiments, disclosed are kits comprising a compound selected from:

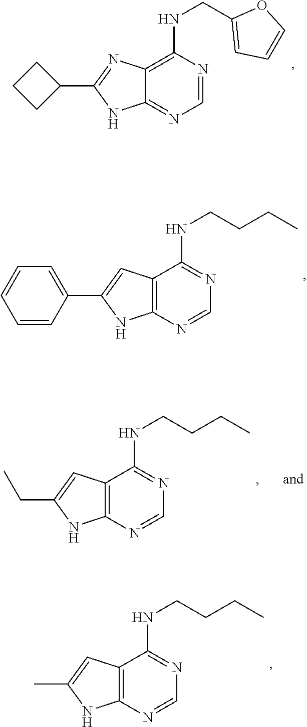

or a pharmaceutically acceptable salt thereof, and one or more of: (a) at least one agent known for the treatment of a neurodegenerative disorder, a mitochondrial disorder, a fibrosis, and cardiomyopathy; (b) instructions for administering the compound in connection with the neurodegenerative disorder, a mitochondrial disorder, a fibrosis, or cardiomyopathy; and/or (c) instructions for treating the disorder.

In some embodiments, disclosed are kits comprising a compound selected from:

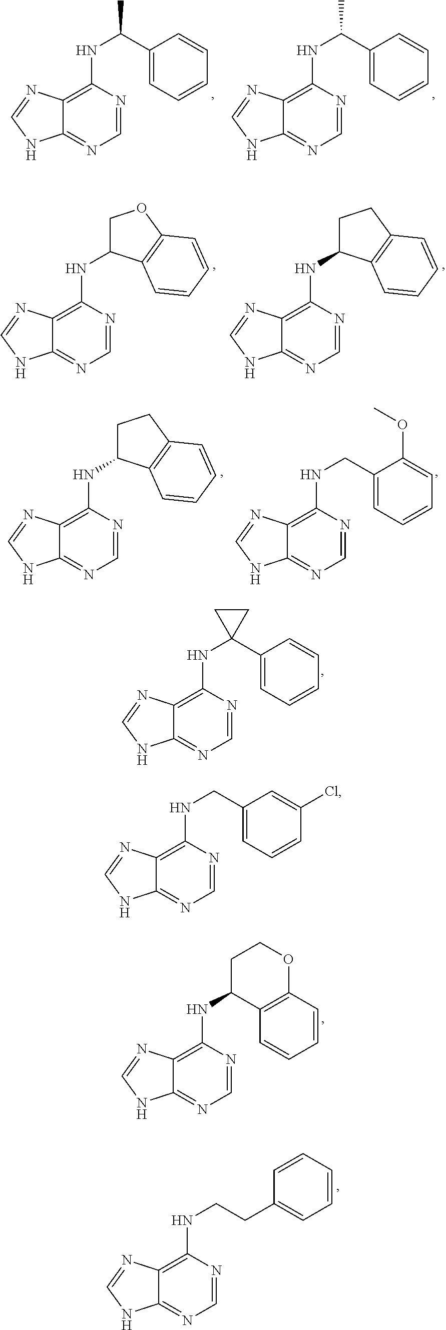

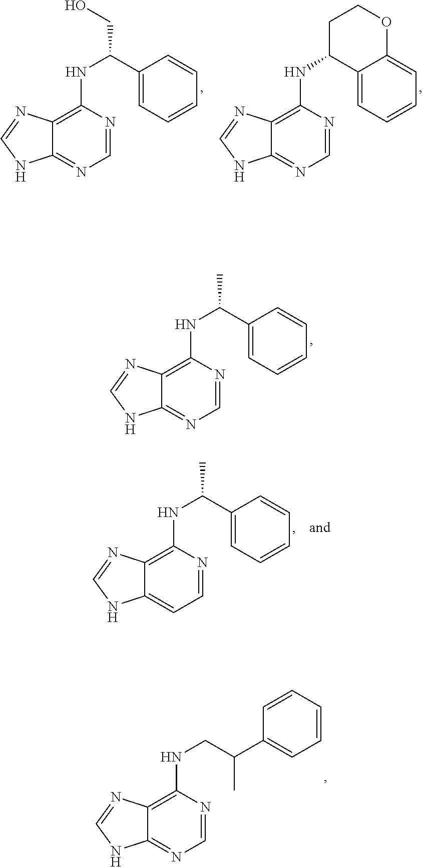

or a pharmaceutically acceptable salt thereof, and one or more of: (a) at least one agent known for the treatment of a neurodegenerative disorder, a mitochondrial disorder, a fibrosis, and cardiomyopathy; (b) instructions for administering the compound in connection with the neurodegenerative disorder, a mitochondrial disorder, a fibrosis, or cardiomyopathy; and/or (c) instructions for treating the disorder.

In some embodiments, disclosed are kits comprising a compound having a structure represented by Formula I:

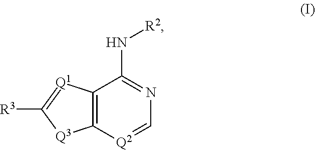

wherein Q¹ is N or CH and R³ is a 3- to 6-membered cycloalkyl, C1-C4 haloalkyl, C1-C4 cyanoalkyl, C1-C4 hydroxyalkyl, C1-C4 haloalkoxy, CF₃, CBr₃ or CCl₃; wherein Q² is CH or N; wherein Q³ is CH₂ or NH; R¹ is (C₁-C₆)alkyl, halo(C₁-C₄)alkyl, (C₁-C₄)alkoxy, halo(C₁-C₄)alkoxy, 5- or 6-membered heteroaryl, or phenyl, wherein said C₁-C₆alkyl and halo(C₁-C₄)alkyl are each optionally and independently substituted with a OR^{a} group, and wherein said phenyl and 5- or 6-membered heteroaryl are each optionally and independently substituted with 1 to 3 groups independently selected from R^{b}; R^{a}, when present, is H, (C₁-C₄)alkyl, or (C₁-C₄)alkoxy; each occurrence of R^{b}, when present, is independently halo, halo(C₁-C₄)alkyl, (C₁-C₄)alkoxy, or halo(C₁-C₄)alkoxy; R² is (C₁-C₆)alkyl, a 9-membered oxygen-containing fused heterocycle, or a 9- to 10-membered carbocycle, wherein said (C₁-C₆)alkyl is optionally substituted with 1 or 2 groups independently selected from R^{c}, and wherein said 9-membered oxygen-containing fused heterocycle and 9- to 10-membered carbocycle are each optionally and independently substituted with 1 to 3 groups independently selected from R^{d}; each occurrence of R^{c}, when present, is phenyl, 3- or 4-membered cycloalkyl, or 5- or 6-membered heteroaryl, wherein said phenyl and 5- or 6-membered heteroaryl are each optionally and independently substituted with 1 to 3 groups independently selected from R^{e}; each occurrence of R^{d} and R^{e}, when present, is independently halo, halo(C₁-C₄)alkyl, (C₁-C₄)alkoxy, or halo(C₁-C₄)alkoxy; and R³ is hydrogen, halogen, (C₁-C₄)alkyl, or 3- to 6-membered cycloalkyl, or pharmaceutically acceptable salts thereof, and one or more of: (a) at least one agent known for the treatment of a neurodegenerative disease, a mitochondrial disease, fibrosis, and/or cardiomyopathy; (b) instructions for administering the compound in connection with treating a neurodegenerative disease, a mitochondrial disease, fibrosis, and/or cardiomyopathy; and (c) instructions for treating a neurodegenerative disease, a mitochondrial disease, fibrosis, and/or cardiomyopathy.

In further embodiments, the agent is known for the treatment of a neurodegenerative disorder. Examples of agents known for the treatment of neurodegenerative disorders include, but are not limited to, cholinesterase inhibitor, an antidepressant, memantine, rilutek, radicava, levodopa, carbidopa, a dopamine agonist, a MAO-B inhibitor, a catechol-O-methyltransferase inhibitor, an anticholinergic, spinraza, tetrabenazine, an antipsychotic agent, levetiracetam, clonazepam, an antipsychotic agent, a mood-stabilizing agent, and amantadine.

In further embodiments, the agent is known for the treatment of a mitochondrial disease. Examples of agents known for the treatment of mitochondrial diseases include, but are not limited to, vitamins and supplements such as coenzyme Q10, B complex vitamins (e.g., thiamine (B1) and riboflavin (B2)), alpha lipoic acid, L-camitine (Camitor), creatine, and L-arginine.

In further embodiments, the agent is known for the treatment of fibrosis such as, for example, idiopathic pulmonary fibrosis (IPF), non-alcoholic fatty liver disease (NASH), liver fibrosis, heart fibrosis, mediastinal fibrosis, bone marrow fibrosis, retroperitoneal cavity fibrosis, and renal fibrosis. Examples of agents known for the treatment of fibrosis include, but are not limited to, pirfenidone, nintedanib, a prostaglandin such as latanoprost and bimaotoprost, a beta blocker such as timolol and betaxolol, an alpha-adrenergic agonist such as apraclonidine and brimonidine, a carbonic anhydrase inhibitor such as dorzolamide and brinzolamide, a moitic or cholinergic agent such as pilocarpine, a diuretic, an angiotenisin-converting enzyme (ACE) inhibitor, an angiotensin II receptor blocker, an anti-inflammatory agent, and an anti-fibrotic agent.

In further embodiments, the agent is known for the treatment of cardiomyopathy. Examples of agents known for the treatment of cardiomyopathy include, but are not limited to, ACE inhibitors, angiotensin II receptor blockers, beta blockers, calcium channel blockers, digoxin, and antiarrhythmics. In various embodiments, the agent known for the treatment of cardiomyopathy is a medical device such as, for example, an implantable cardioverter-defibrillator (ICD), a ventricular assist device (VAD), or a pacemaker. In further embodiments, the at least one compound and the at least one agent are co-formulated. In further embodiments, the at least one compound and the at least one agent are co-packaged.

In further embodiments, the compound and the agent are administered sequentially. In still further embodiments, the compound and the agent are administered simultaneously.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is understood that the disclosed kits can be prepared from the disclosed compounds, products, and pharmaceutical compositions. It is also understood that the disclosed kits can be employed in connection with the disclosed methods of using.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

All publications and patent applications cited in this specification are herein incorporated by reference in their entireties, and for any and all purposes, as if each individual publication or patent application were specifically and individually indicated to be incorporated by reference. In the event of an inconsistency between the present disclosure and any publications or patent application incorporated herein by reference, the present disclosure controls.

### F. Exemplification

Representative examples of the disclosed compounds are illustrated in the following non-limiting methods, schemes, and examples.

### 1. General Experimental Method

General starting materials used were obtained from commercial sources or prepared in other examples, unless otherwise noted. All temperatures are in degrees Celsius (° C.) and are uncorrected. Reagent grade chemicals and anhydrous solvent were purchased from commercial sources and unless otherwise mentioned, were used without further purification. The names of the products were determined using the naming software included in Biovia electronic lab notebook. Silica gel chromatography was performed on Teledyne Isco instruments using pre-packaged disposable SiO₂ stationary phase columns with eluent flow rate range of 15 to 200 mL/min, UV detection (254 and 280 nm). Reverse phase preparative HPLC was carried out using C18 columns, UV detection (214 and 254 nm) eluting with gradients of MeCN in H₂O (0.03% (NH₄)₂CO₃/0.375% NH₄OH, high pH) or MeCN in H₂O (0.1% HCOOH, low pH). The analytical HPLC chromatograms were performed using an Agilent 1100 series instrument with DAD detector (190 nm to 300 nm). The mass spectra were recorded with a Waters Micromass ZQ detector at 130° C. The mass spectrometer was equipped with an electrospray ion source (ESI) operated in a positive ion mode and was set to scan between m/z 150-750 with a scan time of 0.3 s. Products and intermediates were analyzed by HPLC/MS on a Gemini-NX (5 μM, 2.0×30 mm) using a high pH buffer gradient of 5% to 100% of MeCN in H₂O (0.03% (NH₄)₂CO₃/0.375% NH₄OH) over 2.5 min at 1.8 mL/min for a 3.5 min run (B05) and EVO C18 (5 μM, 3.0×50 mm) using a low pH buffer gradient of 5% to 100% of MeCN in H₂O (0.1% HCOOH) over 2.5 min at 2.2 mL/min for a 3.5 min run (A05). The ¹H NMR spectra were recorded on a Bruker UltraShield 500 MHz/54 mm instrument (BZH 43/500/70B, D221/54-3209). The chemical shifts are referenced to solvent peaks, which in ¹H NMR appear at 7.26 ppm for CDCl₃, 2.50 for DMSO-d6, and 3.31 ppm for CD₃OD.

The following abbreviations have the indicated meanings:

aq aqueous;

(Bpin)₂ bis(pinacolato)diboron;

Comins' reagent N-bis(trifluoromethanesulfonimide);

DBDMH 1,3-dibromo-5,5-dimethylhydantoin

DMF N,N-dimethyl formamide;

DMSO dimethyl sulfoxide;

Et₂O diethyl ether;

EtOAc ethyl acetate;

EtOH ethanol;

eq. or equiv. equivalent

h hour(s);

HPLC high performance liquid chromatography;

LCMS liquid chromatography mass spectrometry

LiHMDS lithium bis(trimethylsilyl)amide

MeOH methanol;

m minute(s);

MS mass spectrometry

NaHMDS sodium bis(trimethylsilyl)amide

NMP N-methylpyrrolidone

NMR nuclear magnetic resonance;

23° C. room temperature;

sat. saturated;

SFC supercritical fluid chromatography;

THF tetrahydrofuran;

OTf trifluoromethanesulfonate.

### 2. Synthesis of N-butyl-8-cyclopropyl-9H-purin-6-amine (EP-0034886)

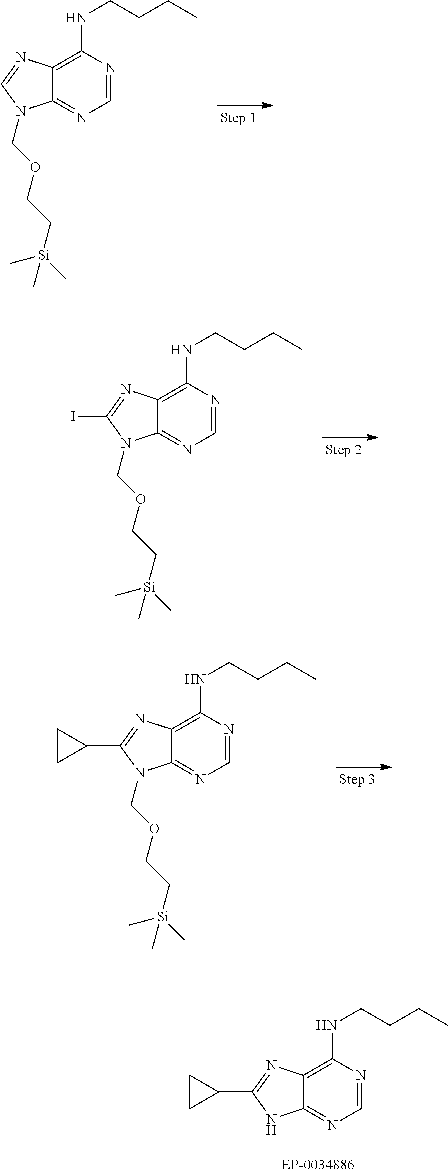 [SMILES: CCCCNc1ncnc2c1ncn2COCC[Si](C)(C)C ; CCCCNc1ncnc2c1nc(I)n2COCC[Si](C)(C)C ; CCCCNc1ncnc2c1nc(C1CC1)n2COCC[Si](C)(C)C ; CCCCNc1ncnc2[nH]c(C3CC3)nc12]

### a. Step 1: N-butyl-8-iodo-9-(2-trimethylsilylethoxymethyl)purin-6-amine

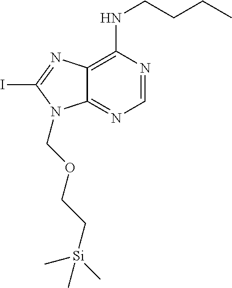 [SMILES: CCCCNc1ncnc2c1nc(I)n2COCC[Si](C)(C)C]

N-Butyl-9-(2-trimethylsilylethoxymethyl)purin-6-amine (150 mg, 0.47 mmol) was dissolved in THF (7.50 mL) and cooled to −78° C. under a nitrogen atmosphere before lithium diisopropylamide (1.00 M in THF, 2.33 mL, 2.33 mmol) was added dropwise. The mixture was stirred for 1 h at −78° C. Iodine (0.22 g, 0.86 mol) in THF (2.00 mL) was added dropwise, and the solution was stirred at −78° C. for 16 h. The solution was diluted with sat. NH₄Cl (15.0 mL). The layers were separated, and the aqueous layer was extracted with EtOAc (3×20.0 mL). The combined organic layers were washed with sat. Na₂S₂O₃ (50.0 mL), brine (50.0 mL), dried over MgSO₄, filtered, and concentrated under reduced pressure. The residue was purified by silica gel chromatography (12 g cartridge) eluting with hexanes and EtOAc (0-60%) to afford the title compound (48 mg, 23%) as an oil. ¹H NMR (500 MHz, CDCl₃) δ 8.32 (s, 1H), 5.67 (s, 1H), 5.51 (s, 2H), 3.69-3.59 (m, 4H), 1.70-1.62 (m, 2H), 1.45 (dd, J=15.1, 7.5 Hz, 2H), 1.00-0.90 (m, 5H), −0.02-−0.05 (m, 9H); m/z (ES⁺): [M+H]⁺=447.3; HPLC (B05) t_{R}=2.14 m.

### b. Step 2: N-butyl-8-cyclopropyl-9-(2-trimethylsilylethoxymethyl)purin-6-amine

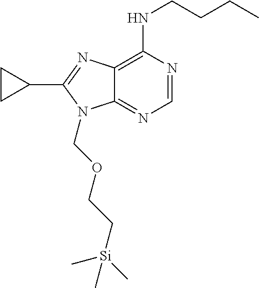 [SMILES: CCCCNc1ncnc2c1nc(C1CC1)n2COCC[Si](C)(C)C]

N-Butyl-8-iodo-9-(2-trimethylsilylethoxymethyl)purin-6-amine (48.0 mg, 0.11 mmol) was dissolved in 1,4-dioxane (1.00 mL) in a 2-mL microwave vial, and cyclopropylboronic acid (18.7 μL, 0.16 mmol), potassium phosphate tribasic (68.3 mg, 0.32 mmol), and Pd(dppf)Cl₂ (3.93 mg, 0.006 mmol) were added. The solution was degassed with N₂ for 15 min before being irradiated in a microwave to 150° C. for 2 h. The solution was diluted with sat. NH₄Cl (10.0 mL). The layers were separated, and the aqueous layer was extracted with EtOAc (3×10.0 mL). The combined organic layers were washed with brine (50.0 mL), dried over Na₂SO₄, filtered, and concentrated under reduced pressure. The residue was purified by silica gel chromatography (4 g cartridge) eluting with hexanes and EtOAc (0-70%) to afford the title compound (24 mg, 62%) as an oil. ¹H NMR (500 MHz, CDCl₃) δ 8.29 (s, 1H), 5.64 (s, 2H), 3.63 (s, 2H), 3.64-3.58 (m, 2H), 2.17 (tt, J=8.2, 5.0 Hz, 1H), 1.72-1.63 (m, 2H), 1.45 (dd, J=15.0, 7.4 Hz, 2H), 1.22-1.09 (m, 4H), 0.95 (dt, J=16.4, 7.7 Hz, 5H), −0.02-−0.06 (m, 9H); m/z (ES⁺): [M+H]⁺=361.6; HPLC (A05) t_{R}=1.90 m.

### c. Step 3: N-butyl-8-cyclopropyl-9H-purin-6-amine (EP-0034886)

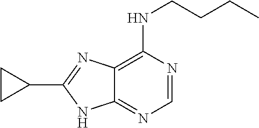 [SMILES: CCCCNc1ncnc2[nH]c(C3CC3)nc12]

N-Butyl-8-cyclopropyl-9-(2-trimethylsilylethoxymethyl)purin-6-amine (24.0 mg, 0.06 mmol) was dissolved in THF (0.50 mL), and TBAF (1.00 M in THF, 79.7 μL, 0.08 mmol) was added. The solution was stirred under a nitrogen atmosphere at 75° C. for 4 h. The solution was diluted with water (5.00 mL). The layers were separated, and the aqueous layer was extracted with EtOAc (3×10.0 mL). The combined organic layers were washed with brine (20.0 mL), dried over Na₂SO₄, filtered, and concentrated under reduced pressure. The residue was purified by silica gel chromatography (4 g cartridge) eluting with hexanes and EtOAc (0-100%) to afford the title compound (5.0 mg, 33%) as a solid. ¹H NMR (400 MHz, CD₃OD) δ 8.23 (s, 1H), 3.61 (s, 2H), 2.21-2.13 (m, 1H), 1.75-1.64 (m, 2H), 1.48 (dq, J=14.8, 7.5 Hz, 2H), 1.23-1.15 (m, 2H), 1.13 (dd, J=7.7, 2.9 Hz, 2H), 1.00 (t, J=7.4 Hz, 3H); m/z (ES⁺): [M+H]⁺=231.7; HPLC (B05) t_{R}=1.56 m.

### 3. Synthesis of 8-cyclobutyl-N-(2-furylmethyl)-9H-purin-6-amine (EP-0035338)

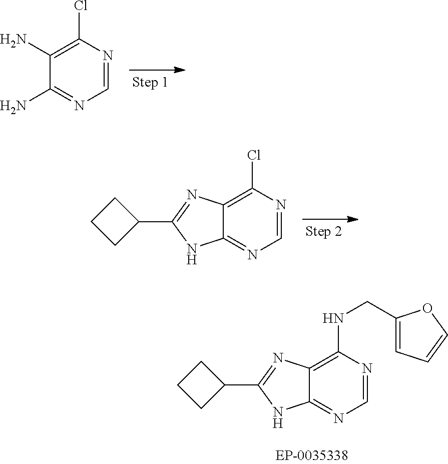 [SMILES: Nc1ncnc(Cl)c1N ; Clc1ncnc2[nH]c(C3CCC3)nc12 ; c1coc(CNc2ncnc3[nH]c(C4CCC4)nc23)c1]

### a. Step 1: 6-chloro-8-cyclobutyl-9H-purine

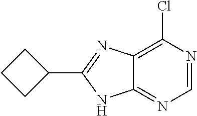 [SMILES: Clc1ncnc2[nH]c(C3CCC3)nc12]

To a solution of 6-chloropyrimidine-4,5-diamine (345 mg, 2.39 mmol) in POCl₃ (9.20 mL) were added NH₄Cl (766 mg, 14.3 mmol) and cyclobutanecarboxylic acid (239 mg, 2.39 mmol). The mixture was stirred at 110° C. for 16 h. The solution was diluted with water (200 mL) and sat. K₂CO₃ (100 mL). The aqueous layer was extracted with DCM (4×50.0 mL), and the combined organic extracts were dried (MgSO₄), filtered, and concentrated under reduced pressure. The residue was purified by silica gel chromatography (40 g silica cartridge) eluting with DCM and MeOH (0-10%) affording the title compound (430 mg, 86%) as a solid. m/z (ESI⁺): [M+H]⁺=209.3; HPLC (A05) t_{R}=1.92 m.

### b. Step 2: 8-cyclobutyl-N-(2-furylmethyl)-9H-purin-6-amine (EP-0035338)

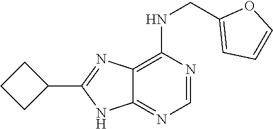 [SMILES: c1coc(CNc2ncnc3[nH]c(C4CCC4)nc23)c1]

6-Chloro-8-cyclobutyl-9H-purine (35.0 mg, 0.17 mmol) was dissolved in EtOH (1.50 mL) in a 2 mL microwave vial before 2-furylmethylamine (0.02 mL, 0.25 mmol) and DIPEA (0.04 mL, 0.22 mmol) were added. The solution was irradiated in a microwave at 140° C. for 1.2 h. The mixture was concentrated under reduced pressure, and the residue was purified by silica gel chromatography (12 g silica cartridge) eluting with DCM and MeOH (0-10%) to afford the title compound as a solid (17.0 mg, 38%). ¹H NMR (400 MHz, DMSO-d6) δ 12.68 (s, 1H), 8.14 (s, 1H), 7.86 (s, 1H), 7.53 (s, 1H), 6.35 (s, 1H), 6.22 (s, 1H), 4.69 (s, 2H), 3.66 (p, J=8.9 Hz, 1H), 2.44-2.26 (m, 4H), 2.10-1.97 (m, 1H), 1.94-1.83 (m, 1H); m/z (ES⁺): [M+H]⁺=270.2; HPLC (A05) t_{R}=2.06 m.

### 4. N-butyl-8-cyclobutyl-9H-purin-6-amine (EP-0035339)

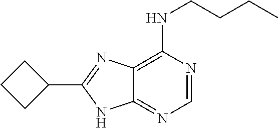 [SMILES: CCCCNc1ncnc2[nH]c(C3CCC3)nc12]

6-Chloro-8-cyclobutyl-9H-purine (28.0 mg, 0.13 mmol) was dissolved in EtOH (1.50 mL), and n-butylamine (19.9 μL, 0.20 mmol) and DIPEA (30.4 □L, 0.17 mmol) were added. The solution was irradiated in a microwave at 120° C. for 1.2 h. The mixture was concentrated under reduced pressure, and the residue was purified by silica gel chromatography (12 g silica cartridge) eluting with DCM and MeOH (0-10%) to afford the title compound as a solid (17.0 mg, 52%). ¹H NMR (400 MHz, DMSO-d6) δ 12.58 (s, 1H), 8.10 (s, 1H), 7.37 (s, 1H), 3.64 (p, J=8.7 Hz, 1H), 3.46 (s, 2H), 2.44-2.23 (m, 4H), 2.14-1.93 (m, 1H), 1.93-1.77 (m, 1H), 1.67-1.48 (m, 2H), 1.40-1.26 (m, 2H), 0.90 (t, J=7.4 Hz, 3H); m/z (ES⁺): [M+H]⁺=245.9; HPLC (A05) t_{R}=2.14 m.

### 5. Synthesis of N-butyl-5-(trifluoromethyl)-7H-pyrrolo[2,3-d]pyrimidin-4-amine (EP-0035507)

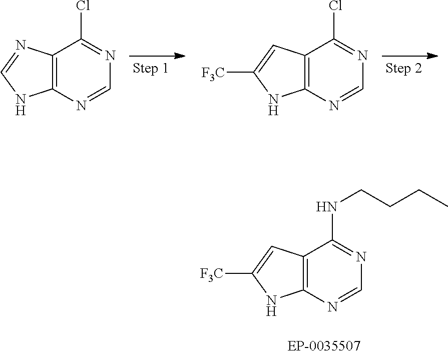 [SMILES: Clc1ncnc2[nH]cnc12 ; FC(F)(F)c1cc2c(Cl)ncnc2[nH]1 ; CCCCNc1ncnc2[nH]c(C(F)(F)F)cc12]

### a. Step 1: 4-chloro-5-(trifluoromethyl)-7H-pyrrolo[2,3-d]pyrimidine

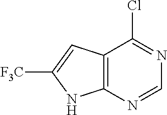 [SMILES: FC(F)(F)c1cc2c(Cl)ncnc2[nH]1]

To a solution of 4-chloro-7H-pyrrolo[2,3-d]pyrimidine (11.0 g, 71.6 mmol) and trifluoromethanesulfinic acid, sodium salt (33.5 g, 215 mmol) in a mixture of DCM (250 mL) and water (100 mL) was added tert-butyl hydroperoxide (46.1 mL, 358 mmol) at 0° C. at a speed of 17 mL/hr. The solution was stirred at this temperature for 1 h and warmed to room temperature for 96 h. The mixture was diluted with sat. sodium bicarbonate (100 mL), and the aqueous phase was extracted with DCM (3×70.0 mL). The combined organic extracts were dried (MgSO₄), filtered, and concentrated under reduced pressure. The mixture was purified by silica gel chromatography (120 g silica cartridge) eluting with hexane and EtOAc (0-50%) to afford the title compound (4.85 g, 31%) as a solid. m/z (ES⁺): [M+H]⁺=222.7; HPLC (A05) t_{R}=2.30 m.

### b. Step 2: N-butyl-5-(trifluoromethyl)-7H-pyrrolo[2,3-d]pyrimidin-4-amine (EP-0035507)

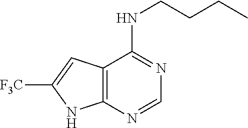 [SMILES: CCCCNc1ncnc2[nH]c(C(F)(F)F)cc12]

To a solution of 4-chloro-5-(trifluoromethyl)-7H-pyrrolo[2,3-d]pyrimidine (16.0 mg, 72.2 μmol) in EtOH (1.50 mL) in a 2 mL microwave vial were added n-butylamine (10.7 μL, 0.11 mmol) and DIPEA (31.6 μL, 0.18 mmol). The solution was stirred at 110° C. for 14 h. The solution was concentrated under reduced pressure, and the residue was purified by silica gel chromatography (12 g silica cartridge) eluting with DCM and MeOH (0-10%) to afford the title compound as a solid (11.0 mg, 59%). ¹H NMR (400 MHz, DMSO-d6) δ 12.69 (s, 1H), 8.20 (s, 1H), 7.75 (s, 1H), 7.13 (s, 1H), 3.57-3.39 (m, 2H), 1.69-1.45 (m, 2H), 1.46-1.26 (m, 2H), 0.91 (t, J=7.3 Hz, 3H); m/z (ES⁺): [M+H]⁺=259.7; HPLC (A05) t_{R}=2.31 m.

### 6. N-benzyl-5-(trifluoromethyl)-7H-pyrrolo[2,3-d]pyrimidin-4-amine (EP-0035640)

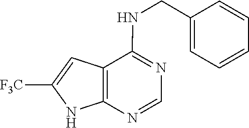 [SMILES: FC(F)(F)c1cc2c(NCc3ccccc3)ncnc2[nH]1]

4-Chloro-5-(trifluoromethyl)-7H-pyrrolo[2,3-d]pyrimidine (30.0 mg, 0.14 mmol) was dissolved in EtOH (0.48 mL), and benzylamine (16.3 mL, 0.149 mmol) and DIPEA (0.05 mL, 0.27 mmol) were added. The solution was heated at 150° C. for 16 h. The solution was concentrated under reduced pressure, and the residue was purified by silica gel chromatography (12 g cartridge) eluting with DCM and MeOH (0-6%) to afford the title compound (23.9 mg, 60%) as a solid. ¹H NMR (500 MHz, DMSO) δ 12.78 (s, 1H), 8.35 (t, J=5.9 Hz, 1H), 8.23 (s, 1H), 7.39-7.28 (m, 4H), 7.24 (dt, J=9.2, 4.3 Hz, 1H), 7.19 (s, 1H), 4.73 (d, J=6.0 Hz, 2H); m/z (ES⁺): [M+H]⁺=293.7; HPLC (B05) t_{R}=2.43 min.

### 7. N-[(2-methoxyphenyl)methyl]-5-(trifluoromethyl)-7H-pyrrolo[2,3-d]pyrimidin-4-amine (EP-0035764)

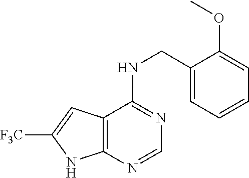 [SMILES: COc1ccccc1CNc1ncnc2[nH]c(C(F)(F)F)cc12]

4-Chloro-5-(trifluoromethyl)-7H-pyrrolo[2,3-d]pyrimidine (30.0 mg, 0.14 mmol) was dissolved in EtOH (0.48 mL), and (2-methoxyphenyl)methanamine (0.02 mL, 0.14 mmol) and DIPEA (0.05 mL, 0.27 mmol) were added. The solution was heated at 150° C. for 16 h. The solution was concentrated under reduced pressure, and the residue was purified by silica gel chromatography (12 g cartridge) eluting with DCM and MeOH (0-6%) to afford the title compound (22.5 mg, 52%) as a solid. ¹H NMR (500 MHz, DMSO-d6) δ 12.71 (s, 1H), 8.20 (s, 1H), 8.17 (t, J=5.7 Hz, 1H), 7.27-7.18 (m, 3H), 7.01 (d, J=7.5 Hz, 1H), 6.87 (td, J=7.4, 1.0 Hz, 1H), 4.68 (d, J=5.9 Hz, 2H), 3.83 (s, 3H); m/z (ES⁺): [M+H]⁺=323.5; HPLC (B05) t_{R}=2.49 min.

### 8. N-(1-phenylcyclopropyl)-5-(trifluoromethyl)-7H-pyrrolo[2,3-d]pyrimidin-4-amine (EP-0035788)

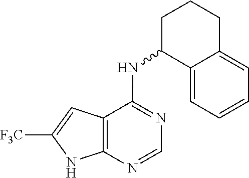 [SMILES: FC(F)(F)c1cc2c(NC3CCCc4ccccc43)ncnc2[nH]1]

4-Chloro-5-(trifluoromethyl)-7H-pyrrolo[2,3-d]pyrimidine (30.0 mg, 0.14 mmol) was dissolved in EtOH (0.48 mL), and tetralin-1-amine (0.02 mL, 0.14 mmol) and DIPEA (0.05 mL, 0.27 mmol) were added. The solution was heated at 150° C. for 16 h. The solution was concentrated under reduced pressure, and the residue was purified by silica gel chromatography (12 g cartridge) eluting with DCM and MeOH (0-6%) to afford the title compound (20.4 mg, 45%) as a solid. ¹H NMR (500 MHz, DMSO-d6) δ 12.73 (s, 1H), 8.27 (s, 1H), 8.14 (d, J=8.5 Hz, 1H), 7.27-7.08 (m, 5H), 5.66-5.56 (m, 1H), 2.90-2.72 (m, 2H), 2.11-1.90 (m, 2H), 1.89-1.73 (m, 2H); m/z (ES⁺): [M+H]⁺=333.9; HPLC (B05) t_{R}=2.75 min.

### 9. N-[(2,6-dimethoxyphenyl)methyl]-5-(trifluoromethyl)-7H-pyrrolo[2,3-d]pyrimidin-4-amine (EP-0035855)

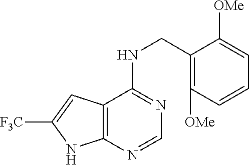 [SMILES: COc1cccc(OC)c1CNc1ncnc2[nH]c(C(F)(F)F)cc12]

4-Chloro-5-(trifluoromethyl)-7H-pyrrolo[2,3-d]pyrimidine (16.0 mg, 72.2 μmol) was dissolved in EtOH (1.50 mL), and (2,6-dimethoxyphenyl)methanamine (12.1 mg, 72.2 μmol) and DIPEA (31.6 μL, 0.18 mmol) were added. The solution was stirred at 110° C. for 15 h. The solution was concentrated under reduced pressure, and the residue was purified by silica gel chromatography (12 g silica cartridge) eluting with DCM and MeOH (0-10%) to afford the title compound as a solid (15.0 mg, 59%). ¹H NMR (400 MHz, DMSO-d6) δ 12.64 (s, 1H), 8.27 (s, 1H), 7.50 (s, 1H), 7.38-7.18 (m, 2H), 6.70 (d, J=8.4 Hz, 2H), 4.59 (d, J=4.1 Hz, 2H); m/z (ES⁺): [M+H]⁺=253.9; HPLC (A05) t_{R}=2.49 m.

### 10. N-(pyrimidin-5-ylmethyl)-5-(trifluoromethyl)-7H-pyrrolo[2,3-d]pyrimidin-4-amine (EP-0035910)

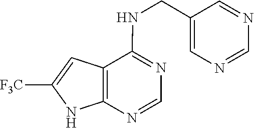 [SMILES: FC(F)(F)c1cc2c(NCc3cncnc3)ncnc2[nH]1]

4-Chloro-5-(trifluoromethyl)-7H-pyrrolo[2,3-d]pyrimidine (30.0 mg, 0.14 mmol) was dissolved in EtOH (0.48 mL), and pyrimidin-5-ylmethanamine (0.02 mL, 0.16 mmol) and DIPEA (0.05 mL, 0.27 mmol) were added. The solution was heated at 150° C. for 16 h. The solution was concentrated under reduced pressure, and the residue was purified by silica gel chromatography (12 g cartridge) eluting with DCM and MeOH (0-6%) to afford the title compound (28.1 mg, 71%) as a solid. ¹H NMR (500 MHz, DMSO-d6) δ 12.85 (s, 1H), 9.08 (s, 1H), 8.81 (s, 2H), 8.45 (t, J=5.8 Hz, 1H), 8.26 (s, 1H), 7.14 (d, J=1.2 Hz, 1H), 4.74 (d, J=5.8 Hz, 2H); m/z (ES⁺): [M+H]⁺=295.2; HPLC (B05) t_{R}=2.05 min.

### 11. N-chroman-4-yl-5-(trifluoromethyl)-7H-pyrrolo[2,3-d]pyrimidin-4-amine (EP-0035987)

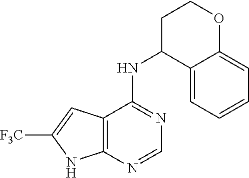 [SMILES: FC(F)(F)c1cc2c(NC3CCOc4ccccc43)ncnc2[nH]1]

4-Chloro-5-(trifluoromethyl)-7H-pyrrolo[2,3-d]pyrimidine (30.0 mg, 0.14 mmol) was dissolved in EtOH (0.48 mL), and chroman-4-amine (0.02 mL, 0.16 mmol) and DIPEA (0.05 mL, 0.27 mmol) were added. The solution was heated at 150° C. for 16 h. The solution was concentrated under reduced pressure, and the residue was purified by silica gel chromatography (12 g cartridge) eluting with DCM and MeOH (0-6%) to afford the title compound (19.5 mg, 43%) as a solid. ¹H NMR (500 MHz, DMSO-d6) δ 12.79 (s, 1H), 8.30 (s, 1H), 8.22 (s, 1H), 7.18 (ddd, J=11.2, 9.6, 4.3 Hz, 3H), 6.86 (ddd, J=14.2, 10.2, 4.7 Hz, 2H), 5.59 (dd, J=13.3, 5.7 Hz, 1H), 4.31-4.23 (m, 2H), 2.17 (dq, J=18.9, 5.5 Hz, 1H), 2.09-2.01 (m, 1H); m/z (ES⁺): [M+H]⁺=335.2; HPLC (B05) t_{R}=2.46 min.

### 12. N-isochroman-4-yl-5-(trifluoromethyl)-7H-pyrrolo[2,3-d]pyrimidin-4-amine (EP-0036023)

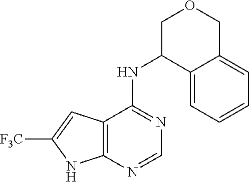 [SMILES: FC(F)(F)c1cc2c(NC3COCc4ccccc43)ncnc2[nH]1]

4-Chloro-5-(trifluoromethyl)-7H-pyrrolo[2,3-d]pyrimidine (30.0 mg, 0.14 mmol) was dissolved in EtOH (0.48 mL), and isochroman-4-ylammonium chloride (30.0 mg, 0.16 mmol) and DIPEA (0.05 mL, 0.27 mmol) were added. The solution was heated at 150° C. for 16 h. The solution was concentrated under reduced pressure, and the residue was purified by silica gel chromatography (12 g cartridge) eluting with DCM and MeOH (0-6%) to afford the title compound (30.5 mg, 67%) as a solid. ¹H NMR (400 MHz, DMSO-d6) δ 12.73 (s, 1H), 8.26 (s, 1H), 8.16 (d, J=8.2 Hz, 1H), 7.32-7.16 (m, 4H), 7.14-7.03 (m, 1H), 5.55-5.49 (m, 1H), 4.74 (q, J=15.2 Hz, 2H), 3.97 (dd, J=11.4, 4.4 Hz, 1H), 3.82 (dd, J=11.4, 5.4 Hz, 1H); m/z (ES⁺): [M+H]⁺=335.4; HPLC (B05) t_{R}=2.41 min.

### 13. Synthesis of 8-cyclopropyl-N-[(2-methoxy-4-pyridyl)methyl]-9H-purin-6-amine (EP-0036032)

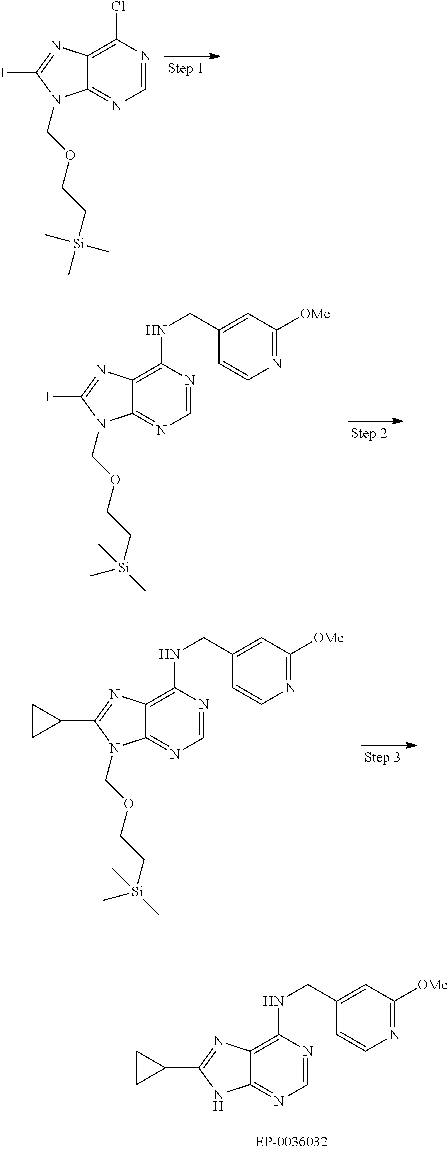 [SMILES: C[Si](C)(C)CCOCn1c(I)nc2c(Cl)ncnc21 ; COc1cc(CNc2ncnc3c2nc(I)n3COCC[Si](C)(C)C)ccn1 ; COc1cc(CNc2ncnc3c2nc(C2CC2)n3COCC[Si](C)(C)C)ccn1 ; COc1cc(CNc2ncnc3[nH]c(C4CC4)nc23)ccn1]

### a. Step 1: 8-iodo-N-(pyrimidin-5-ylmethyl)-9-(2-trimethylsilylethoxymethyl)purin-6-amine

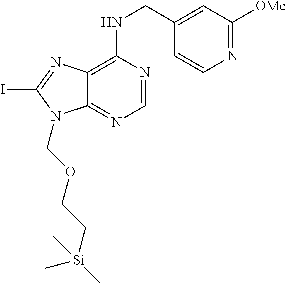 [SMILES: COc1cc(CNc2ncnc3c2nc(I)n3COCC[Si](C)(C)C)ccn1]

2-[(6-Chloro-8-iodo-purin-9-yl)methoxy]ethyl-trimethyl-silane (610 mg, 1.49 mmol), pyrimidin-5-ylmethanamine (259 mg, 2.38 mmol), and DIPEA (0.65 mL, 3.74 mmol) were dissolved in EtOH (10.0 mL). The mixture was heated to 110° C. and stirred for 16 h. The mixture was concentrated under reduced pressure, and the residue was purified by silica gel chromatography (25 g silica cartridge) eluting with hexanes and EtOAc (0-100%) to afford the title compound as a solid (645 mg, 90%).

m/z (ES⁺): [M+H]⁺=484.2 HPLC; (A05) t_{R}=2.25 m.

### b. Step 2: 8-cyclopropyl-N-(pyrimidin-5-ylmethyl)-9-(2-trimethylsilylethoxymethyl)purin-6-amine

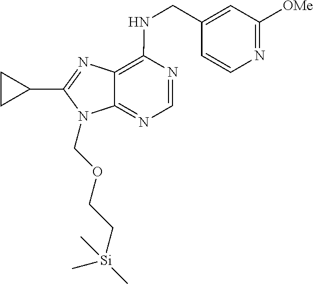 [SMILES: COc1cc(CNc2ncnc3c2nc(C2CC2)n3COCC[Si](C)(C)C)ccn1]

8-Iodo-N-(pyrimidin-5-ylmethyl)-9-(2-trimethylsilylethoxymethyl)purin-6-amine (100 mg, 0.21 mmol) was dissolved in 1,4-dioxane (3.00 mL), and cyclopropylboronic acid (35.5 mg, 0.41 mmol), tripotassium; phosphate (66.0 mg, 0.31 mmol), and Pd(dppf)Cl₂ (7.57 mg, 0.01 mmol) were added. The mixture was evacuated and backfilled with N₂ for 15 m before being irradiated in a microwave to 150° C. for 2 h. The solution was diluted with sat. NH₄Cl (5.00 mL). The layers were separated, and the aqueous layer was extracted with EtOAc (3×10.0 mL). The combined organic layers were washed with brine (50.0 mL), dried over Na₂SO₄, filtered, and concentrated under reduced pressure. The residue was purified by silica gel chromatography (12 g silica cartridge) eluting with DCM and MeOH (0-10%) to afford the title compound as an oil (50.0 mg, 61%).

m/z (ES⁺): [M+H]⁺=398.4; HPLC (A05) t_{R}=2.38 m.

### c. Step 3: EP-0036032, 8-cyclopropyl-N-[(2-methoxy-4-pyridyl)methyl]-9H-purin-6-amine

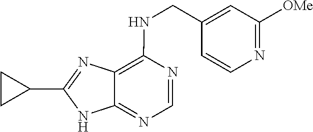 [SMILES: COc1cc(CNc2ncnc3[nH]c(C4CC4)nc23)ccn1]

TFA (3.00 mL) was added to a solution of 8-cyclopropyl-N-[(2-methoxy-4-pyridyl)methyl]-9-(2-trimethylsilylethoxymethyl)purin-6-amine (120 mg, 0.28 mmol) in dry DCM (2.00 mL). The solution was stirred at room temperature for 16 h under N₂. The solution was diluted with NaOH (2.5 M, 20.0 mL), and the aqueous phase was extracted with EtOAc (3×15.0 mL). The combined organic phases were concentrated under reduced pressure, and the residue was purified by silica gel chromatography (25 g silica cartridge) eluting with hexanes and EtOAc (0-100%) and by reverse phase chromatography (25 g C18 cartridge) eluting with water and MeOH (0-80%) to afford the title compound as a solid (35.5 mg, 43%). ¹H NMR (400 MHz, DMSO-d6) δ 12.69 (s, 1H), 8.08-8.02 (m, 2H), 8.00 (s, 1H), 6.91 (d, J=5.2 Hz, 1H), 6.66 (s, 1H), 4.63 (s, 2H), 3.79 (s, 3H), 2.07 (s, 1H), 1.11-0.96 (m, 4H); m/z (ES⁺): [M+H]⁺=297.2; HPLC (A05) t_{R}=2.00 m.

### 14. Synthesis of 8-cyclopropyl-N-(pyrimidin-5-ylmethyl)-9H-purin-6-amine (EP-0036050)

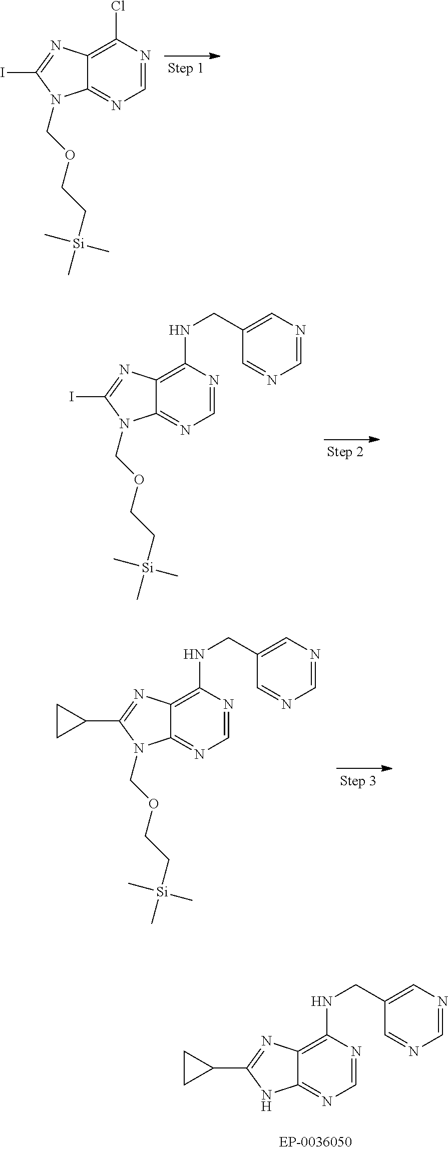 [SMILES: C[Si](C)(C)CCOCn1c(I)nc2c(Cl)ncnc21 ; C[Si](C)(C)CCOCn1c(I)nc2c(NCc3cncnc3)ncnc21 ; C[Si](C)(C)CCOCn1c(C2CC2)nc2c(NCc3cncnc3)ncnc21 ; c1ncc(CNc2ncnc3[nH]c(C4CC4)nc23)cn1]

### a. Step 1: 8-iodo-N-(pyrimidin-5-ylmethyl)-9-(2-trimethylsilylethoxymethyl)purin-6-amine

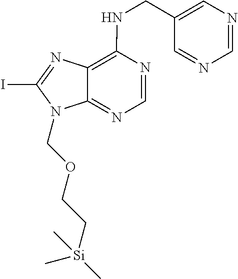 [SMILES: C[Si](C)(C)CCOCn1c(I)nc2c(NCc3cncnc3)ncnc21]

2-[(6-Chloro-8-iodo-purin-9-yl)methoxy]ethyl-trimethyl-silane (610 mg, 1.49 mmol), pyrimidin-5-ylmethanamine (259 mg, 2.38 mmol), and DIPEA (0.65 mL, 3.74 mmol) were dissolved in EtOH (10.0 mL). The mixture was heated to 110° C. and stirred for 16 h. The solution was concentrated under reduced pressure, and the residue was purified by silica gel chromatography (25 g silica cartridge) eluting with hexanes and EtOAc (0-100%) to afford the title compound as a solid (645 mg, 90%). m/z (ES⁺): [M+H]⁺=484.2; HPLC (A05) t_{R}=2.25 m.

### b. Step 2: 8-cyclopropyl-N-(pyrimidin-5-ylmethyl)-9-(2-trimethylsilylethoxymethyl)purin-6-amine

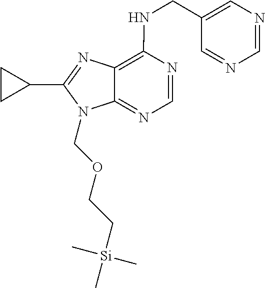 [SMILES: C[Si](C)(C)CCOCn1c(C2CC2)nc2c(NCc3cncnc3)ncnc21]

8-Iodo-N-(pyrimidin-5-ylmethyl)-9-(2-trimethylsilylethoxymethyl)purin-6-amine (100 mg, 0.21 mmol) was dissolved in 1,4-dioxane (3.00 mL), and cyclopropylboronic acid (35.5 mg, 0.41 mmol), tripotassium; phosphate (65.9 mg, 0.31 mmol), and Pd(dppf)Cl₂ (7.57 mg, 0.01 mmol) were added. The mixture was evacuated and backfilled with N₂ for 15 m before being irradiated in a microwave at 150° C. for 2 h. The solution was diluted with sat. NH₄Cl (5.00 mL). The layers were separated, and the aqueous layer was extracted with EtOAc (3×10.0 mL). The combined organic layers were washed with brine (50.0 mL), dried over Na₂SO₄, filtered, and concentrated under reduced pressure. The residue was purified by silica gel chromatography (12 g silica cartridge) eluting with DCM and MeOH (0-10%) to afford the title compound as an oil (50.0 mg, 61%).

m/z (ES⁺): [M+H]⁺=398.4; HPLC (A05) t_{R}=2.38 m.

### c. Step 3: 8-cyclopropyl-N-(pyrimidin-5-ylmethyl)-9H-purin-6-amine (EP-0036050)

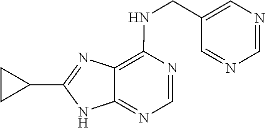 [SMILES: c1ncc(CNc2ncnc3[nH]c(C4CC4)nc23)cn1]

To a solution of 8-cyclopropyl-N-(pyrimidin-5-ylmethyl)-9-(2-trimethylsilylethoxymethyl)purin-6-amine (intermediate 5) (50.0 mg, 0.126 mmol) dissolved in dry DCM (2.00 mL) was added TFA (3.00 mL). The solution was stirred at room temperature for 16 h under N₂. The mixture was diluted with NaOH (2.5 M, 5.00 mL), and the aqueous phase was extracted with EtOAc (3×5.00 mL). The combined organic phases were concentrated under reduced pressure, and the residue was purified by silica gel chromatography (25 g Cis cartridge) eluting with water and MeOH (0-80%) to afford the title compound as a solid (12.0 mg, 36%). ¹H NMR (500 MHz, DMSO-D6) δ 12.63 (s, 1H), 9.04 (s, 1H), 8.77 (s, 2H), 8.11 (s, 1H), 8.04 (s, 1H), 4.69 (s, 2H), 2.07 (m, 1H), 1.09-0.96 (m, 4H). m/z (ES⁺): [M+H]⁺=268.1; HPLC (A05) t_{R}=1.82 m.

### 15. Synthesis of N-(pyrimidin-5-ylmethyl)-3-(trifluoromethyl)-1H-pyrrolo[3,2-c]pyridin-4-amine (EP-0036061)

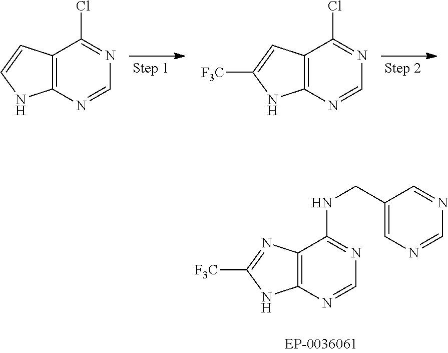 [SMILES: Clc1ncnc2[nH]ccc12 ; FC(F)(F)c1cc2c(Cl)ncnc2[nH]1 ; FC(F)(F)c1nc2c(NCc3cncnc3)ncnc2[nH]1]

### a. Step 1: 4-chloro-3-(trifluoromethyl)-1H-pyrrolo[3,2-c]pyridine

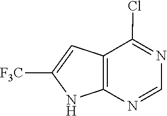 [SMILES: FC(F)(F)c1cc2c(Cl)ncnc2[nH]1]

Tert-butyl hydroperoxide (3.80 mL, 29.5 mmol) was added to a solution of 4-chloro-1H-pyrrolo[3,2-c]pyridine (900 mg, 5.90 mmol) and trifluoromethanesulfinic acid, sodium salt (3.15 g, 20.2 mmol) in TPGS-750-M (30.0 mL, 2% wt solution) at 0° C. The solution was stirred for 30 m and warmed to room temperature for 96 h. The mixture was diluted with sat. sodium bicarbonate (50.0 mL), and the aqueous phase was extracted with DCM (3×50.0 mL). The combined organic extracts were dried (MgSO₄), filtered, and concentrated under reduced pressure. The residue was purified by silica gel chromatography eluting with hexane and EtOAc (0-50%) to afford the title compound (190 mg, 12%) as a solid. ¹H NMR (400 MHz, DMSO-d6) δ 13.21 (s, 1H), 8.15 (d, J=5.8 Hz, 1H), 7.52 (dd, J=5.8, 0.8 Hz, 1H), 7.22-7.15 (m, 1H); m/z (ES⁺): [M+H]⁺=221.0; HPLC (A05) t_{R}=2.02 m.

### b. Step 2: N-(pyrimidin-5-ylmethyl)-3-(trifluoromethyl)-1H-pyrrolo[3,2-c]pyridin-4-amine (EP-0036061)

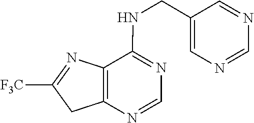 [SMILES: FC(F)(F)C1=Nc2c(ncnc2NCc2cncnc2)C1]

To a solution of 4-chloro-3-(trifluoromethyl)-1H-pyrrolo[3,2-c]pyridine (33.0 mg, 0.15 mmol), pyrimidin-5-ylmethanamine (22.1 mg, 0.23 mmol) and K₂CO₃ (66.2 mg, 0.48 mmol) in dry, degassed 1,4-dioxane (2.00 mL) was added Xphos Pd G2 (15.2 mg, 18.0 μmol). The solution was stirred under nitrogen at 100° C. for 15 h. The mixture was filtered over Celite, rinsing with DCM (10.0 mL), and the filtrate was concentrated under reduced pressure. The residue was purified by reverse phase column chromatography eluting with water (1% ammonium formate) and ACN (0-100%), followed by prep. HPLC (BEH C18 30×150 mm AmBicarb and ACN 25-45%) to afford the title compound as a solid (3.50 mg, 8%). ¹H NMR (400 MHz, DMSO-d6) δ 12.37 (s, 1H), 9.04 (s, 1H), 8.78 (s, 2H), 7.71 (d, J=5.8 Hz, 1H), 7.58 (s, 1H), 7.21 (s, 1H), 6.67 (d, J=6.0 Hz, 1H), 4.67 (d, J=5.8 Hz, 2H); m/z (ES⁺): [M+H]⁺=294.1; HPLC (A05) t_{R}=1.57 m.

### 16. Alternative Synthesis of Compounds

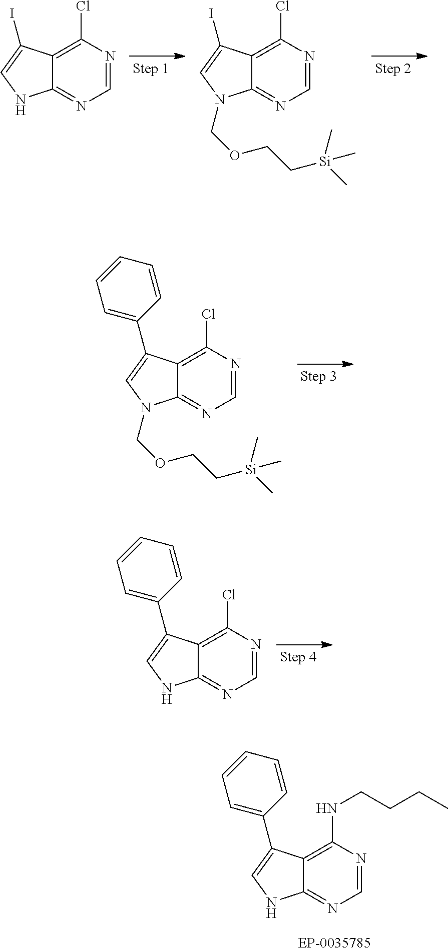 [SMILES: Clc1ncnc2[nH]cc(I)c12 ; C[Si](C)(C)CCOCn1cc(I)c2c(Cl)ncnc21 ; C[Si](C)(C)CCOCn1cc(-c2ccccc2)c2c(Cl)ncnc21 ; Clc1ncnc2[nH]cc(-c3ccccc3)c12 ; CCCCNc1ncnc2[nH]cc(-c3ccccc3)c12]

### a. Step 1: 2-[(4-chloro-5-iodo-pyrrolo[2,3-d]pyrimidin-7-yl)methoxy]ethyl-trimethyl-silane

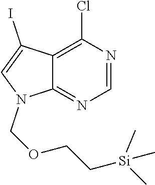 [SMILES: C[Si](C)(C)CCOCn1cc(I)c2c(Cl)ncnc21]

To 4-chloro-5-iodo-7H-pyrrolo[2,3-d]pyrimidine (7.08 g, 25.3 mmol) in dry DMF (100 mL) was added NaH (1.11 g, 27.9 mmol) at 0° C., under N₂. The solution was stirred for 30 m followed by the addition of 2-(chloromethoxy)ethyl-trimethyl-silane (6.73 mL, 38.0 mmol), and the solution was stirred at room temperature for 16 h. The mixture was diluted with NaOH (1M, 100 mL), filtered, and the resulting solid was dried under reduced pressure affording the title product (10.0 g, 89%). m/z (ES⁺): [M+H]⁺=410.1; HPLC (A05) t_{R}=2.73 m.

### b. Step 2: 2-[(4-chloro-5-phenyl-pyrrolo[2,3-d]pyrimidin-7-yl)methoxy]ethyl-trimethyl-silane

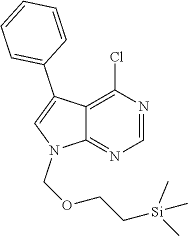 [SMILES: C[Si](C)(C)CCOCn1cc(-c2ccccc2)c2c(Cl)ncnc21]

To a solution of 2-[(4-chloro-5-iodo-pyrrolo[2,3-d]pyrimidin-7-yl)methoxy]ethyl-trimethyl-silane (700 mg, 1.71 mmol) in dry 1,4-dioxane (15.0 mL) was added K₂CO₃ (708 mg, 5.13 mmol), phenylboronic acid (229 mg, 1.88 mmol), and Pd(dppf)Cl₂ (150 mg, 0.21 mmol) in a microwave vial. The solution was degassed, and backfilled with N₂. The solution was stirred at 90° C. for 16 h. The mixture was filtered over Celite rinsing with MeOH (15.0 mL), the filtrate was concentrated under reduced pressure and purified by flash chromatography (25 g silica cartridge) eluting with hexanes and EtOAc (0-80%) to afford the title product (475 mg, 77%) as a solid. m/z (ES⁺): [M+H]⁺=360.9; HPLC (A05) t_{R}=2.92 m.

### c. Step 3: 4-chloro-5-phenyl-7H-pyrrolo[2,3-d]pyrimidine

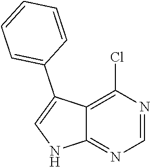 [SMILES: Clc1ncnc2[nH]cc(-c3ccccc3)c12]

To a solution of 2-[(4-chloro-5-phenyl-pyrrolo[2,3-d]pyrimidin-7-yl)methoxy]ethyl-trimethyl-silane (370 mg, 1.03 mmol) dissolved in dry DCM (5.00 mL), was added TFA (5.00 mL). The solution was stirred at room temperature for 16 h under N₂. The solution was concentrated under reduced pressure, and the residue was dissolved in MeOH (5.00 mL) and NH₄OH (3.00 mL). This solution was stirred at room temperature for 6 h and was then concentrated under reduced pressure to afford the title product as a solid (43 mg). m/z (ES⁺): [M+H]⁺=230.7; HPLC (A05) t_{R}=2.42 m.

### d. Step 4: EP-0035785, N-butyl-5-phenyl-7H-pyrrolo[2,3-d]pyrimidin-4-amine

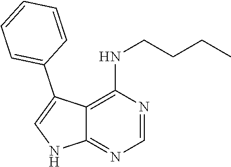 [SMILES: CCCCNc1ncnc2[nH]cc(-c3ccccc3)c12]

4-Chloro-5-phenyl-7H-pyrrolo[2,3-d]pyrimidine (43.0 mg, 0.19 mmol) was dissolved in EtOH (1.50 mL) in a 5-mL microwave vial before butan-1-amine (13.7 mg, 0.19 mmol) and DIPEA (82.0 μL, 0.47 mmol) were added. The solution was capped and stirred at 110° C. for 15 h. The solution was concentrated under reduced pressure and the residue was purified by flash chromatography (12 g silica cartridge) eluting with DCM and MeOH (0-10%) to afford the title product as a solid (12.0 mg, 24%). ¹H NMR (400 MHz, DMSO-d6) δ 11.79 (s, 1H), 8.18 (s, 1H), 7.55-7.42 (m, 4H), 7.42-7.30 (m, 1H), 7.21 (d, J=2.5 Hz, 1H), 5.23 (t, J=5.5 Hz, 1H), 3.47-3.39 (m, 2H), 1.54-1.40 (m, 2H), 1.34-1.20 (m, 2H), 0.86 (t, J=7.3 Hz, 3H); m/z (ES⁺): [M+H]⁺=267.8; HPLC (A05) t_{R}=2.49 m.

### 17. N-benzyl-5-phenyl-7H-pyrrolo[2,3-d]pyrimidin-4-amine (EP-0035786)

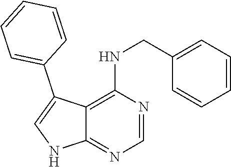 [SMILES: c1ccc(CNc2ncnc3[nH]cc(-c4ccccc4)c23)cc1]

4-Chloro-5-phenyl-7H-pyrrolo[2,3-d]pyrimidine (43.0 mg, 0.19 mmol) was dissolved in EtOH (1.50 mL) in a 5-mL microwave vial before phenylmethanamine (20.1 mg, 0.19 mmol) and DIPEA (82.0 μL, 0.47 mmol) were added. The solution was capped and stirred at 110° C. for 15 h. The solution was concentrated under reduced pressure and the residue was purified by washing the resulting solid with acetonitrile (15.0 mL) and filtrating the solid to afford the title product as a solid (12.0 mg, 21%). 1H NMR (400 MHz, DMSO-d6) δ 11.86 (s, 1H), 8.19 (s, 1H), 7.51-7.45 (m, 2H), 7.45-7.39 (m, 2H), 7.34-7.28 (m, 5H), 7.26-7.19 (m, 2H), 5.80-5.74 (m, 1H), 4.68 (d, J=5.8 Hz, 2H); m/z (ES⁺): [M+H]⁺=301.3; HPLC (A05) t_{R}=2.50 m.

### 18. N-(2-furylmethyl)-5-phenyl-7 h-pyrrolo[2,3-d]pyrimidin-4-amine (EP-0035787)

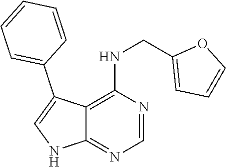 [SMILES: c1ccc(-c2c[nH]c3ncnc(NCc4ccco4)c23)cc1]

4-Chloro-5-phenyl-7H-pyrrolo[2,3-d]pyrimidine (47.0 mg, 0.21 mmol) was dissolved in EtOH (1.50 mL) in a 5-mL microwave vial before 2-furylmethanamine (19.9 mg, 0.21 mmol) and DIPEA (89.7 μL, 0.52 mmol) were added. The solution was capped and stirred at 110° C. for 15 h. The solution was concentrated under reduced pressure and the residue was purified by flash chromatography (12 g silica cartridge) eluting with DCM and MeOH (0-10%) to afford the title product as a solid (16.0 mg, 27%). 1H NMR (400 MHz, DMSO-d6) δ 11.89 (s, 1H), 8.23 (s, 1H), 7.56 (dd, J=1.8, 0.9 Hz, 1H), 7.49-7.43 (m, 4H), 7.38-7.30 (m, 1H), 7.26 (d, J=2.5 Hz, 1H), 6.37 (dd, J=3.2, 1.9 Hz, 1H), 6.23 (dd, J=3.2, 0.8 Hz, 1H), 5.64 (t, J=5.6 Hz, 1H), 4.68 (d, J=5.6 Hz, 2H); m/z (ES⁺): [M+H]⁺=291.1; HPLC (A05) tR=2.44 m.

### 19. N-(1-phenylcyclopropyl)-6-(trifluoromethyl)-7H-pyrrolo[2,3-d]pyrimidin-4-amine (EP-0035788)

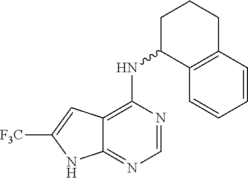 [SMILES: FC(F)(F)c1cc2c(NC3CCCc4ccccc43)ncnc2[nH]1]

4-Chloro-6-(trifluoromethyl)-7H-pyrrolo[2,3-d]pyrimidine (30.0 mg, 0.14 mmol) was dissolved in EtOH (0.48 mL) in a 2-mL microwave vial before tetralin-1-amine (0.02 mL, 0.14 mmol) and DIPEA (0.05 mL, 0.27 mmol) were added. The solution was then heated at 150° C. for 16 h. The solution was concentrated under reduced pressure and the residue was purified by flash chromatography (12 g cartridge) eluting with DCM and MeOH (0-6%) to afford the title product (20.4 mg, 45%) as a solid. 1H NMR (500 MHz, DMSO-d6) δ 12.73 (s, 1H), 8.27 (s, 1H), 8.14 (d, J=8.5 Hz, 1H), 7.27-7.08 (m, 5H), 5.66-5.56 (m, 1H), 2.90-2.72 (m, 2H), 2.11-1.90 (m, 2H), 1.89-1.73 (m, 2H); m/z (ES⁺): [M+H]⁺=333.9; HPLC (B05) t_{R}=2.75 min.

### 20. N-(1-methyl-1-phenyl-ethyl)-6-(trifluoromethyl)-7H-pyrrolo[2,3-d]pyrimidin-4-amine (EP-0035836)

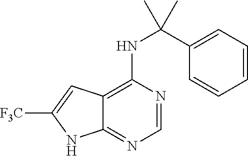 [SMILES: CC(C)(Nc1ncnc2[nH]c(C(F)(F)F)cc12)c1ccccc1]

4-Chloro-6-(trifluoromethyl)-7H-pyrrolo[2,3-d]pyrimidine (30.0 mg, 0.14 mmol) was dissolved in EtOH (0.48 mL) in a 2-mL microwave vial before 2-phenylpropan-2-amine (0.02 mL, 0.16 mmol) and DIPEA (0.05 mL, 0.27 mmol) were added. The solution was then heated at 150° C. for 16 h. The solution was concentrated under reduced pressure and the residue was purified by flash chromatography (12 g cartridge) eluting with DCM and MeOH (0-6%) to afford the title product (5.00 mg, 12%) as a solid. ¹H NMR (500 MHz, DMSO-d6) δ 12.66 (s, 1H), 7.95 (s, 1H), 7.75 (s, 1H), 7.40 (s, 1H), 7.35 (dd, J=8.4, 1.2 Hz, 2H), 7.24 (dd, J=10.5, 5.0 Hz, 2H), 7.13 (dd, J=10.3, 4.2 Hz, 1H), 1.78 (s, 6H); m/z (ES⁺): [M+H]⁺=321.7; HPLC (B05) t_{R}=2.63 min.

### 21. N-[(3,5-difluorophenyl)methyl]-6-(trifluoromethyl)-7H-pyrrolo[2,3-d]pyrimidin-4-amine (EP-0035837)

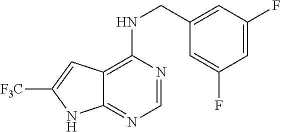 [SMILES: Fc1cc(F)cc(CNc2ncnc3[nH]c(C(F)(F)F)cc23)c1]

4-Chloro-6-(trifluoromethyl)-7H-pyrrolo[2,3-d]pyrimidine (30.0 mg, 0.14 mmol) was dissolved in EtOH (0.48 mL) in a 2-mL microwave vial before (3,5-difluorophenyl)methanamine (0.02 mL, 0.14 mmol) and DIPEA (0.05 mL, 0.27 mmol) were added. The solution was then heated at 150° C. for 16 h. The solution was concentrated under reduced pressure and the residue was purified by flash chromatography (12 g cartridge) eluting with DCM and MeOH (0-6%) to afford the title product (28.6 mg, 64%) as a solid. ¹H NMR (500 MHz, DMSO-d6) δ 12.61 (s, 1H), 8.40 (s, 1H), 8.23 (s, 1H), 7.16 (s, 1H), 7.13-7.00 (m, 3H), 4.74 (d, J=6.0 Hz, 2H); m/z (ES⁺): [M+H]⁺=329.9; HPLC (B05) t_{R}=2.57 min.

### 22. N-[(3,5-dichlorophenyl)methyl]-6-(trifluoromethyl)-7H-pyrrolo[2,3-d]pyrimidin-4-amine (EP-0035838)

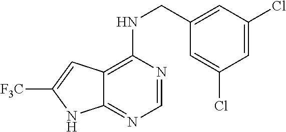 [SMILES: FC(F)(F)c1cc2c(NCc3cc(Cl)cc(Cl)c3)ncnc2[nH]1]

4-Chloro-6-(trifluoromethyl)-7H-pyrrolo[2,3-d]pyrimidine (30.0 mg, 0.14 mmol) was dissolved in EtOH (0.48 mL) in a 2-mL microwave vial before (3,5-dichlorophenyl)methanamine (0.02 mL, 0.14 mmol) and DIPEA (0.05 mL, 0.27 mmol) were added. The solution was then heated at 150° C. for 16 h. The solution was concentrated under reduced pressure and the residue was purified by flash chromatography (12 g cartridge) eluting with DCM and MeOH (0-6%) to afford the title product (23.1 mg, 63%) as a solid. ¹H NMR (500 MHz, DMSO-d6) δ 12.34 (s, 1H), 8.42 (t, J=6.0 Hz, 1H), 8.24 (s, 1H), 7.48 (t, J=1.9 Hz, 1H), 7.39 (t, J=2.8 Hz, 2H), 7.17 (d, J=1.0 Hz, 1H), 4.73 (d, J=6.0 Hz, 2H); m/z (ES⁺): [M+H]⁺=361.8; HPLC (B05) t_{R}=2.75 min.

### 23. N-(1-phenylethyl)-6-(trifluoromethyl)-7H-pyrrolo[2,3-d]pyrimidin-4-amine (EP-0035839)

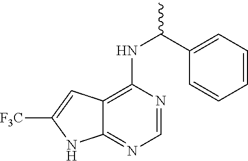 [SMILES: CC(Nc1ncnc2[nH]c(C(F)(F)F)cc12)c1ccccc1]

4-Chloro-6-(trifluoromethyl)-7H-pyrrolo[2,3-d]pyrimidine (30.0 mg, 0.14 mmol) was dissolved in EtOH (0.48 mL) in a 2-mL microwave vial before 1-phenylethanamine (0.02 mL, 0.14 mmol) and DIPEA (0.05 mL, 0.27 mmol) were added. The solution was then heated at 150° C. for 16 h. The solution was concentrated under reduced pressure and the residue was purified by flash chromatography (12 g cartridge) eluting with DCM and MeOH (0-6%) to afford the title product (8.90 mg, 22%) as a solid. ¹H NMR (500 MHz, DMSO-d6) δ 12.56 (s, 1H), 8.17 (s, 1H), 8.14 (d, J=8.1 Hz, 1H), 7.40 (d, J=7.3 Hz, 2H), 7.33-7.27 (m, 3H), 7.21 (t, J=7.3 Hz, 1H), 5.55-5.43 (m, 1H), 1.52 (d, J=7.0 Hz, 3H); m/z (ES⁺): [M+H]⁺=307.8; HPLC (B05) t_{R}=2.60 min.

### 24. 1-[4-(butylamino)-7H-pyrrolo[2,3-d]pyrimidin-5-yl]-2,2,2-trifluoro-ethanol (EP-0035851)

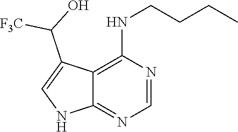 [SMILES: CCCCNc1ncnc2[nH]cc(C(O)C(F)(F)F)c12]

To a solution of 1-[4-(butylamino)-7-(2-trimethylsilylethoxymethyl)pyrrolo[2,3-d]pyrimidin-5-yl]-2,2,2-trifluoro-ethanol (40.0 mg, 0.005 mmol) dissolved in dry DCM (5.00 mL), was added TFA (5.00 mL). The solution was stirred at room temperature for 15 h, under N₂. The solution was concentrated under reduced pressure, and the residue was dissolved in MeOH (5.00 mL) and NH₄OH (5.00 mL). This solution was stirred at room temperature for 6 h, and was then concentrated under reduced pressure, and the residue was purified by flash chromatography (12 g silica cartridge) eluting with DCM and MeOH (0-10%) to afford the title product (8.00 mg, 58%) as a solid. ¹H NMR (400 MHz, DMSO-d6) δ 11.64 (s, 1H), 8.11 (s, 1H), 7.81 (d, J=5.1 Hz, 1H), 7.37 (t, J=5.4 Hz, 1H), 7.24 (d, J=2.5 Hz, 1H), 5.44-5.24 (m, 1H), 3.59-3.38 (m, 2H), 1.61-1.45 (m, 2H), 1.46-1.28 (m, 2H), 0.98-0.81 (m, 3H); m/z (ES⁺): [M+H]⁺=288.1; HPLC (A05) t_{R}=2.23 m.

### 25. Synthesis of N-[(1R)-tetralin-1-yl]-6-(trifluoromethyl)-7H-pyrrolo[2,3-d]pyrimidin-4-amine

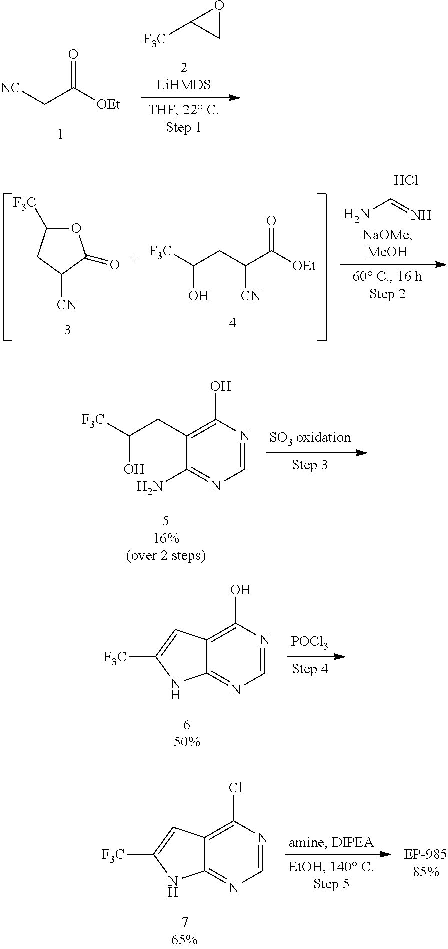

### a. Step 1: 2-oxo-5-(trifluoromethyl)tetrahydrofuran-3-carbonitrile and ethyl 2-cyano-5,5,5-trifluoro-4-hydroxy-pentanoate

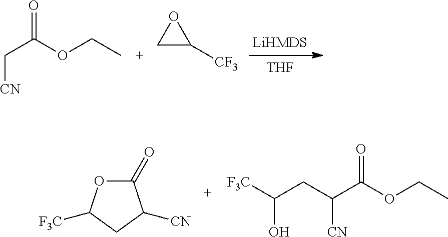

Ethyl 2-cyanoacetate (329 mL, 3.09 mol) was added dropwise at 0° C. under inert atmosphere to a solution of LiHMDS (1 M in THF, 3.09 L, 3.09 mol). The mixture was stirred for 30 min, and 2-(trifluoromethyl)oxirane (266 mL, 3.09 mol) was added dropwise. The solution was warmed to 23° C. over 4 h and stirred for an additional 20 h. The mixture was cooled to 0° C., and 3M aq. HCl (2.00 L) was slowly added over 2.5 h. The mixture was warmed to 23° C., and EtOAc (2.50 L) was added. The mixture was stirred for 1 h, and the layers were separated. The aqueous layer was extracted with EtOAc (2×500 mL). The combined organic layers were dried over Na₂SO₄, filtered, and concentrated under reduced pressure. The residue was dissolved in MeOH (250 mL) and concentrated under reduced pressure. This sequence was repeated five times. The residue was used in the next reaction without further purification (616 g).

### b. Step 2: [6-hydroxy-5-(3,3,3-trifluoro-2-hydroxy-propyl)pyrimidin-4-yl]ammonium chloride

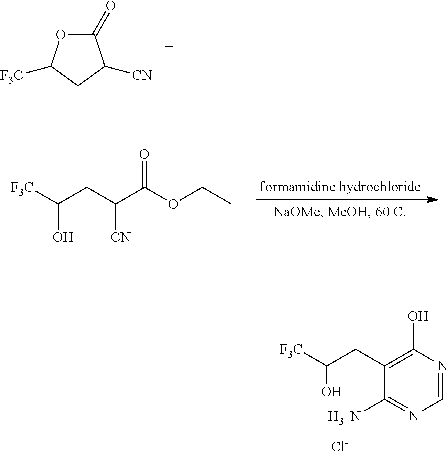

2-Oxo-5-(trifluoromethyl)tetrahydrofuran-3-carbonitrile (616 g, 3.09 mol) was dissolved in MeOH (2.10 L), and formamidine hydrochloride (249 g, 3.09 mol) and NaOMe (1.06 L, 3.09 mol) were added. The mixture was heated to 60° C. and stirred for 24 h. The mixture was cooled to 23° C., and SiO₂ (500 g) was added. The mixture was concentrated under reduced pressure to dryness. The residue was stirred in a mixture of EtOAc/DCM 7/3 (5.00 L) and stirred for 5 h. The mixture was filtered, and the solid was stirred in MeOH (5.00 L) for 16 h. The mixture was filtered, and the solid was washed with MeOH (2.00 L). The filtrate was concentrated under reduced pressure. The solid was dissolved in dioxane (2.00 L) and filtered. HCl in dioxane (4 M, 800 mL) was slowly added to the filtrate at 0° C. The mixture was concentrated under reduced pressure to a volume of 1 L and filtered. The solid was washed with dioxane (200 mL) and dried under reduced pressure for 48 h at 40° C. to afford the title compound as a solid (118 g, 16% over 2 steps). ¹H NMR (500 MHz, DMSO-d6) (OH signals not visible) δ 8.21 (s, 1H), 7.19 (br, 3H), 4.16-4.07 (m, 1H), 2.67 (dd, J=14.2, 3.4 Hz, 1H), 2.56 (dd, J=14.2, 9.5 Hz, 1H). m/z: (ES⁺) [M−H]⁺=224.2; LCMS (A05); t_{R}=0.87 m.

### c. Step 3: 6-(trifluoromethyl)-7H-pyrrolo[2,3-d]pyrimidin-4-ol

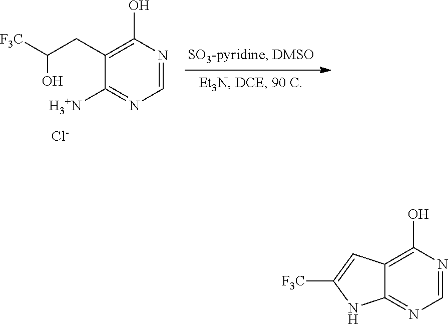

Sulfur trioxide pyridine complex (337 g, 2.12 mol) was added to a mixture of [6-hydroxy-5-(3,3,3-trifluoro-2-hydroxy-propyl)pyrimidin-4-yl]ammonium chloride (118 g, 0.455 mol) and anhydrous TEA (0.369 L, 2.65 mol) in anhydrous DCE (1.50 L) and anhydrous DMSO (0.376 L, 5.29 mol) at 22° C. under nitrogen. The mixture heated to 90° C. and stirred for 2 h. The mixture was diluted with water (6.80 L). The aqueous phase was washed with DCM (2×6.00 L) and extracted with EtOAc (3×3.40 L). The combined organic phases were dried over Na₂SO₄, filtered, and concentrated under reduced pressure. The residue was stirred in brine (800 mL), filtered, and the solid was washed with water (500 mL) and dried to provide the title compound as a solid (46.4 g, 50.0%). ¹H NMR (400 MHz, DMSO-d6) δ 13.16 (s, 1H), 12.11 (s, 1H), 8.01 (d, J=3.7 Hz, 1H), 7.01 (s, 1H). ¹⁹F NMR (376 MHz, DMSO-d6) δ −59.03 (d, J=0.8 Hz). m/z: (ES⁻) [M−H]⁻=202.08; LCMS (A05); t_{R}=1.83 m.

### d. Step 4: 4-chloro-6-(trifluoromethyl)-7H-pyrrolo[2,3-d]pyrimidine

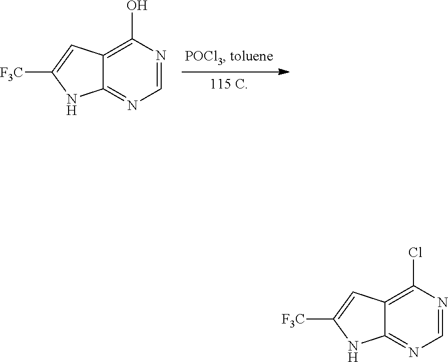 [SMILES: Oc1ncnc2[nH]c(C(F)(F)F)cc12 ; FC(F)(F)c1cc2c(Cl)ncnc2[nH]1]

POCl₃ (60.0 mL, 655 mmol) was added to a mixture of 6-(trifluoromethyl)-7H-pyrrolo[2,3-d]pyrimidin-4-ol (95.0%, 45.9 g, 215 mmol) in anhydrous dimethylformamide (1.66 mL, 21.5 mmol) and anhydrous toluene (1.20 L) at 22° C. under nitrogen. The mixture was heated to 125° C. and stirred for 24 h. The mixture cooled to 22° C. and concentrated under reduced pressure. The residue was diluted in EtOAc (1.00 L), and the mixture was added dropwise to an ice/water bath with vigorous stirring. The mixture was stirred for 2 h, and the layers were separated. The aqueous phase was extracted with EtOAc (1.00 L), and the combined organic phases were dried over Na₂SO₄, filtered, and concentrated under reduced pressure to provide the title compound as a solid (37.9 g, 68%, 85% purity). ¹H NMR (400 MHz, DMSO-d6) δ 13.92 (s, 1H), 8.80 (s, 1H), 7.31 (s, 1H). ¹⁹F NMR (376 MHz, DMSO-d6) δ −60.23 (s). m/z: (ES⁺) [M+H]⁺=333.20; LCMS (A05); t_{R}=2.59 m.

### e. Step 5: N-[(1R)-tetralin-1-yl]-6-(trifluoromethyl)-7H-pyrrolo[2,3-d]pyrimidin-4-amine

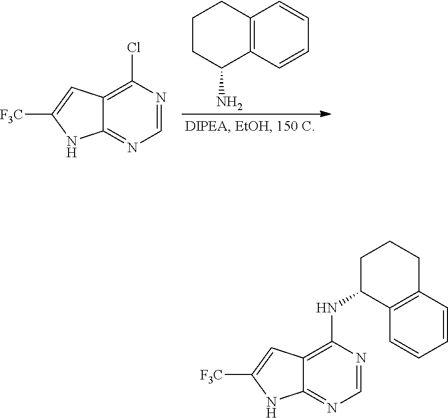 [SMILES: FC(F)(F)c1cc2c(Cl)ncnc2[nH]1 ; N[C@@H]1CCCc2ccccc21 ; FC(F)(F)c1cc2c(N[C@@H]3CCCc4ccccc43)ncnc2[nH]1]

(1R)-Tetralin-1-amine (9.55 g, 64.9 mmol) and DIPEA (14.1 mL, 82.6 mmol) were added to a solution of 4-chloro-6-(trifluoromethyl)-7H-pyrrolo[2,3-d]pyrimidine (13.1 g, 59.0 mmol) in EtOH (49.0 mL). The pressure vessel was sealed, and the mixture was heated to 150° C. for 16 h. The mixture was cooled to 23° C. and concentrated under reduced pressure. The residue was dissolved in hot MeOH (270 mL). Activated charcoal (13.0 g) was added, and the mixture was stirred at room temperature for 20 min. The mixture was filtered over Celite, and the pad was washed with Et₂O (2.5 L). The filtrate was concentrated under reduced pressure and dissolved in hot MeOH (270 mL). Water (810 mL) was added dropwise. The suspension was filtered, and the solid was dissolved in hot MeOH and filtered. The filtrate was set aside at ambient temperature until crystals formed. The crystals were filtered and set aside. The filtrate was concentrated, and the residue was dissolved in hot MeOH. The mixture was set aside at ambient temperature until crystals formed. The crystals were collected in the same manner, and the sequence was repeated a final time. The combined crystals were powdered in a mortar and pestle and dried in a vacuum oven at 50° C. for 16 h to afford the title product as a solid (10.6 g, 53%). ¹H NMR (400 MHz, DMSO-d6) δ 12.75 (s, 1H), 8.27 (s, 1H), 8.14 (d, J=8.7 Hz, 1H), 7.23 (s, 1H), 7.22-7.08 (m, 4H), 5.60 (br s, 1H), 2.91-2.71 (m, 2H), 2.10-1.90 (m, 2H), 1.90-1.69 (m, 2H). m/z (ES⁺) [M+H]⁺=333.1; HPLC (C18 5-100% ACN/AmForm 10 mM pH4) t_{R}=1.70 min.

### 26. Evaluation of N-Containing Heteroaryl Analogs for PINK1 Kinase Activity

TABLE 1

| | | | Toxic- |
|---|---|---|---|
| | | | ity (% |
| | | | posi- |
| | | | tive |
| | | Poten- | for |
| | | cy at | DAPI |
| | | 1 μM | take- |
| | | F/O | up @ |
| No. | Compound | toxin | 50 μM) |
| kinetin | 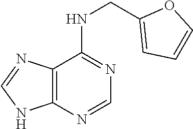 [SMILES: c1coc(CNc2ncnc3[nH]cnc23)c1] | n/d | 4.9% |
| 1 (EP- 0035507) | 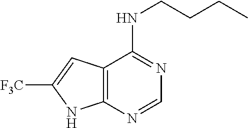 [SMILES: CCCCNc1ncnc2[nH]c(C(F)(F)F)cc12] | 42.3% | 4.6% |
| 2 | 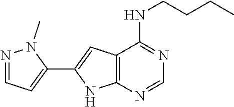 [SMILES: CCCCNc1ncnc2[nH]c(-c3ccnn3C)cc12] | 15.0% | 94.6% |
| 3 | 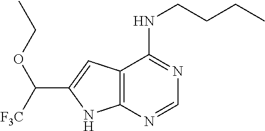 [SMILES: CCCCNc1ncnc2[nH]c(C(OCC)C(F)(F)F)cc12] | 25.9% | 15.8% |
| 4 | 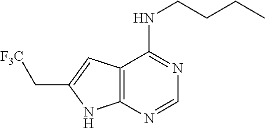 [SMILES: CCCCNc1ncnc2[nH]c(CC(F)(F)F)cc12] | 13.9% | 24.8% |
| 5 | 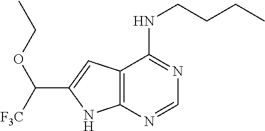 [SMILES: CCCCNc1ncnc2[nH]c(C(OCC)C(F)(F)F)cc12] | 17.9% | 47.4% |
| 6 | 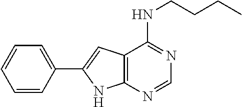 [SMILES: CCCCNc1ncnc2[nH]c(-c3ccccc3)cc12] | 52.0% | 90.0% |
| 7 | 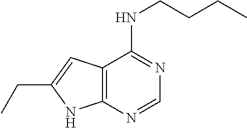 [SMILES: CCCCNc1ncnc2[nH]c(CC)cc12] | 14.0% | 93.4% |
| 8 | 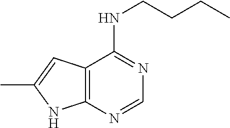 | 43.4% | 15.4% |

*Potency is defined as the EC₅₀ of mitophagy activation OR % activation of mitophagy at 6.3 μM treatment of the compound.

TABLE 2

| | | | Toxicity (% |
|---|---|---|---|
| | | Potency at | positive for |
| | | 1 μM | DAPI |
| | | F/O | takeup @ |
| No. | Compound | toxin* | 50 μM) |
| 9 (EP- 0035640) | 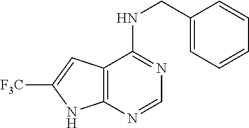 [SMILES: FC(F)(F)c1cc2c(NCc3ccccc3)ncnc2[nH]1] | 35.1% | 50.4% |
| 10 | 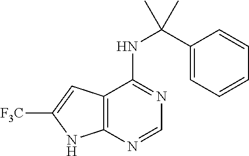 [SMILES: CC(C)(Nc1ncnc2[nH]c(C(F)(F)F)cc12)c1ccccc1] | 17.0% | 5.6% |
| 11 | 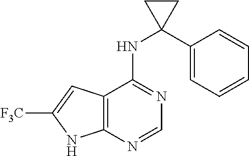 [SMILES: FC(F)(F)c1cc2c(NC3(c4ccccc4)CC3)ncnc2[nH]1] | 20.8% | 80.5% |
| 12 (EP- 0036084) | 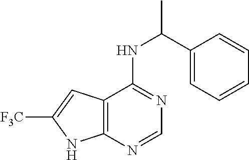 [SMILES: CC(Nc1ncnc2[nH]c(C(F)(F)F)cc12)c1ccccc1] | 88.4% | 2.4% |
| 13 (EP- 0035941) | 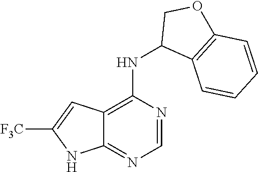 [SMILES: FC(F)(F)c1cc2c(NC3COc4ccccc43)ncnc2[nH]1] | 52.5% | 39.7% |
| 14 | 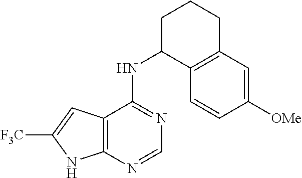 [SMILES: COc1ccc2c(c1)CCCC2Nc1ncnc2[nH]c(C(F)(F)F)cc12] | 29.8% | 95.5% |
| 15 (EP- 0035985) | 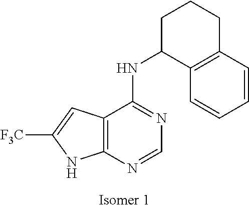 [SMILES: FC(F)(F)c1cc2c(NC3CCCc4ccccc43)ncnc2[nH]1] | 55.5% EC₅₀ = 0.44 ± 0.12 μM from N = 128 runs | 2.1% |
| 16 | 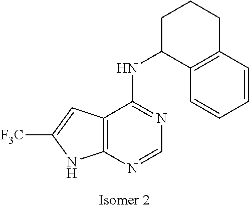 [SMILES: FC(F)(F)c1cc2c(NC3CCCc4ccccc43)ncnc2[nH]1] | 10.1% | 4.5% |
| 17 | 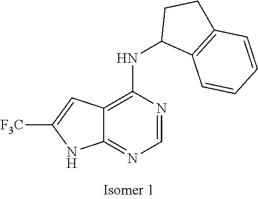 [SMILES: FC(F)(F)c1cc2c(NC3CCc4ccccc43)ncnc2[nH]1] | 14.8% | 2.3% |
| 18 | 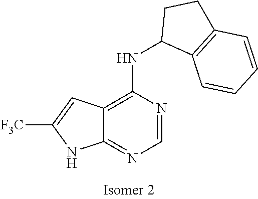 [SMILES: FC(F)(F)c1cc2c(NC3CCc4ccccc43)ncnc2[nH]1] | 14.3% | 1.7% |

*Potency is defined as the EC₅₀ of mitophagy activation OR % activation of mitophagy at 1.6 μM treatment of the compound.

TABLE 3

| | | | Toxicity (% |
|---|---|---|---|
| | | Potency | positive for |
| | | at 1 μM | DAPI |
| | | F/O | takeup @ |
| No. | Compound | toxin* | 50 μM) |
| 6-benzyl- amino- purine (6BAP) | 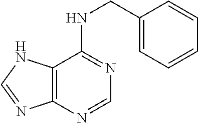 [SMILES: c1ccc(CNc2ncnc3nc[nH]c23)cc1] | n.d. | |
| 19 (EP- 0035910) | 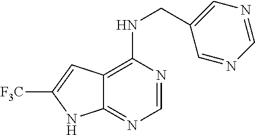 [SMILES: FC(F)(F)c1cc2c(NCc3cncnc3)ncnc2[nH]1] | 50.3% | 1.7% |
| 20 | 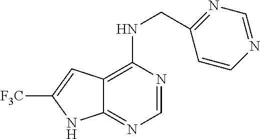 [SMILES: FC(F)(F)c1cc2c(NCc3ccncn3)ncnc2[nH]1] | 7.9% | 1.5% |
| 21 | 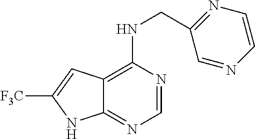 [SMILES: FC(F)(F)c1cc2c(NCc3cnccn3)ncnc2[nH]1] | 9.1% | 0.9% |
| 22 | 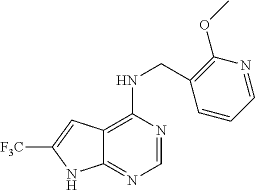 [SMILES: COc1ncccc1CNc1ncnc2[nH]c(C(F)(F)F)cc12] | 18.5% | 7.6% |
| 23 | 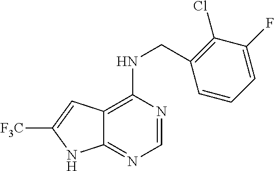 [SMILES: Fc1cccc(CNc2ncnc3[nH]c(C(F)(F)F)cc23)c1Cl] | 87.7% | 4.5% |
| 24 | 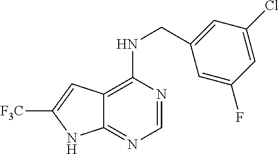 [SMILES: Fc1cc(Cl)cc(CNc2ncnc3[nH]c(C(F)(F)F)cc23)c1] | 51.2% | 4.8% |
| 25 | 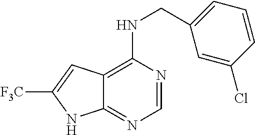 [SMILES: FC(F)(F)c1cc2c(NCc3cccc(Cl)c3)ncnc2[nH]1] | 49.9% | 20.3% |
| 26 (EP- 0036336) | 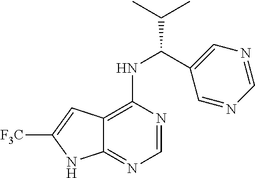 [SMILES: CC(C)[C@@H](Nc1ncnc2[nH]c(C(F)(F)F)cc12)c1cncnc1] | EC₅₀ = 6.9 μM | 6.8% |
| 27 (EP- 0036296) | 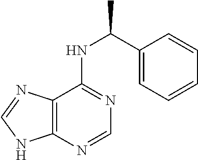 [SMILES: C[C@H](Nc1ncnc2[nH]cnc12)c1ccccc1] | EC₅₀ = 2.0 μM | 11.7% |
| 28 (EP- 0036329) | 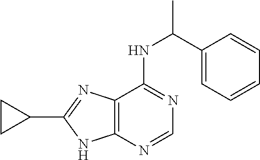 [SMILES: CC(Nc1ncnc2[nH]c(C3CC3)nc12)c1ccccc1] | EC₅₀ = 6.3 μM | 18.3% |
| 29 (EP- 0034886) | 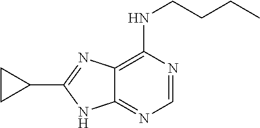 [SMILES: CCCCNc1ncnc2[nH]c(C3CC3)nc12] | EC₅₀ = 18.8 μM | 16.2% |
| 30 (EP- 0035338) | 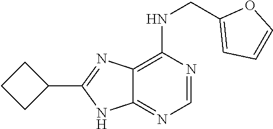 [SMILES: c1coc(CNc2ncnc3[nH]c(C4CCC4)nc23)c1] | >50 μM | 3.0% |
| 31 (EP- 0035339) | 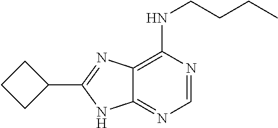 [SMILES: CCCCNc1ncnc2[nH]c(C3CCC3)nc12] | >50 μM | 6.1% |
| 32 (EP- 0035788) | 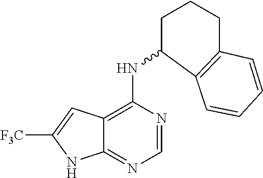 [SMILES: FC(F)(F)c1cc2c(NC3CCCc4ccccc43)ncnc2[nH]1] | >50 μM | 27.1% |
| 33 (EP- 0035987) | 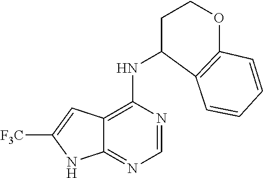 [SMILES: FC(F)(F)c1cc2c(NC3CCOc4ccccc43)ncnc2[nH]1] | EC₅₀ = 3.5 μM | 22.3% |
| 34 (EP- 0036023) | 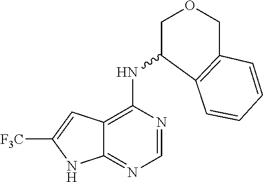 [SMILES: FC(F)(F)c1cc2c(NC3COCc4ccccc43)ncnc2[nH]1] | >50 μM | 42.9% |
| 35 (EP- 0036032 | 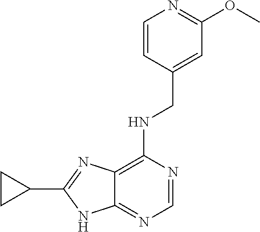 [SMILES: COc1cc(CNc2ncnc3[nH]c(C4CC4)nc23)ccn1] | >50 μM | 7.1% |
| 36 (EP- 0036050) | 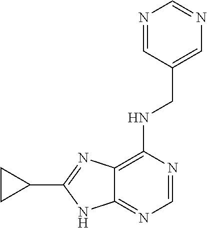 [SMILES: c1ncc(CNc2ncnc3[nH]c(C4CC4)nc23)cn1] | >50 μM | 7.1% |
| 37 (EP- 0036081) | 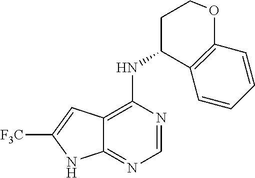 [SMILES: FC(F)(F)c1cc2c(N[C@@H]3CCOc4ccccc43)ncnc2[nH]1] | EC₅₀ = 6.3 μM | 21.1 |
| 38 (EP- 0036082) | 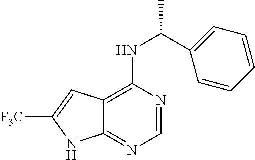 [SMILES: C[C@@H](Nc1ncnc2[nH]c(C(F)(F)F)cc12)c1ccccc1] | EC₅₀ = 3.1 μM | 63.7% |
| 39 (EP- 0036078) | 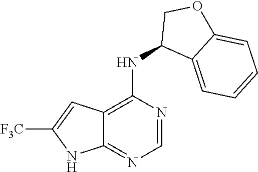 [SMILES: FC(F)(F)c1cc2c(N[C@H]3COc4ccccc43)ncnc2[nH]1] | EC₅₀ = 1.4 μM | 22.8% |
| 40 (EP- 0036083) | 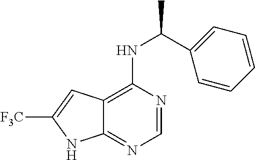 [SMILES: C[C@H](Nc1ncnc2[nH]c(C(F)(F)F)cc12)c1ccccc1] | EC₅₀ = 0.3 μM | 90.8% |
| 41 (EP- 0036079) | 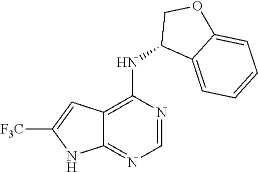 [SMILES: FC(F)(F)c1cc2c(N[C@@H]3COc4ccccc43)ncnc2[nH]1] | EC₅₀ = 14.9 μM | 29.2% |
| 42 (EP- 0036080) | 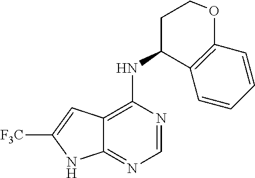 [SMILES: FC(F)(F)c1cc2c(N[C@H]3CCOc4ccccc43)ncnc2[nH]1] | EC₅₀ = 5.6 μM | 37.7% |
| 43 (EP- 0036193) | 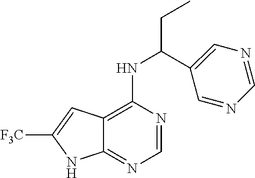 [SMILES: CCC(Nc1ncnc2[nH]c(C(F)(F)F)cc12)c1cncnc1] | EC₅₀ = 18.8 μM | 15.9% |
| 44 (EP- 0036194) | 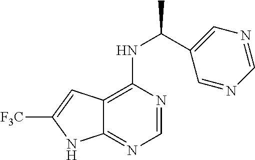 [SMILES: C[C@H](Nc1ncnc2[nH]c(C(F)(F)F)cc12)c1cncnc1] | EC₅₀ = 3.1 μM | 23.7% |
| 45 (EP- 0036404) | 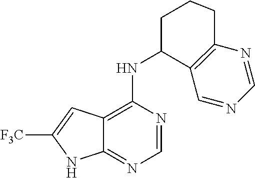 [SMILES: FC(F)(F)c1cc2c(NC3CCCc4ncncc43)ncnc2[nH]1] | >50 μM | 12.0% |
| 46 (EP- 0036438) | 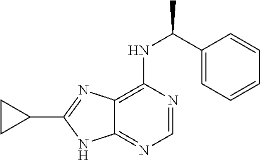 [SMILES: C[C@H](Nc1ncnc2[nH]c(C3CC3)nc12)c1ccccc1] | EC₅₀ = 3.2 μM | 27.6% |
| 47 (EP- 0036439) | 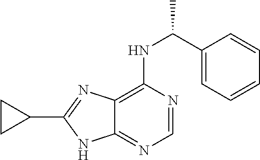 [SMILES: C[C@@H](Nc1ncnc2[nH]c(C3CC3)nc12)c1ccccc1] | >50 μM | 12.3% |
| 48 (EP- 0035764) | 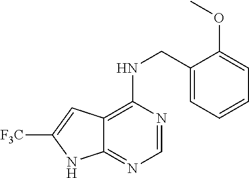 [SMILES: COc1ccccc1CNc1ncnc2[nH]c(C(F)(F)F)cc12] | EC₅₀ = 2.0 μM | 24.7% |
| 49 (EP- 0036061) | 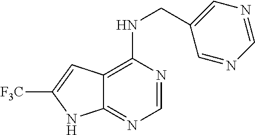 [SMILES: FC(F)(F)c1cc2c(NCc3cncnc3)ncnc2[nH]1] | EC₅₀ = 11.7 μM | 23.0% |
| 50 (EP- 0036198) | 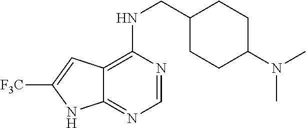 [SMILES: CN(C)C1CCC(CNc2ncnc3[nH]c(C(F)(F)F)cc23)CC1] | >50 μM | 5.2% |
| 51 (EP- 0035855) | 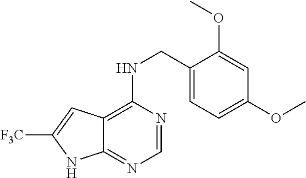 [SMILES: COc1ccc(CNc2ncnc3[nH]c(C(F)(F)F)cc23)c(OC)c1] | EC₅₀ = 2.1 μM | 42.6% |
| 52 (EP- 0036297) | 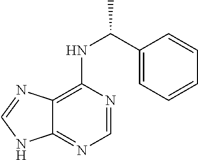 [SMILES: C[C@@H](Nc1ncnc2[nH]cnc12)c1ccccc1] | >50 μM | 10.1% |
| 53 (EP- 0036405) | 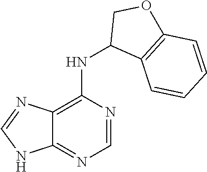 [SMILES: c1ccc2c(c1)OCC2Nc1ncnc2[nH]cnc12] | EC₅₀ = 6.1 μM | 21.0% |
| 54 (EP- 0036406) | 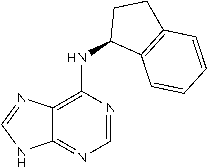 [SMILES: c1ccc2c(c1)CC[C@@H]2Nc1ncnc2[nH]cnc12] | EC₅₀ = 4.4 μM | 16.2% |
| 55 (EP- 0036407) | 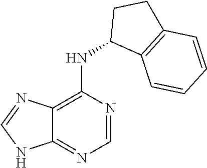 [SMILES: c1ccc2c(c1)CC[C@H]2Nc1ncnc2[nH]cnc12] | >50 μM | 10.3% |
| 56 (EP- 0036408) | 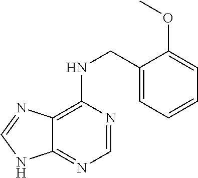 [SMILES: COc1ccccc1CNc1ncnc2[nH]cnc12] | EC₅₀ = 10.8 μM | 12.4% |
| 57 (EP- 0036409) | 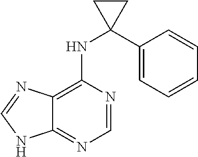 [SMILES: c1ccc(C2(Nc3ncnc4[nH]cnc34)CC2)cc1] | >50 μM | 13.4% |
| 58 (EP- 0036411) | 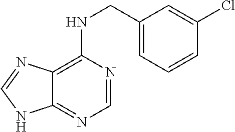 [SMILES: Clc1cccc(CNc2ncnc3[nH]cnc23)c1] | EC₅₀ = 16.0 μM | 12.5% |
| 59 (EP- 0036413) | 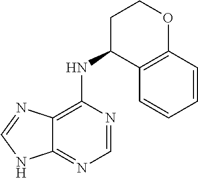 [SMILES: c1ccc2c(c1)OCC[C@@H]2Nc1ncnc2[nH]cnc12] | EC₅₀ = 1.8 μM | 17.0% |
| 60 (EP- 0036414) | 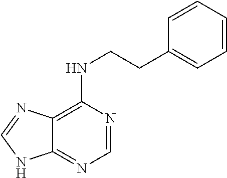 [SMILES: c1ccc(CCNc2ncnc3[nH]cnc23)cc1] | EC₅₀ = 2.8 μM | 19.7 |
| 61 (EP- 0036451) | 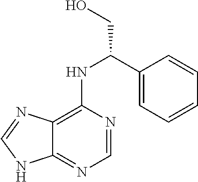 [SMILES: OC[C@@H](Nc1ncnc2[nH]cnc12)c1ccccc1] | >50 μM | 8.9% |
| 62 (EP- 0036453) | 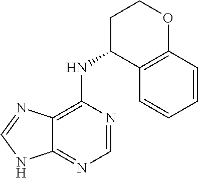 [SMILES: c1ccc2c(c1)OCC[C@H]2Nc1ncnc2[nH]cnc12] | >50 μM | 16.0% |
| 63 (EP- 0036422) | 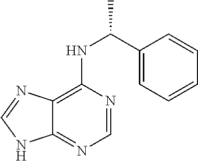 [SMILES: C[C@@H](Nc1ncnc2[nH]cnc12)c1ccccc1] | EC₅₀ = 47.4 μM | 11.5% |
| 64 (EP- 0036425) | 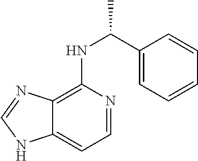 [SMILES: C[C@@H](Nc1nccc2[nH]cnc12)c1ccccc1] | >50 μM | 7.6% |
| 65 (EP- 0036426) | 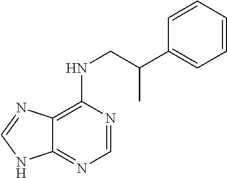 [SMILES: CC(CNc1ncnc2[nH]cnc12)c1ccccc1] | EC₅₀ = 3.6 μM | 28.9% |
| 66 (EP- 0036002) | 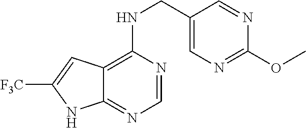 [SMILES: COc1ncc(CNc2ncnc3[nH]c(C(F)(F)F)cc23)cn1] | EC₅₀ = 10.6 μM | 5.8% |
| 67 (EP- 0036004) | 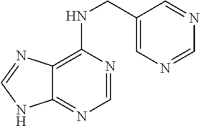 [SMILES: c1ncc(CNc2ncnc3[nH]cnc23)cn1] | EC₅₀ = 4.4 μM | 5.3% |
| 68 (EP- 0036022 | 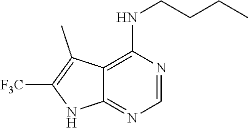 [SMILES: CCCCNc1ncnc2[nH]c(C(F)(F)F)c(C)c12] | >50 μM | 11.9 |
| 69 (EP- 0036025) | 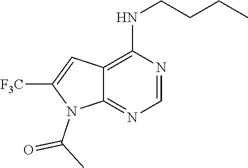 [SMILES: CCCCNc1ncnc2c1cc(C(F)(F)F)n2C(C)=O] | >50 μM | 36.6% |
| 70 (EP- 0036195) | 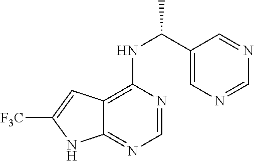 [SMILES: C[C@@H](Nc1ncnc2[nH]c(C(F)(F)F)cc12)c1cncnc1] | EC₅₀ = 21.6 μM | 22.9% |
| 71 (EP- 0036202) | 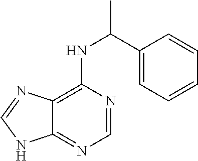 [SMILES: CC(Nc1ncnc2[nH]cnc12)c1ccccc1] | EC₅₀ = 3.5 μM | 18.2% |
| 72 (EP- 0036410) | 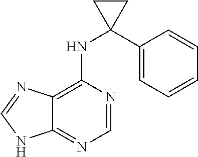 [SMILES: c1ccc(C2(Nc3ncnc4[nH]cnc34)CC2)cc1] | >50 μM | 13.4% |
| 73 (EP- 0036428) | 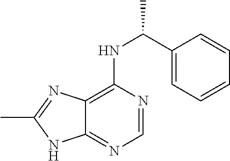 [SMILES: Cc1nc2c(N[C@H](C)c3ccccc3)ncnc2[nH]1] | >50 μM | 9.7% |
| 74 (EP- 0036437) | 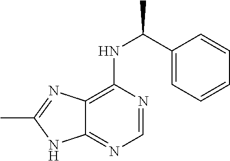 [SMILES: Cc1nc2c(N[C@@H](C)c3ccccc3)ncnc2[nH]1] | EC₅₀ = 4.6 μM | 31.9% |
| 75 (EP- 0036463) | 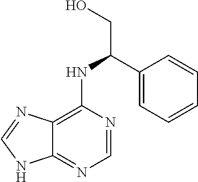 [SMILES: OC[C@H](Nc1ncnc2[nH]cnc12)c1ccccc1] | EC₅₀ = 3.5 μM | 14.5% |
| 76 (EP- 0036468) | 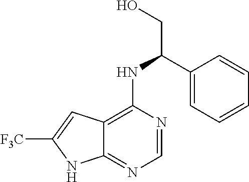 [SMILES: OC[C@H](Nc1ncnc2[nH]c(C(F)(F)F)cc12)c1ccccc1] | EC₅₀ = 14.7 μM | 30.4% |
| 77 (EP- 0036477) | 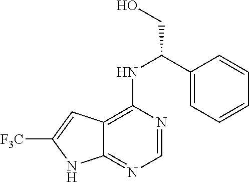 [SMILES: OC[C@@H](Nc1ncnc2[nH]c(C(F)(F)F)cc12)c1ccccc1] | EC₅₀ = 4.4 μM | 30.3 |
| 78 (EP- 0036837) | 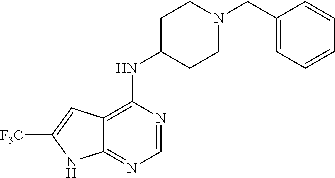 [SMILES: FC(F)(F)c1cc2c(NC3CCN(Cc4ccccc4)CC3)ncnc2[nH]1] | EC₅₀ = 21.4 μM | 43.9% |
| 79 (EP- 0036847) | 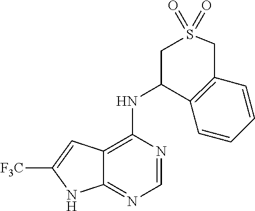 [SMILES: O=S1(=O)Cc2ccccc2C(Nc2ncnc3[nH]c(C(F)(F)F)cc23)C1] | >50 μM | 13.2% |
| 80 (EP- 0036848) | 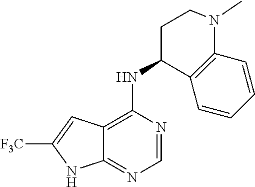 [SMILES: CN1CC[C@H](Nc2ncnc3[nH]c(C(F)(F)F)cc23)c2ccccc21] | EC₅₀ = 1.9 μM | 28.1% |
| 81 (EP- 0037056) | 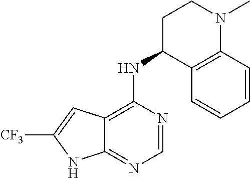 [SMILES: CN1CC[C@H](Nc2ncnc3[nH]c(C(F)(F)F)cc23)c2ccccc21] | EC₅₀ = 1.7 μM | 59.0 |
| 82 (EP- 0037059) | 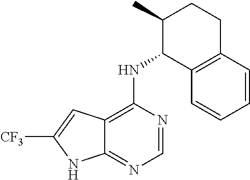 [SMILES: C[C@H]1CCc2ccccc2[C@@H]1Nc1ncnc2[nH]c(C(F)(F)F)cc12] | EC₅₀ = 1.0 μM | 28.3% |
| 83 (EP- 0037085) | 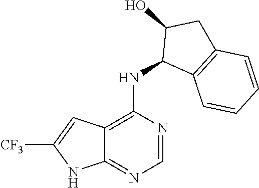 [SMILES: O[C@H]1Cc2ccccc2[C@H]1Nc1ncnc2[nH]c(C(F)(F)F)cc12] | >50 μM | 21.2% |
| 84 (EP- 0037092) | 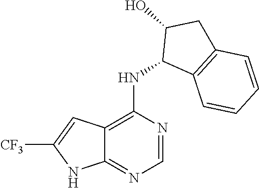 [SMILES: O[C@@H]1Cc2ccccc2[C@@H]1Nc1ncnc2[nH]c(C(F)(F)F)cc12] | EC₅₀ = 9.0 μM | 35.6% |
| 85 (EP- 0037094) | 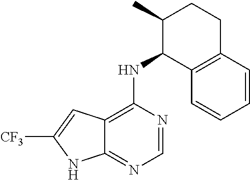 [SMILES: C[C@H]1CCc2ccccc2[C@H]1Nc1ncnc2[nH]c(C(F)(F)F)cc12] | EC₅₀ = 1.1 μM | 31.4% |
| 86 (EP- 0037130) | 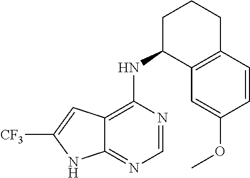 [SMILES: COc1ccc2c(c1)[C@@H](Nc1ncnc3[nH]c(C(F)(F)F)cc13)CCC2] | EC₅₀ = 4.0 μM | 3.2% |
| 87 (EP- 0037131) | 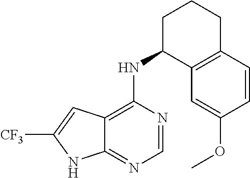 [SMILES: COc1ccc2c(c1)[C@@H](Nc1ncnc3[nH]c(C(F)(F)F)cc13)CCC2] | EC₅₀ = 9.5 μM | 5.9% |
| 88 (EP- 0037154) | 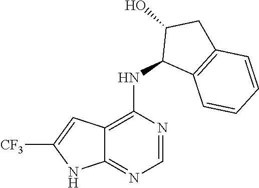 [SMILES: O[C@@H]1Cc2ccccc2[C@H]1Nc1ncnc2[nH]c(C(F)(F)F)cc12] | EC₅₀ = 15.1 μM | 7.6% |
| 90 (EP- 0037155) | 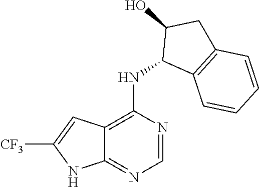 [SMILES: O[C@H]1Cc2ccccc2[C@@H]1Nc1ncnc2[nH]c(C(F)(F)F)cc12] | EC₅₀ = 18.5 μM | 14.4% |
| 91 (EP- 0037178) | 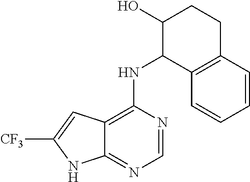 [SMILES: OC1CCc2ccccc2C1Nc1ncnc2[nH]c(C(F)(F)F)cc12] | >50 μM | 12.8% |
| 92 (EP- 0037214) | 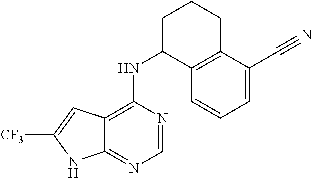 [SMILES: N#Cc1cccc2c1CCCC2Nc1ncnc2[nH]c(C(F)(F)F)cc12] | EC₅₀ = 3.0 μM | 71.2% |
| 93 (EP- 0037845) | 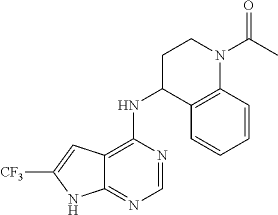 [SMILES: CC(=O)N1CCC(Nc2ncnc3[nH]c(C(F)(F)F)cc23)c2ccccc21] | EC₅₀ = 17.3 μM | 6.9 |
| 94 (EP- 0037852) | 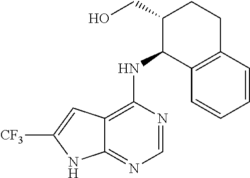 [SMILES: OC[C@@H]1CCc2ccccc2[C@H]1Nc1ncnc2[nH]c(C(F)(F)F)cc12] | EC₅₀ = 1.1 μM | 59.5% |
| 95 (EP- 0037853) | 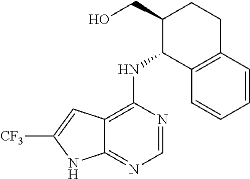 [SMILES: OC[C@H]1CCc2ccccc2[C@@H]1Nc1ncnc2[nH]c(C(F)(F)F)cc12] | EC₅₀ = 5.5 μM | 30.9% |
| 96 (EP- 0037861) | 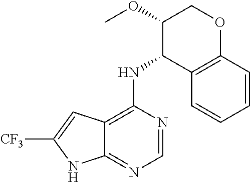 [SMILES: CO[C@@H]1COc2ccccc2[C@@H]1Nc1ncnc2[nH]c(C(F)(F)F)cc12] | EC₅₀ = 1.6 μM | 12.1% |
| 97 (EP- 0037862) | 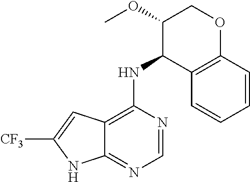 [SMILES: CO[C@@H]1COc2ccccc2[C@H]1Nc1ncnc2[nH]c(C(F)(F)F)cc12] | EC₅₀ = 6.3 μM | 10.6% |
| 98 (EP- 0037863) | 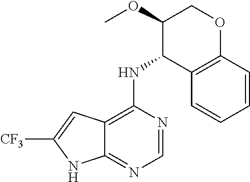 [SMILES: CO[C@H]1COc2ccccc2[C@@H]1Nc1ncnc2[nH]c(C(F)(F)F)cc12] | EC₅₀ = 4.0 μM | 15.9% |
| 99 (EP- 0037871) | 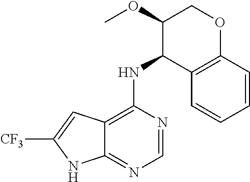 [SMILES: CO[C@H]1COc2ccccc2[C@H]1Nc1ncnc2[nH]c(C(F)(F)F)cc12] | EC₅₀ = 13.3 μM | 3.6% |
| 100 (EP- 0037880) | 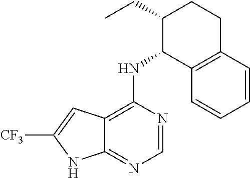 [SMILES: CC[C@@H]1CCc2ccccc2[C@@H]1Nc1ncnc2[nH]c(C(F)(F)F)cc12] | EC₅₀ = 1.8 μM | 8.7% |
| 101 (EP- 0037881) | 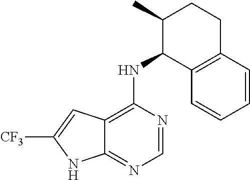 [SMILES: C[C@H]1CCc2ccccc2[C@H]1Nc1ncnc2[nH]c(C(F)(F)F)cc12] | >50 μM | 12.0% |
| 102 (EP- 0037882) | 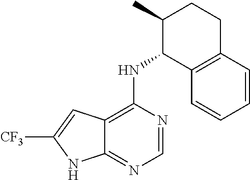 [SMILES: C[C@H]1CCc2ccccc2[C@@H]1Nc1ncnc2[nH]c(C(F)(F)F)cc12] | EC₅₀ = 0.6 μM | 13.3% |
| 103 (EP- 0037883) | 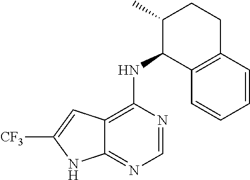 [SMILES: C[C@@H]1CCc2ccccc2[C@H]1Nc1ncnc2[nH]c(C(F)(F)F)cc12] | EC₅₀ = 4.2 μM | 23.8 |
| 104 (EP- 0037962) | 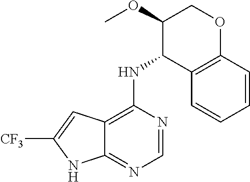 [SMILES: CO[C@H]1COc2ccccc2[C@@H]1Nc1ncnc2[nH]c(C(F)(F)F)cc12] | EC₅₀ = 1.5 μM | 7.7% |
| 105 (EP- 0037963) | 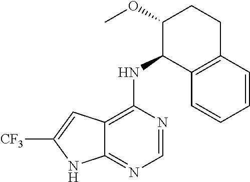 [SMILES: CO[C@@H]1CCc2ccccc2[C@H]1Nc1ncnc2[nH]c(C(F)(F)F)cc12] | EC₅₀ = 22.1 μM | 18.4% |
| 107 (EP- 0038205) | 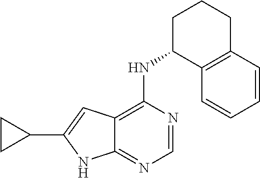 [SMILES: c1ccc2c(c1)CCC[C@H]2Nc1ncnc2[nH]c(C3CC3)cc12] | EC₅₀ = 0.48 μM | 16.6% |
| 108 (EP- 0038252) | 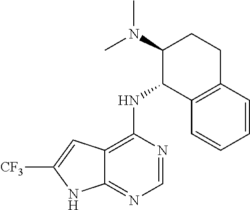 [SMILES: CN(C)[C@H]1CCc2ccccc2[C@@H]1Nc1ncnc2[nH]c(C(F)(F)F)cc12] | >50 μM | 79.3% |
| 109 (EP- 0038313) | 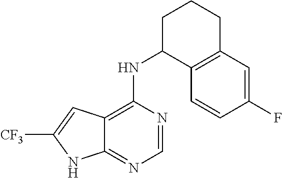 [SMILES: Fc1ccc2c(c1)CCCC2Nc1ncnc2[nH]c(C(F)(F)F)cc12] | >50 μM | 30.1% |
| 110 (EP- 0038508) | 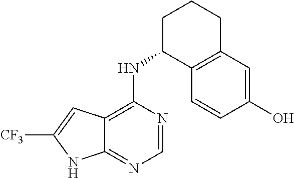 [SMILES: Oc1ccc2c(c1)CCC[C@H]2Nc1ncnc2[nH]c(C(F)(F)F)cc12] | EC₅₀ = 4.4 μM | 11.0% |
| 111 (EP- 0039729) | 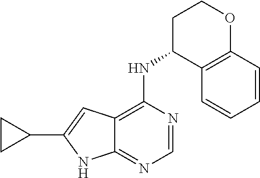 [SMILES: c1ccc2c(c1)OCC[C@H]2Nc1ncnc2[nH]c(C3CC3)cc12] | EC₅₀ = 2.0 μM | 17.3% |

*Potency is defined as the EC₅₀ of mitophagy activation OR % activation of mitophagy at 6.3 μM treatment of the compound.

TABLE 3
SUPPLEMENT A.

| | Potency | Toxicity |
|---|---|---|
| | (EC₅₀ Max | (% positive for DAPI |
| No. | Mitophagy) (μM)* | take-up at 50 μM)* |
| EP-0035338 | >25 | 3.04 |
| EP-0035339 | >25 | 6.13 |
| EP-0035764 | 2.01 | 10.93 |
| EP-0035788 | >25 | 27.12 |
| EP-0035855 | 2.08 | 42.56 |
| EP-0035987 | 3.24 | 26.04 |
| EP-0036002 | 10.59 | 5.76 |
| EP-0036004 | 4.42 | 5.33 |
| EP-0036022 | >25 | 11.94 |
| EP-0036023 | >25 | 42.92 |
| EP-0036023 | >25 | 36.65 |
| EP-0036032 | >25 | 7.06 |
| EP-0036050 | >25 | 7.06 |
| EP-0036061 | 11.67 | 22.97 |
| EP-0036078 | 1.49 | 27.32 |
| EP-0036079 | 14.93 | 29.25 |
| EP-0036080 | 5.64 | 37.66 |
| EP-0036081 | 6.28 | 21.05 |
| EP-0036082 | 3.62 | 52.58 |
| EP-0036083 | 0.02 | 92.03 |
| EP-0036193 | 18.79 | 15.86 |
| EP-0036194 | 3.15 | 23.68 |
| EP-0036195 | 21.58 | 22.95 |
| EP-0036198 | 0.01 | 5.16 |
| EP-0036202 | 2.69 | 22.24 |
| EP-0036296 | 2.09 | 13.52 |
| EP-0036297 | >25 | 10.14 |
| EP-0036329 | 6.32 | 18.26 |
| EP-0036336 | 6.85 | 6.82 |
| EP-0036404 | >25 | 11.97 |
| EP-0036405 | 6.11 | 20.95 |
| EP-0036406 | 4.38 | 16.16 |
| EP-0036407 | >25 | 10.31 |
| EP-0036408 | 10.79 | 12.42 |
| EP-0036409 | n/a | n/a |
| EP-0036410 | >25 | 13.38 |
| EP-0036411 | 15.96 | 12.54 |
| EP-0036413 | 1.69 | 19.76 |
| EP-0036414 | 2.26 | 23.09 |
| EP-0036422 | >25 | 11.51 |
| EP-0036425 | >25 | 7.63 |
| EP-0036426 | 3.61 | 28.92 |
| EP-0036428 | >25 | 9.69 |
| EP-0036437 | 4.65 | 31.93 |
| EP-0036438 | 3.17 | 27.61 |
| EP-0036439 | >25 | 12.33 |
| EP-0036451 | >25 | 8.9 |
| EP-0036453 | >25 | 15.96 |
| EP-0036463 | 3.48 | 14.49 |
| EP-0036468 | 14.69 | 30.45 |
| EP-0036477 | 4.41 | 30.27 |
| EP-0036837 | 21.43 | 43.86 |
| EP-0036847 | >25 | 13.18 |
| EP-0036848 | 1.81 | 8.7 |

*As measured in Table 3 above.

TABLE 3
SUPPLEMENT B.

| | Mitophagy EC50 | Max cell death at | Cell death |
|---|---|---|---|
| Molecule | (HeLa mKeima | 25 uM compound, | at 25 μM |
| Name | assay) (uM) | 1 uM FO (%) | (no FO) (%) |
| EP-0035985 | 0.44 ± 0.12 μM | 10.7 | 2.59 |
| | from N = 128 runs | | |
| EP-0036081 | 6.25 | 21 | 3.04 |
| EP-0037056 | 1.33 | 16 | 2.51 |
| EP-0037059 | 1.02 | 26 | 4.9 |
| EP-0037085 | Minimal activity | 21.2 | 2.75 |
| EP-0037092 | 9.04 | 35.6 | 5.5 |
| EP-0037094 | 1.08 | 31.4 | 7.13 |
| EP-0037130 | 3.99 | 3.18 | 1.18 |
| EP-0037131 | 9.46 | 5.9 | 1.2 |
| EP-0037154 | 15.1 | 7.59 | 2.35 |
| EP-0037155 | 18.5 | 14.4 | 2.51 |
| EP-0037178 | Minimal activity | 12.8 | 3.79 |
| EP-0037214 | 2.98 | 71.2 | 57.1 |
| EP-0037845 | 17.3 | 6.92 | 2.69 |
| EP-0037852 | 1.15 | 59.535 | 27 |
| EP-0037853 | 5.55 | 30.87 | 9.83 |
| EP-0037861 | 1.63 | 12.11 | 3.4 |
| EP-0037862 | 6.32 | 10.645 | 2.61 |
| EP-0037863 | 4.00 | 15.865 | 3.66 |
| EP-0037871 | 13.34 | 3.555 | 2.49 |
| EP-0037880 | 1.81 | 8.735 | 5.04 |
| EP-0037881 | Minimal activity | 12 | 6.45 |
| EP-0037882 | 0.59 | 13.32 | 3.79 |
| EP-0037883 | 4.18 | 23.8 | 17.9 |
| EP-0037962 | 1.48 | 7.67 | 5.15 |
| EP-0037963 | 22.1 | 18.4 | 15.3 |
| EP-0037965 | 2.78 | 25.1 | 3.16 |
| EP-0038205 | 0.47 | 14.9 | 9.32 |
| EP-0038252 | 0.81 | 79.29 | 4.33 |
| EP-0038313 | 1.06 | 30.1 | 3.27 |
| EP-0038508 | 4.38 | 11 | 3.32 |
| EP-0039729 | 1.98 | 17.3 | 7.26 |
| | Mitotox safety | | |
| | margin | | |
| Molecule | (Therapeutic | Solubility | |
| Name | window) | (μM in 1× PBS) | Ratio** |
| EP-0035985 | ++++ | 1 | 40.02 |
| EP-0036081 | + | 150 | 8.85 |
| EP-0037056 | ++++ | 74.8 | 30 |
| EP-0037059 | + | 1 | 8.69 |
| EP-0037085 | | 194 | NA |
| EP-0037092 | | 194 | NA |
| EP-0037094 | + | NA | 7.74 |
| EP-0037130 | | 1.2 | NA |
| EP-0037131 | | 1.2 | NA |
| EP-0037154 | + | 19.2 | 1.66 |
| EP-0037155 | + | 20.1 | 1.35 |
| EP-0037178 | | 1 | NA |
| EP-0037214 | + | 1 | 8.39 |
| EP-0037845 | | 1.32 | NA |
| EP-0037852 | + | 12.9 | 7.25 |
| EP-0037853 | | 9.07 | NA |
| EP-0037861 | | 23.8 | NA |
| EP-0037862 | | 16.4 | NA |
| EP-0037863 | | 16.3 | NA |
| EP-0037871 | | 20.9 | NA |
| EP-0037880 | ++ | 9.39 | 13.8 |
| EP-0037881 | | 7.79 | NA |
| EP-0037882 | ++++ | 1 | 42.16 |
| EP-0037883 | | 1 | NA |
| EP-0037962 | | 1.31 | NA |
| EP-0037963 | | 1 | NA |
| EP-0037965 | | 2.37 | NA |
| EP-0038205 | ++++ | 1 | 53.2 |
| EP-0038252 | | 190 | NA |
| EP-0038313 | | 1 | NA |
| EP-0038508 | | 102 | NA |
| EP-0039729 | | | NA |

* + = ratio <10; ++ = ratio 10< × <20; +++ = ratio 20< × <30; ++++ = ratio 30<
**Ratio = highest concentration with no observable mitotoxicity (μM) divided by the EC₅₀ (μM) of the compound

TABLE 4

| | | Toxicity |
|---|---|---|
| | Potency | (% positive for DAPI |
| Compound | (EC₅₀ Max Mitophagy) (μM) | takeup @ 50 μM) |
| 35169 | >25 | 5.4 |
| 34884 | >25 | 3.7 |
| 34886 | 9.1 | 9.1 |
| 35339 | >25 | 2.6 |
| 35418 | >25 | 3.3 |
| 35476 | >25 | 4.0 |
| 35536 | >25 | 3.5 |
| 35571 | >25 | 2.0 |
| 35574 | >25 | 2.3 |
| 35507 | 4.9 | 5.3 |
| 35985 | 0.44 ± 0.12 μM from N = 128 runs | 2.4 |

### 27. Inspection of Crystallization and Round Cells

Briefly, the Hela MKYP (Mito-Keima/YFP-Parkin) cells were seeded at 10K cells/well. EP/MTK compounds were added at seeding (cells were still in suspension). The cells were incubated with the EP/MTK compounds for 16 hours, then 1 μM FCCP/oligomycin was added for 6 hours. Prior to harvesting, cells were scored by eye under 20× magnification for the presence or absence of crystalline or aggregated compound, or round cells.

TABLE 4

| μM | Log [M] |
|---|---|
| 50.000 | −4.3 |
| 25.000 | −4.6 |
| 12.5000 | −4.9 |
| 6.250 | −5.2 |
| 3.125 | −5.5 |
| 1.563 | −5.8 |
| 0.781 | −6.1 |
| 0.391 | −6.4 |
| 0.195 | −6.7 |
| 0.098 | −7.0 |
| 0.049 | −7.3 |
| 0.024 | −7.6 |
| 0.01 (used for 0) | −8 |

Data corresponding to the visual inspection of crystallization (1=yes crystals; 0=no crystals) is shown in Tables 5A-5D below.

TABLE 5A

| | Compound No. | | | | |
|---|---|---|---|---|---|
| μM | 36296 | 36329 | 36337 | 6BAP | 35910 |
| 50.0 | 0 | 0 | 0 | 0 | 0 |
| 25.0 | 0 | 0 | 0 | 0 | 0 |
| 12.5 | 0 | 0 | 0 | 0 | 0 |
| 6.3 | 0 | 0 | 0 | 0 | 0 |
| 3.1 | 0 | 0 | 0 | 0 | 0 |
| 1.6 | 0 | 0 | 0 | 0 | 0 |

TABLE 5B

| | Compound No. | | | | | | | |
|---|---|---|---|---|---|---|---|---|
| μM | Kinetin | 35910 | 36002 | 36004 | 36022 | 35985 | 36023 | 36025 |
| 50.0 | 0 | 0 | 1 | 0 | 0 | 0 | 1 | 0 |
| 25.0 | 0 | 0 | 1 | 0 | 0 | 0 | 0 | 0 |
| 12.5 | 0 | 0 | 0 | 0 | 0 | 0 | 0 | 0 |
| 6.3 | 0 | 0 | 0 | 0 | 0 | 0 | 0 | 0 |
| 3.1 | 0 | 0 | 0 | 0 | 0 | 0 | 0 | 0 |
| 1.6 | 0 | 0 | 0 | 0 | 0 | 0 | 0 | 0 |

TABLE 5C

| | Compound No. | | | | | | | |
|---|---|---|---|---|---|---|---|---|
| μM | Kinetin | 35910 | 36193 | 36194 | 36195 | 36198 | 36202 | 36082 |
| 50.0 | 0 | 0 | 0 | 0 | 0 | 0 | 0 | 0 |
| 25.0 | 0 | 0 | 0 | 0 | 0 | 0 | 0 | 0 |
| 12.5 | 0 | 0 | 0 | 0 | 0 | 0 | 0 | 0 |
| 6.3 | 0 | 0 | 0 | 0 | 0 | 0 | 0 | 0 |
| 3.1 | 0 | 0 | 0 | 0 | 0 | 0 | 0 | 0 |
| 1.6 | 0 | 0 | 0 | 0 | 0 | 0 | 0 | 0 |

TABLE 5D

| | Compound No. | | | | |
|---|---|---|---|---|---|
| μM | Kinetin | 35910 | 36202 | 36296 | 36297 |
| 50.0 | 0 | 0 | 0 | 0 | 0 |
| 25.0 | 0 | 0 | 0 | 0 | 0 |
| 12.5 | 0 | 0 | 0 | 0 | 0 |
| 6.3 | 0 | 0 | 0 | 0 | 0 |
| 3.1 | 0 | 0 | 0 | 0 | 0 |
| 1.6 | 0 | 0 | 0 | 0 | 0 |

Data corresponding to the inspection of round cells (1=many round cells; 0=normal amount of round cells) is shown in Tables 6A-6D below.

TABLE 6A

| | Compound No. | | | | |
|---|---|---|---|---|---|
| μM | 36296 | 36329 | 36337 | 6BAP | 35910 |
| 50.0 | 0 | 0 | 0 | 0 | 0 |
| 25.0 | 0 | 0 | 0 | 0 | 0 |
| 12.5 | 0 | 0 | 0 | 0 | 0 |
| 6.3 | 0 | 0 | 0 | 0 | 0 |
| 3.1 | 0 | 0 | 0 | 0 | 0 |
| 1.6 | 0 | 0 | 0 | 0 | 0 |

TABLE 6B

| | Compound No. | | | | | | | |
|---|---|---|---|---|---|---|---|---|
| μM | Kinetin | 35910 | 36002 | 36004 | 36022 | 35985 | 36023 | 36025 |
| 50.0 | 0 | 0 | 1 | 0 | 0 | 0 | 1 | 0 |
| 25.0 | 0 | 0 | 1 | 0 | 0 | 0 | 0 | 0 |
| 12.5 | 0 | 0 | 0 | 0 | 0 | 0 | 0 | 0 |
| 6.3 | 0 | 0 | 0 | 0 | 0 | 0 | 0 | 0 |
| 3.1 | 0 | 0 | 0 | 0 | 0 | 0 | 0 | 0 |
| 1.6 | 0 | 0 | 0 | 0 | 0 | 0 | 0 | 0 |

TABLE 6C

| | Compound No. | | | | | | | |
|---|---|---|---|---|---|---|---|---|
| μM | Kinetin | 35910 | 36193 | 36194 | 36195 | 36198 | 36202 | 36082 |
| 50.0 | 0 | 0 | 0 | 0 | 0 | 0 | 0 | 1 |
| 25.0 | 0 | 0 | 0 | 0 | 0 | 0 | 0 | 0 |
| 12.5 | 0 | 0 | 0 | 0 | 0 | 0 | 0 | 0 |
| 6.3 | 0 | 0 | 0 | 0 | 0 | 0 | 0 | 0 |
| 3.1 | 0 | 0 | 0 | 0 | 0 | 0 | 0 | 0 |
| 1.6 | 0 | 0 | 0 | 0 | 0 | 0 | 0 | 0 |

TABLE 6D

| | Compound No. | | | | |
|---|---|---|---|---|---|
| μM | Kinetin | 35910 | 36202 | 36296 | 36297 |
| 50.0 | 0 | 0 | 0 | 0 | 0 |
| 25.0 | 0 | 0 | 0 | 0 | 0 |
| 12.5 | 0 | 0 | 0 | 0 | 0 |
| 6.3 | 0 | 0 | 0 | 0 | 0 |
| 3.1 | 0 | 0 | 0 | 0 | 0 |
| 1.6 | 0 | 0 | 0 | 0 | 0 |

### 28. Human Phospho-Ubiquitin (pS65) ub2 Assay

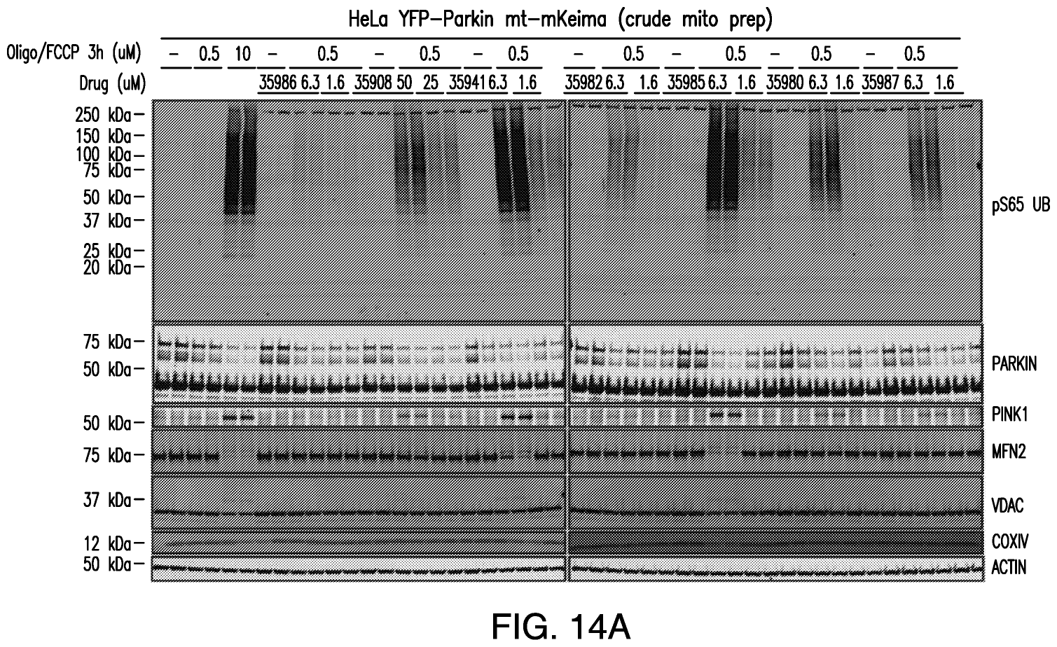

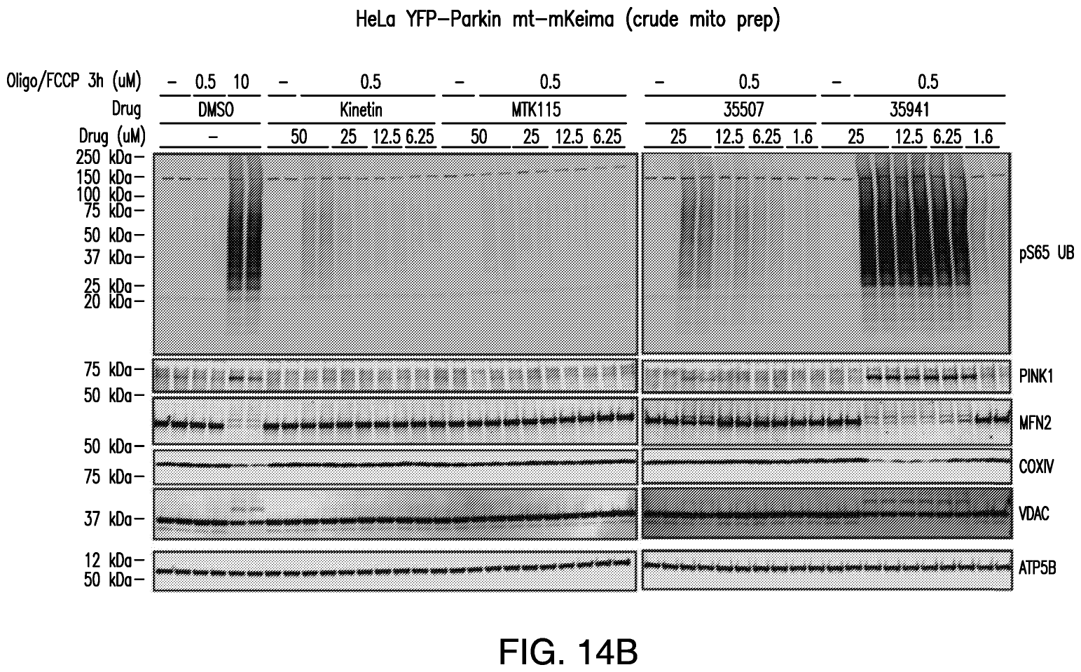

Briefly, HeLa MKYP cells were plated at 1,300,000 cells/plate in 10 cm plates in 10 mL of medium containing compound at various concentrations. Following 16 hrs of incubation, cells were treated with 0.5 uM FCCP/oligomycin for 2 hours, then harvested. Mitochondria were then isolated according to published protocols (Ordureau et al, 2014; https://doi.org/10.1016/j.molcel.2014.09.007). Equal amounts of samples were loaded on 26 well gradient gels, and a western blot analysis was performed using commercially available antibodies for various markers, including pho. Western blots illustrating the results of in vitro PINK1 kinase assays are shown in FIG. 14A and FIG. 14B.

### 29. Human Mitophagy Assay

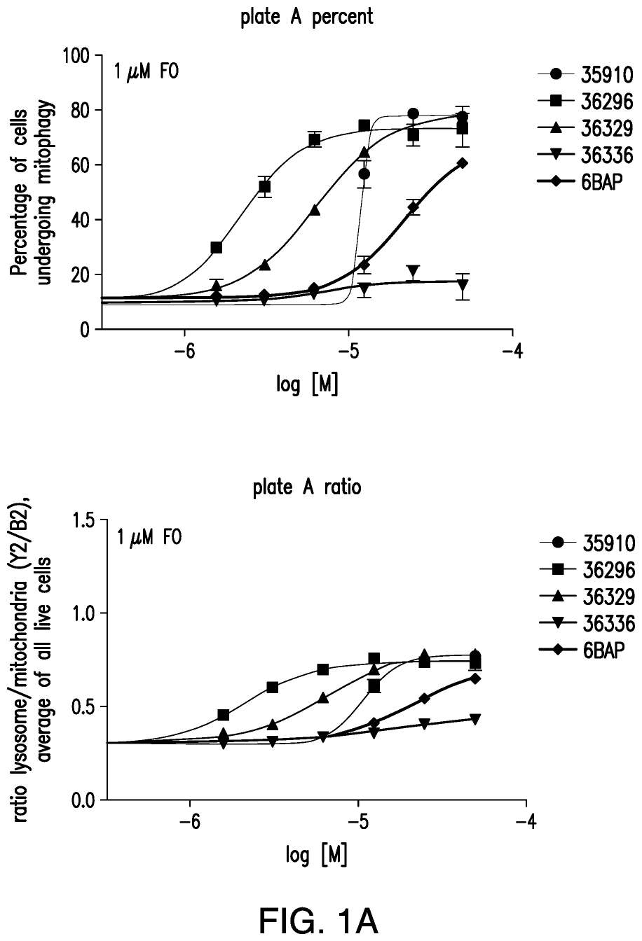

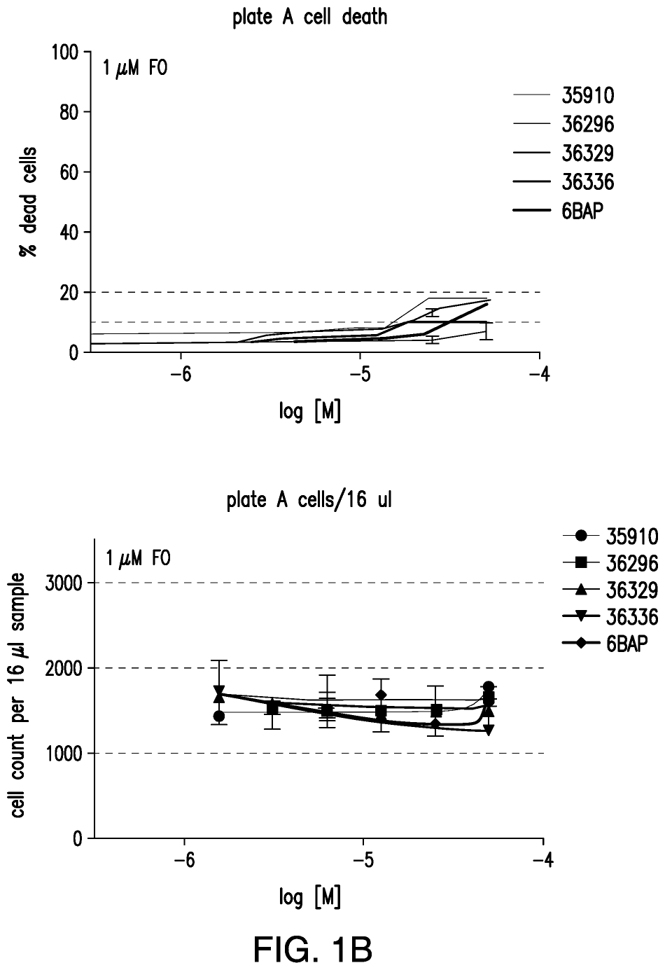

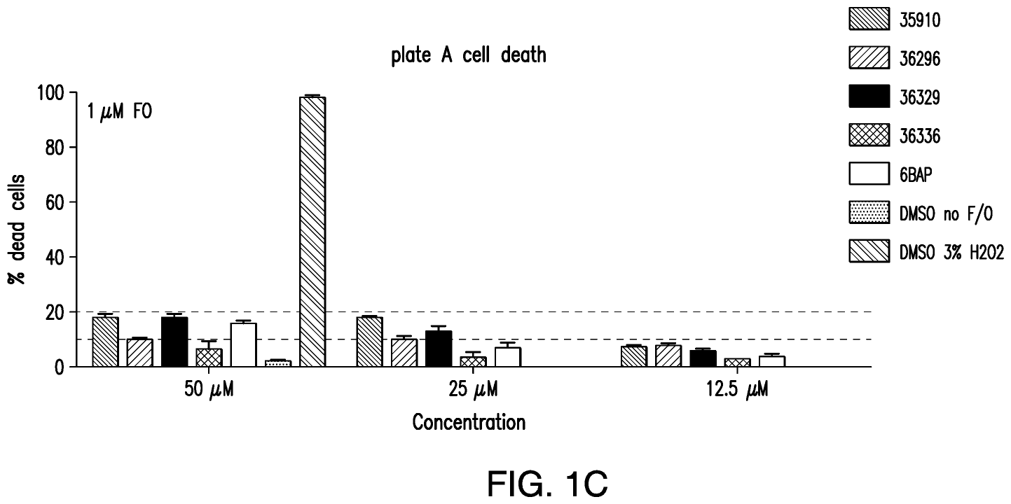

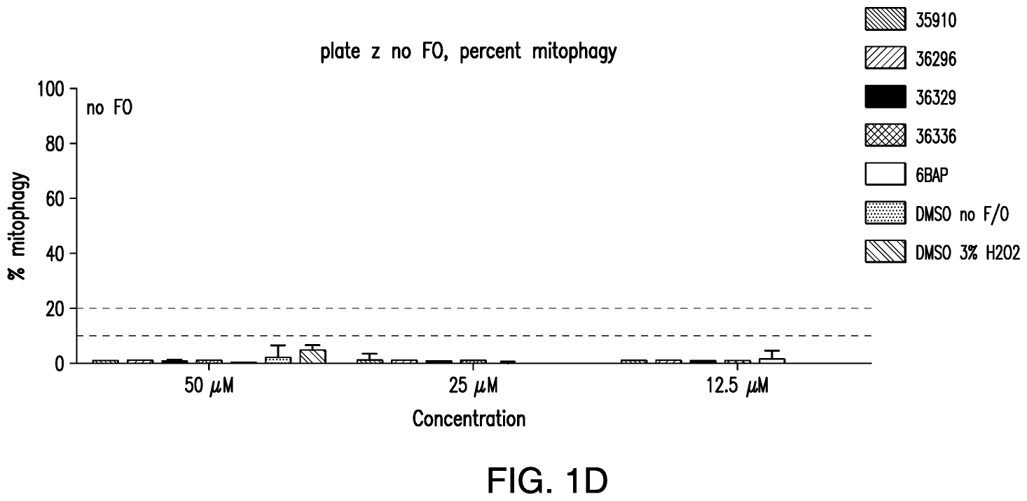

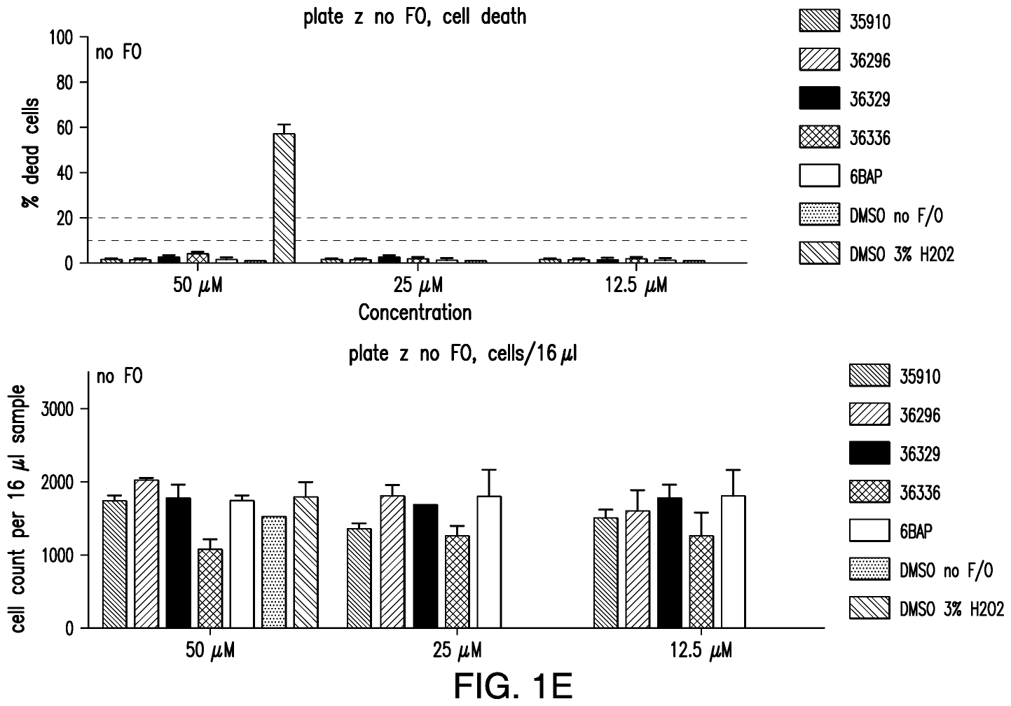

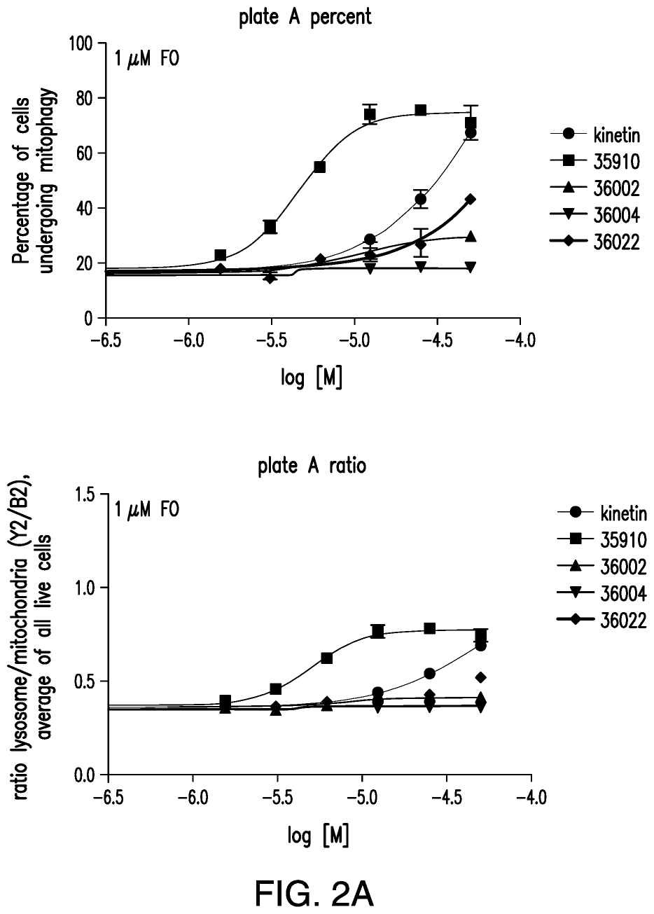

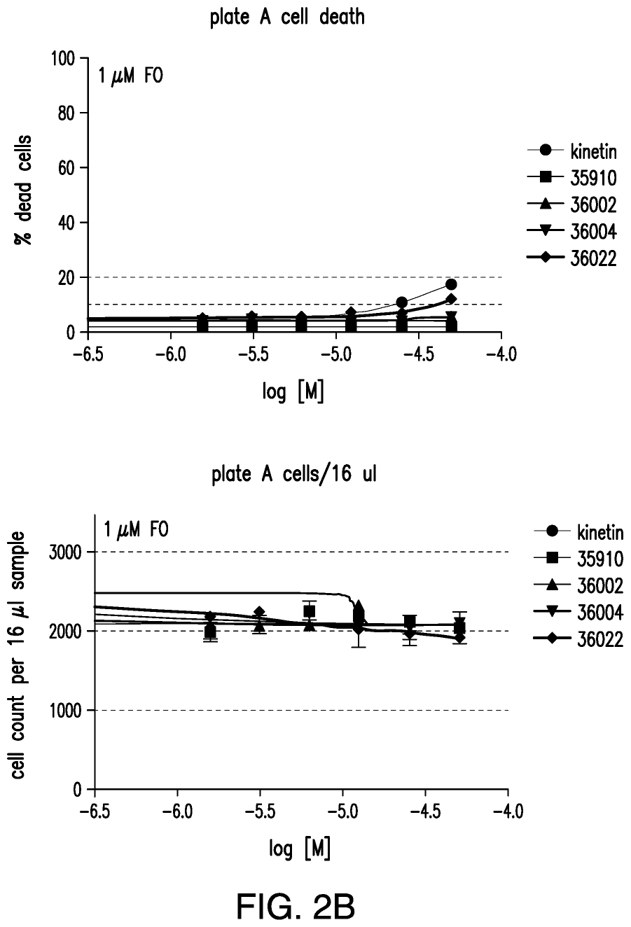

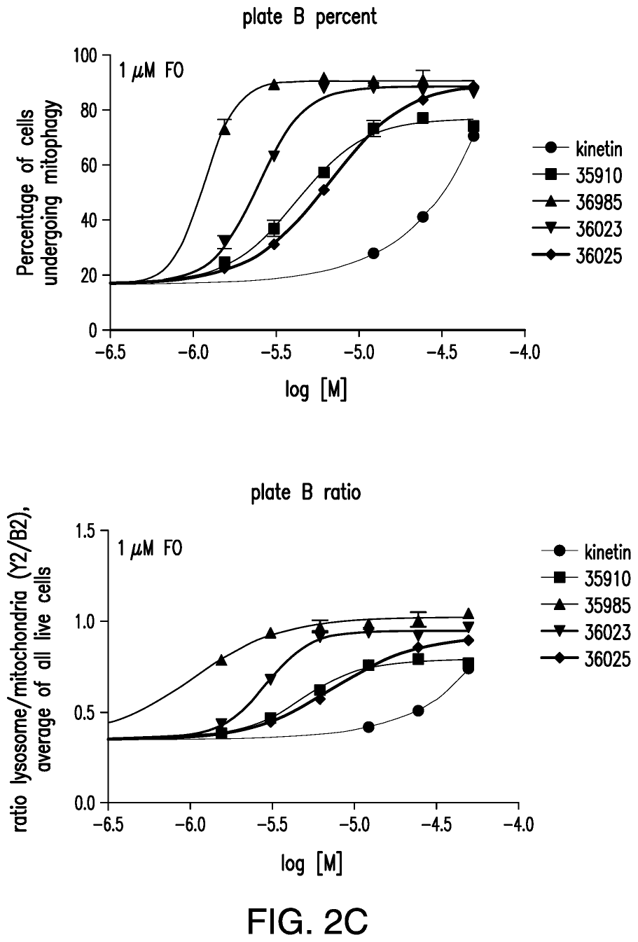

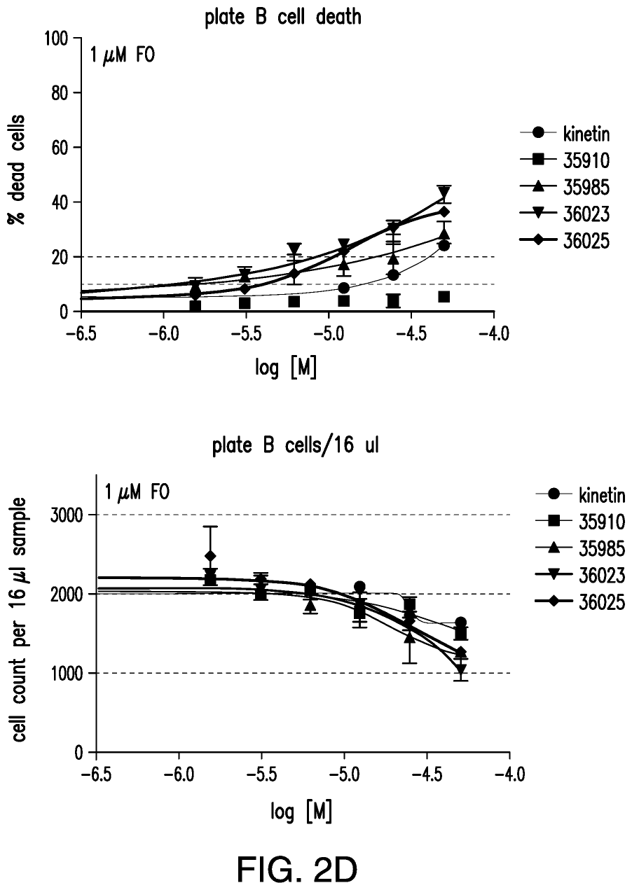

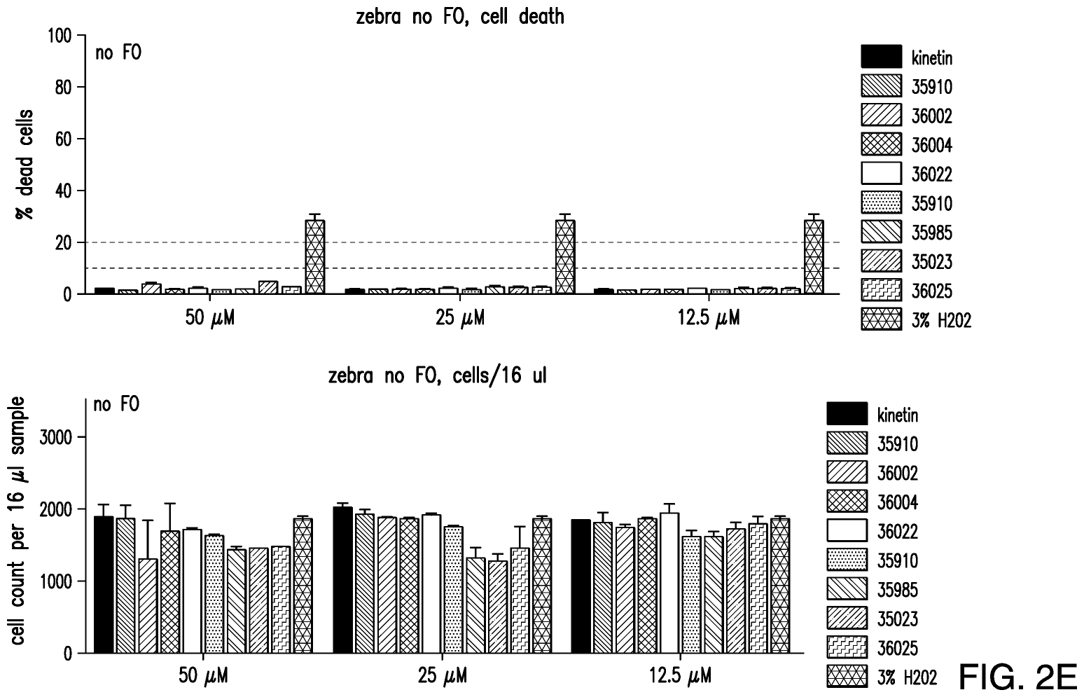

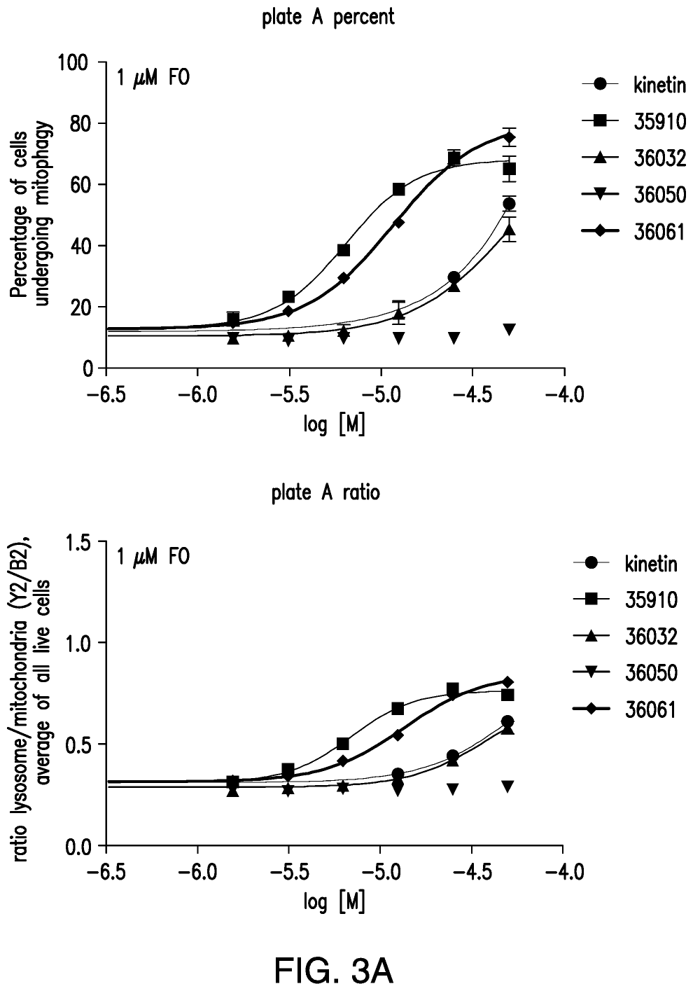

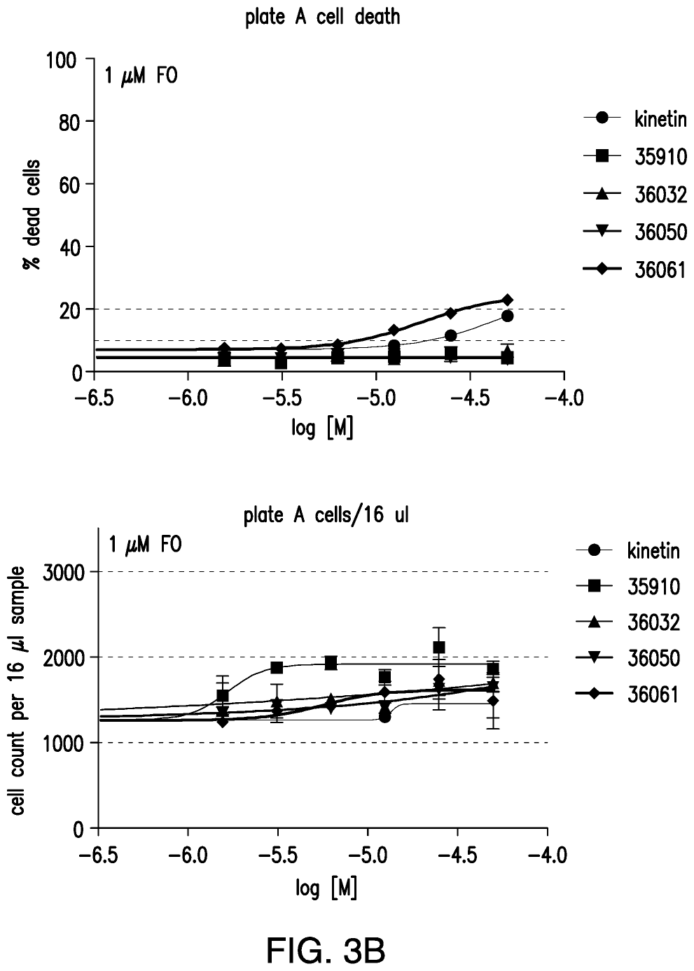

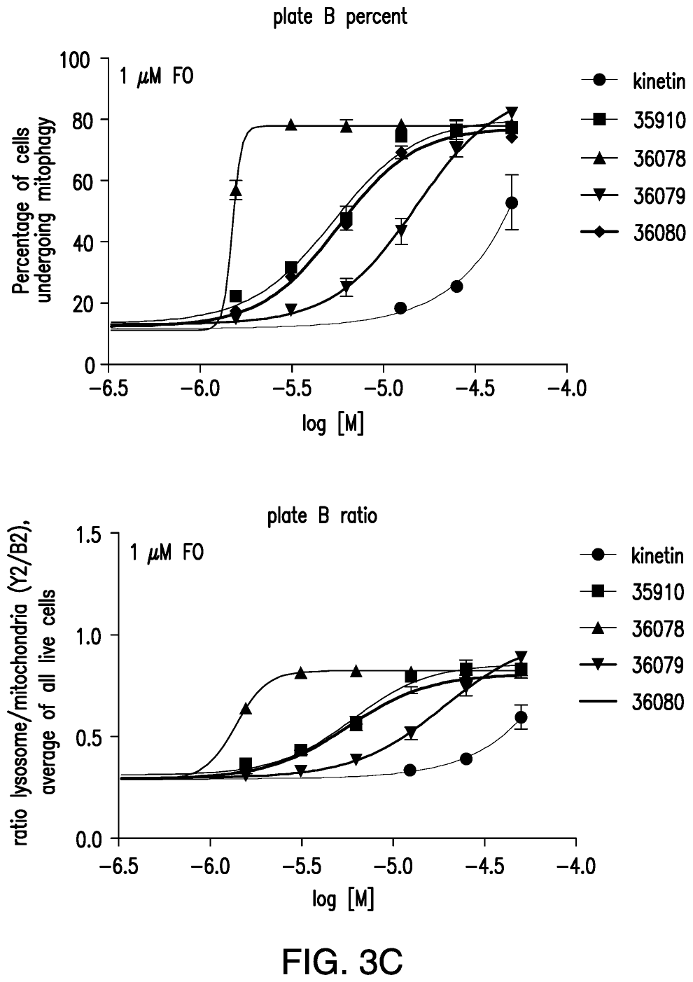

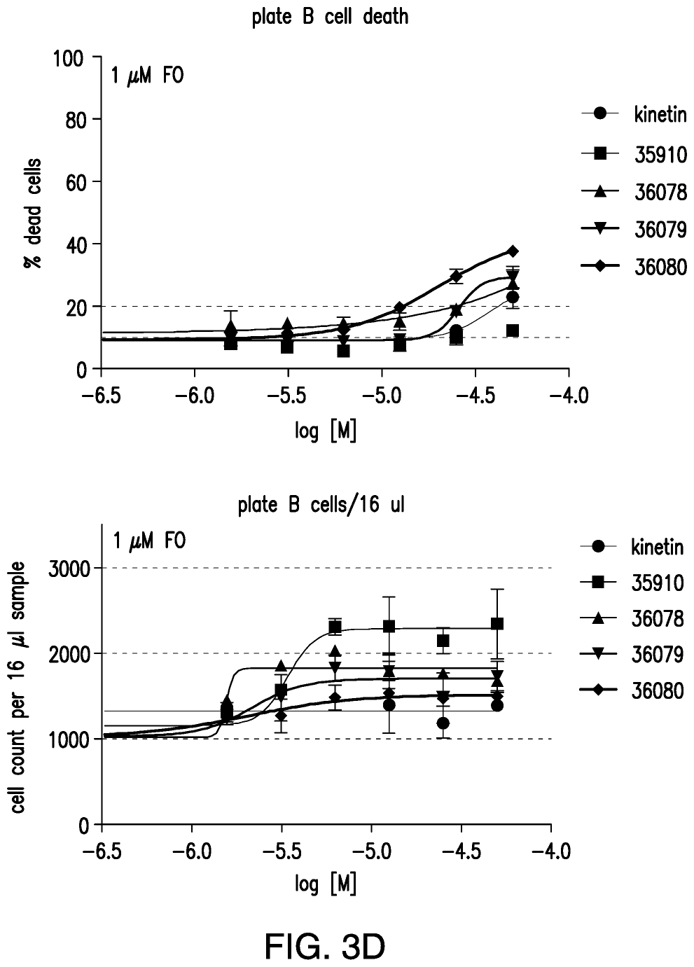

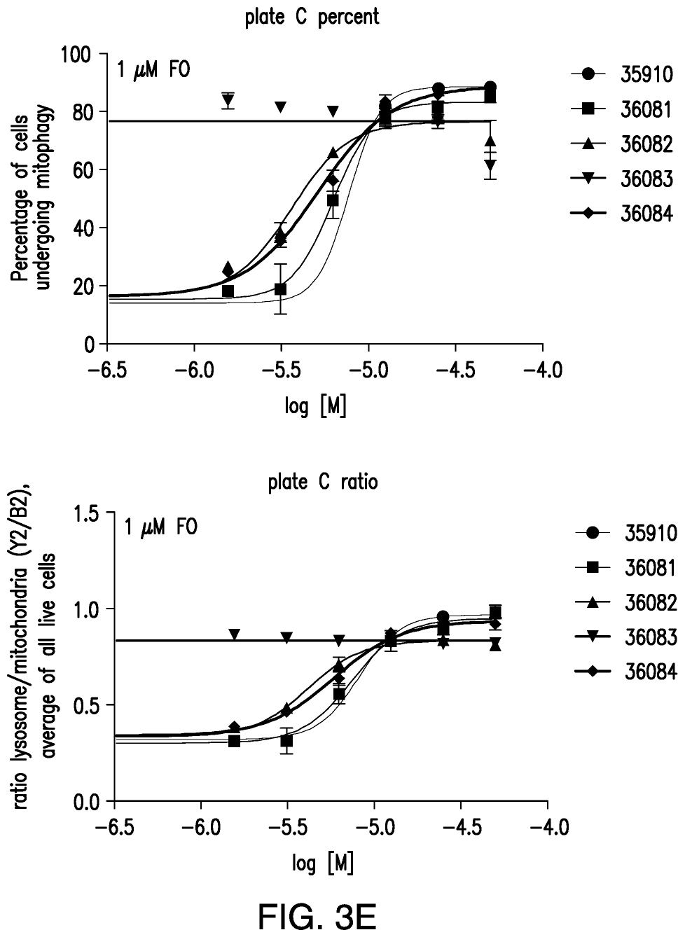

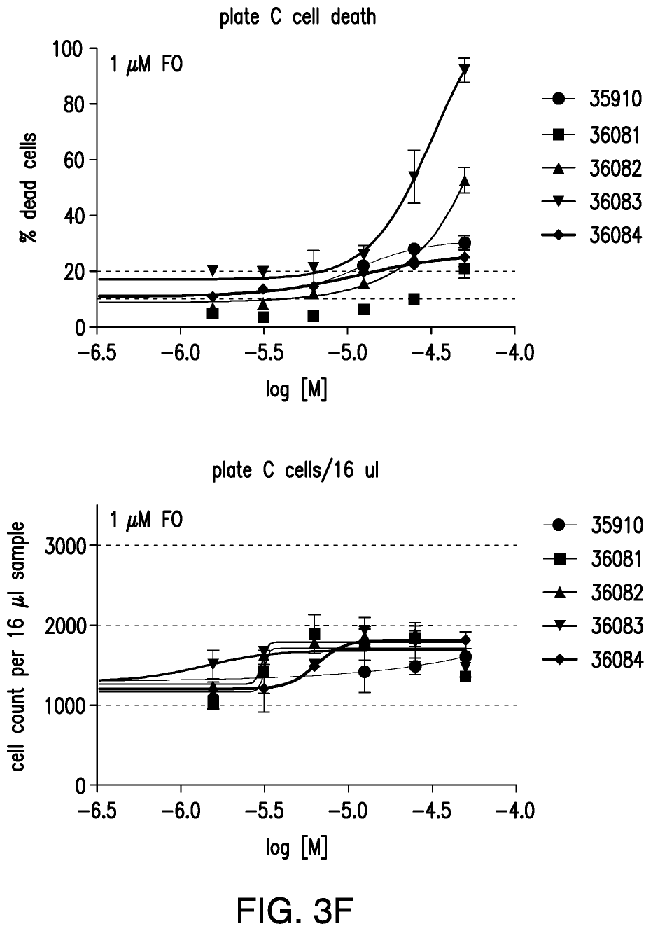

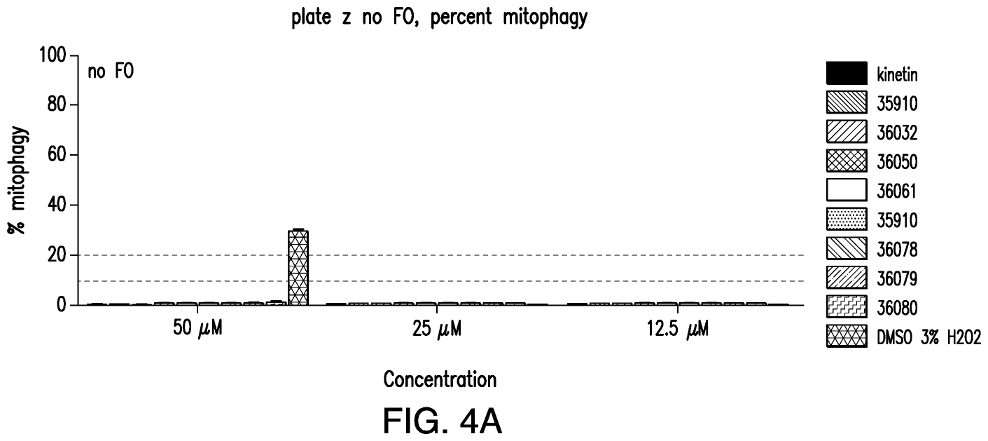

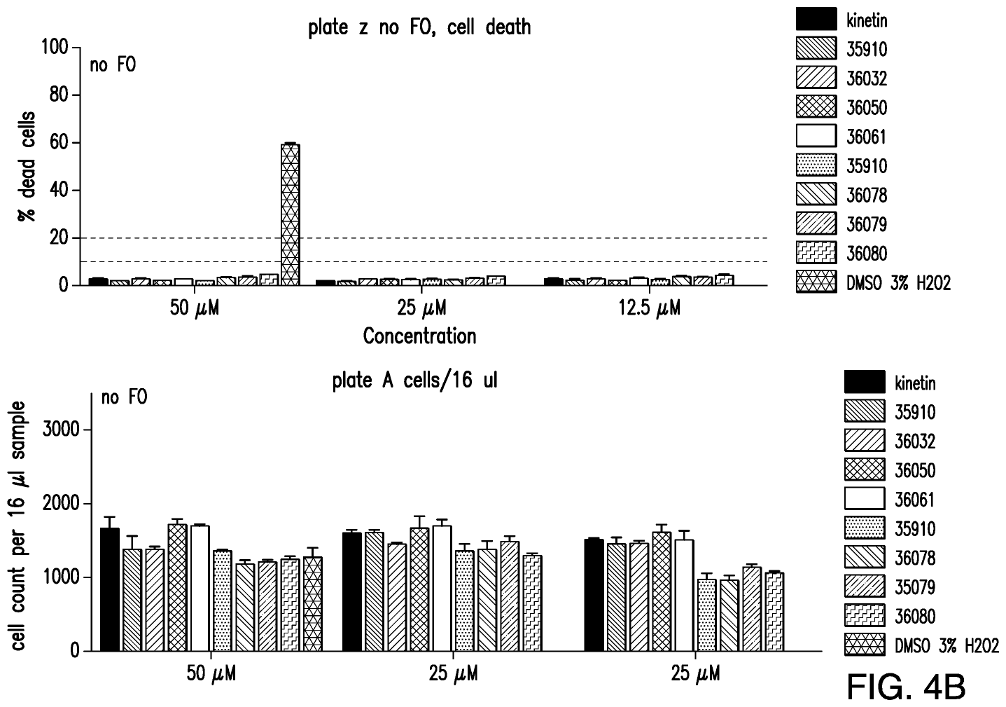

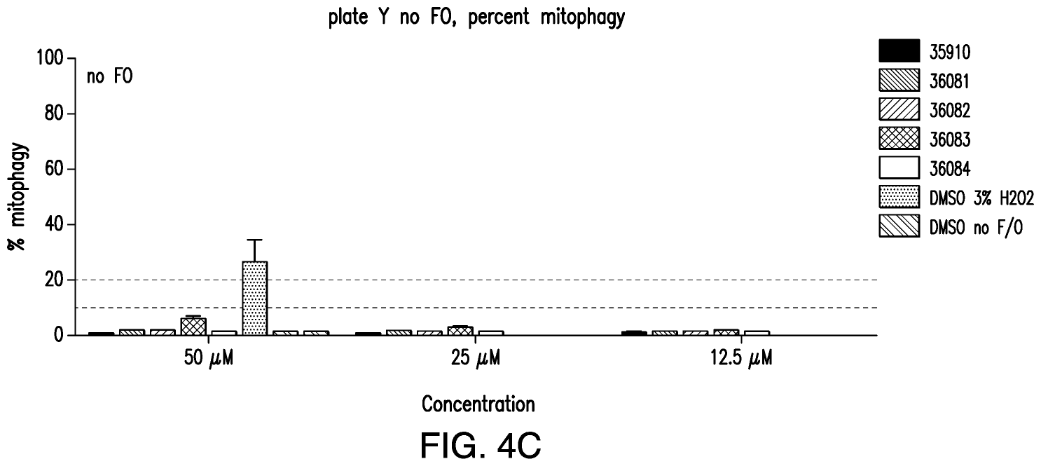

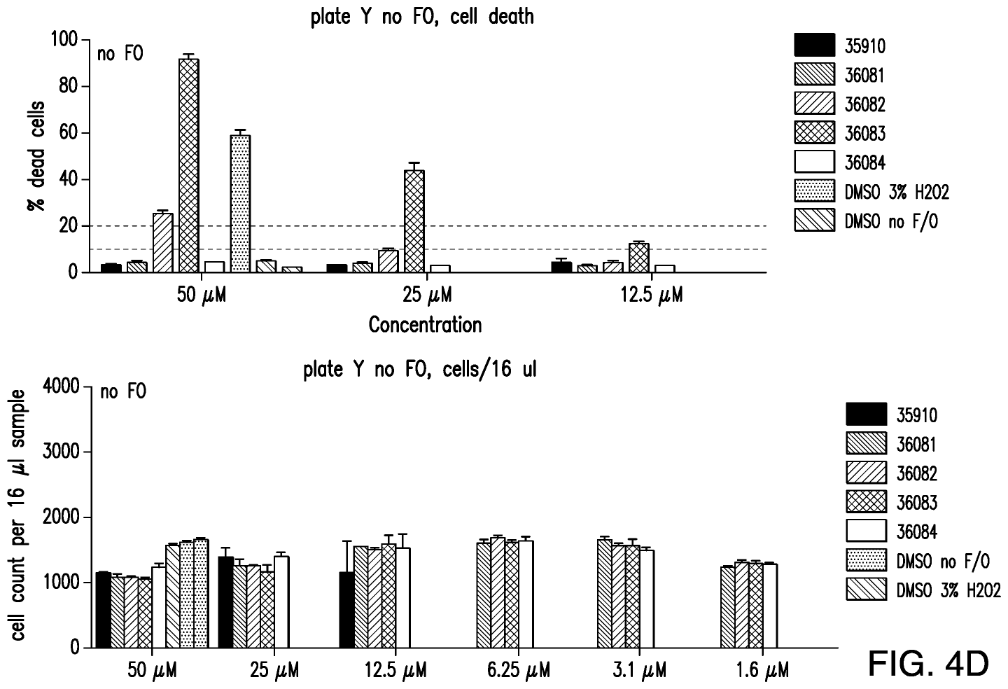

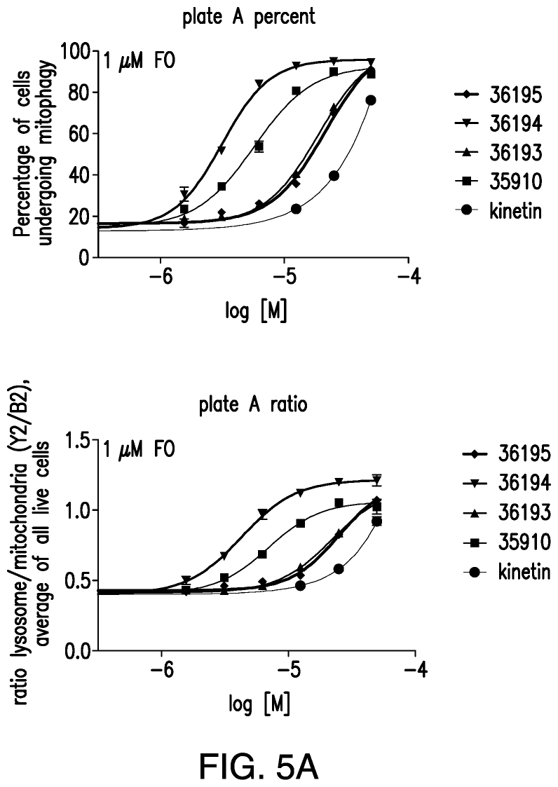

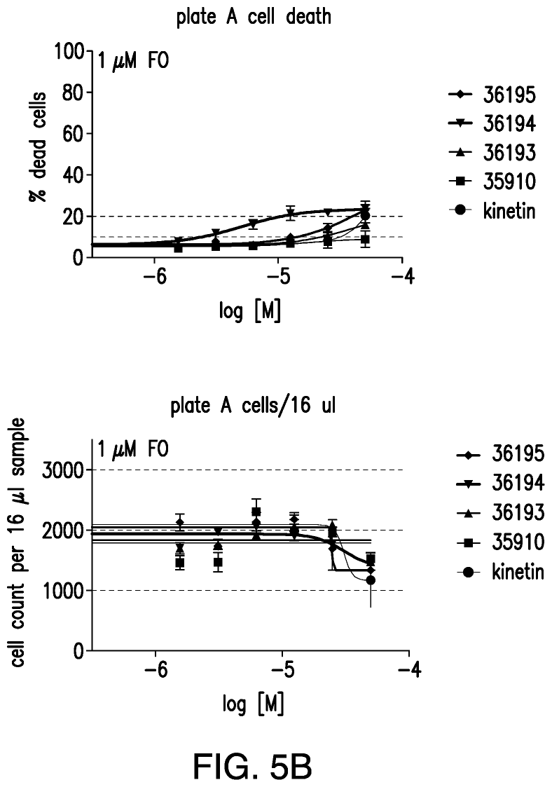

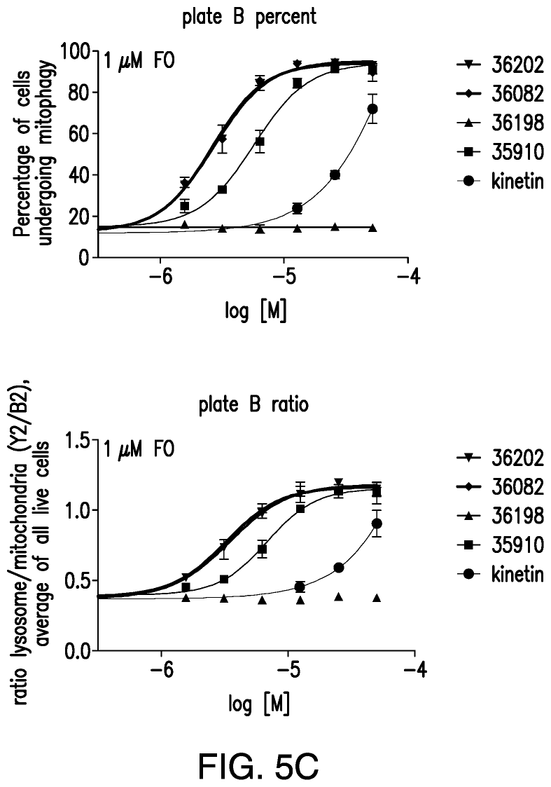

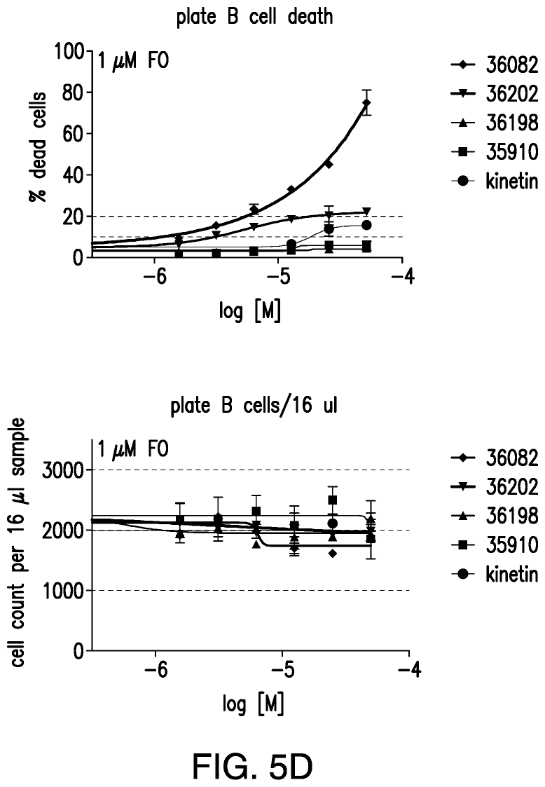

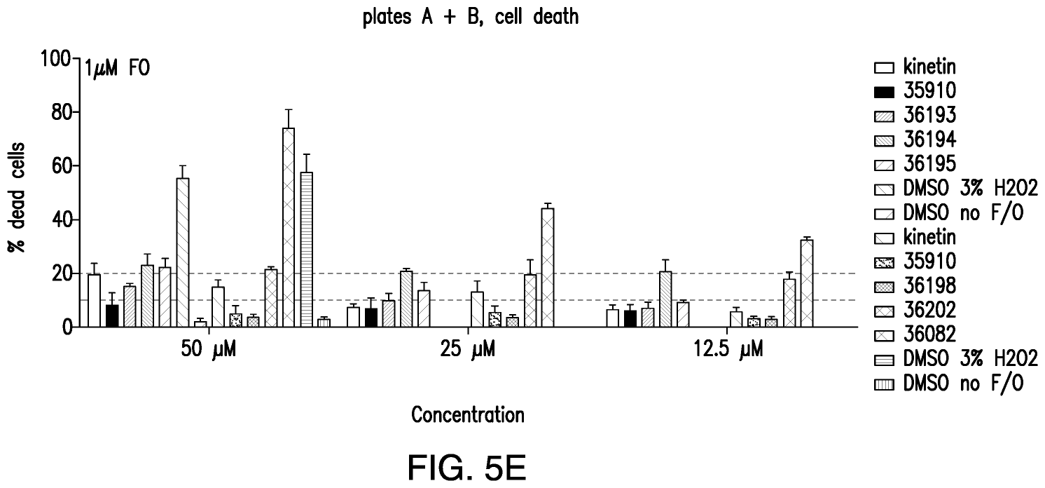

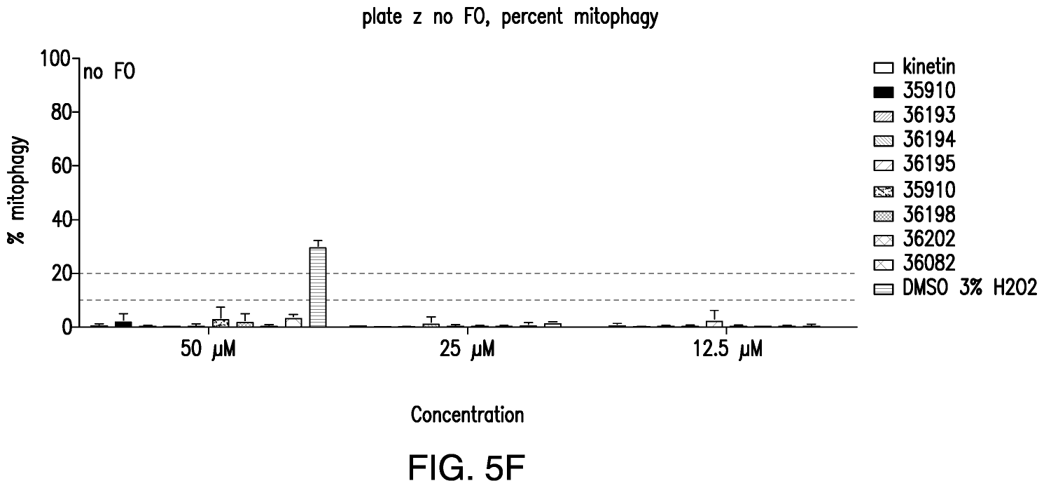

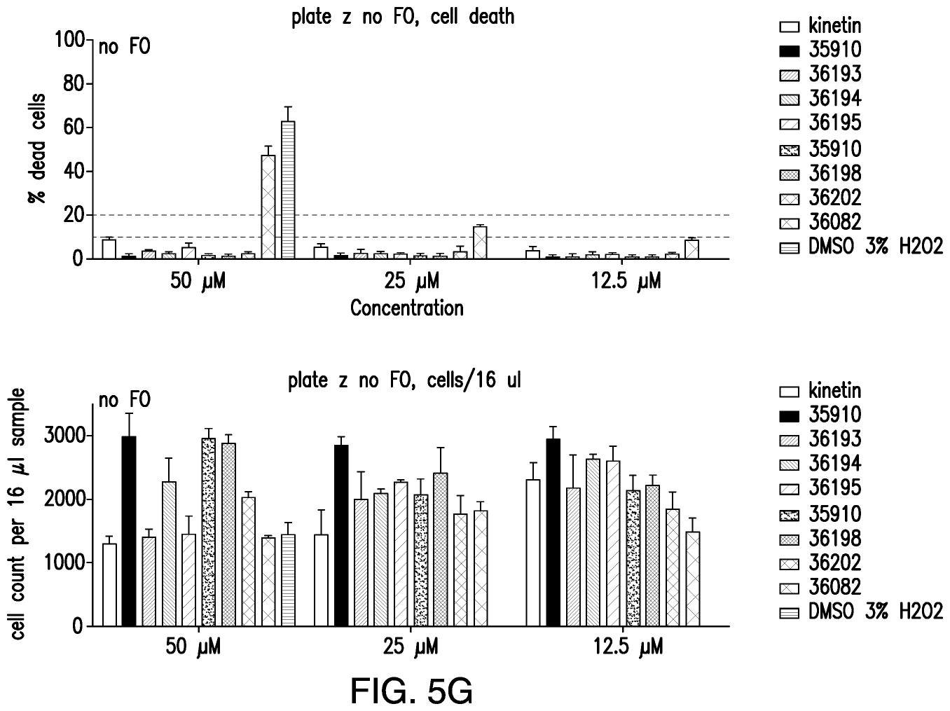

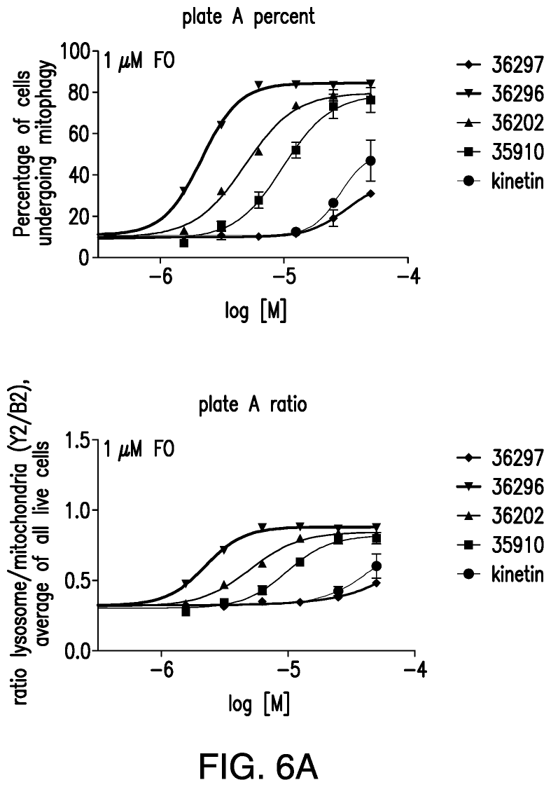

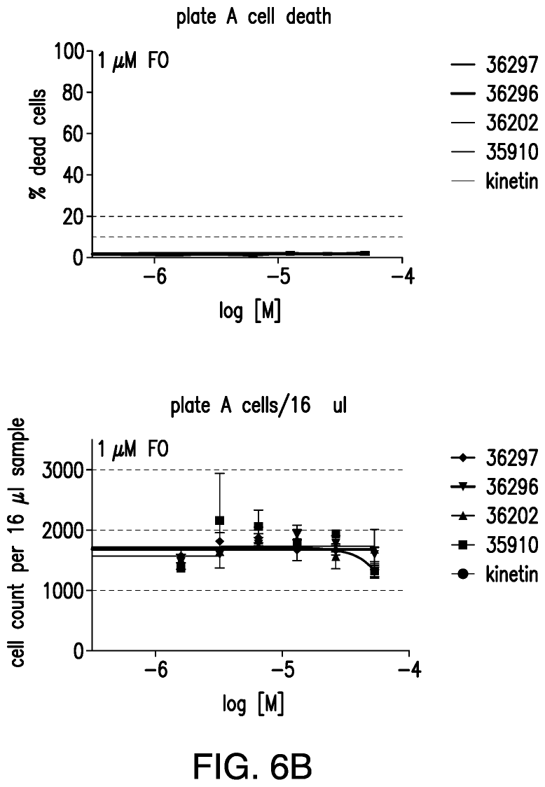

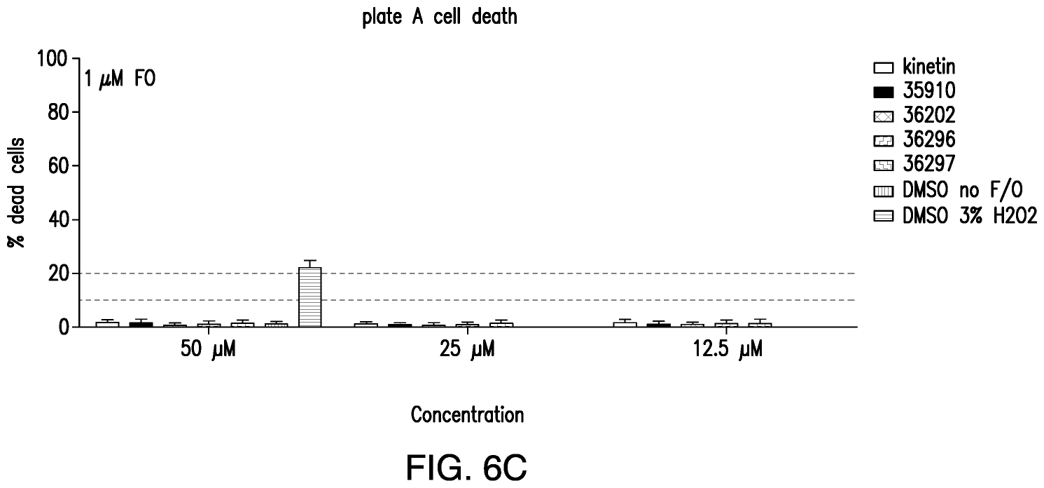

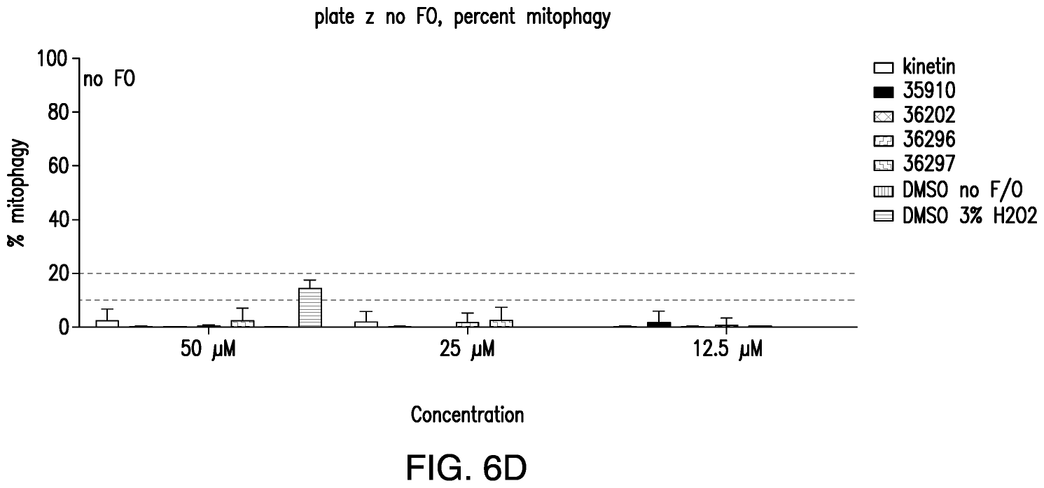

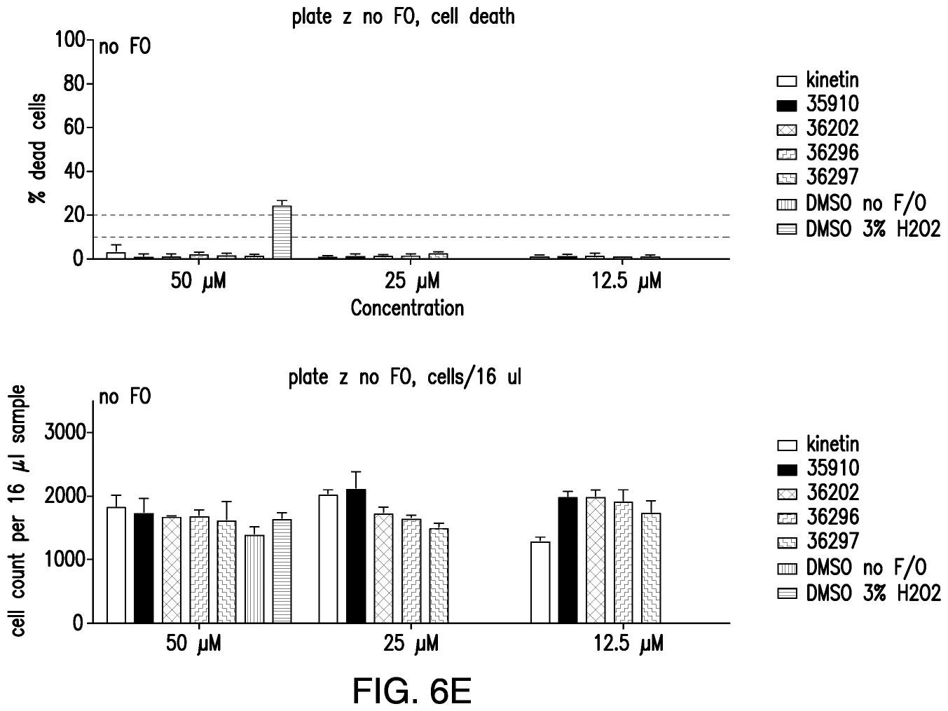

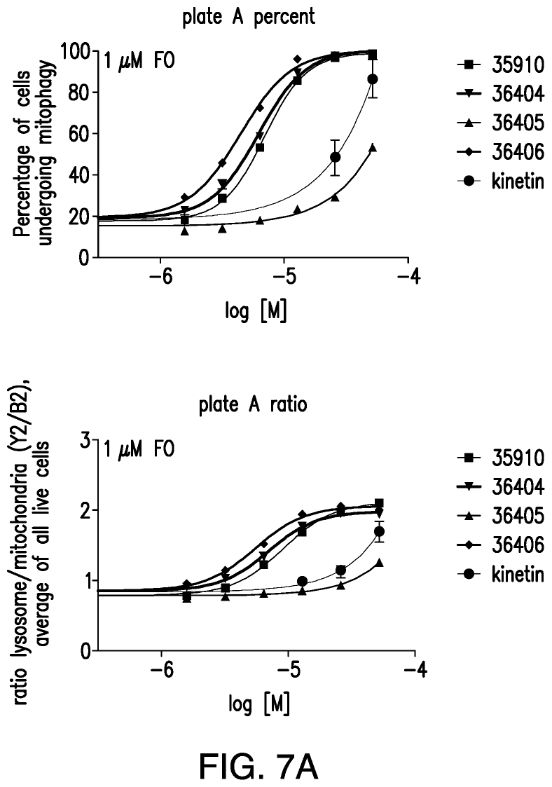

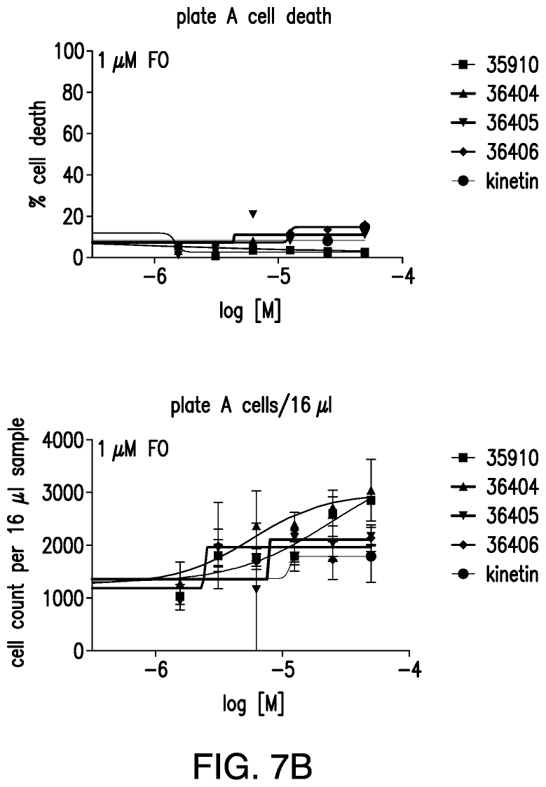

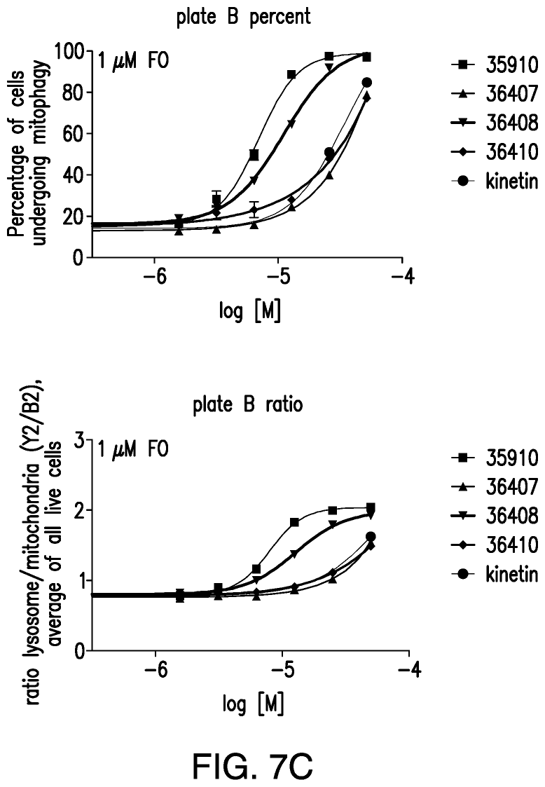

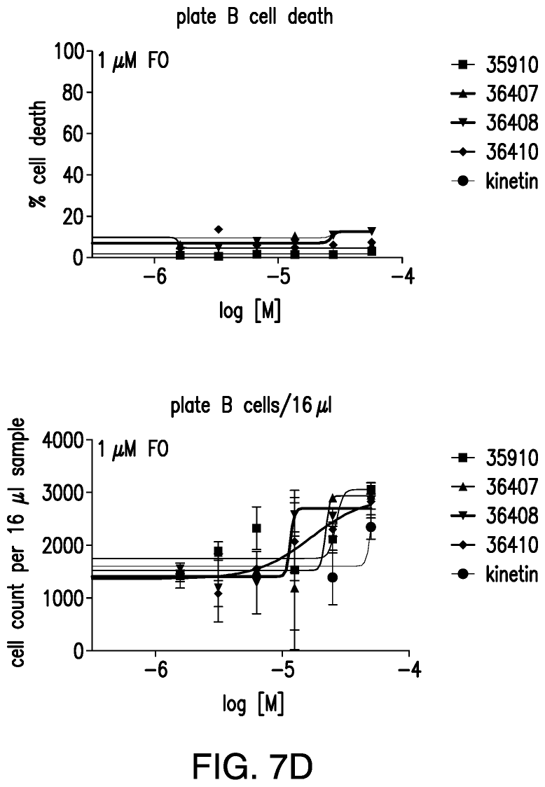

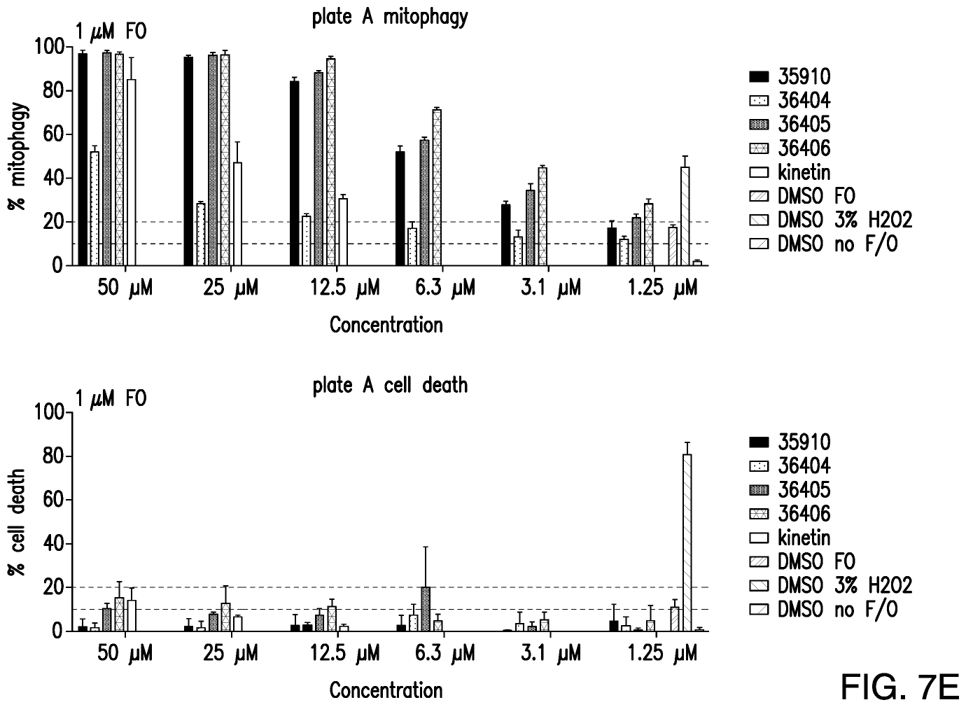

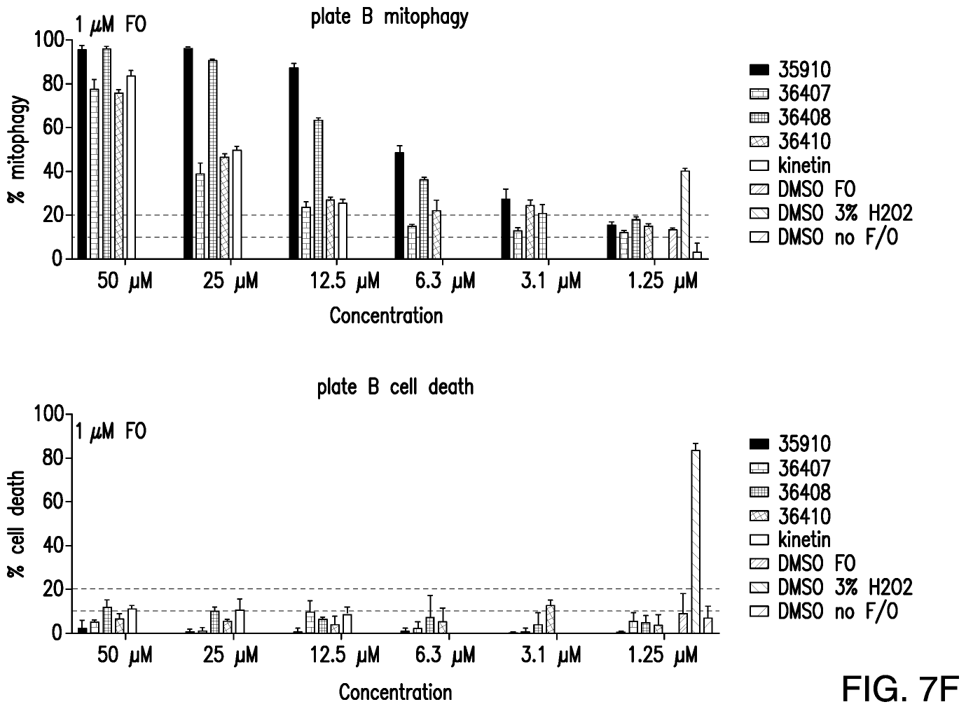

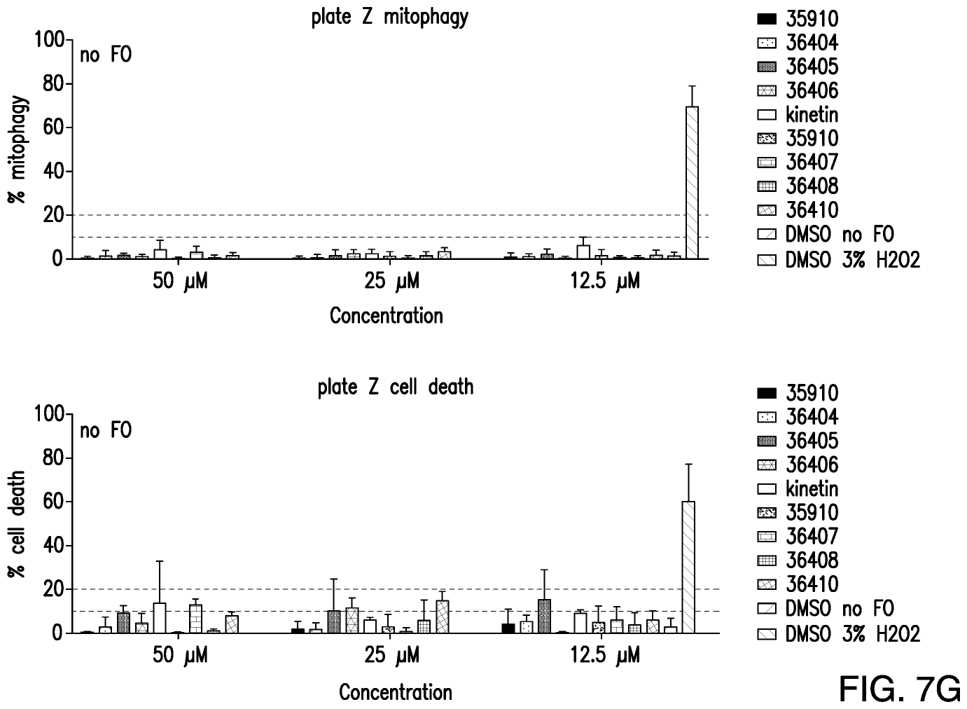

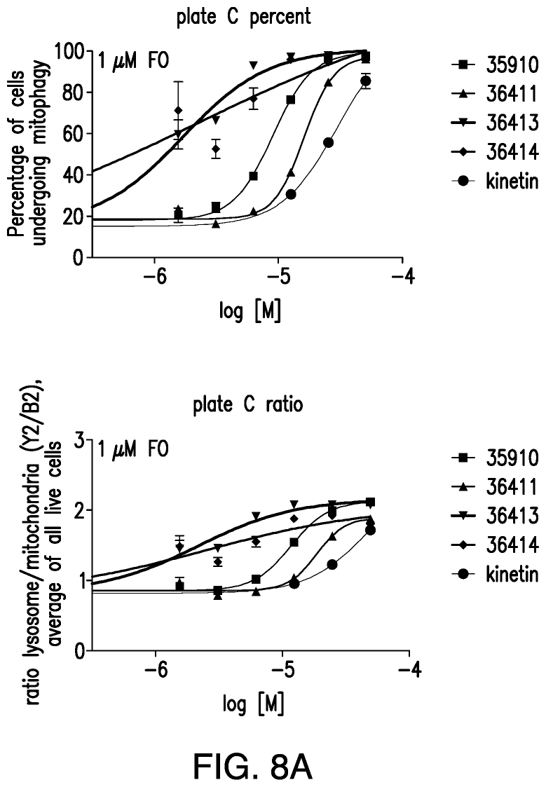

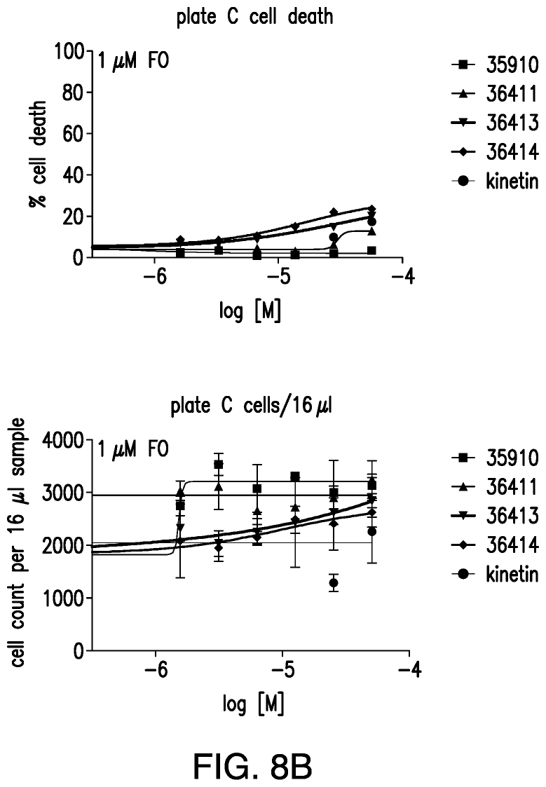

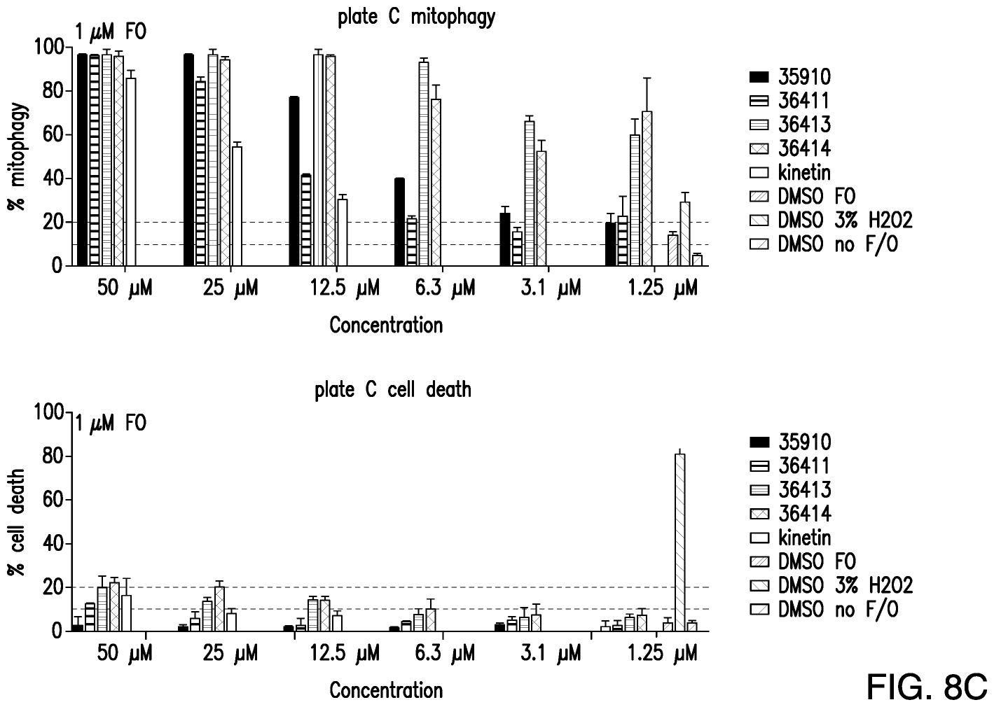

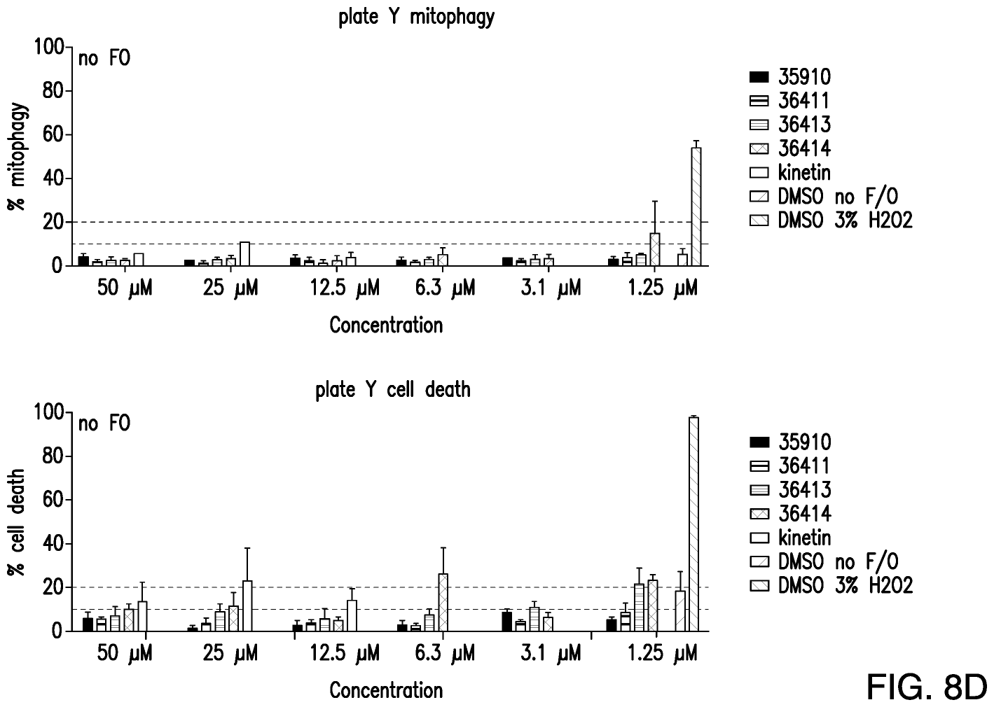

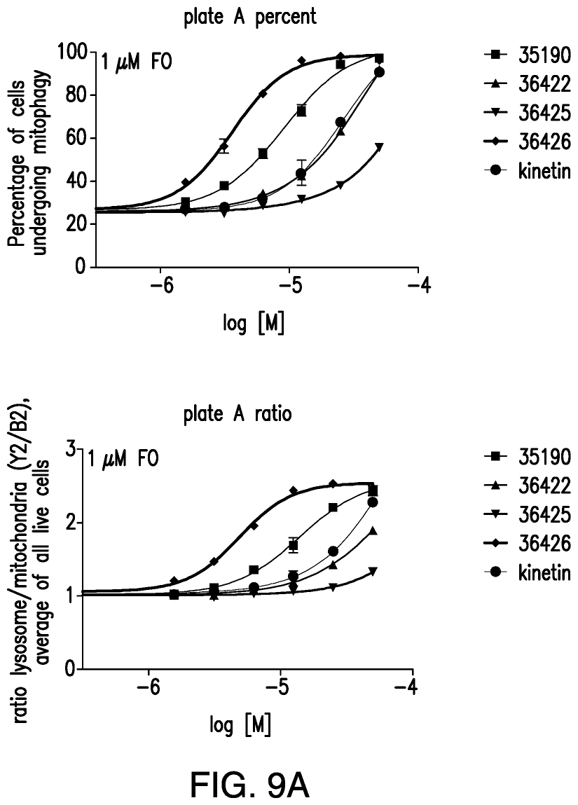

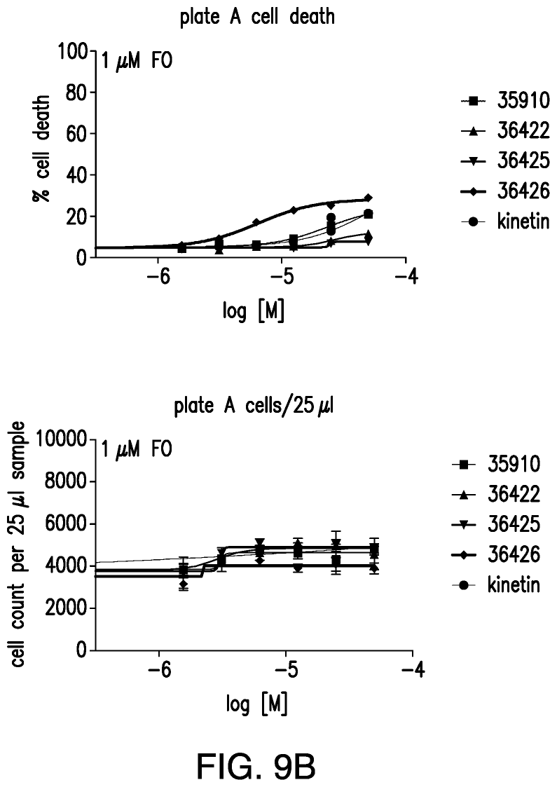

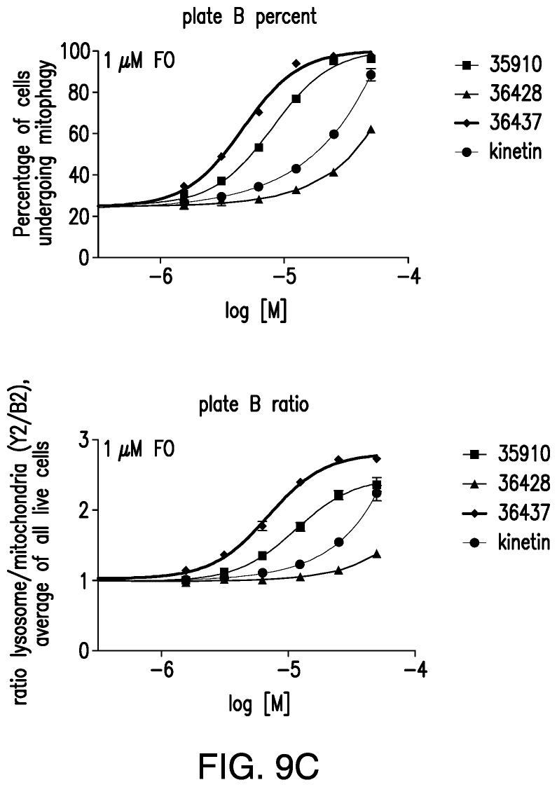

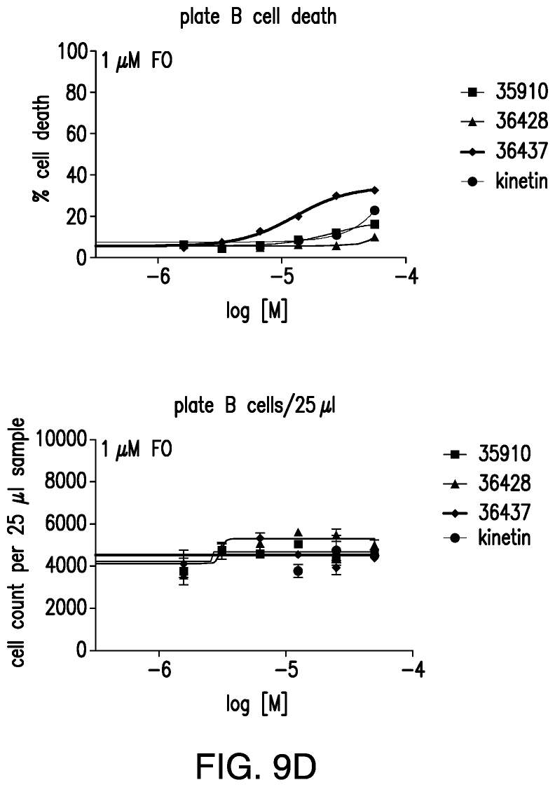

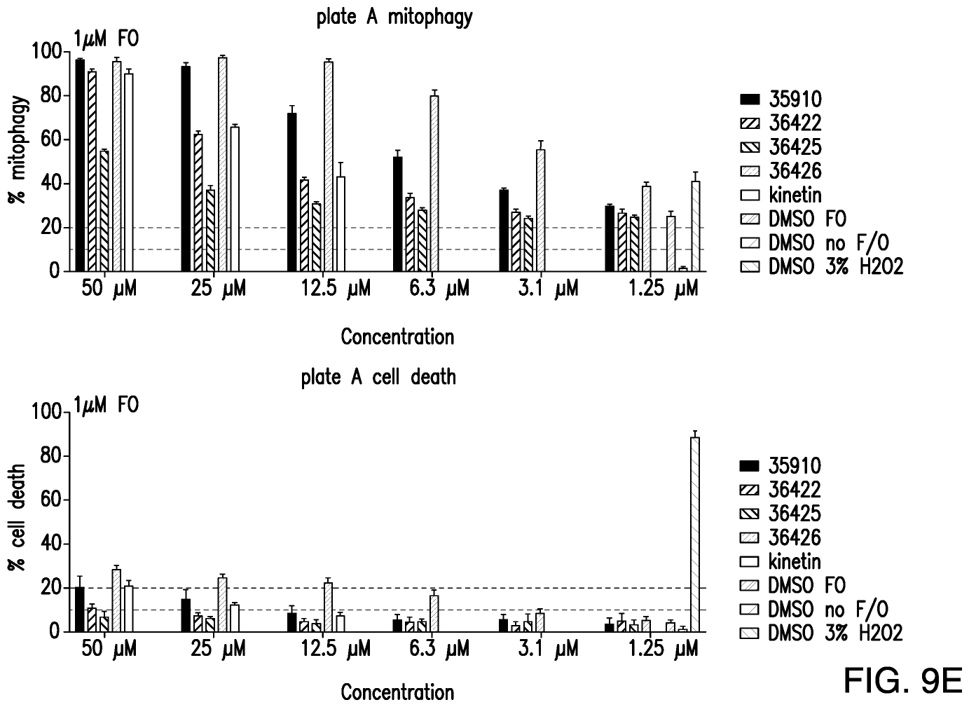

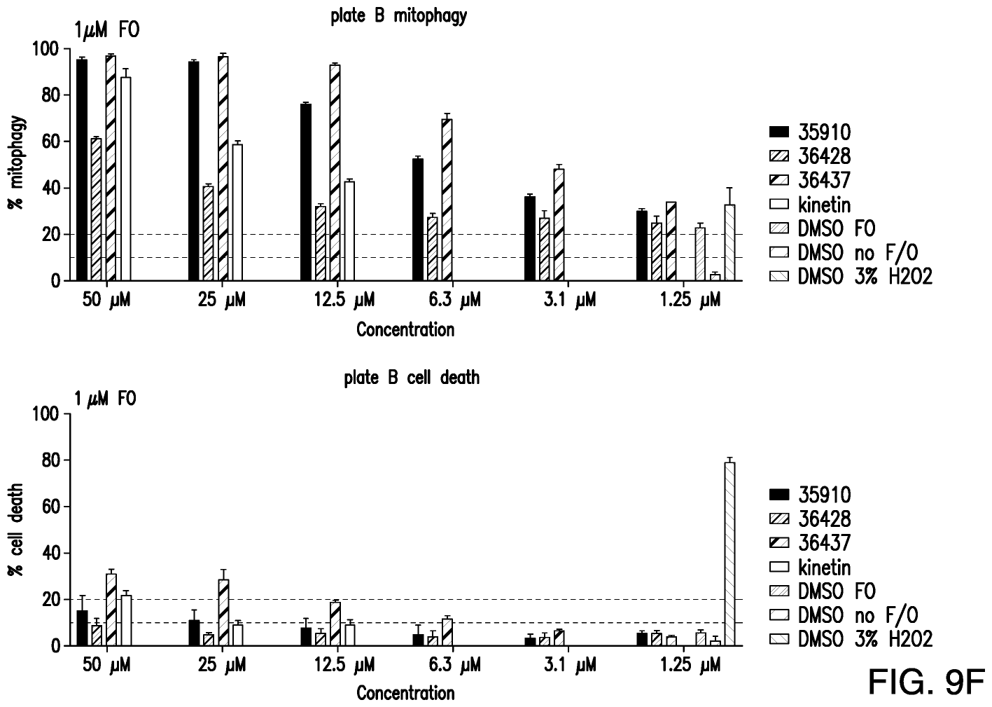

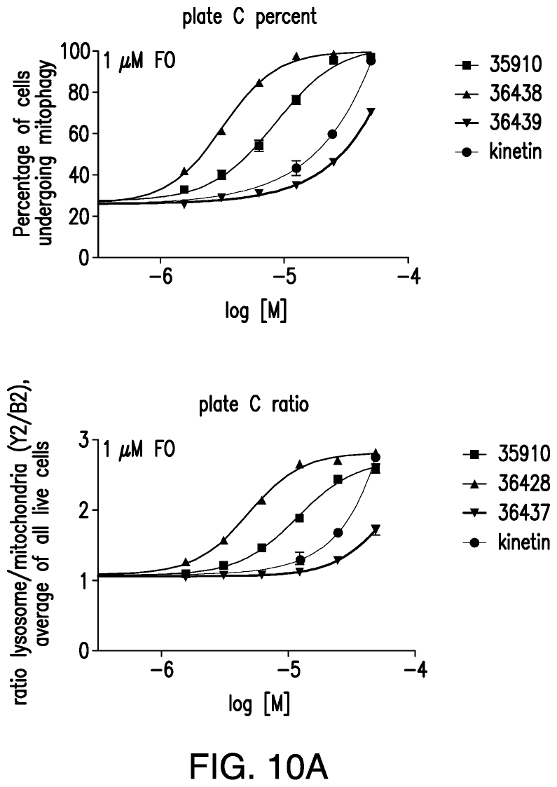

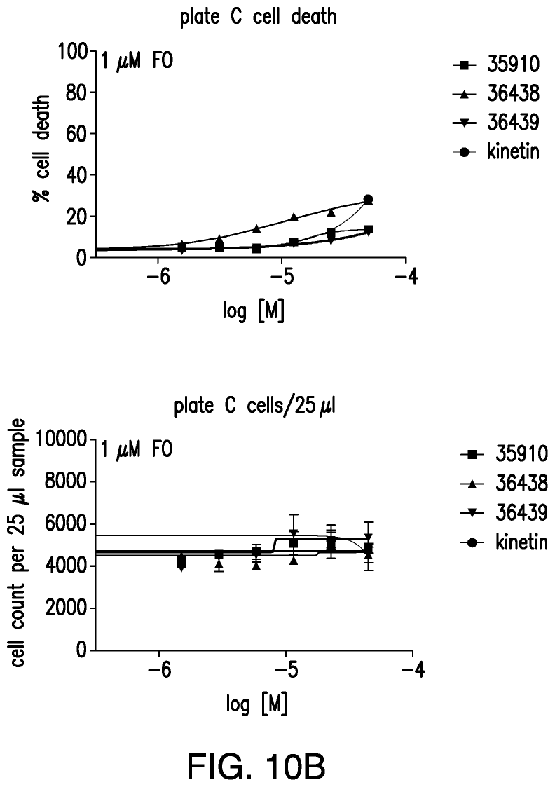

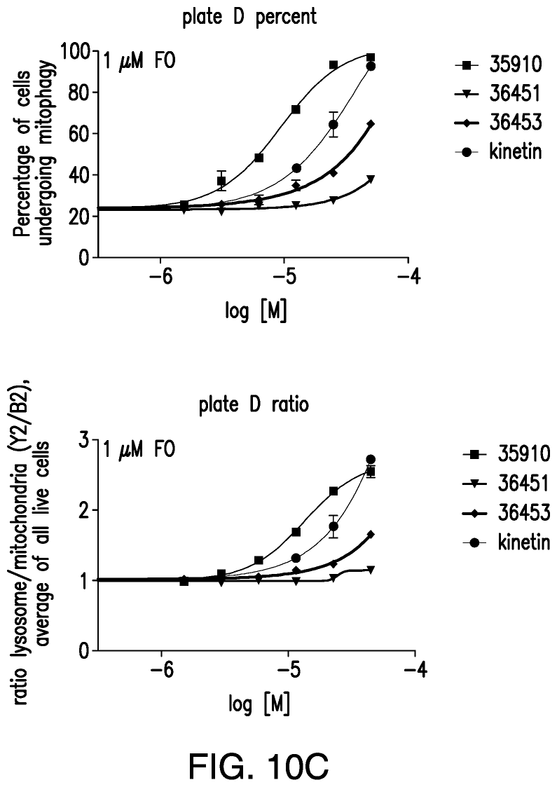

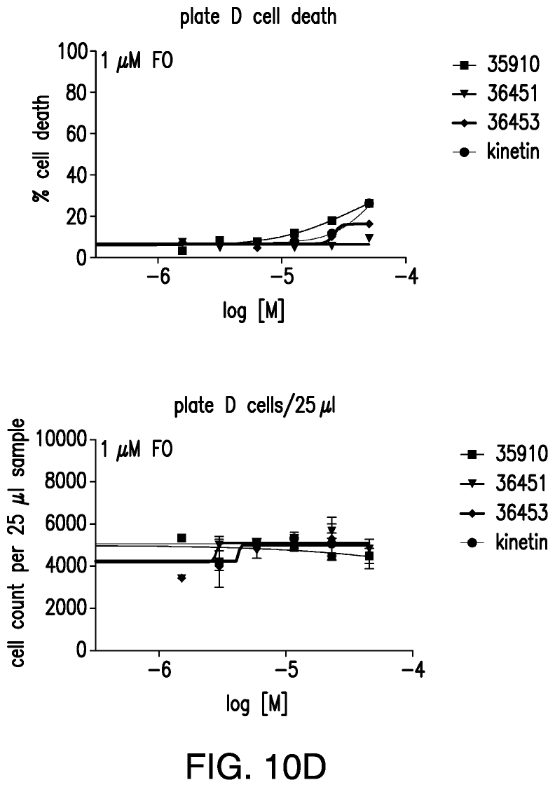

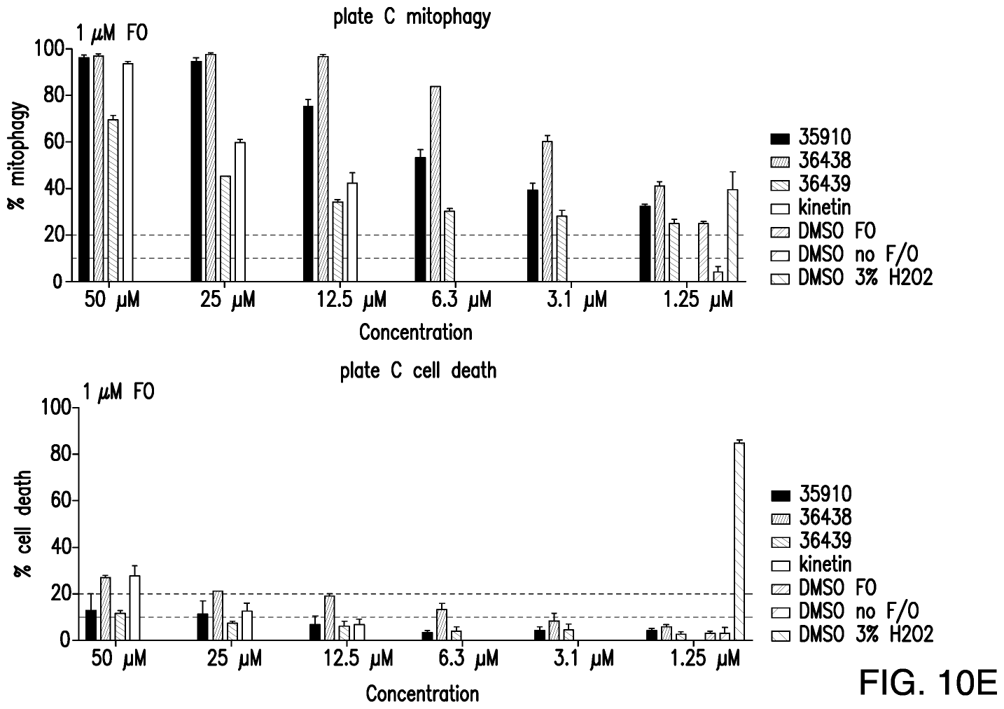

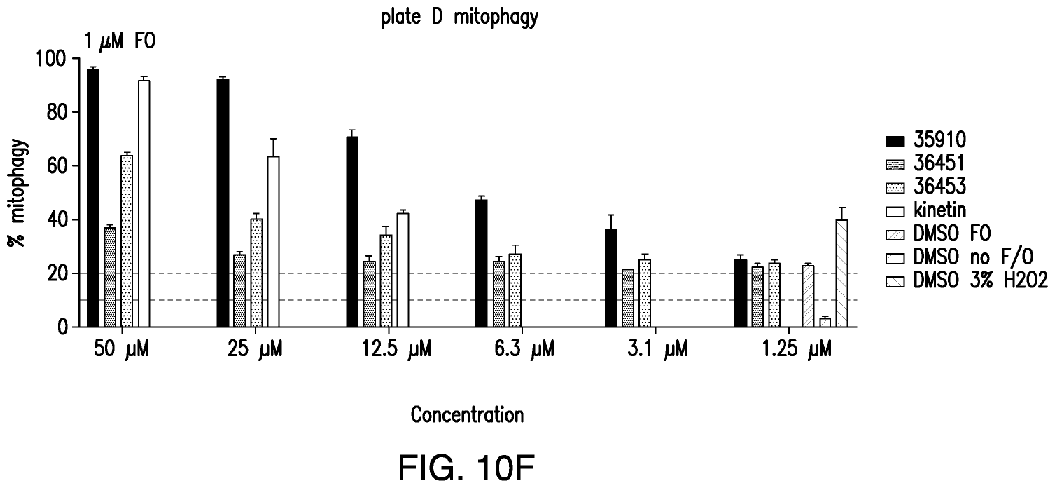

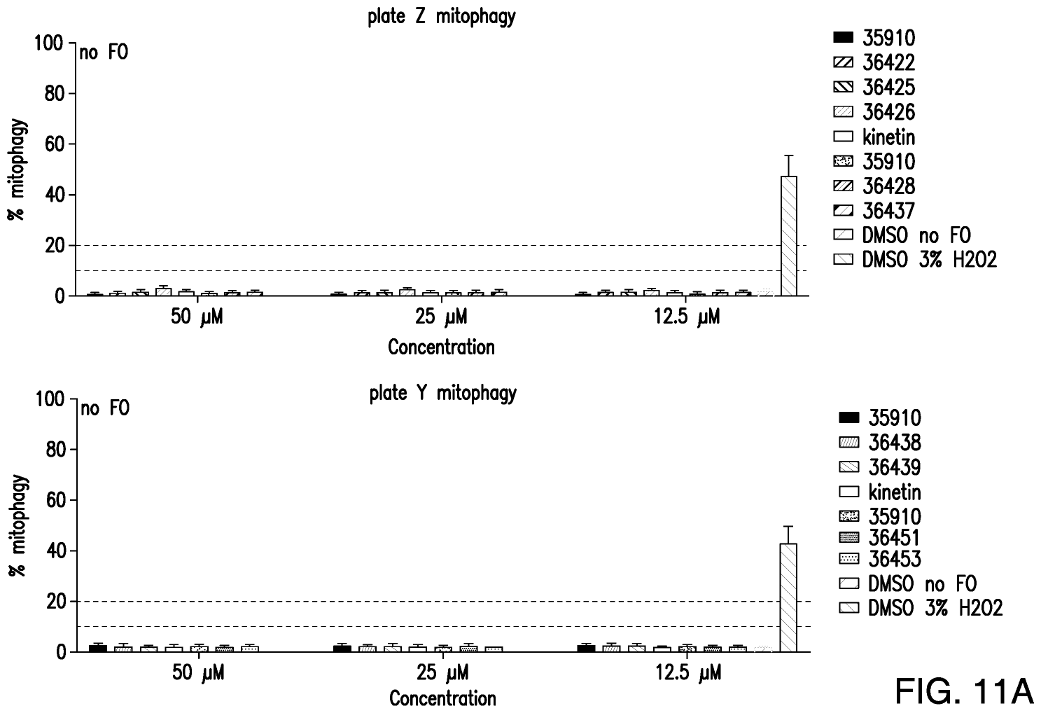

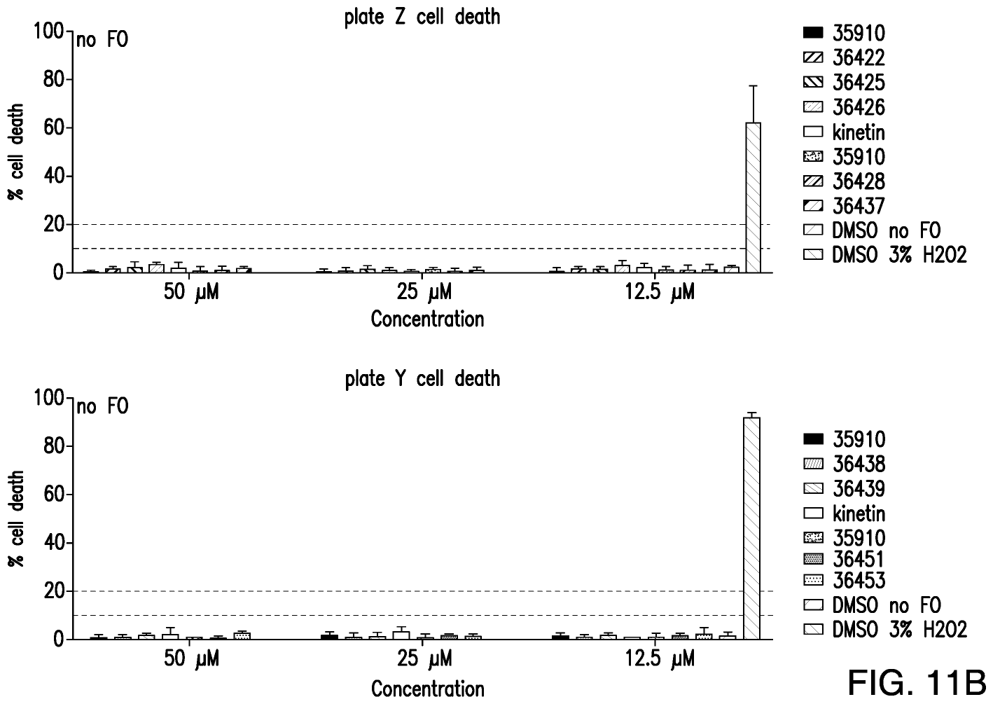

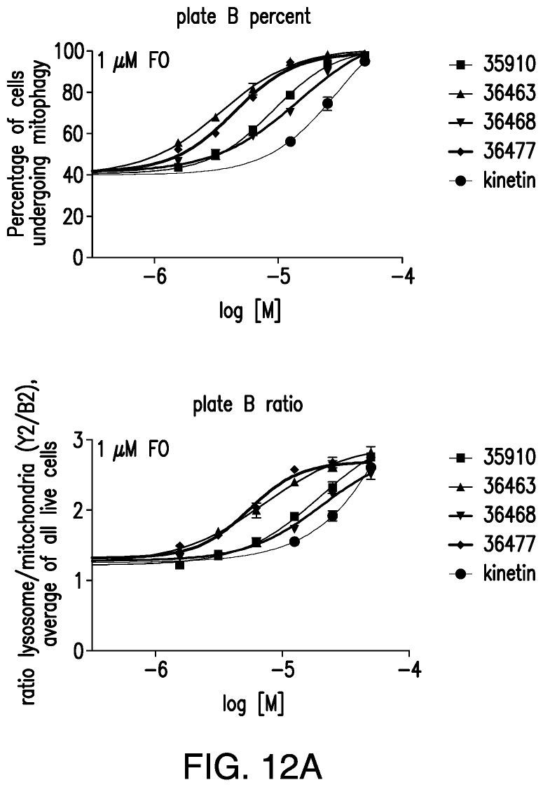

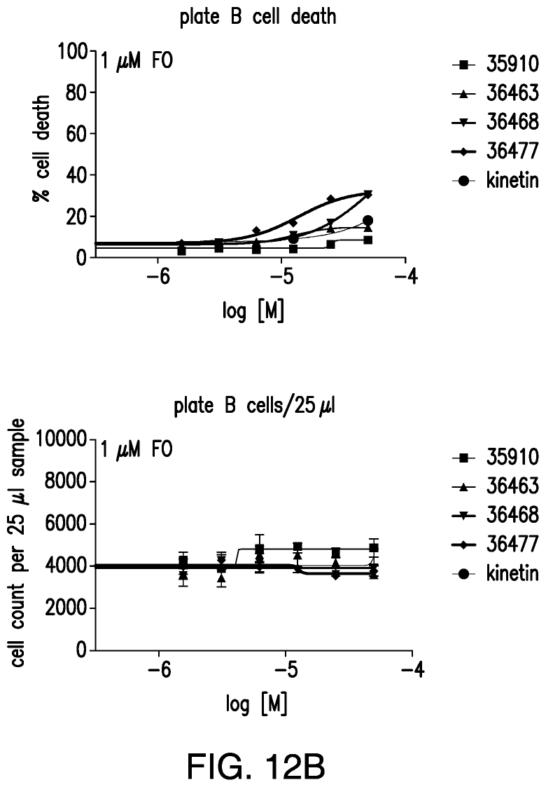

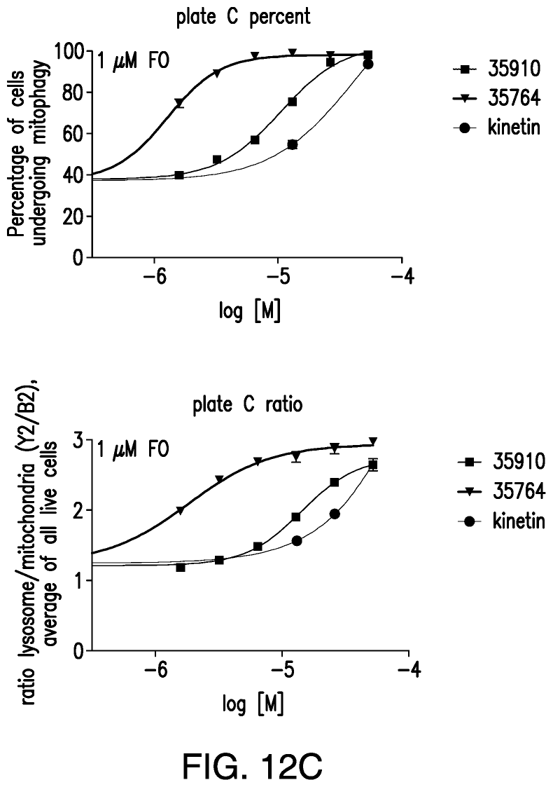

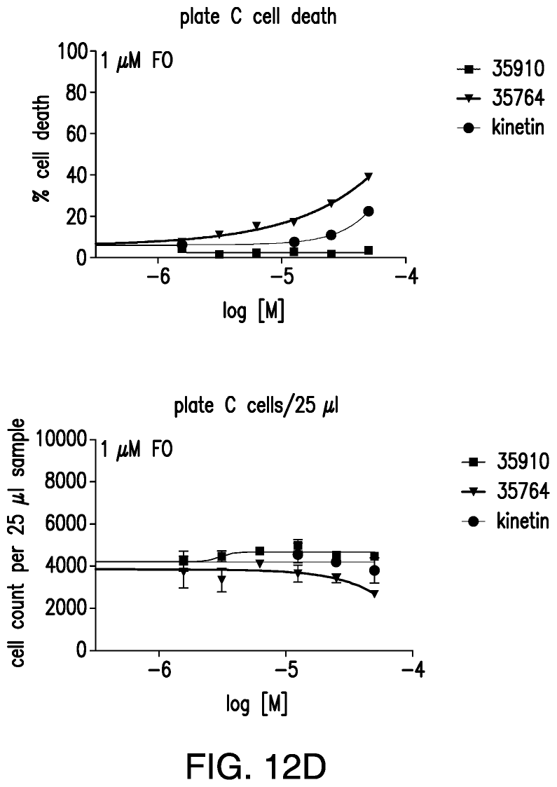

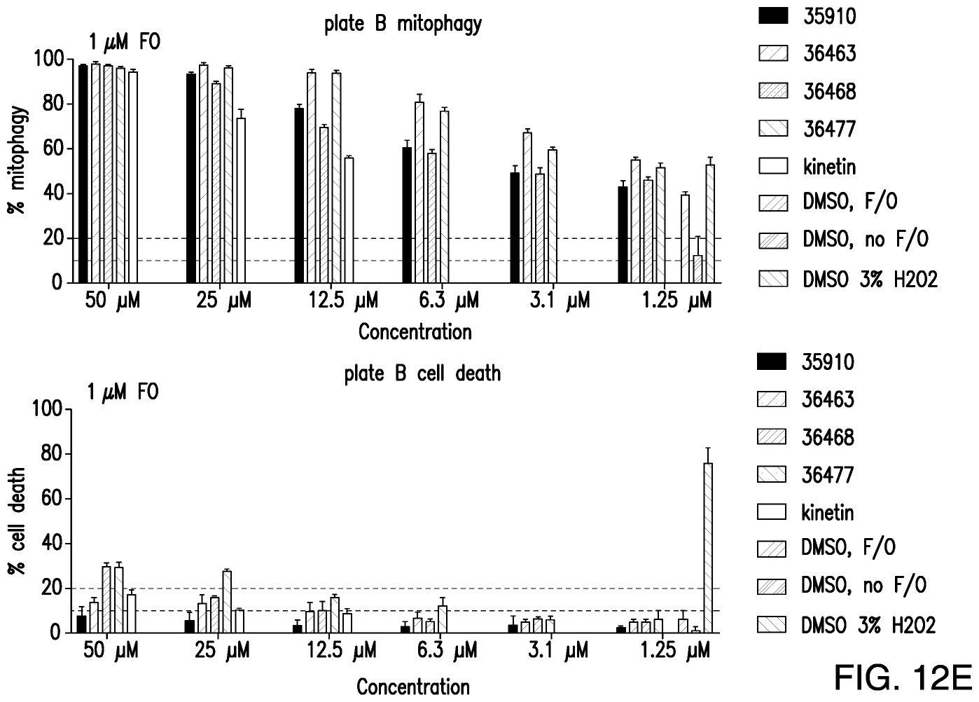

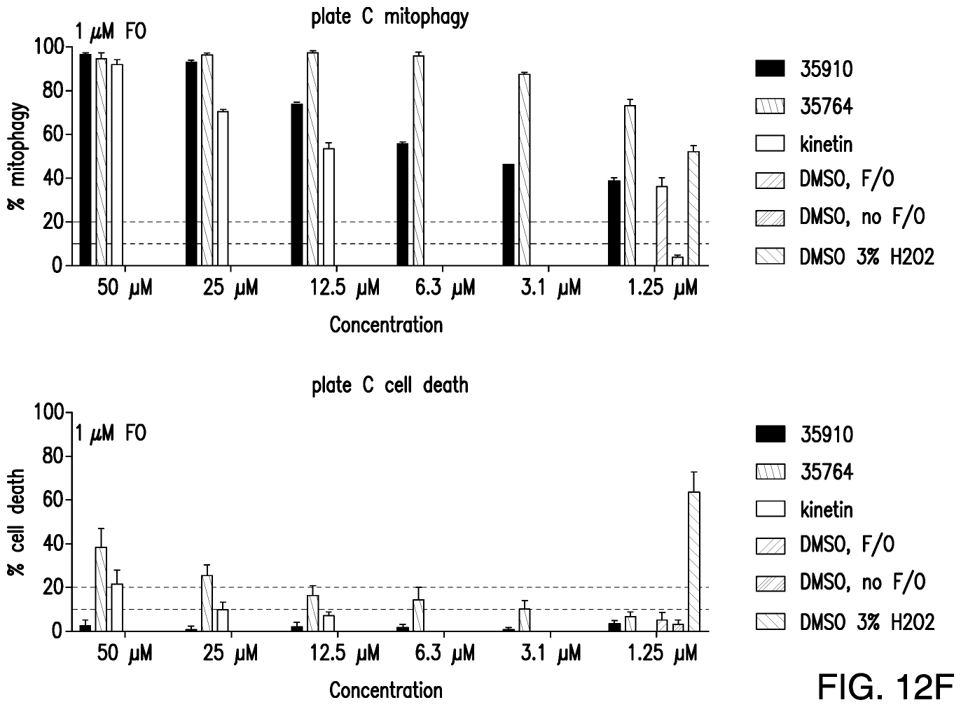

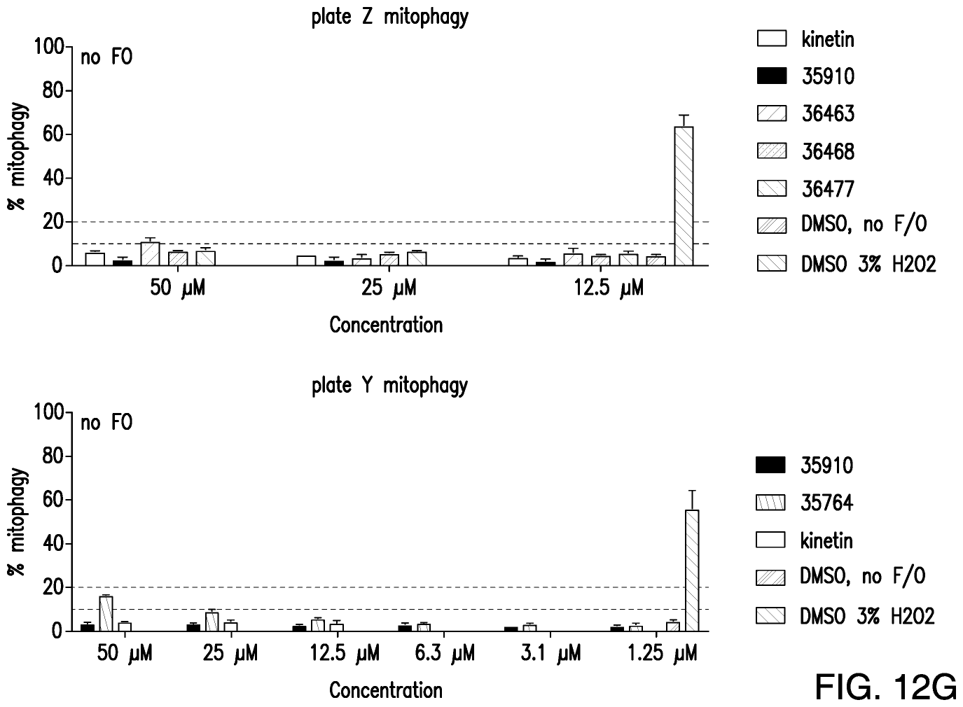

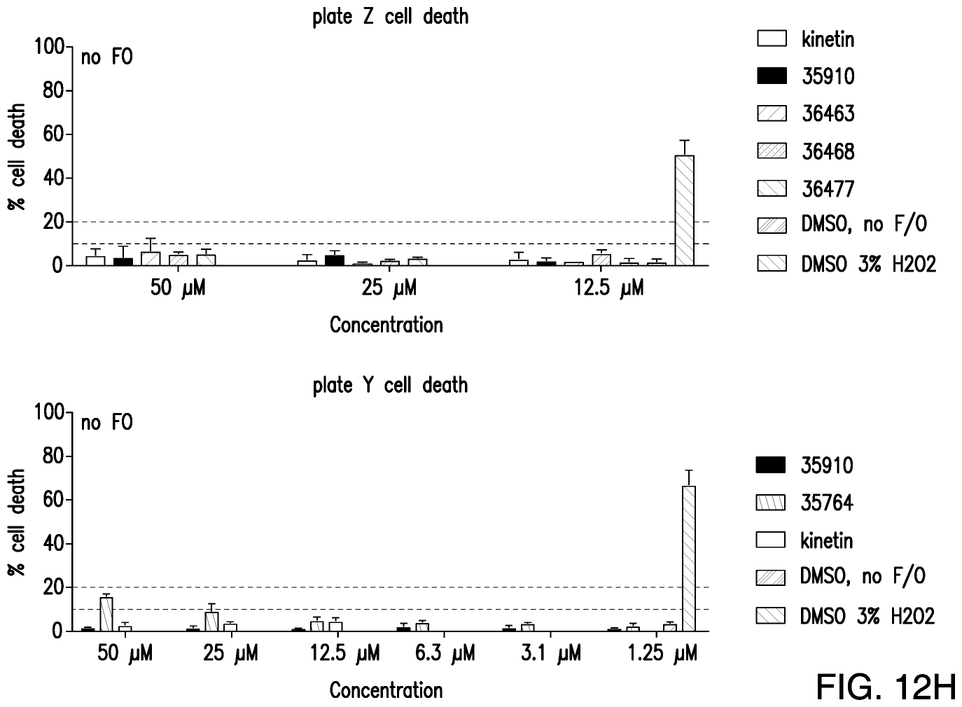

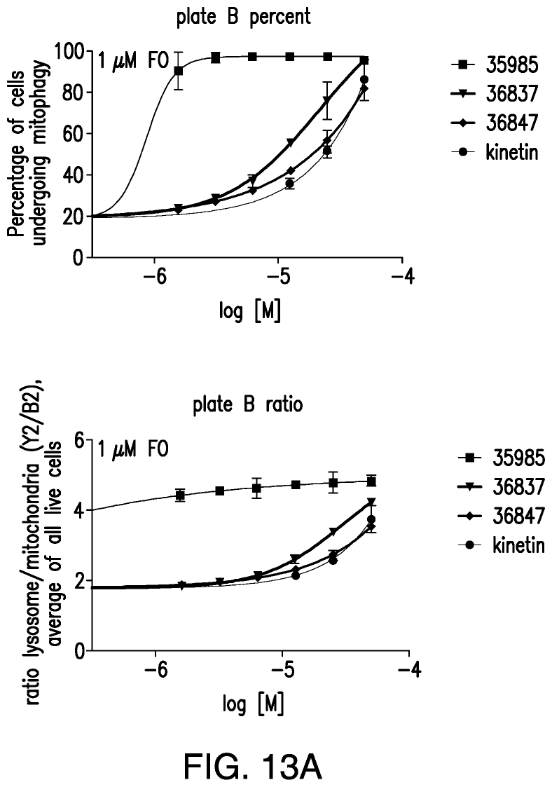

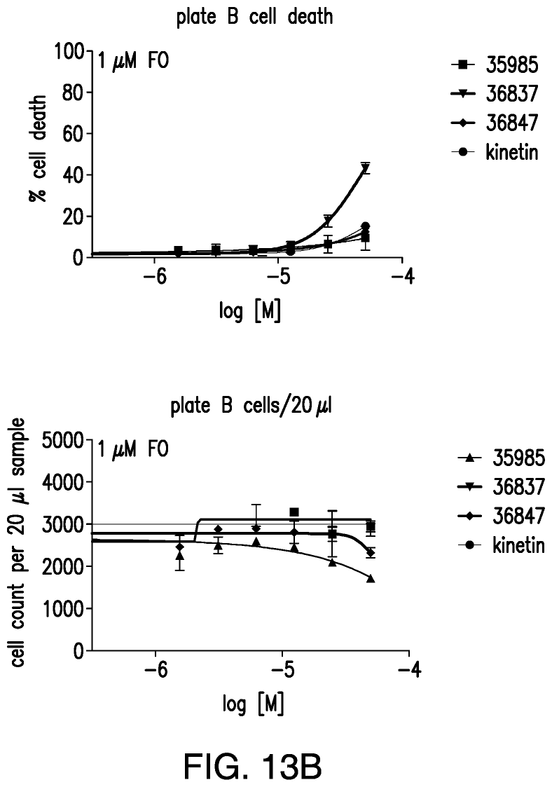

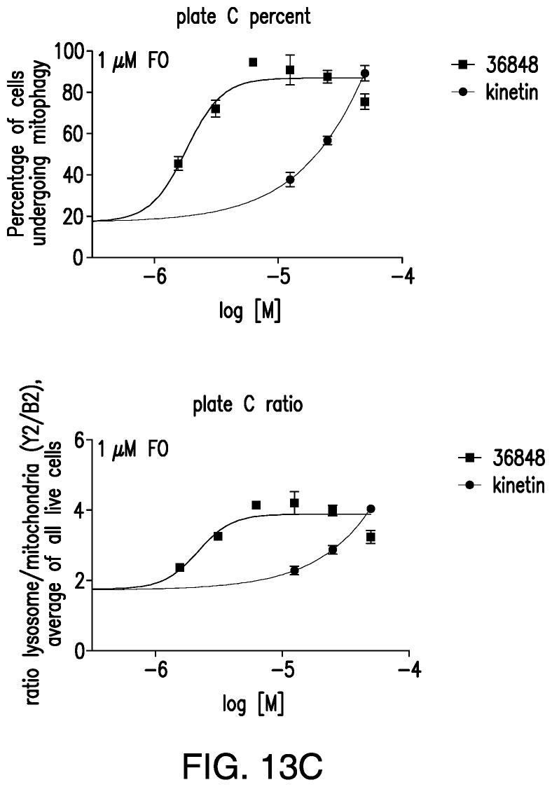

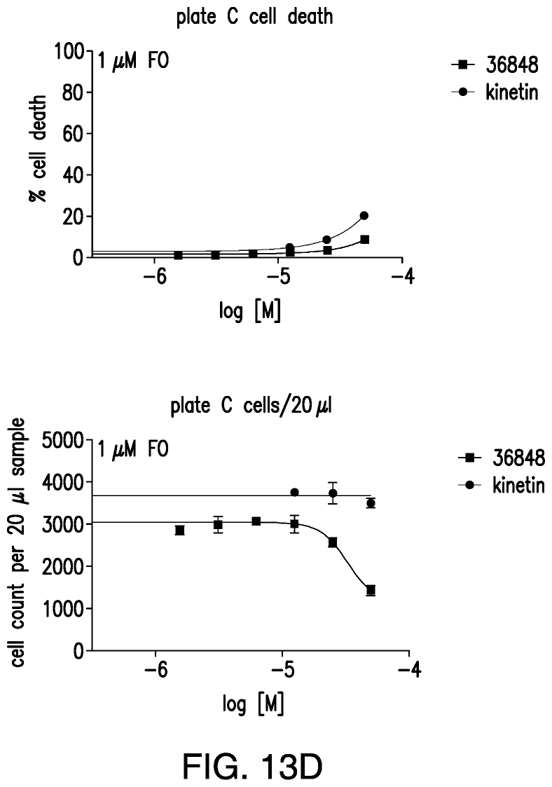

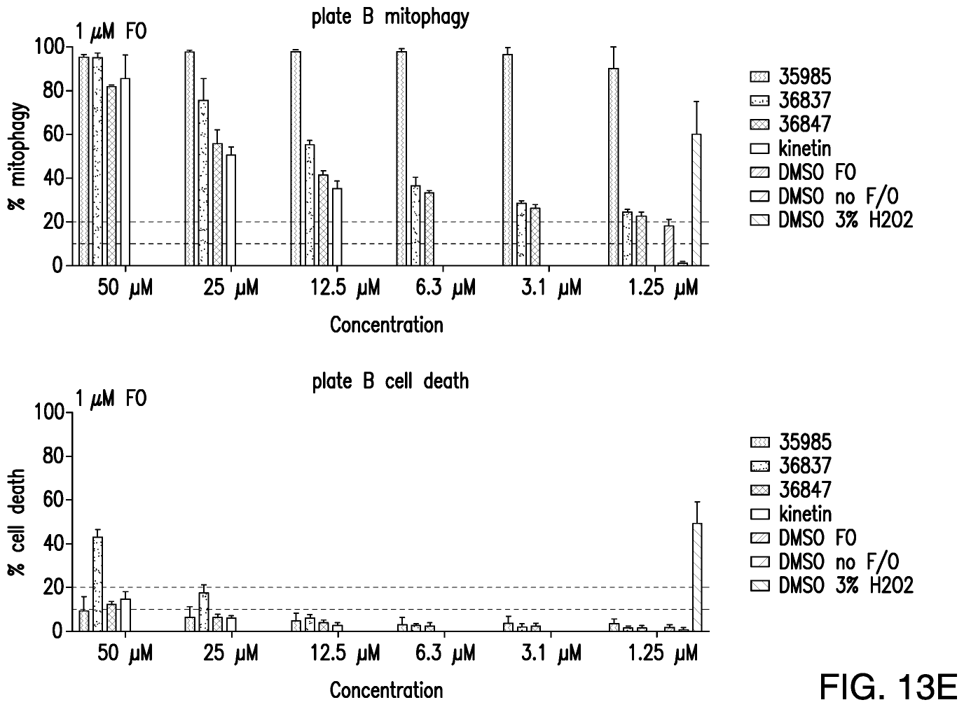

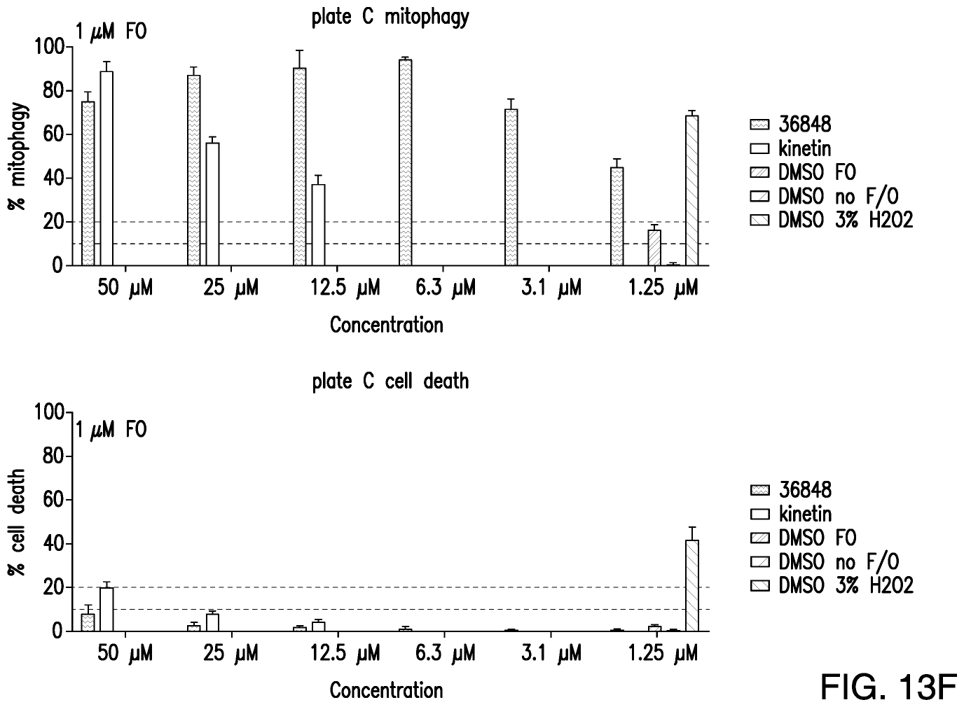

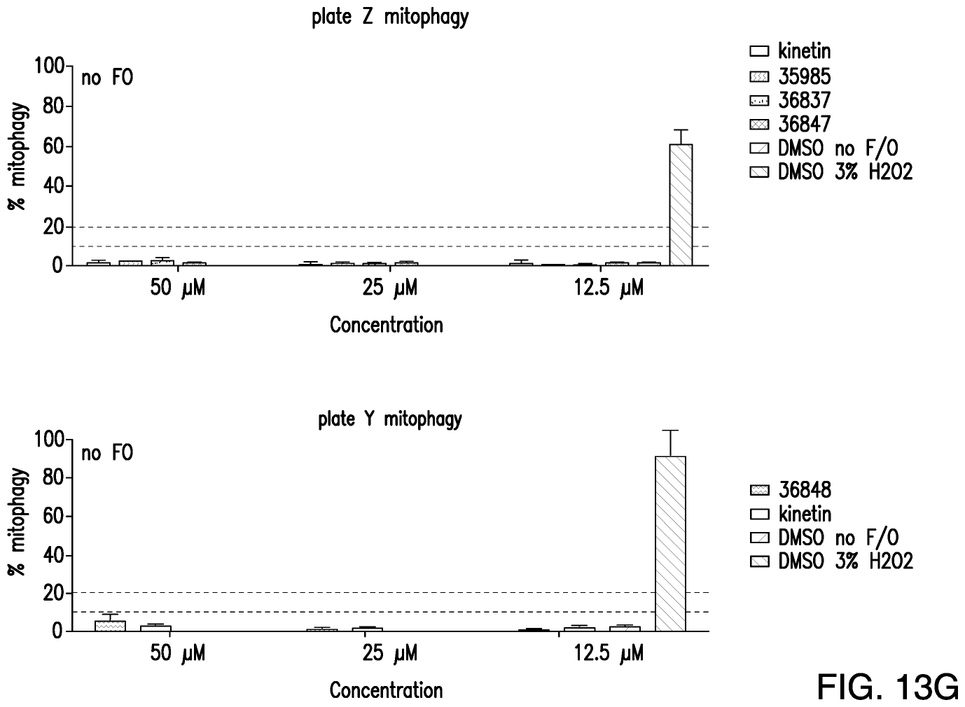

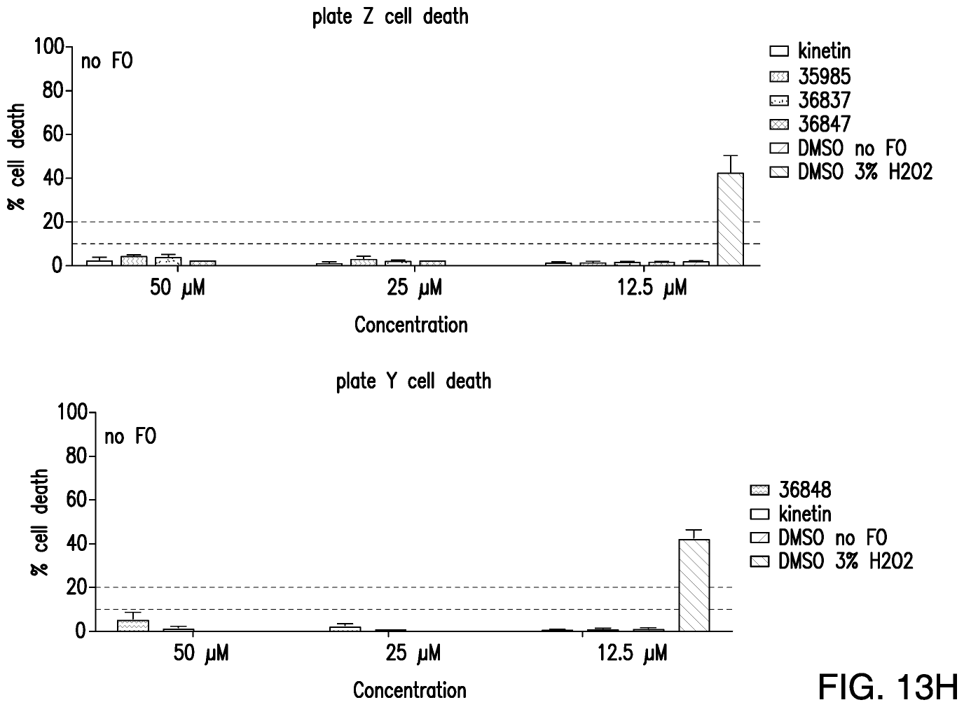

Briefly, HeLa MKYP cells were plated at 10,000 cells/well in 96 well plates along with compounds at various concentrations. Following 16 hrs of incubation, cells were treated with 1 uM FCCP/oligomycin for 6 hours, then analyzed via FACS for the presence of mitochondria in lysosomes (as determined by an emissions spectrum shift from the pH-sensitive mtKeima tag). Examples are shown in FIG. 1 through FIG. 13 below.

### 30. Lipopolysaccharide (LPS) Assay

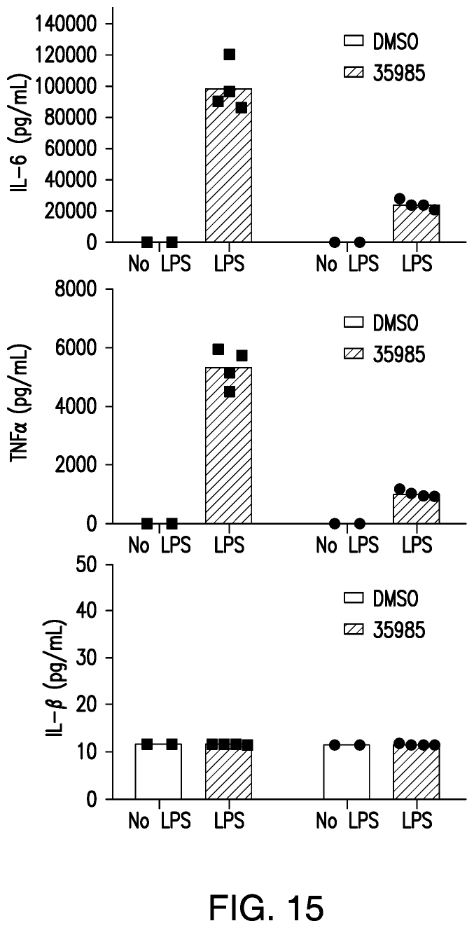

Briefly, P0 to P2 mice were sacrificed and their cortical tissue dissected and plated according to stardard methods to obtain primary mixed cortical cultures. Cultures were maintained for 14 days. On or around Day 15, MTK compound was added and allowed to incubate for 24 hours. After incubation with compound, the cells were challenged with 100 ng/ml LPS. 24 hours after challenge initiation, cellular media was collected for analysis of cytokine levels via ELISA. A commercial ELISA kit for IL-6, TNF-α, and IL1-β was used. The activity of exemplary compounds in a LPS assay is shown in FIG. 15.

### 31. Ornithine Carbamoyltransferase (dOTC) Assay

The expression of a deletion mutant of dOTC yields Triton X-100 insoluble protein aggregates in the mitochondrial matrix. This misfolded protein expression is capable of recruiting PINK1/Parkin to mitochondria without depolarizing the inner mitochondrial membrane. Thus, without wishing to be bound by theory, it may represent a more physiological mechanism of PINK1 stabilization.

Here, HeLa cells stably expressing YFP parkin, containing doxycycline inducible expression of dOTC, were obtained. The cells were seeded at 20000 cells/well plus doxycycline (1 μg/mL) plus MTK on a 96-well plate. On Day 3, the cells were fixed and permeabilized and bound with OTC antibody. DAPI and cell mask were added. There was no wash off of dox. The results were imaged at 40×, non-confocal. 85-600 cells were analyzed per well. Each condition had 1-3 wells.

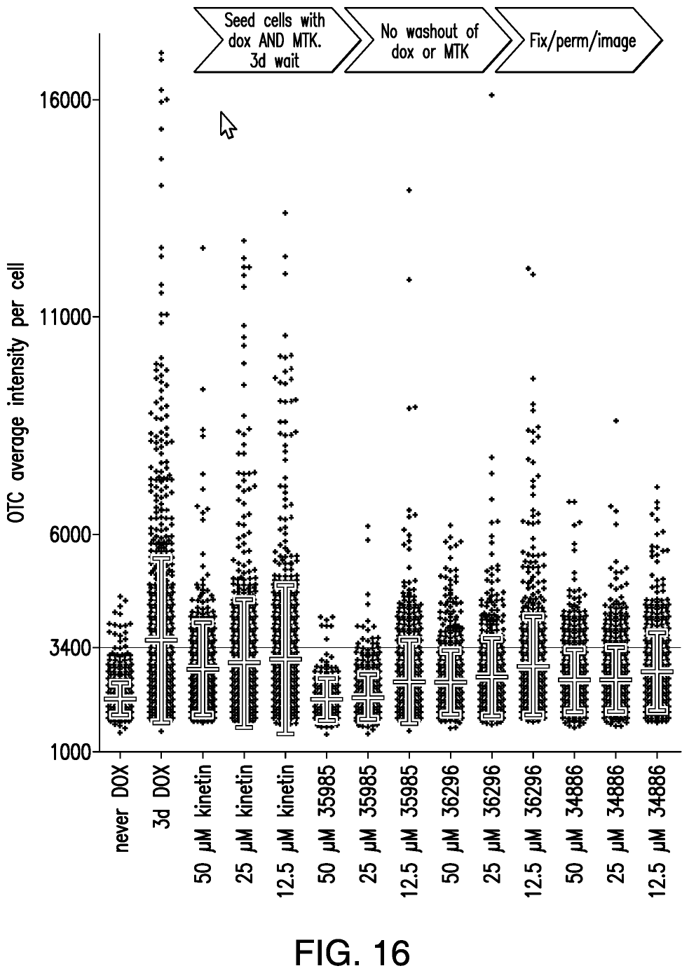

The results of the dOTC assay are shown in FIG. 16. After three days of treatment with DOX and 50 μM EP-0035985, cell counts were lower, though this effect does not appear to be driven by cell death. MTK was very effective at reducing OTC signal; however, some dOTC could be seen remaining in the cells by eye, so it is not completely eliminated.

### 32. pS65 Ubiquitin ELISA Assay

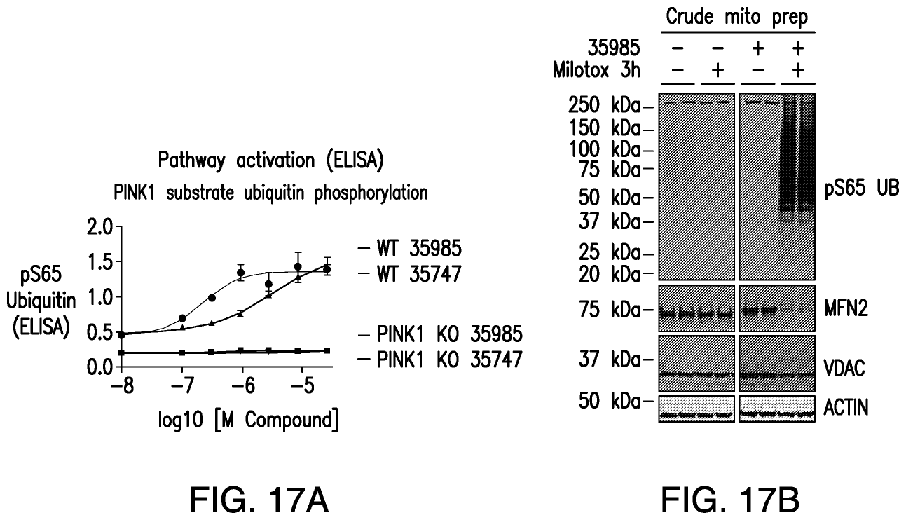

HeLa-MKYP cells were plated at 10,000 cells/well in 96-well plates along with compounds at various concentrations. Following 16 hours of incubation, cells were treated with 1 μM FCCP/oligomycin (F/O) along with compounds at various doses for 2-3 hours. Appropriate controls were included on each plate to obtain a maximum signal from treatment with 10 μM F/O or, and a minimum signal from cells treated with no F/O with DMSO for each cell type tested (HeLa-MKYP WT or PINK1^{KO}). At the desired timepoint, media was removed and cells were frozen prior to lysis. Cells lysates were denatured by boiling, prior to analysis by a custom ELISA assay using commercially available antibodies (using anti-phospho-ubiquitin as a capturing antibody, and anti-total ubiquitin as a detection antibody). Purified pS65 ubiquitin was used to generate a standard curve and determine timing for ELISA reaction development. Representative data are shown in FIG. 17A.

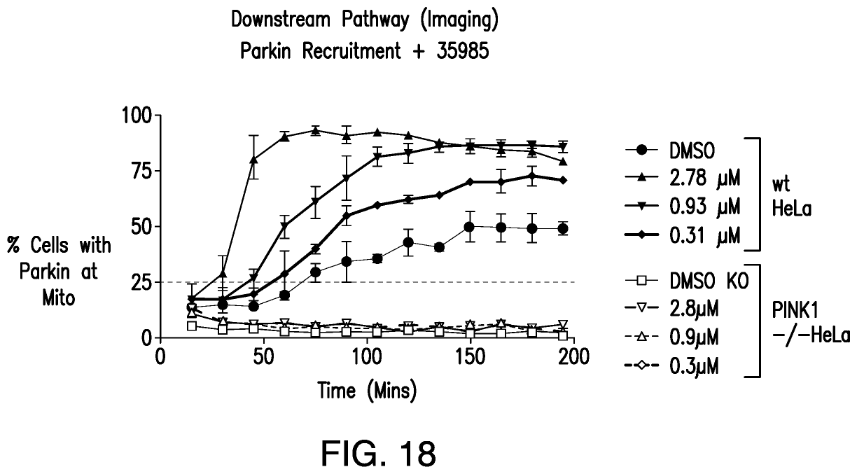

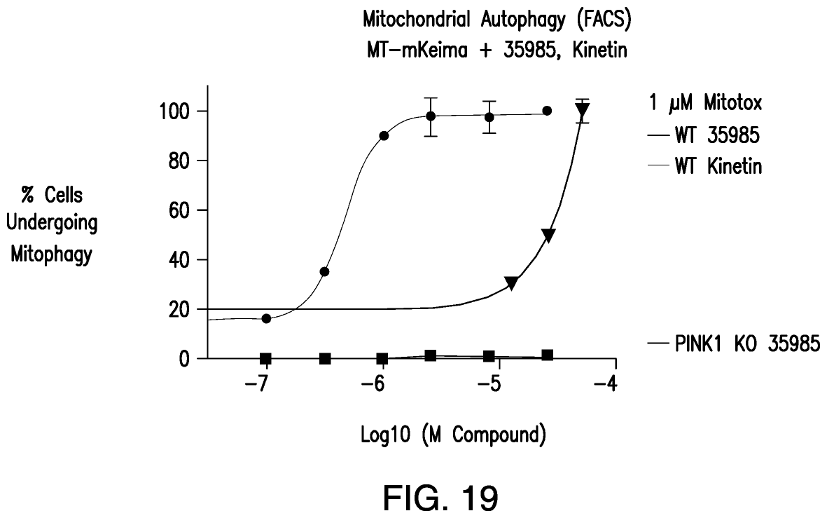

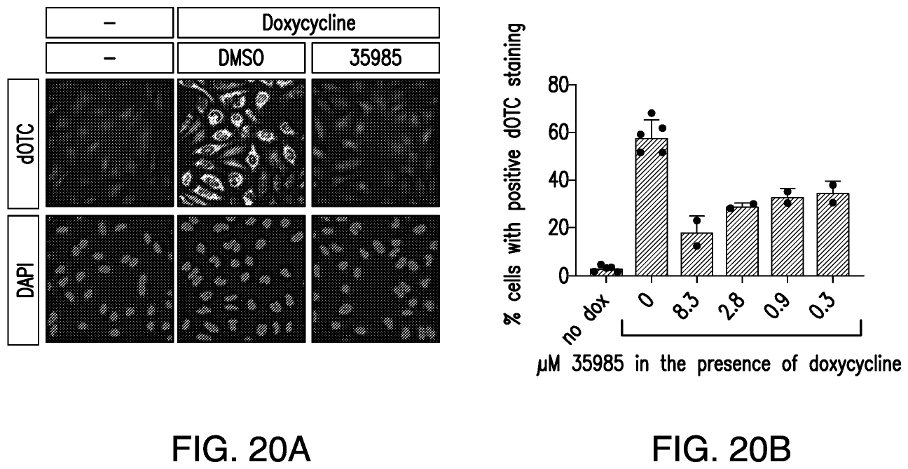

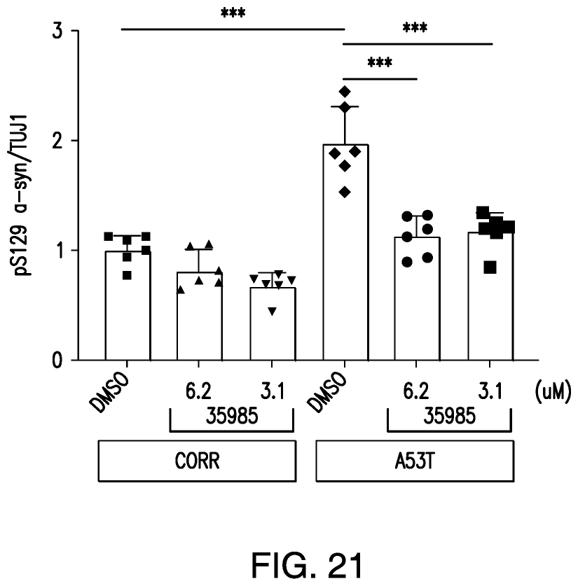

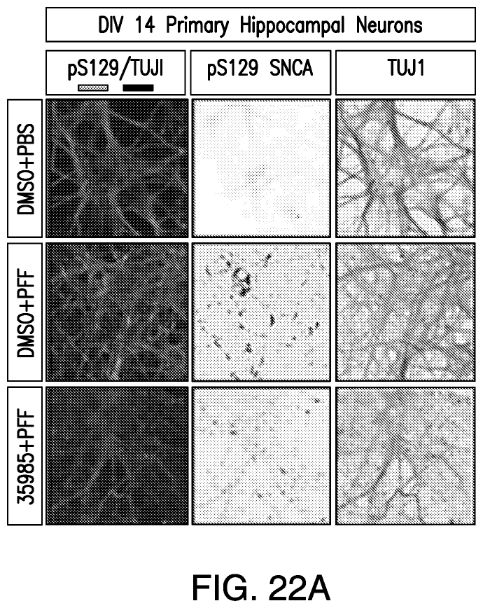

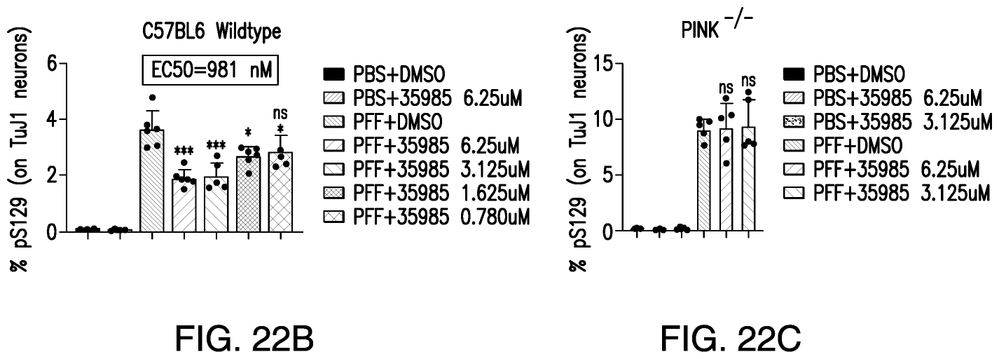

When the PINK1/parkin pathway is activated by FCCP, PINK1 is stabilized on the mitochondrial membrane, and its activity leads to the recruitment of parkin to the mitochondria (Narendra et al., PLoS Biol 2010; Narendra et al., J. Cell Biol 2008; Vives-Bauza et al., PNAS 2009). The aim of this assay is to test how MTK compounds affect the speed of parkin recruitment to the mitochondria. Briefly, HeLa-MKYP cells were plated at 7,000 cells/well in 96-well plates along with compounds at various concentrations. Following 16 hours of incubation, cells were treated in phenol-free media with 1 μM FCCP/oligomycin (F/O) along with compounds at various doses for 2-3 hours. Immediately following addition of new media, the cell treatment plate was transferred to a CO₂ and temperature-regulated chamber in a high-content imaging microscope [Molecular Devices], to acquire images from live cells at regular intervals during the treatment timecourse. Appropriate controls were included on each plate to obtain a maximum signal from treatment with 2 μM F/O, or a minimum signal from cells treated with no F/O with DMSO for each cell type tested (HeLa-MKYP WT or PINK1^{KO}). Images were analyzed using MetaExpress 6 software package, and automatically processed after setting thresholds to define overlap between the YFP-parkin signal and red mitochondrial signal based on controls. The percent of cells with parkin recruitment to mitochondria is represented for each dose of MTK-compound tested in each cell type, as shown in FIG. 18.

### 33. Preformed α-syn Amyloid Fibril (PFF) Primary Neuron Model

Briefly, primary hippocampal neurons were derived and cultured following standard protocols. At DIV-7, the cultures were treated with PFFs. At DIV-10, the cultures began treatment with a Mitokinin compound or vehicle controls, such treatment continuing for the duration of the experiment. The cultures were fixed and stained for alpha synuclein, TUJ1, and MAP2 at DIV-14, and analysis performed by unbiased imaging on an ImageXpress Confocal microscope and quantified by Molecular Devices' MetaXpress software.

### 34. Preformed α-Syn Amyloid Fibril (PFF) Mouse Model

Animals. C57BL6 mice were obtained from the Jackson Laboratories.

Injection material and stereotaxic injections. Purification of recombinant α-synuclein proteins and in vitro fibril assembly was performed as previously described (K. C. Luk et al., Intracerebral inoculation of pathological α-synuclein initiates a rapidly progressive neurodegenerative α-synucleinopathy in mice. J. Exp. Med. 209, 975 (2012)) from full-length wildtype mouse α-synuclein (5 mg/mL). Assembly reactions were agitated in an Eppendorf orbital mixer (1,000 rpm at 37° C.) and α-synuclein pre-formed fibrils (PFFs) harvested after 5 d. Preparations were diluted in sterile PBS and sonicated briefly with a hand-held probe sonicator (Fisher Scientific Model 120) prior to injection. Mice between 2 and 3 months of age were anesthetized with ketamine hydrochloride (100 mg/kg, i.p.) and xylazine (10 mg/kg, i.p.) and stereotaxically injected in one hemisphere of the striatum with PFFs (5 μg). Control animals received sterile PBS. To target the striatum, a single needle insertion (co-ordinates: +1.0 mm relative to Bregma, +2.0 mm from midline) into the right forebrain was used (+2.6 mm beneath the dura). Injections were performed using a motorized injector at a rate of 0.2 μL per min (2.5 μL total per site) with the needle in place for >5 min at each target. Animals were monitored regularly following recovery from surgery. Starting 7 days after surgery, a daily oral dose of EP-0035985 or vehicle control was administered, continuing for the duration of the study. The animals were sacrificed at 30 days post injection by overdose with isofluorane, and their brains removed following transcardial perfusion with PBS. Brains were sectioned into striatum and ventral midbrain sections, and immediately frozen and stored at −80° C. until used.

Biochemical analysis. Dissected brain regions of interest were extracted as in Yun, P. S., et al Nat Med. 2018 July; 24(7):931-938. Briefly, sections were homogenized in brain lysis buffer containing 10 mM Tris-HCl, pH 7.4, 150 mM NaCl, 5 mM EDTA, 0.5% IGEPAL® CA-630 (Nonidet P-40), 1× Halt™ Protease and Phosphatase Inhibitor Cocktail. 0.2 mL of buffer was used per tissue section. Samples were homogenized using a mortar pestle and then 0.3 mL brain lysis buffer was added before clearing at 300×g for 3 minutes at 4° C. Supernatant was then transferred to a new tube and cleared at 22,000×g for 20 min at 4° C. The supernatant should be transferred and the pellet washed with 0.3 mL brain lysis buffer before repeating the clearing step. Subsequent to this the pellet should be further homogenized (sonicating for 20 s at 20% amplitude on ice) in 0.2 mL per sample SDS-Brain lysis buffer containing 10 mM Tris-HCl, pH 7.4, 150 mM NaCl, 5 mM EDTA, 0.5% Nonidet P-40, 1% SDS, 0.5% sodium deoxycholate. Protein concentrations were determined using the BCA assay (Pierce) and samples (10 μg total protein) were separated on SDS-polyacrylamide gels (4-20% gradient) and transferred onto nitrocellulose membranes. Blots were blocked in 5% BSA in TBST and probed using various primary antibodies. Target antigens were detected using an Odyssey FC scanner (LiCor) following incubation with the appropriate infrared secondary antibodies.

### 35. In Vivo Pharmacokinetic Properties of EP-0035985

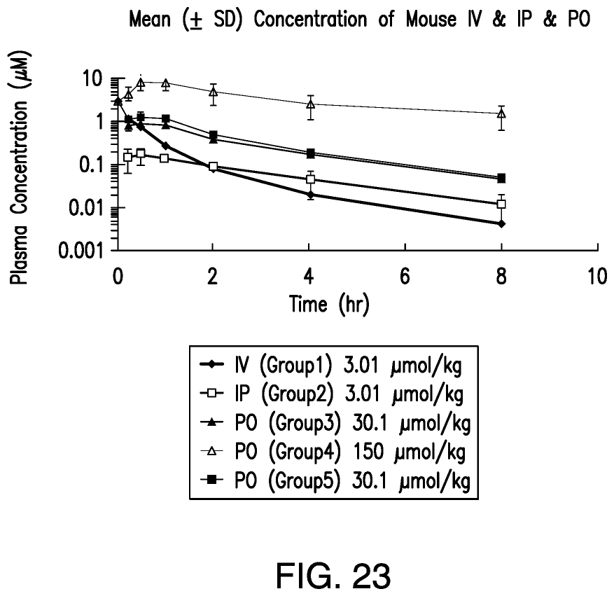

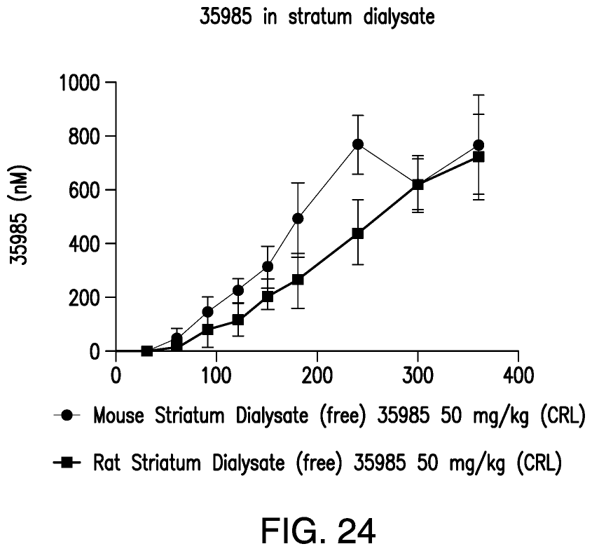

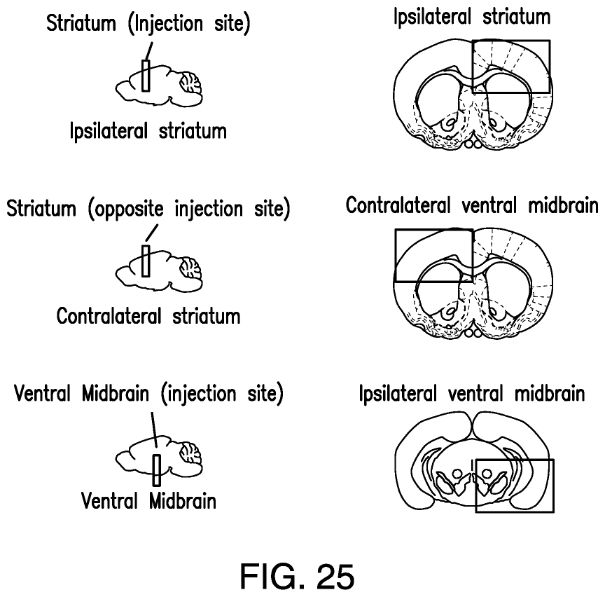

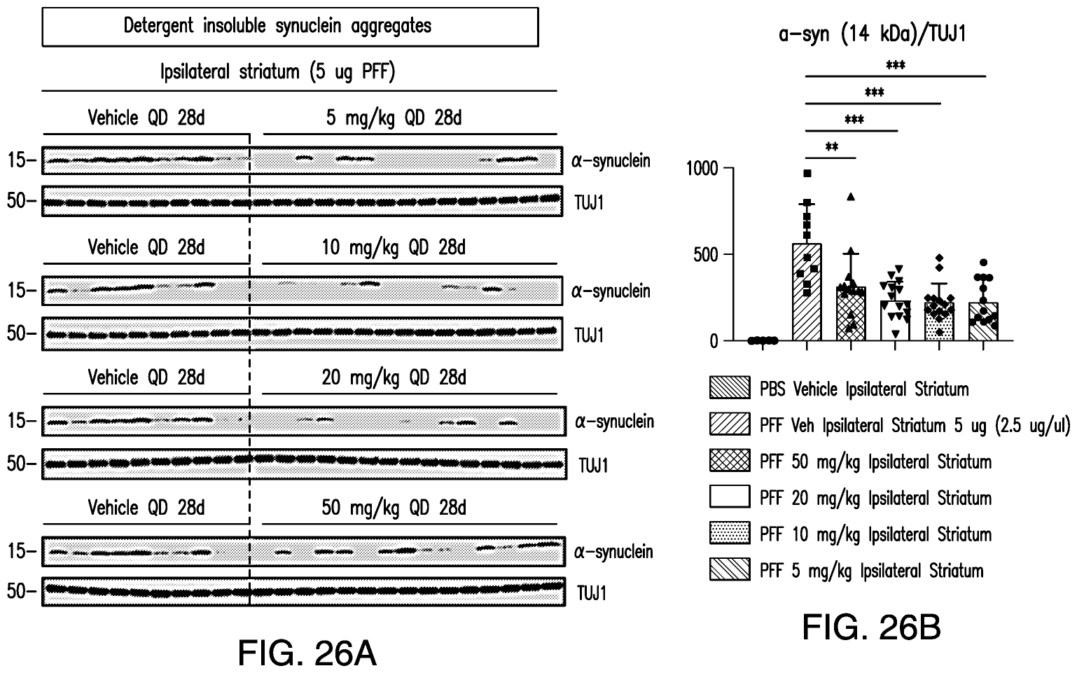

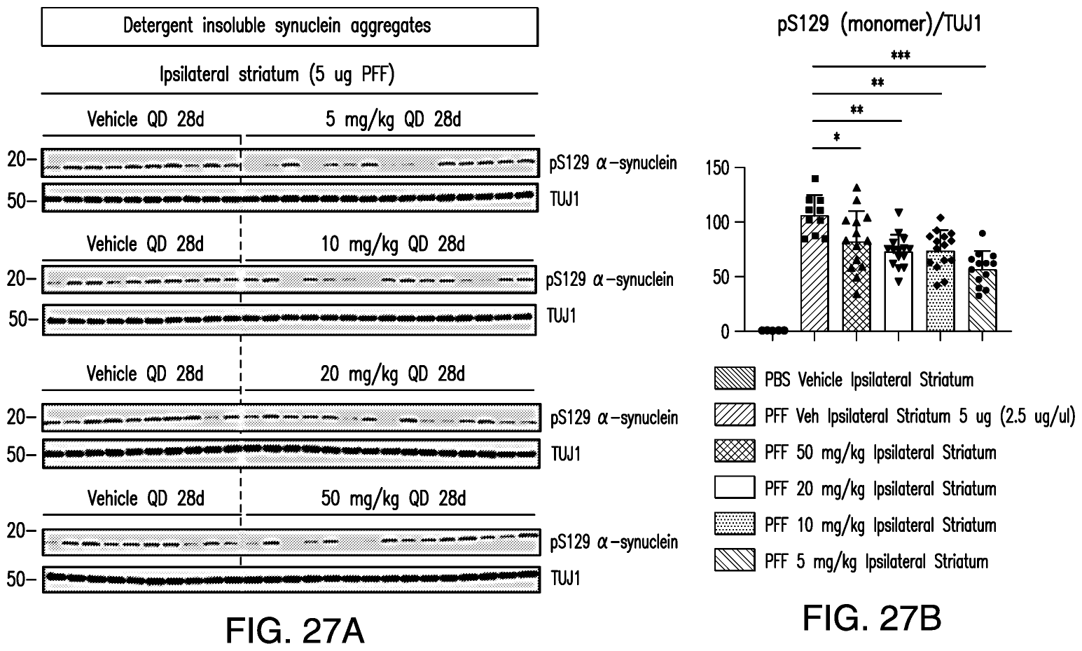

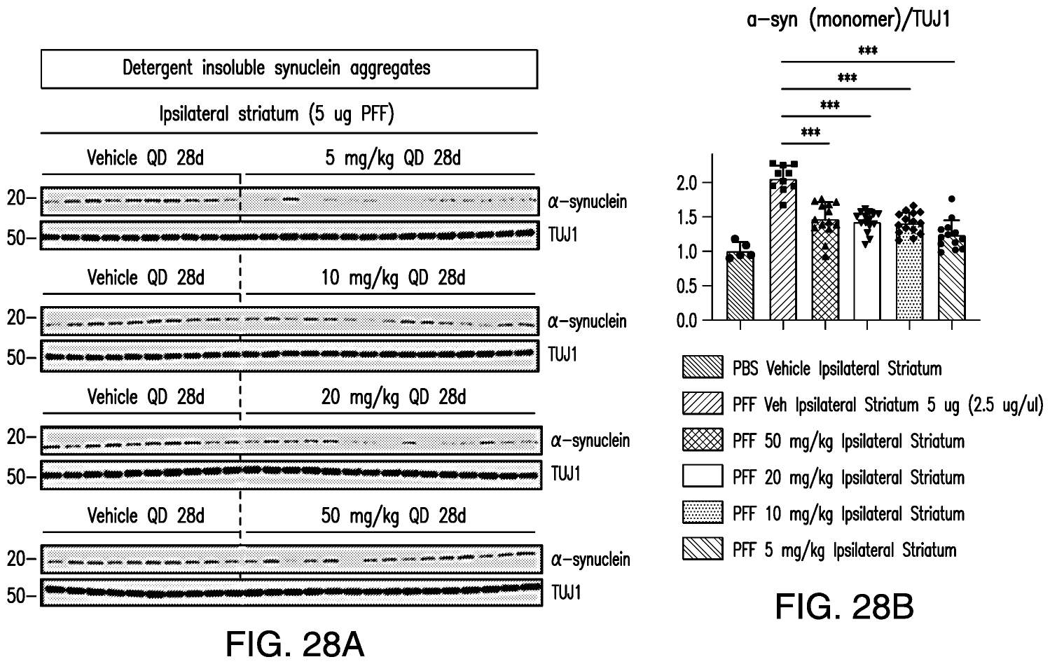

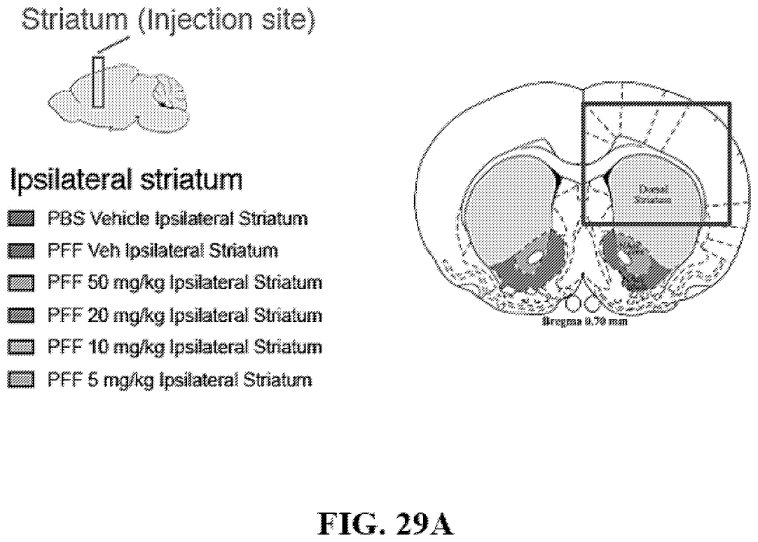

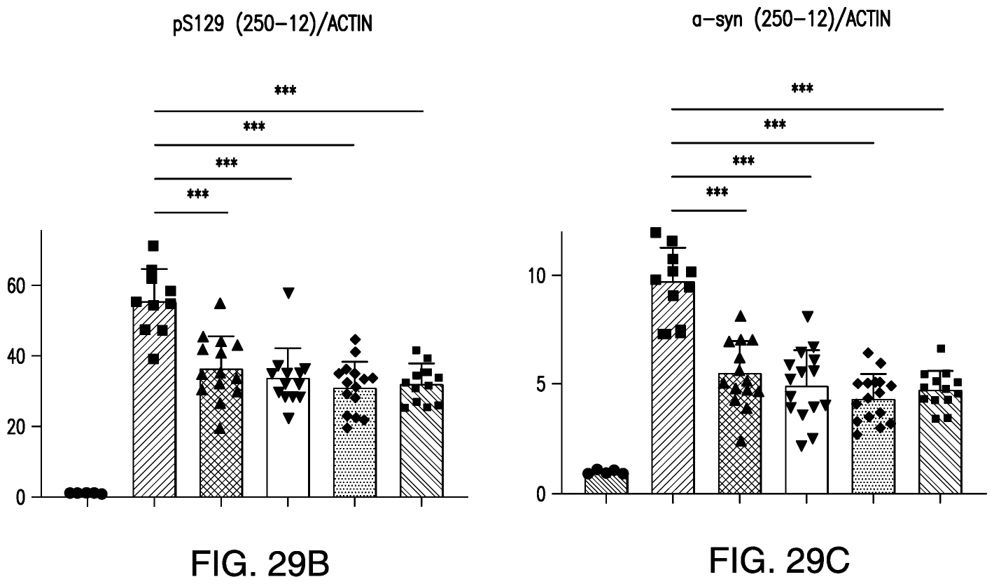

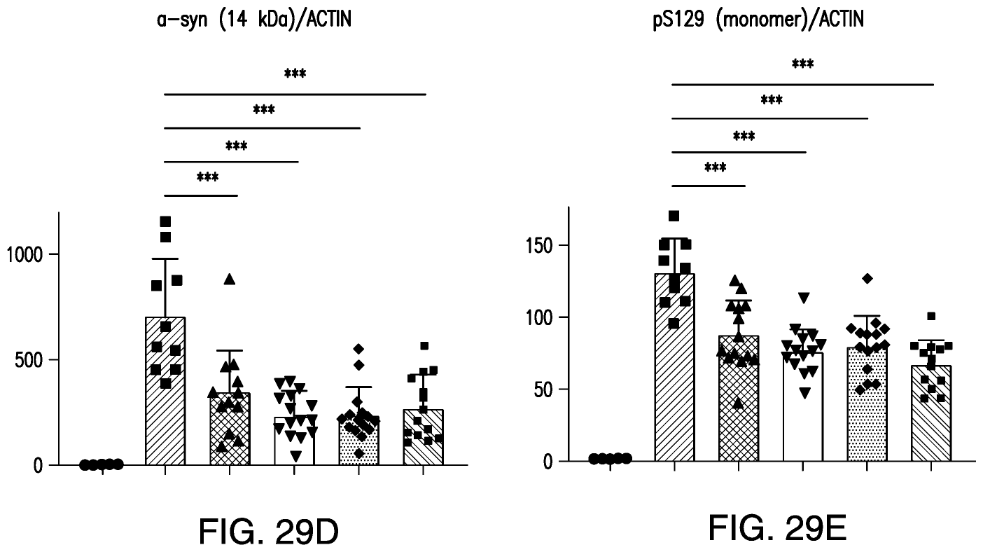

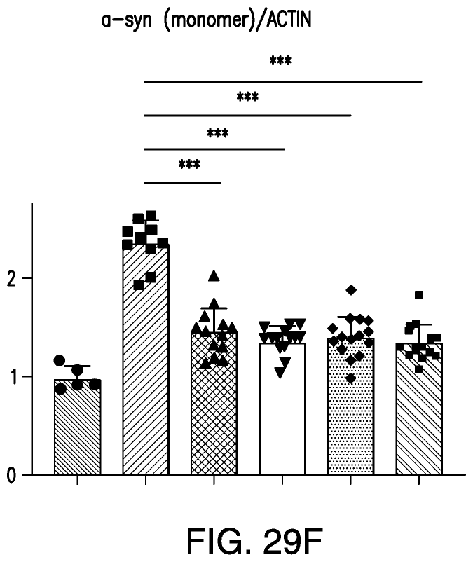

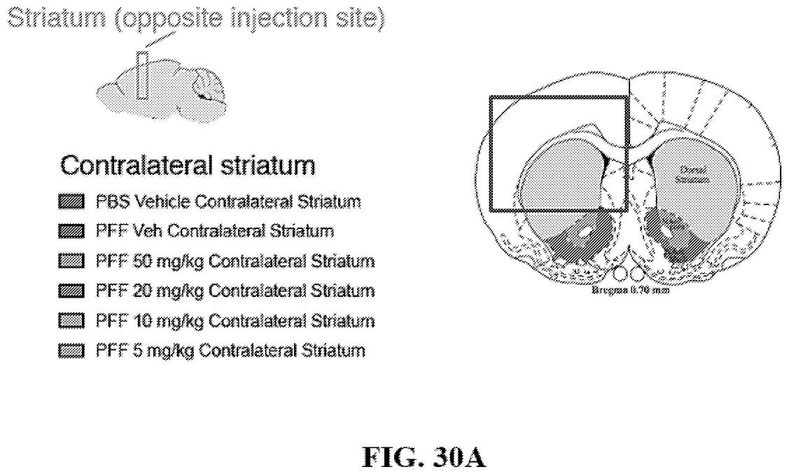

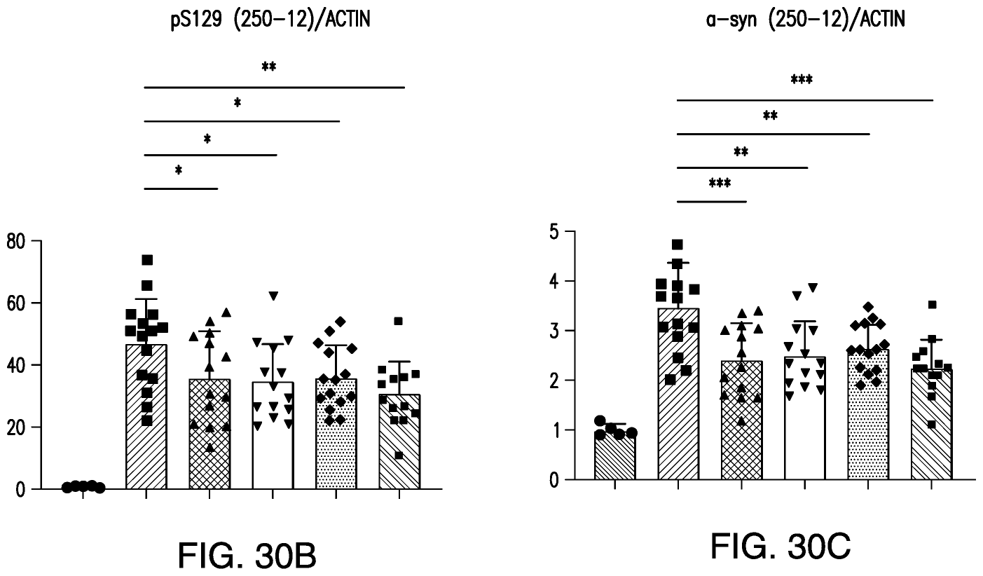

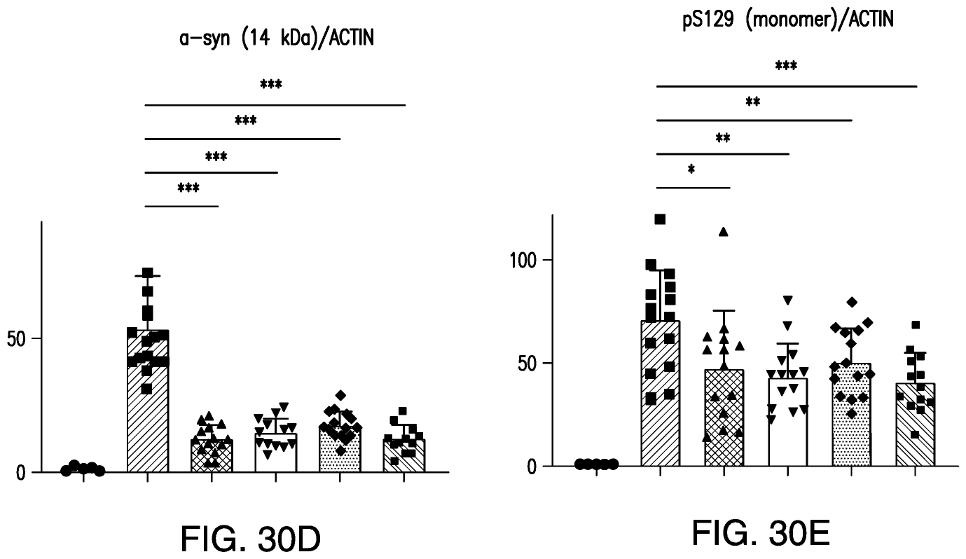

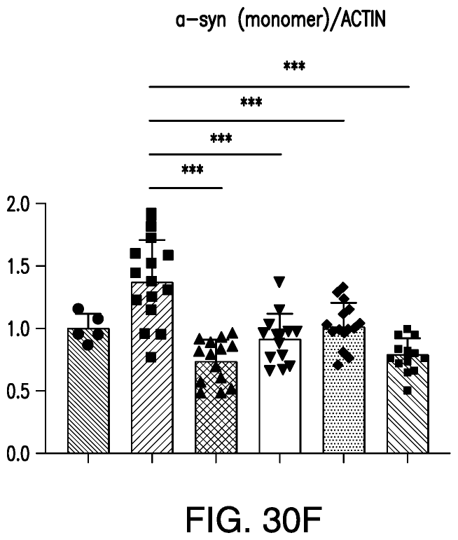

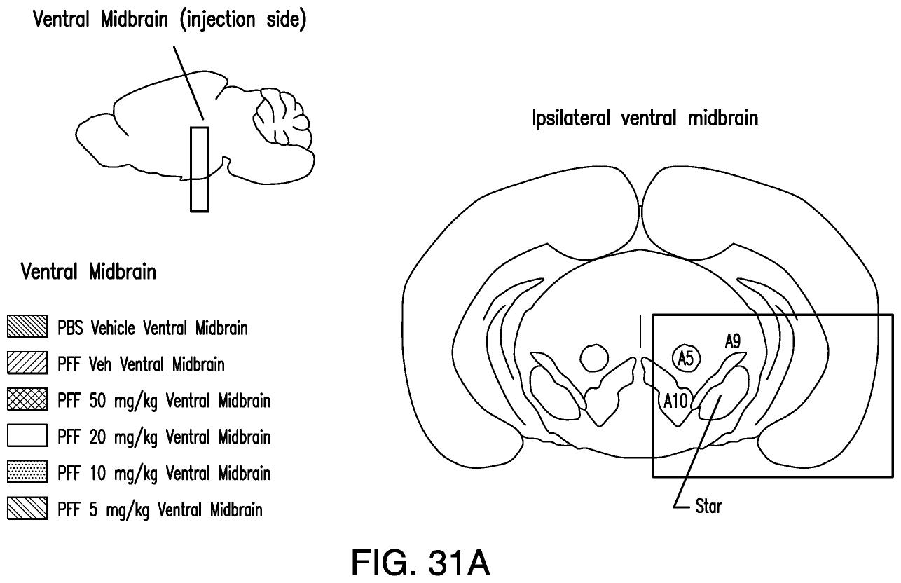

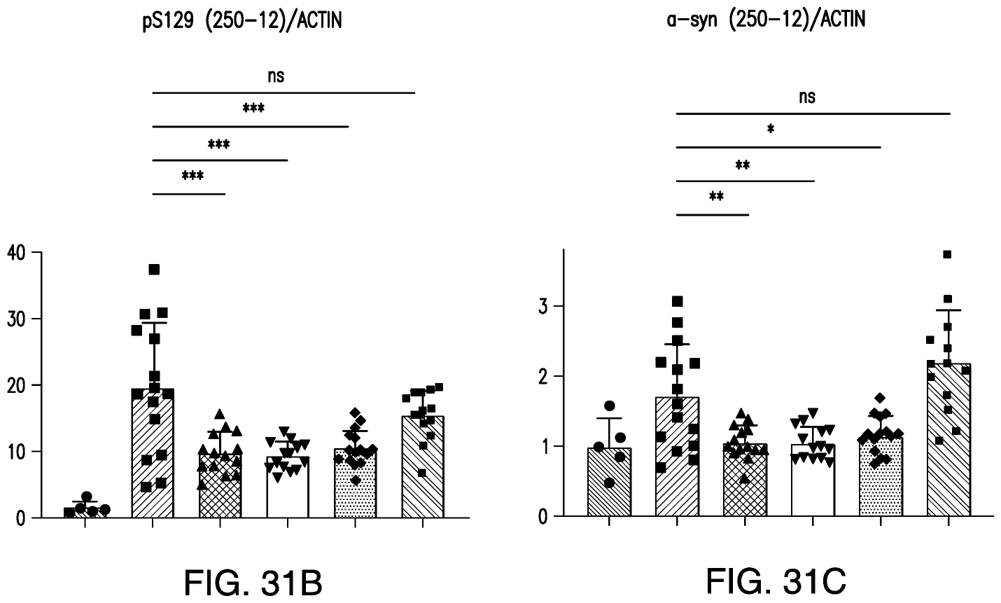

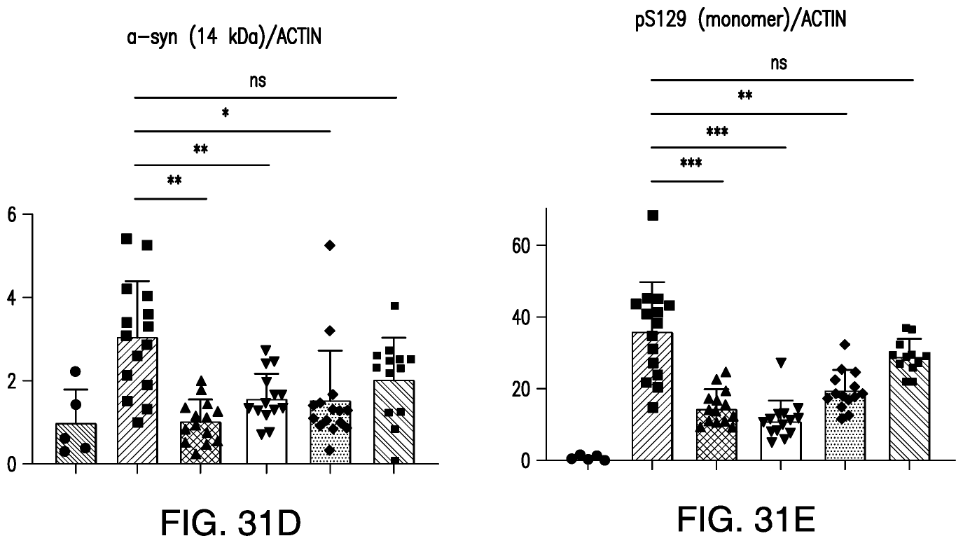

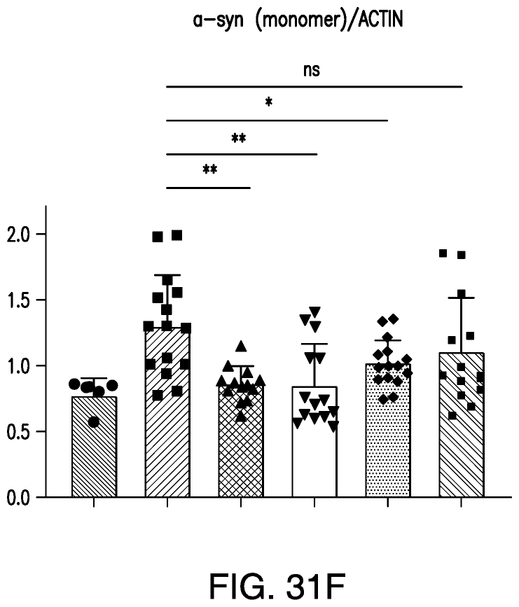

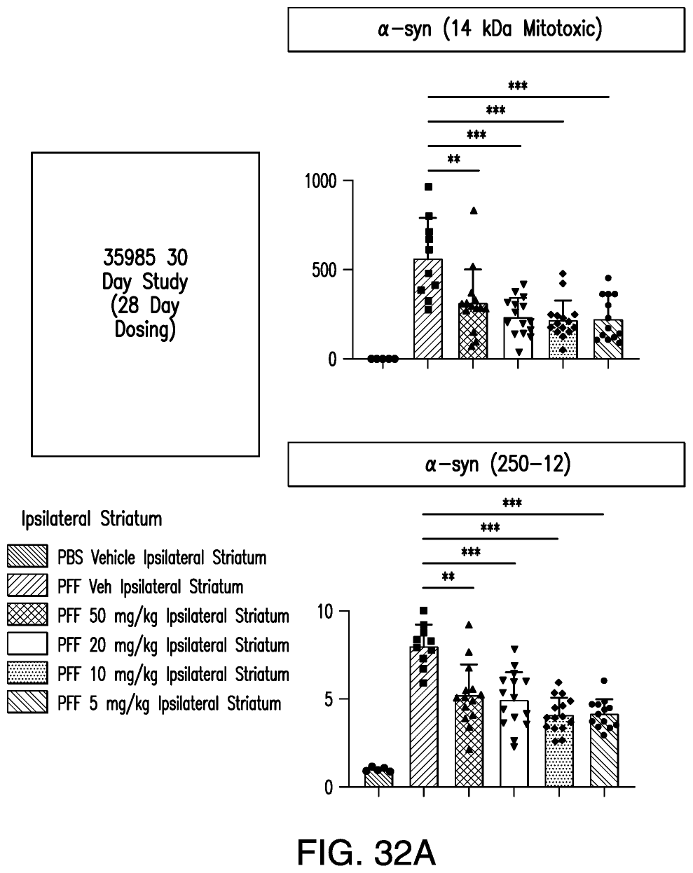

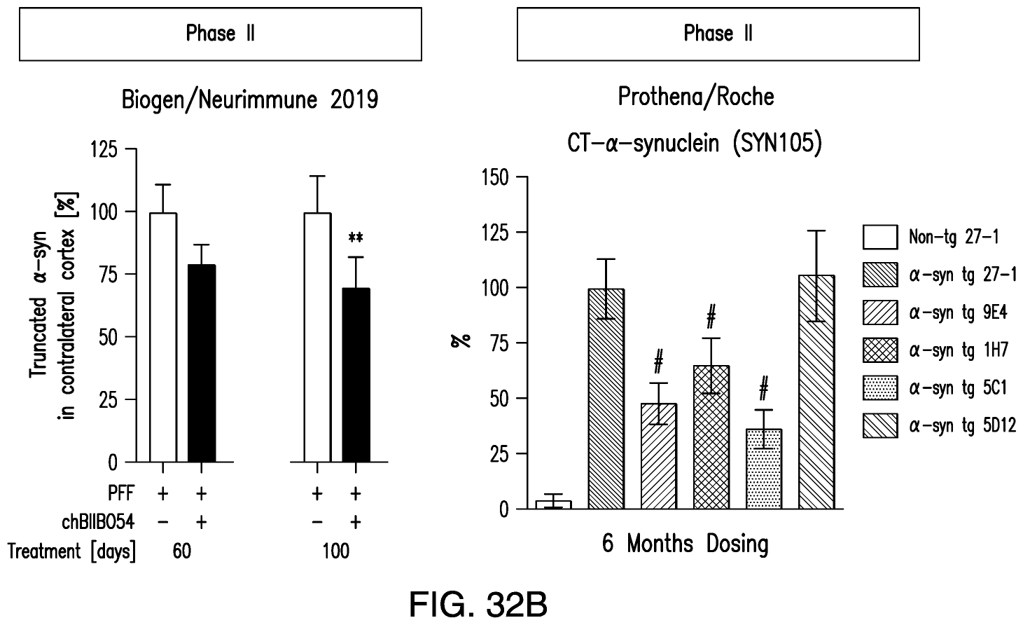

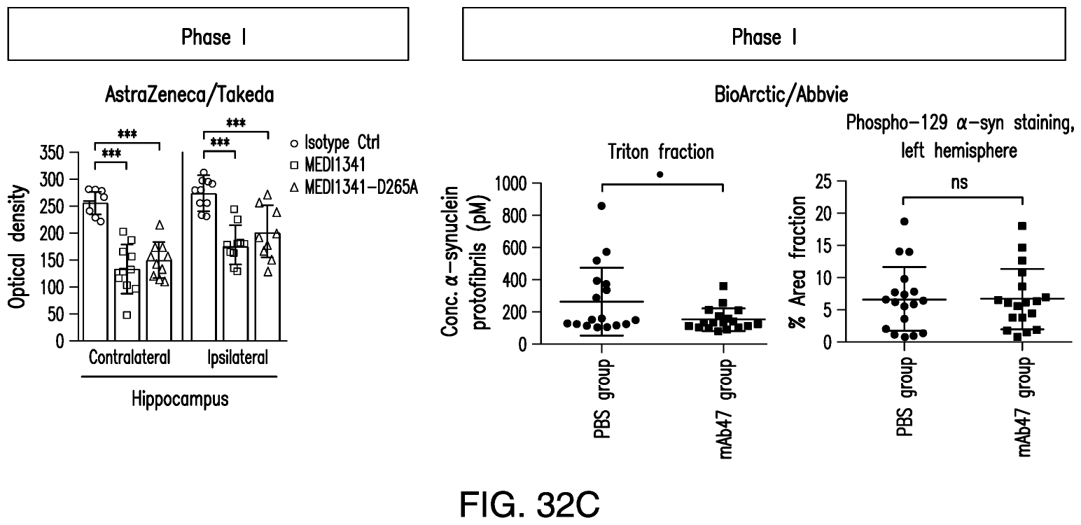

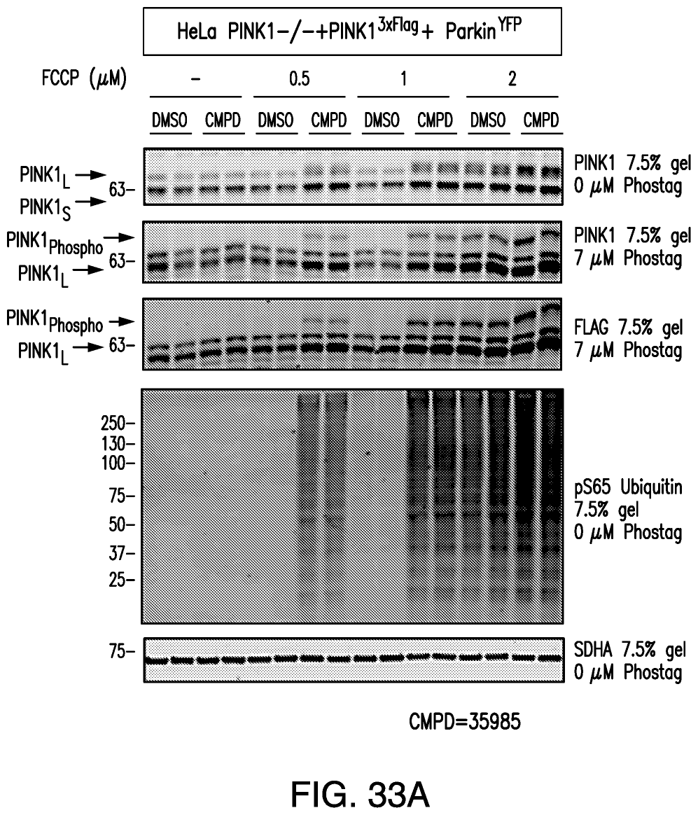

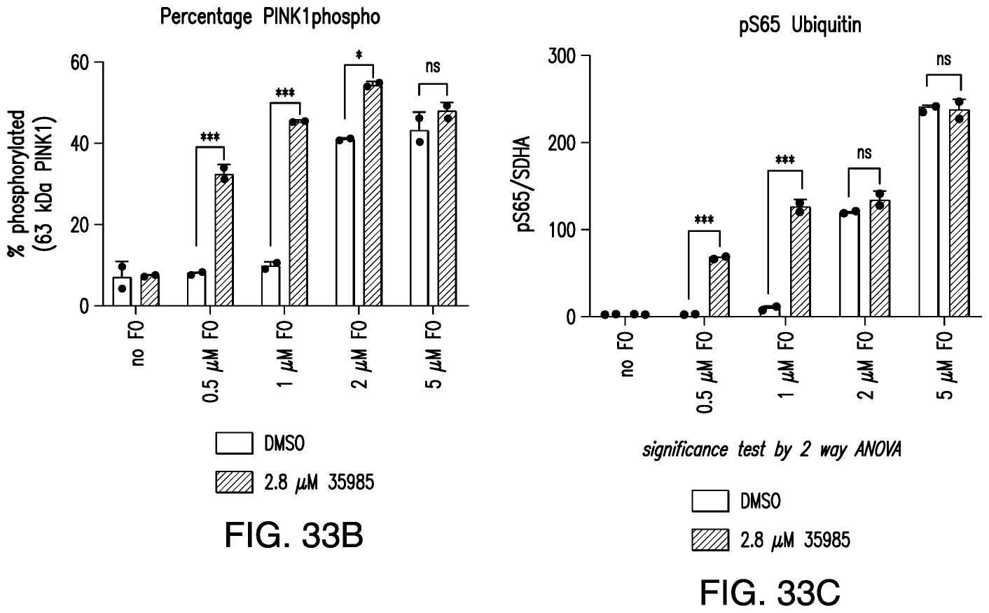

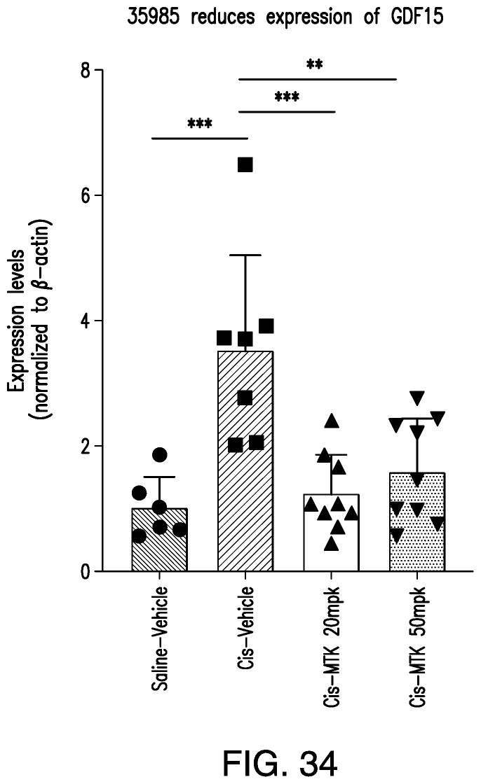

Referring to FIG. 23 and Table 7 below, in vivo dosing of EP-0035985 demonstrates good bioavailability.

TABLE 7

| | Cmax | | AUC (obs) | AUC (inf) | |
|---|---|---|---|---|---|
| Cohort | (μM) | T_{1/2} (hr) | (hr*μM) | (hr*μM) | F % |
| IV (1 mg/kg) | 1.45 | 1.4 | 1.4 | 1.5 | |
| PO (10 mg/kg) | 1.08 | 2.0 | 2.4 | 2.6 | 17.4% |
| PO (50 mg/kg) | 9.80 | 3.9 | 27.4 | 35.5 | 37.9% |

### 36. In Vivo Summary of EP-0035985

Referring to Tables 8A-C below, an in vivo summary of EP-0035985 IV at 1 mg/kg (8A), PO 10 mg/kg (8B), and a tissue distribution, mouse (8C) is shown below.

TABLE 8A

| | | | AUCₗₐₛₜ | AUC_{lnf} | | | | | |
|---|---|---|---|---|---|---|---|---|---|
| | T_{1/2} | C₀ | (hr*ng/ | (hr*ng/ | AUC_{Extr} | V_{z} | Vₛₛ | CL | MRT_{lnf} |
| species | (hr) | (ng/mL) | mL) | mL) | (%) | (L/Kg) | (L/Kg) | (mL/min/kg) | (hr) |
| Mouse | 1.4 | 1569.0 | 481.3 | 486.9 | 1.1 | 4.0 | 1.5 | 34.3 | 0.7 |
| rat | 3.6 | 1667.7 | 837.5 | 837.5 | 2.5 | 6.0 | 2.4 | 19.5 | 2.0 |

TABLE 8B

| | | | AUCₗₐₛₜ | AUC_{lnf} | | | | | |
|---|---|---|---|---|---|---|---|---|---|
| | T_{1/2} | C₀ | (hr*ng/ | (hr*ng/ | AUC_{Extr} | V_{z} | Vₛₛ | CL | MRT_{lnf} |
| species | (hr) | (ng/mL) | mL) | mL) | (%) | (L/Kg) | (L/Kg) | (mL/min/kg) | (hr) |
| Mouse | 2.0 | 0.5 | 357.3 | 803.3 | 848.1 | 5.4 | 2.5 | 84.8 | 17.4 |
| rat | 3.7 | 2.6 | 692.4 | 7348.4 | 7562.3 | 2.2 | 7.9 | 756.2 | 88.1 |

TABLE 8C

| | Brain | Brain | Brain | Plasma | Plasma | Plasma | Kidney | Kidney | Kidney |
|---|---|---|---|---|---|---|---|---|---|
| | 0.5 h | 1 h | 2 h | 0.5 h | 1 h | 2 h | 0.5 | 1 | 2 |
| Dose | (ng/g) | (ng/g) | (ng/g) | (ng/g) | (ng/g) | (ng/g) | (ng/g) | (ng/g) | (ng/g) |
| 10 | 526 | 737 | 435 | 372 | 276 | 146 | 1050 | 1290 | 893 |
| mg/kg | | | | | | | | | |
| 50 | 3342 | 6367 | 3985 | 1903 | 2080 | 1423 | 6433 | 11517 | 7483 |
| mg/kg | | | | | | | | | |

### 37. Cisplatin Mouse Model of Mitochondrial Dysfunction

Cisplatin is a chemotherapeutic agent reported in the literature to induce mitochondrial damage (see: Yang, Z., et al. “Cisplatin Preferentially Binds Mitochondrial DNA and Voltage-Dependent Anion Channel Protein in the Mitochondrial Membrane of Head and Neck Squamous Cell Carcinoma: Possible Role in Apoptosis.” Clinical Cancer Research 12, no. 19 (2006), 5817-5825). Growth and Differentiation Factor 15 (GDF15) is an established biomarker of mitochondrial disease and certain neurodegenerative diseases (see: Montero, R, et al. “GDF-15 Is Elevated in Children with Mitochondrial Diseases and Is Induced by Mitochondrial Dysfunction.” PLOS ONE 11, no. 2 (2016); Nohara, S, et al. “GDF-15, a mitochondrial disease biomarker, is associated with the severity of multiple sclerosis”, Journal of Neurological Sciences, Vol 405 (2019)). Briefly, mice approximately 10 weeks in age were challenged with a single intraperitoneal dose of cisplatin (10 mg/kg), then treated daily via oral gavage with either compound 35985 at various doses or vehicle control. On day 7, the animals were sacrificed. Kidneys were removed and homogenized. Following published methods, qPCR was performed on the kidney samples to determine expression levels of GDF15.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A compound having a structure represented by a formula: 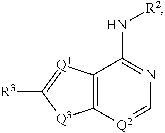
wherein Q¹ is N or CH and R³ is a 3- to 6-membered cycloalkyl, a C1-C6 haloalkyl, C1-C6 haloalkoxy, or C1-C6 halohydroxyalkyl;
or wherein Q¹ is CR¹ and R³ is hydrogen;
R¹ is C1-C6 haloalkyl, C1-C6 haloalkoxy, C1-C6 halohydroxyalkyl, or a structure represented by a formula: 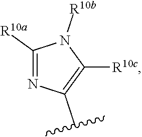
wherein each of R^{10a}, R^{10b}, and R^{10c}, when present, is independently selected from hydrogen and C1-C4 alkyl;
wherein Q² is CH or N;
wherein Q³ is CH₂ or NH;
wherein R² is C1-C6 alkyl, —CR^{11a}R^{11b}Cy¹, or Cy¹;
wherein each of R^{11a} and R^{11b}, when present, is independently selected from hydrogen, C1-C5 alkyl, and C1-C4 hydroxyalkyl;
or wherein each of R^{11a} and R^{11b} together comprise a 3-membered cycloalkyl;
wherein Cy¹, when present, is selected from a 3- to 10-membered carbocycle, a 3- to 10-membered heterocycle, a 6- to 10-membered aryl, and a 6- to 10-membered heteroaryl, and is substituted with 0, 1, 2, 3, or 4 groups independently selected from halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino,
provided that when R¹ is C1-C6 haloalkyl and R² is Cy¹, then Cy¹ is not a 6-membered carbocycle or a 9-membered heteroaryl, and
provided that when R² is —CR^{11a}R^{11b}Cy¹ or Cy¹, one or both of R^{11a} and R^{11b}, when present, is hydrogen, and Cy¹ is a 6-membered aryl or furanyl, then Q¹ is CH and R³ is not a C1-C6 haloalkyl,
or a pharmaceutically acceptable salt thereof.

2. The compound of claim 1, wherein Q¹ is N and R³ is a 3- to 6-membered cycloalkyl.

3. The compound of claim 1, wherein Q¹ is N and R³ is a C1-C6 haloalkyl, C1-C6 haloalkoxy, or C1-C6 halohydroxyalkyl.

4. (canceled)

5. The compound of claim 1, wherein Q¹ is CH and R³ is a C1-C6 haloalkyl, C1-C6 haloalkoxy, or C1-C6 halohydroxyalkyl.

6. (canceled)

7. The compound of claim 1, wherein Q¹ is CR¹ and R³ is hydrogen.

8. The compound of claim 1, wherein Q² is N.

9. The compound of claim 1, wherein Q³ is NH.

10. The compound of claim 1, wherein R² is C1-C6 alkyl.

11. (canceled)

12. The compound of claim 1, wherein R² is —CR^{11a}R^{11b}Cy¹ or Cy¹.

13-21. (canceled)

22. The compound of claim 1, wherein Cy¹, when present, is a structure represented by a formula selected from: 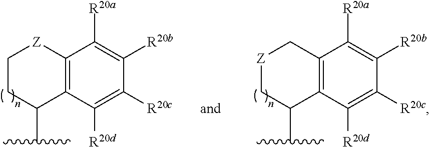
wherein Z is O, CH₂, or NR³⁰;
wherein R³⁰, when present, is selected from —C(O)(C1-C4 alkyl), C1-C4 alkyl, and C2-C4 alkenyl;
wherein n is 0 or 1; and
wherein each of R^{20a}, R^{20b}, R^{20c}, and R^{20d} is independently selected from hydrogen, halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino.

23-24. (canceled)

25. The compound of claim 1, wherein the compound has a structure represented by a formula selected from:

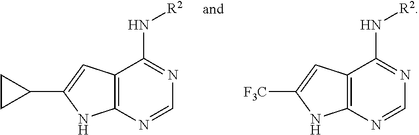

26. (canceled)

27. The compound of claim 1, wherein the compound has a structure represented by a formula: 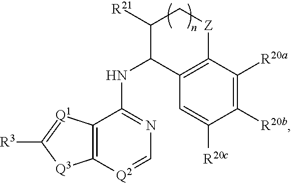
wherein Z is O, CH₂, or NR³⁰;
wherein R³⁰, when present, is selected from —C(O)(C1-C4 alkyl), C1-C4 alkyl, and C2-C4 alkenyl;
wherein n is 0 or 1;
wherein each of R^{20a}, R^{20b}, R^{20c}, and R^{20d} is independently selected from hydrogen, halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino; and
wherein R²¹ is selected from hydrogen, halogen, —CN, —NH₂, —OH, —NO₂, —C(O)(C1-C4 alkyl), C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, and (C1-C4)(C1-C4) dialkylamino.

28-29. (canceled)

30. The compound of claim 1, wherein the compound has a structure selected from:

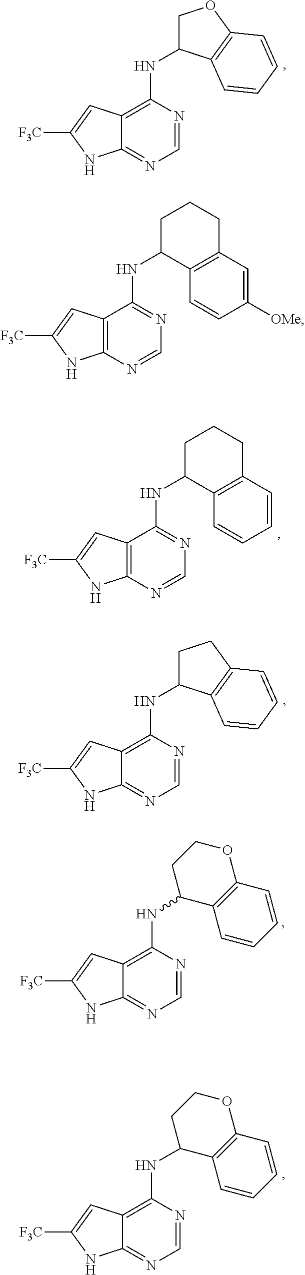 [SMILES: FC(F)(F)c1cc2c(NC3COc4ccccc43)ncnc2[nH]1 ; COc1ccc2c(c1)CCCC2Nc1ncnc2[nH]c(C(F)(F)F)cc12 ; FC(F)(F)c1cc2c(NC3CCCc4ccccc43)ncnc2[nH]1 ; FC(F)(F)c1cc2c(NC3CCc4ccccc43)ncnc2[nH]1 ; FC(F)(F)c1cc2c(NC3CCOc4ccccc43)ncnc2[nH]1 ; FC(F)(F)c1cc2c(NC3CCOc4ccccc43)ncnc2[nH]1]

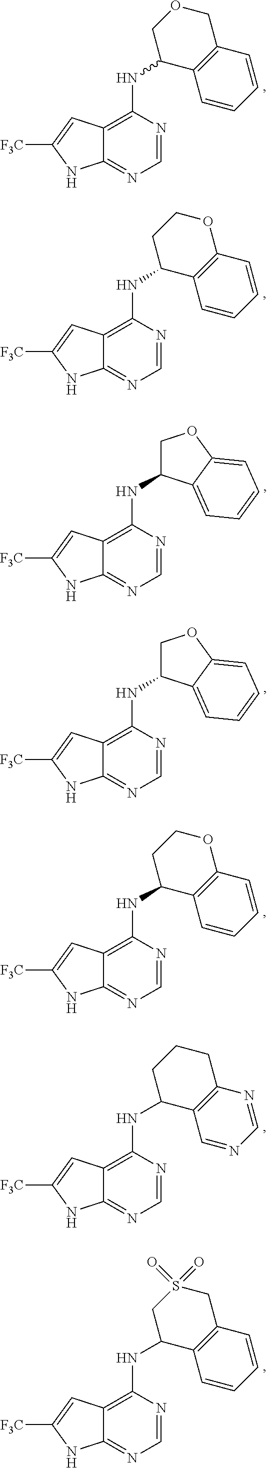

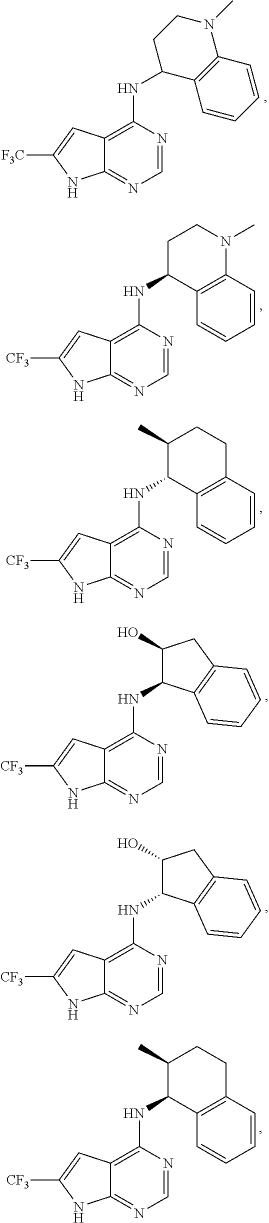

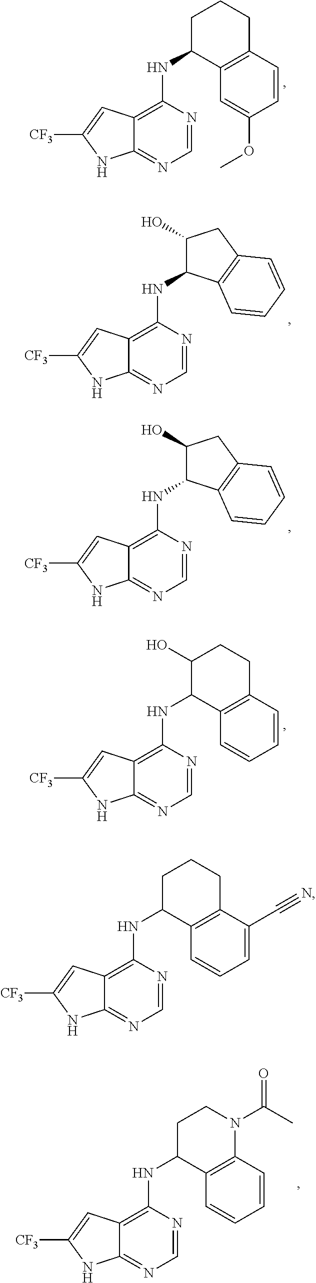

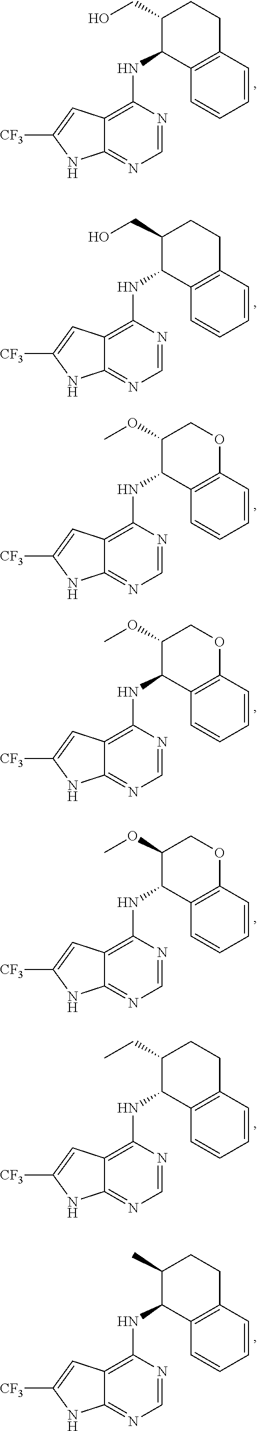

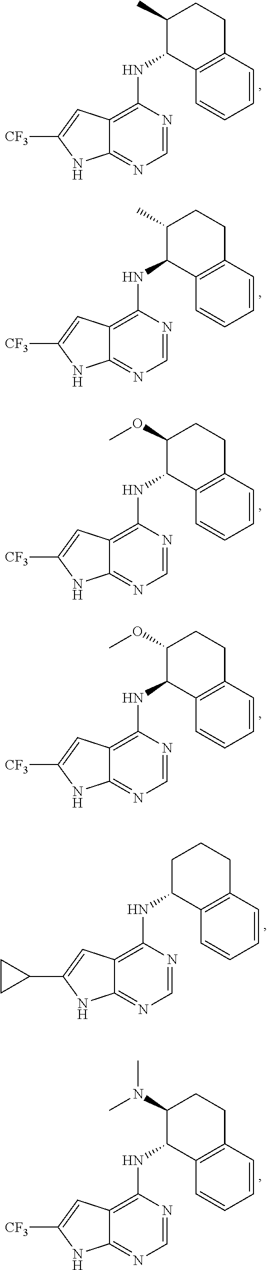

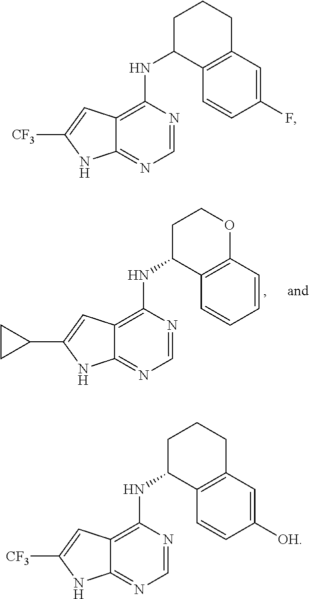 [SMILES: Fc1ccc2c(c1)CCCC2Nc1ncnc2[nH]c(C(F)(F)F)cc12 ; c1ccc2c(c1)OCC[C@H]2Nc1ncnc2[nH]c(C3CC3)cc12 ; Oc1ccc2c(c1)CCC[C@H]2Nc1ncnc2[nH]c(C(F)(F)F)cc12]

31. (canceled)

32. The compound of claim 1, wherein the compound has a structure selected from: 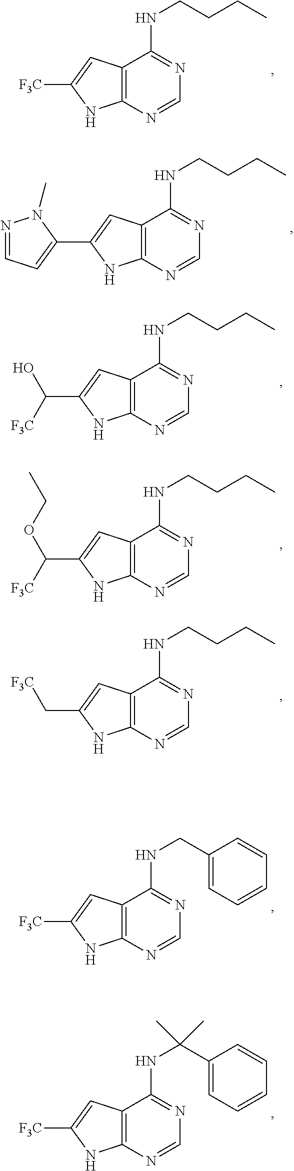 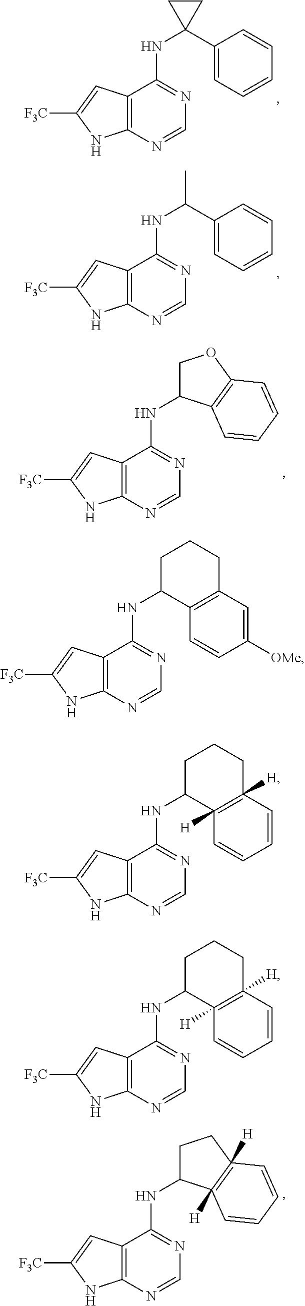 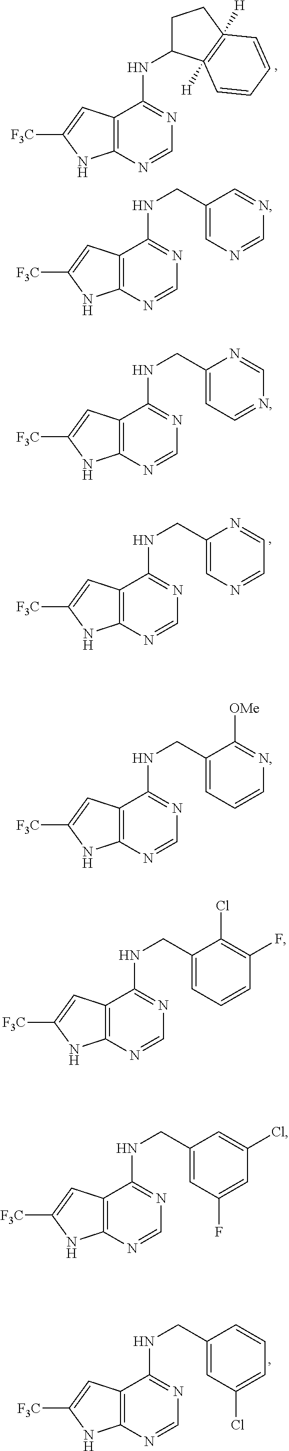 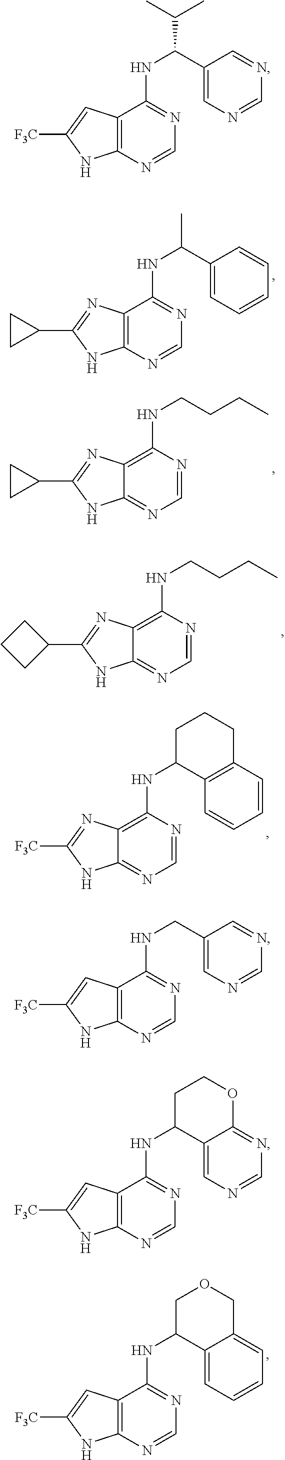 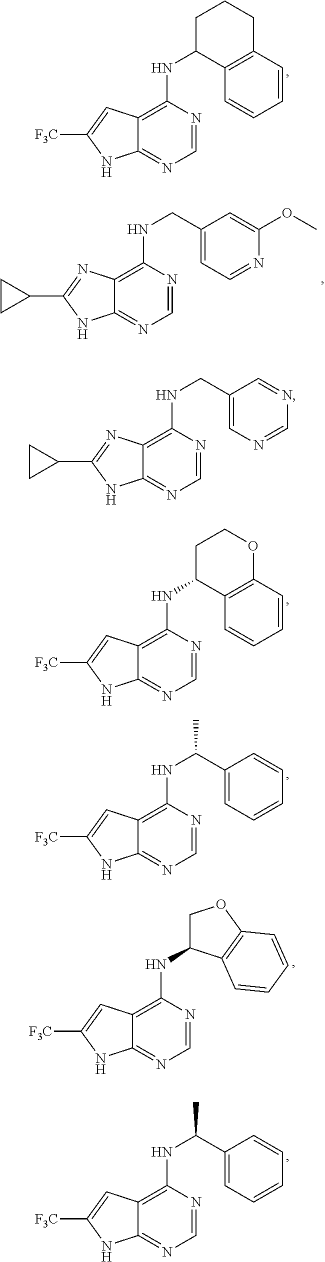 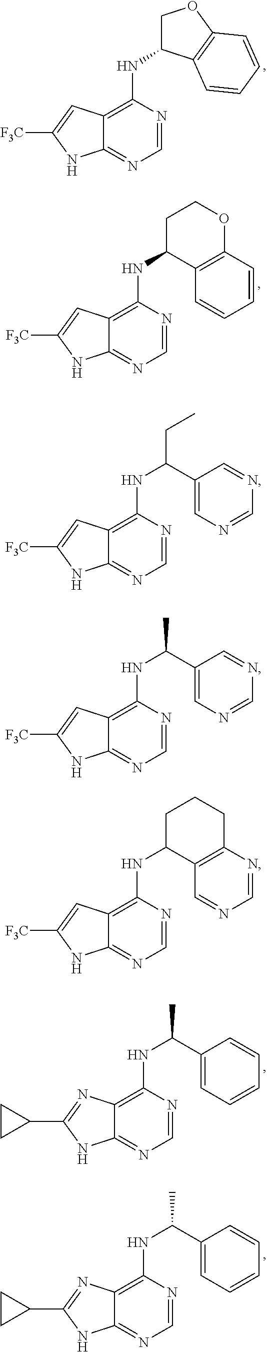 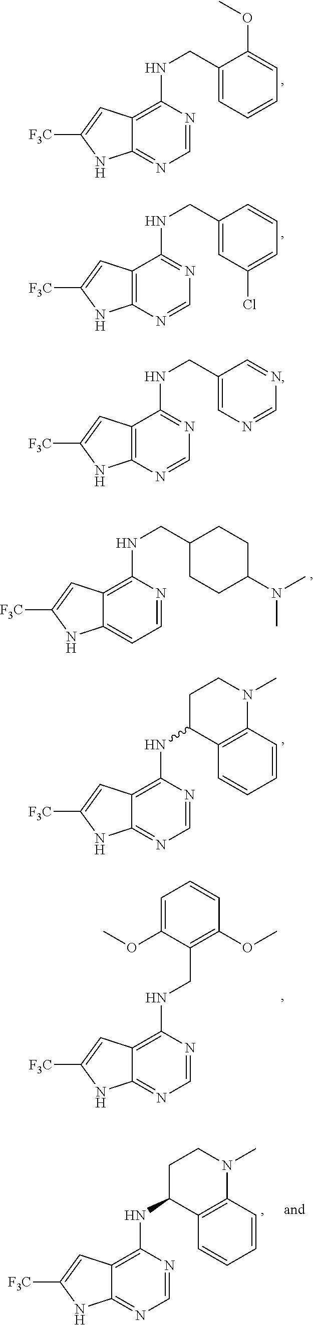 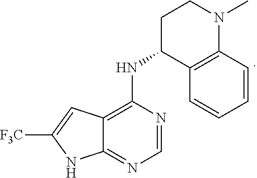 [SMILES: CN1CC[C@@H](Nc2ncnc3[nH]c(C(F)(F)F)cc23)c2ccccc21]
or a pharmaceutically acceptable salt thereof.

33. The compound of claim 1, wherein the compound has a structure selected from. 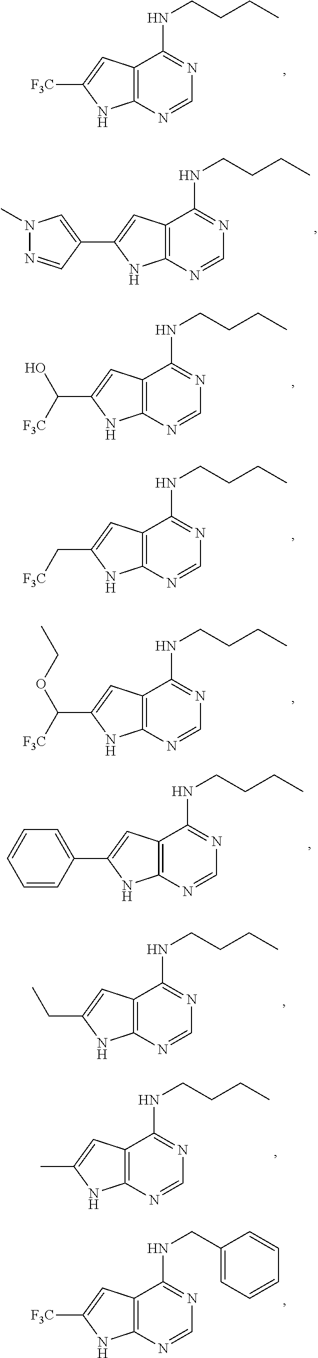 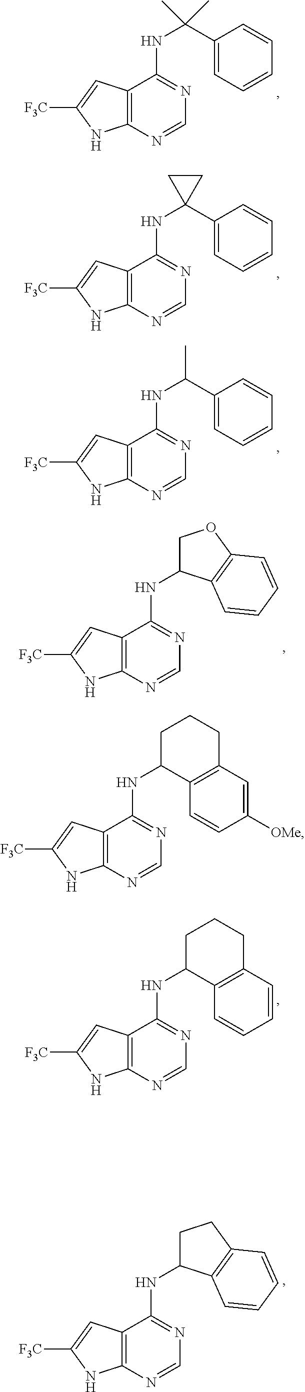 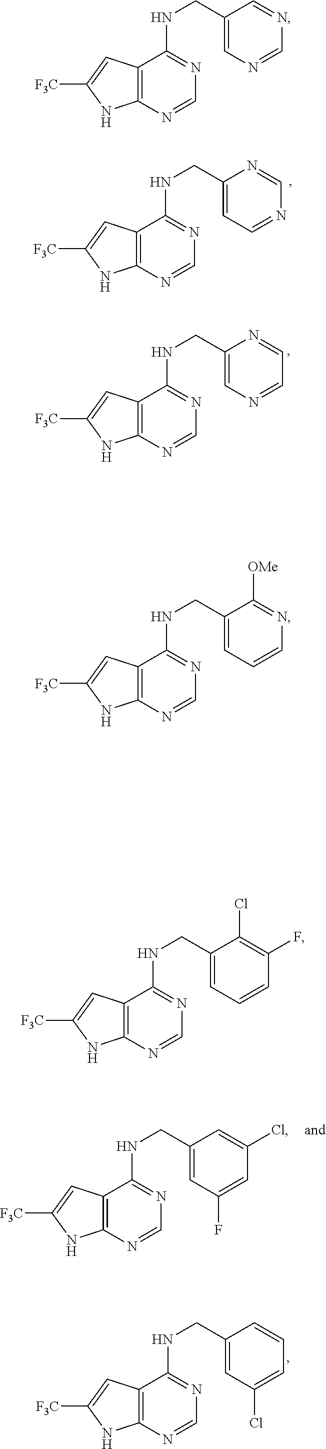
or a pharmaceutically acceptable salt thereof.

34. The compound of claim 1, wherein the compound has a structure selected from: 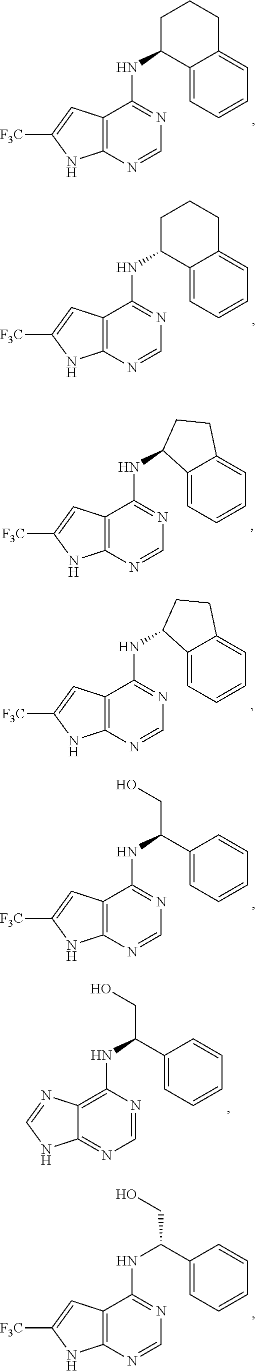 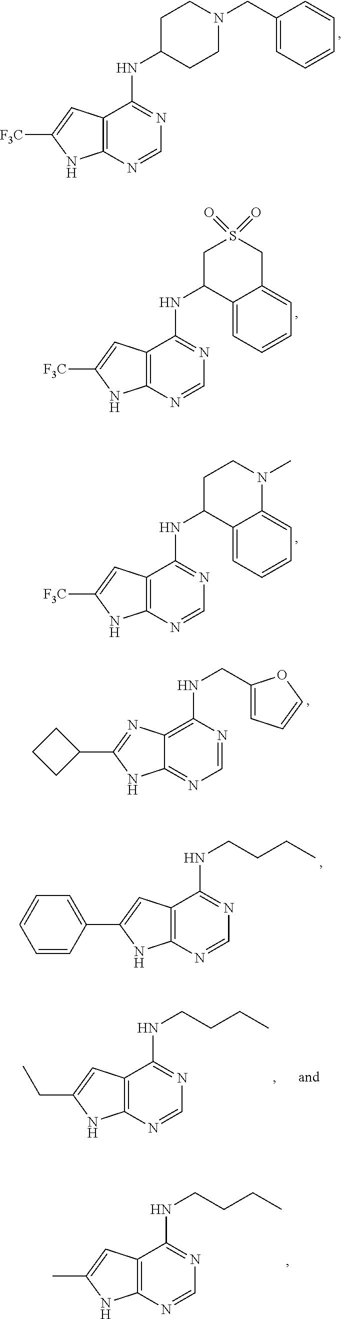
or a pharmaceutically acceptable salt thereof.

35-40. (canceled)

41. A pharmaceutical composition comprising a therapeutically effective amount of the compound of claim 1, and a pharmaceutically acceptable carrier.

42-43. (canceled)

44. A method of modulating PINK1 kinase activity in at least one cell, the method comprising contacting the cell with an effective amount of the compound of claim 1.

45. The method of claim 44, wherein the cell is mammalian.

46-48. (canceled)

49. A method of treating a disorder in a subject in need thereof, the method comprising administering to the subject in need thereof an effective amount of the compound of claim 1, wherein the disorder is a neurodegenerative disorder, a mitochondrial disorder, a fibrosis, or cardiomyopathy.

50-54. (canceled)

55. The method of claim 49, wherein the neurodegenerative disorder is Parkinson's disease, Huntington's disease, or amyotrophic lateral sclerosis.

56-71. (canceled)
